(19) United States
(12) Patent Application Publication
(10) Pub. No.: US 2005/0085531 A1
(43) Pub. Date: Apr. 21, 2005

(54) Thiophene-based compounds exhibiting ATP-utilizing enzyme inhibitory activity, and compositions, and uses thereof

(76) Inventors: Carl Nicholas Hodge, Los Gatos, CA (US); William P. Janzen, Chapel Hill, NC (US); Kevin Peter Williams, Chapel Hill, NC (US); Lynn A. Cheatham, Durham, NC (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/957,570

(22) Filed: Sep. 30, 2004

Related U.S. Application Data
(60) Provisional application No. 60/508,393, filed on Oct. 3, 2003.

(51) Int. Cl. A61K 31/381; C07D 49/02; C07D 333/38

(52) U.S. Cl. 514/444; 514/447; 549/59; 549/61; 549/65

(57) ABSTRACT

Thiophene-based compounds exhibiting ATP-utilizing enzyme inhibitory activity, methods of using compounds exhibiting ATP-utilizing enzyme inhibitory activity, and compositions comprising compounds exhibiting ATP-utilizing enzyme inhibitory activity, are disclosed.

This application claims priority benefit of U.S. Provisional Application No. 60/508,393 filed Oct. 3, 2003.

Enzymes are macromolecules, usually proteins, which function as biocatalysts by increasing the rate of a biochemical reaction. Generally, an enzyme is highly specific, both in the type of biochemical reaction catalyzed and for the type of substrate, or reactant.

ATP-utilizing enzymes catalyze the transfer of a phosphate group from an adenosine triphosphate (ATP) molecule to a biomolecule such as a protein or carbohydrate. Examples of ATP-utilizing enzymes include, but are not limited to, synthetases, ligases, synapsins, phosphatases, and kinases.

Protein kinases encompass a large family of functionally and structurally related enzymes that are responsible for the control of a wide variety of cellular processes including signal transduction, metabolism, transcription, cell cycle progression, cytoskeletal rearrangement and cell movement, apoptosis, and differentiation. In general, protein kinases control protein activity by catalyzing the addition of a negatively charged phosphate group from a phosphate-containing molecule such as cyclic adenosine monophosphate (cAMP), adenosine diphosphate (ADP), and ATP, to other proteins. Protein phosphorylation in turn can modulate or regulate the functioning of a target protein. Protein phosphorylation is known to play a role in intercellular communication during development, in physiological responses and in homeostasis, and in the functioning of the nervous and immune systems.

The unregulated phosphorylation of proteins is known to be a cause of, or associated with the etiology of major diseases, such as Alzheimer's disease, stroke, diabetes, obesity, inflammation, cancer, and rheumatoid arthritis. Deregulated protein kinase activity and over-expression of protein kinases has been implicated in the pathophysiology of a number of important human disorders. Furthermore, genetic mutations in protein kinases are implicated in a number of disorders and many toxins and pathogens exert their effects by altering the phosphorylation of intracellular proteins.

ATP-utilizing enzymes, such as protein kinases, therefore, represent a broad class of pharmacological targets of interest for the treatment of human disease. The identification and development of compounds that selectively inhibit the functioning of ATP-utilizing enzymes is therefore of considerable interest.

Certain aspects of the present disclosure are directed to compounds of Formula (I): 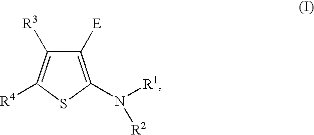
a stereoisomer thereof, a pharmaceutically acceptable salt thereof, a hydrate thereof, or a solvate of any of the foregoing, wherein:
E is chosen from —CN, halogen, —NO₂, and —C(═X)YR⁵; wherein
X is chosen from O, and S;
Y is chosen from —N(R¹⁰)—, O, S, and a direct bond; wherein
R¹⁰ is chosen H, alkyl, and substituted alkyl; and
R⁵ is chosen from H, alkyl, substituted alkyl, arylalkyl, substituted arylalkyl, cycloalkyl, substituted cycloalkyl, heterocycloalkyl, substituted heterocycloalkyl, heteroalkyl, substituted heteroalkyl, heteroarylalkyl, substituted heteroarylalkyl, and when Y is —N(R¹⁰)—, or a direct bond, then R⁵ is additionally chosen from aryl, substituted aryl, heteroaryl, substituted heteroaryl, —N(R⁷)₂, and —OR⁹; wherein
each R⁷ is independently chosen from alkyl, substituted alkyl, aryl, substituted aryl, and H; and
R⁹ is chosen from H, alkyl, and substituted alkyl;
or R⁵ and R¹⁰ together with the atoms to which R⁵ and R¹⁰ are attached form a cycloalkyl, substituted cycloalkyl, heterocycloalkyl, or substituted heterocycloalkyl ring;
R¹ is chosen from H, alkyl, substituted alkyl, cycloalkyl, substituted cycloalkyl, heteroalkyl, and substituted heteroalkyl;
R² is chosen from H, —CHO, alkyl, substituted alkyl, aryl, substituted aryl, cycloalkyl, substituted cycloalkyl, heteroaryl, substituted heteroaryl, heterocycloalkyl, substituted heterocycloalkyl, heteroarylalkyl, substituted heteroarylalkyl, heterocycloalkylalkyl, substituted heterocycloalkylalkyl, alkylsulfonyl, substituted alkylsulfonyl, heteroalkylsulfonyl, substituted heteroalkylsulfonyl, and -ZR⁶, wherein
Z is chosen from carbonyl, —C(O)O—, aminosulfonyl, aminothiocarbonyl, —C(═O)NR¹¹—, sulfonyl, and thiocarbonyl; wherein
R¹¹ is chosen from alkyl, substituted alkyl, cycloalkyl, substituted cycloalkyl, and H; and
R⁶ is chosen from H, —COOH, alkyl, substituted alkyl, aryl, substituted aryl, cycloalkyl, substituted cycloalkyl, heterocycloalkyl, substituted heterocycloalkyl, heteroalkyl, substituted heteroalkyl, heteroaryl, substituted heteroaryl, cycloalkylalkyl, substituted cycloalkylalkyl, heterocycloalkylalkyl, substituted heterocycloalkylalkyl, arylalkyl, substituted arylalkyl, heteroarylalkyl, substituted heteroarylalkyl, bicycloalkyl, substituted bicycloalkyl, bicycloheteroalkyl, and substituted bicycloheteroalkyl;
or R¹ and R², together with the atoms to which R¹ and R² are attached, form a heterocycloalkyl, or substituted heterocycloalkyl ring;
R³ is chosen from H, halogen, —NH₂, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, dialkylamino, and substituted dialkylamino; and
R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
or R³ and R⁴ together with the atoms to which R³ and R⁴ are attached form a cycloalkyl, substituted cycloalkyl, heterocycloalkyl, substituted heterocycloalkyl, bicycloalkyl, substituted bicycloalkyl, bicycloheteroalkyl, or bicycloheteroalkyl ring;
with the provisos that
when E is —CO₂R⁵, then R³ is not H, 2-aminopyrimidine, substituted 2-aminopyrimidine 2-aminopyridine, substituted 2-aminopyridine, aminotriazine, or substituted aminotriazine; and R⁴ is not 2-aminopyrimidine, substituted 2-aminopyrimidine 2-aminopyridine, substituted 2-aminopyridine, aminotriazine, or substituted aminotriazine;
when E is —CN, then R³is not H, 2-aminopyrimidine, substituted 2-aminopyrimidine 2-aminopyridine, substituted 2-aminopyridine, aminotriazine, or substituted aminotriazine; and R⁴ is not H, 2-aminopyrimidine, substituted 2-aminopyrimidine 2-aminopyridine, substituted 2-aminopyridine, aminotriazine, or substituted aminotriazine;
when E is —CN, and R² is —C(═X)NH₂, where X is O or S, then R³ is not unsubstituted phenyl, or a 5 to 7 member heteroaromatic ring containing 1 to 3 heteroatoms chosen from O, N or S; and R⁴is not unsubstituted phenyl, or a 5 to 7 member heteroaromatic ring containing 1 to 3 heteroatoms chosen from O, N or S;

when E is —C(═O)NR⁵R¹⁰, and R³ is H, and R² is C(═O)NR¹²R¹¹, and R¹¹ is H, then R¹² is not alkyl or substituted alkyl; and
when E is —C(═O)NR⁵R¹⁰, and R¹ is H, and R⁵ is H, then R¹⁰ is not H
and wherein the compound of Formula (I), a stereoisomer thereof, a pharmaceutically acceptable salt thereof, a hydrate thereof, or a solvate of any of the foregoing, exhibits ATP-utilizing enzyme inhibitory activity.

Certain aspects of the present disclosure are directed to compounds of Formula (II): 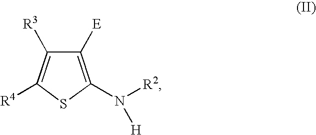
a stereoisomer thereof, a pharmaceutically acceptable salt thereof, a hydrate thereof, or a solvate of any of the foregoing, wherein:
E is chosen from —CN, halogen, —NO₂, and —C(═X)YR⁵; wherein
X is chosen from O, and S;
Y is chosen from —N(R¹⁰)—, O, S, and a direct bond; wherein
R¹⁰ is chosen H, alkyl, and substituted alkyl; and
R⁵ is chosen from H, alkyl, substituted alkyl, arylalkyl, substituted arylalkyl, cycloalkyl, substituted cycloalkyl, heterocycloalkyl, substituted heterocycloalkyl, heteroalkyl, substituted heteroalkyl, heteroarylalkyl, substituted heteroarylalkyl, and when Y is —N(R¹⁰)—, or a direct bond, then R⁵ is additionally chosen from aryl, substituted aryl, heteroaryl, substituted heteroaryl, —N(R⁷)₂, and —OR⁹; wherein
each R⁷ is independently chosen from alkyl, substituted alkyl, aryl, substituted aryl, and H; and
R⁹ is chosen from H, alkyl, and substituted alkyl;
or R⁵ and R¹⁰ together with the atoms to which R⁵ and R¹⁰ form a cycloalkyl, substituted cycloalkyl, heterocycloalkyl, or substituted heterocycloalkyl ring;
R² is chosen from H, —CHO, alkyl, substituted alkyl, aryl, substituted aryl, cycloalkyl, substituted cycloalkyl, heteroaryl, substituted heteroaryl, heterocycloalkyl, substituted heterocycloalkyl, heteroarylalkyl, substituted heteroarylalkyl, heterocycloalkylalkyl, substituted heterocycloalkylalkyl, alkylsulfonyl, substituted alkylsulfonyl, heteroalkylsulfonyl, substituted heteroalkylsulfonyl, and -ZR⁶, wherein
Z is chosen from carbonyl, —C(O)O—, aminosulfonyl, aminothiocarbonyl, —C(═O)NR¹¹—, sulfonyl, and thiocarbonyl; wherein
R¹¹ is chosen from alkyl, substituted alkyl, cycloalkyl, substituted cycloalkyl, and H; and
R⁶ is chosen from H, —COOH, alkyl, substituted alkyl, aryl, substituted aryl, cycloalkyl, substituted cycloalkyl, heterocycloalkyl, substituted heterocycloalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, arylalkyl, substituted arylalkyl, heteroarylalkyl, substituted heteroarylalkyl, bicycloalkyl, substituted bicycloalkyl, bicycloheteroalkyl, and substituted bicycloheteroalkyl;
or R¹ and R², together with the atoms to which R¹ and R² are attached, form a heterocycloalkyl, or substituted heterocycloalkyl ring;
R³ is chosen from H, halogen, —NH₂, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, dialkylamino, and substituted dialkylamino; and
R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
or R³ and R⁴ together with the atoms to which R³ and R⁴ are attached form a cycloalkyl, substituted cycloalkyl, heterocycloalkyl, substituted heterocycloalkyl, bicycloalkyl, substituted bicycloalkyl, bicycloheteroalkyl, or substituted bicycloheteroalkyl ring;
with the provisos that
when E is —CO₂R⁵, then R³ is not H, 2-aminopyrimidine, substituted 2-aminopyrimidine, 2-aminopyridine, substituted 2-aminopyridine, aminotriazine or substituted aminotriazine; and R⁴ is not 2-aminopyrimidine, substituted 2-aminopyrimidine, 2-aminopyridine, substituted 2-aminopyridine, aminotriazine or substituted aminotriazine;
when E is —CN, then R³ is not 2-aminopyrimidine, substituted 2-aminopyrimidine, 2-aminopyridine, substituted 2-aminopyridine, aminotriazine or substituted aminotriazine; and R⁴ is not 2-aminopyrimidine, substituted 2-aminopyrimidine, 2-aminopyridine, substituted 2-aminopyridine, aminotriazine or substituted aminotriazine;
when E is —CN, and R² is —C(═X)NH₂, then R³ is not unsubstituted phenyl or a 5 to 7 membered heteroaromatic ring containing 1 to 3 heteroatoms chosen from O, N or S; and R⁴ is not unsubstituted phenyl or a 5 to 7 membered heteroaromatic ring containing 1 to 3 heteroatoms chosen from O, N or S;
when E is —C(═O)NR⁵R¹⁰, and R³ is H, and R² is —C(═O)NR¹²R¹¹, and R¹¹ is H, then R¹² is not alkyl, or substituted alkyl; and
when E is —C(═O)NR⁵R¹⁰, and R¹ is H, and R⁵ is H, then R¹⁰ is not H;
and wherein the compound of Formula (II), a stereoisomer thereof, a pharmaceutically acceptable salt thereof, a hydrate thereof, or a solvate of any of the foregoing, exhibits ATP-utilizing enzyme inhibitory activity.

Certain aspects of the present disclosure are directed to compounds of Formula (III): 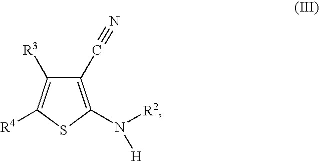
a stereoisomer thereof, a pharmaceutically acceptable salt thereof, a hydrate thereof, or a solvate of any of the foregoing, wherein:
R² is chosen from H, and -ZR⁶, wherein
Z is carbonyl; and
R⁶ is chosen from H, alkyl, substituted alkyl, heteroalkyl, substituted heteroalkyl, aryl, substituted aryl, heteroaryl, substituted heteroaryl, heterocycloalkyl, substituted heterocycloalkyl, heterocycloalkylalkyl, substituted heterocycloalkylalkyl, heteroarylalkyl, and substituted heteroarylalkyl;
R³ is chosen from H, —NH₂, alkyl, and substituted alkyl; and
R⁴ is chosen from H, halogen, alkyl, substituted alkyl, heteroalkyl, substituted heteroalkyl, arylalkyl, substituted arylalkyl, heterocycloalkylalkyl, substituted heterocycloalkylalkyl;
or R³ and R⁴ together with the atoms to which R³ and R⁴ are attached form a cycloalkyl, substituted cycloalkyl, heterocycloalkyl, or substituted heterocycloalkyl ring;
with the provisos that
R³ is not H, 2-aminopyrimidine, substituted 2-aminopyrimidine, 2-aminopyridine, substituted 2-aminopyridine, aminotriazine, or substituted aminotriazine;
R⁴ is not H, 2-aminopyrimidine, substituted 2-aminopyrimidine, 2-aminopyridine, substituted 2-aminopyridine, aminotriazine, or substituted aminotriazine; and
when R² is —C(═X)NH₂, where X is O or S, then R³ is not unsubstituted phenyl, or a 5 to 7 membered heteroaromatic ring containing 1 to 3 heteroatoms chosen from O, N or S; and R⁴ is not unsubstituted phenyl, or a 5 to 7 membered heteroaromatic ring containing 1 to 3 heteroatoms chosen from O, N or S;
and wherein the compound of Formula (III), a stereoisomer thereof, a pharmaceutically acceptable salt thereof, a hydrate thereof, or a solvate of any of the foregoing, exhibits ATP-utilizing enzyme inhibitory activity.

Certain aspects of the present disclosure are directed to compounds of Formula (IV): 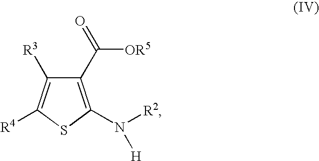
a stereoisomer thereof, a pharmaceutically acceptable salt thereof, a hydrate thereof, or a solvate of any of the foregoing, wherein:
R² is chosen from H, —CHO, heteroarylalkyl, substituted heteroarylalkyl, heterocycloalkylalkyl, substituted heterocycloalkylalkyl, alkylsulfonyl, substituted alkylsulfonyl, and -ZR⁶, wherein
Z is carbonyl; and
R⁶ is chosen from H, —COOH, alkyl, substituted alkyl, aryl, and substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, bicycloalkyl, substituted bicycloalkyl, bicycloheteroalkyl, and substituted bicycloheteroalkyl;
R³is chosen from H, halogen, alkyl, substituted alkyl, aryl, substituted aryl, arylalkyl, substituted arylalkyl, heterocycloalkyl, substituted heterocycloalkyl, heteroaryl, substituted heteroaryl, heteroarylalkyl, substituted heteroarylalkyl, and dialkylamin;
R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, heteroarylalkyl, and substituted heteroarylalkyl;
or R³ and R⁴ together with the atoms to which R³ and R⁴ are attached form a cycloalkyl, substituted cycloalkyl, heterocycloalkyl, substituted heterocycloalkyl, bicycloalkyl, substituted bicycloalkyl, bicycloheteroalkyl, or substituted bicycloheteroalkyl ring; and
R⁵ is chosen from H, alkyl, substituted alkyl, arylalkyl, and substituted arylalkyl;
with the provisos that
R³ is not chosen from H, 2-aminopyrimidine, substituted 2-aminopyrimidine, 2-aminopyridine, substituted 2-aminopyridine, aminotriazine, or substituted aminotriazine; and R⁴ is not chosen from 2-aminopyrimidine, substituted 2-aminopyrimidine, 2-aminopyridine, substituted 2-aminopyridine, aminotriazine, or substituted aminotriazine;
and wherein the compound of Formula (IV), a stereoisomer thereof, a pharmaceutically acceptable salt thereof, a hydrate thereof, or a solvate of any of the foregoing, exhibits ATP-utilizing enzyme inhibitory activity.

Certain aspects of the present disclosure are directed to compounds of Formula (V): 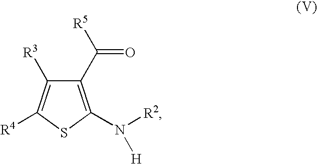
a stereoisomer thereof, a pharmaceutically acceptable salt thereof, a hydrate thereof, or a solvate of any of the foregoing, wherein:
R² is chosen from H, and -ZR⁶ wherein
Z is carbonyl; and
R⁶ is chosen from alkyl, substituted alkyl, heteroalkyl, substituted heteroalkyl, aryl, substituted aryl, heteroaryl, substituted heteroaryl, heterocycloalkylalkyl, and substituted heterocycloalkylalkyl;
R³ is chosen from H, halogen, alkyl, and substituted alkyl;
R⁴ is chosen from H, halogen, alkyl, and substituted alkyl;
or R³ and R⁴ together with the atoms to which R³ and R⁴ are attached form a cycloalkyl, substituted cycloalkyl ring;
R⁵ is chosen from H, aryl, substituted aryl, heteroaryl, and substituted heteroaryl;
and wherein the compound of Formula (V), a stereoisomer thereof, a pharmaceutically acceptable salt thereof, a hydrate thereof, or a solvate of any of the foregoing, exhibits ATP-utilizing enzyme inhibitory activity.

Certain aspects of the present disclosure are directed to compounds of Formula (VI): 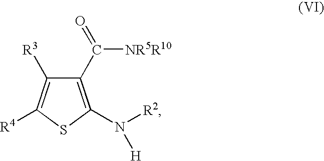
a stereoisomer thereof, a pharmaceutically acceptable salt thereof, a hydrate thereof, or a solvate of any of the foregoing, wherein:
R² is chosen from H, aryl, substituted aryl, heteroaryl, substituted heteroaryl, heterocycloalkyl, substituted heterocycloalkyl, alkylsulfonyl, substituted, alkylsulfonyl, heteroalkylsulfonyl, substituted heteroalkylsulfonyl, and -ZR⁶, wherein
Z is carbonyl; and
R⁶ is chosen from H, alkyl, substituted alkyl, aryl, and substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, arylalkyl, substituted arylalkyl, heteroarylalkyl, and substituted heteroalkyl;
R³ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
R⁴ is chosen from H, alkyl, substituted alkyl, aryl, substituted aryl, arylalkyl, substituted arylalkyl, heteroaryl, substituted heteroaryl, heteroarylalkyl, and substituted heteroarylalkyl;
or R³ and R⁴ together with the atoms to which R³ and R⁴ are attached form a cycloalkyl, substituted cycloalkyl, heterocycloalkyl, or substituted heterocycloalkyl ring;
R⁵ is chosen from H, alkyl, substituted alkyl;
R¹⁰ is chosen from H, alkyl, substituted alkyl, aryl, substituted aryl, arylalkyl, substituted arylalkyl, heteroalkyl, substituted heteroalkyl, heteroalkyl, and substituted heteroalkyl;
or, R⁵ and R¹⁰ together with the atoms to which R⁵ and R¹⁰ are attached form a cycloalkyl, substituted cycloalkyl, heterocycloalkyl, or substituted heterocycloalkyl ring; and
with the provisos that
when R³ is H, and R² is —C(═O)NR¹²R¹¹, and R¹¹ is H, then R¹² is not alkyl or substituted alkyl; and
when R¹ is H, and R⁵ is H, then R¹⁰ is not H;
and wherein the compound of Formula (VI), a stereoisomer thereof a pharmaceutically acceptable salt thereof, a hydrate thereof, or a solvate of any of the foregoing, is an inhibitor of at least one ATP-utilizing enzyme.

Certain aspects of the present disclosure provide compositions comprising at least one compound disclosed herein. In certain embodiments, the compositions comprise at least one compound of the present disclosure, a stereoisomer thereof, a pharmaceutically acceptable salt thereof, a hydrate thereof, or a solvate of any of the foregoing, and a pharmaceutically acceptable diluent, carrier, excipient and/or adjuvant.

Certain aspects of the present disclosure provide methods of treating a disease in a patient in need of such treatment comprising administering to the patient a therapeutically effective amount of at least one compound disclosed herein.

Certain aspects of the present disclosure provide methods of treating a disease regulated by at least one ATP-utilizing enzyme, such as a human protein kinase, in a subject in need of such treatment comprising administering to the subject a therapeutically effective amount of at least one compound disclosed herein.

Certain aspects of the present disclosure provide methods of inhibiting at least one ATP-utilizing enzyme, and more specifically, a human protein kinase, in a subject comprising administering to the subject at least one compound disclosed herein.

Certain aspects of the present disclosure provide methods of inhibiting at least one ATP-utilizing enzyme comprising contacting the ATP-utilizing enzyme with at least one compound disclosed herein.

Certain aspects of the present disclosure provide compounds, stereoisomers thereof, pharmaceutically acceptable salts thereof, hydrates thereof, or solvates of any of the foregoing, which exhibit ATP-utilizing enzyme inhibitory activity, such as, human protein kinase inhibitory activity.

Additional embodiments of the invention are set forth in the description which follows, or may be learned by practice of the invention.

Definitions Used in the Present Disclosure

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements. At the very least, and not as an attempt to limit the application of the doctrine of equivalents to the scope of the claims, each numerical parameter as set forth in the claims should at least be construed in light of the number of reported significant digits and by applying ordinary rounding techniques.

“Acyl” refers to a radical —C(O)R, where R is hydrogen, alkyl, cycloalkyl, heterocycloalkyl, aryl, arylalkyl, heteroalkyl, heteroaryl, and heteroarylalkyl as defined herein. Representative examples include, but are not limited to, formyl, acetyl, cylcohexylcarbonyl, cyclohexylmethylcarbonyl, benzoyl, benzylcarbonyl, and the like.

“Aminoacyl” refers to a radical —NRC(O)R′, where R and R′ are each independently chosen from hydrogen, alkyl, cycloalkyl, heterocycloalkyl, aryl, arylalkyl, heteroalkyl, heteroaryl, heteroarylalkyl, and heterocycloalkyl, as defined herein. Representative examples include, but are not limited to, formylamino, acetylamino, cylcohexylcarbonylamino, cyclohexylmethyl-carbonylamino, benzoylamino, benzylcarbonylamino, and the like.

“Alkanyl” refers to a saturated branched, straight-chain or cyclic alkyl group derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl; butanyls such as butan-1-yl, butan-2-yl (sec-butyl), 2-methyl-propan-1-yl (isobutyl), 2-methyl-propan-2-yl (t-butyl), cyclobutan-1-yl; and the like.

“Alkenyl” refers to an unsaturated branched, straight-chain or cyclic alkyl group having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl; and the like. In certain embodiments, an alkenyl group has from 2 to 20 carbon atoms and in other embodiments, from 2 to 6 carbon atoms.

“Alkoxy” refers to a radical —OR where R represents an alkyl, heteroalkyl, cycloalkyl, heterocycloalkyl, aryl, heteroaryl, cycloalkylalkyl, heterocycloalkylalkyl, arylalkyl or heteroarylalkyl group as defined herein. Representative examples include, but are not limited to, methoxy, ethoxy, propoxy, butoxy, cyclohexyloxy, and the like.

“Alkoxycarbonyl” refers to a radical —CO-alkoxy where alkoxy is as defined herein.

“Alkoxythiocarbonyl” refers to a radical —C(S)-alkoxy where alkoxy is as defined herein.

“Alkyl” refers to a saturated or unsaturated, branched, straight-chain or cyclic monovalent hydrocarbon group derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In certain embodiments, an alkyl group comprises from 1 to 20 carbon atoms. In other embodiments, an alkyl group comprises from 1 to 6 carbon atoms, and is referred to as a lower alkyl group.

“Alkylamino” refers to a radical —NHR where R represents an alkyl, heteroalkyl, cycloalkyl, heterocycloalkyl, aryl, heteroaryl, cycloalkylalkyl, heterocycloalkylalkyl, arylalkyl or heteroarylalkyl group as defined herein. Representative examples include, but are not limited to, methylamino, ethylamino, 1-methylethylamino, cyclohexyl amino, and the like.

“Alkylsulfonyl” refers to a radical —S(O)₂R where R is an alkyl, heteroalkyl, cycloalkyl, heterocycloalkyl, aryl, heteroaryl, cycloalkylalkyl, heterocycloalkylalkyl, arylalkyl or heteroarylalkyl group as defined herein. Representative examples include, but are not limited to methylsulfonyl, ethylsulfonyl, propylsulfonyl, butylsulfonyl, and the like.

“Alkylsulfinyl” refers to a radical —S(O)R where R is an alkyl, heteroalkyl, cycloalkyl, heterocycloalkyl, aryl, heteroaryl, cycloalkylalkyl, heterocycloalkylalkyl, arylalkyl or heteroarylalkyl group as defined herein. Representative examples include, but are not limited to, methylsulfinyl, ethylsulfinyl, propylsulfinyl, butylsulfinyl, and the like.

“Alkylthio” refers to a radical —SR where R is an alkyl, heteroalkyl, cycloalkyl, heterocycloalkyl, aryl, heteroaryl, cycloalkylalkyl, heterocycloalkylalkyl, arylalkyl or heteroarylalkyl group as defined herein that may be optionally substituted as defined herein. Representative examples include, but are not limited to, methylthio, ethylthio, propylthio, butylthio, and the like.

“Alkylthiocarbonyl” refers to a radical —C(S)R, where R is hydrogen, alkyl, heteroalkyl, cycloalkyl, heterocycloalkyl, aryl, heteroaryl, cycloalkylalkyl, heterocycloalkylalkyl, arylalkyl or heteroarylalkyl group as defined herein.

“Alkylamidino” refers to the group —C(NR)NR′R″ where R, R′, and R″ are independently chosen from hydrogen, alkyl, aryl, cycloalkyl, heteroaryl, arylalkyl, heteroarylalkyl, heterocycloalkyl, and heterocycloalkylalkyl, as defined herein, or optionally R′ and R″ together with the nitrogen atom to which R′ and R″ are attached form one or more heterocyclic or substituted heterocyclic rings.

“Alkynyl” refers to an unsaturated branched, straight-chain or cyclic alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 3 to 6 carbon atoms.

“Amino” refers to the radical —NH₂.

“Aminocarbonyl” refers to the group —C(O)NRR′ where R and R′ are independently chosen from hydrogen, alkyl, heteroalkyl, cycloalkyl, heterocycloalkyl, aryl, heteroaryl, cycloalkylalkyl, heterocycloalkylalkyl, arylalkyl or heteroarylalkyl, as defined herein, or optionally R′ and R″ together with the nitrogen atom to which R′ and R″ are attached form one or more heterocyclic or substituted heterocyclic rings.

“Aminocarbonylamino” refers to the group —NRC(O)NR′R″ where R, R′, and R″ are independently chosen from hydrogen, alkyl, heteroalkyl, cycloalkyl, heterocycloalkyl, aryl, heteroaryl, cycloalkylalkyl, heterocycloalkylalkyl, arylalkyl or heteroarylalkyl, as defined herein, or optionally R′ and R″ together with the nitrogen atom to which R′ and R″ are attached form one or more heterocyclic or substituted heterocyclic rings.

“Aminosulfonyl” refers to a radical —S(O₂)NRR′ wherein R and R′ are independently chosen from hydrogen, alkyl, heteroalkyl, cycloalkyl, heterocycloalkyl, aryl, heteroaryl, cycloalkylalkyl, heterocycloalkylalkyl, arylalkyl or heteroarylalkyl as defined herein, or optionally R′ and R″ together with the nitrogen atom to which R′ and R″ are attached form one or more heterocyclic or substituted heterocyclic rings.

“Alkylsulfonylamino” refers to a radical —NRS(O)₂R′ where R and R′ independently represent an alkyl, cycloalkyl, aryl, or heteroaryl group as defined herein.

“Aminothiocarbonyl” refers to the group —C(S)NRR′ where R and R′ independently chosen from hydrogen, alkyl, heteroalkyl, cycloalkyl, heterocycloalkyl, aryl, heteroaryl, cycloalkylalkyl, heterocycloalkylalkyl, arylalkyl or heteroarylalkyl as defined herein, or optionally R′ and R″ together with the nitrogen atom to which R′ and R″ are attached form one or more heterocyclic or substituted heterocyclic rings.

“Aminothiocarbonylamino” refers to the group —NRC(S)NR′R″ where R, R′, and R″ independently chosen from hydrogen, alkyl, heteroalkyl, cycloalkyl, heterocycloalkyl, aryl, heteroaryl, cycloalkylalkyl, heterocycloalkylalkyl, arylalkyl or heteroarylalkyl as defined herein, or optionally R′ and R″ together with the nitrogen atom to which R′ and R″ are attached form one or more heterocyclic or substituted heterocyclic rings.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can comprise from 6 to 20 carbon atoms. In certain embodiments, an aryl group includes an aryl group fused with one or more cycloalkyl or heterocycloalkyl groups as defined herein.

“Arylalkyl” refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, and/or arylalkynyl is used. In certain embodiments, an arylalkyl group can be (C₆₋₃₀) arylalkyl, e.g., the alkanyl, alkenyl or alkynyl moiety of the arylalkyl group can be (C₁₋₁₀) and the aryl moiety can be (C₆₋₂₀).

“Arylalkyloxy“ refers to an arylalkyl-O— group where arylalkyl is as defined herein.

“Aryloxycarbonyl” refers to a radical —C(O)—O-aryl where aryl is as defined herein.

“Bicycloalkyl” refers to a saturated or unsaturated polycyclic group having two bridgehead atoms and three bonds connecting each bridgehead atom, derived by the removal of one hydrogen atom from a single carbon atom of a parent bicycloalkyl group.

“Bicycloheteroalkyl” refers to a saturated or unsaturated bicycloalkyl group in which one or more carbon atoms (and any associated hydrogen atoms) are independently replaced with the same or different heteroatom. Typical heteroatoms to replace the carbon atom(s) include, but are not limited to, N, P, O, S, and Si.

“Carbonyl” refers to a radical —C(O) group.

“Carboxyl” refers to the radical —C(O)OH.

“Cleave” refers to breakage of chemical bonds and is not limited to chemical or enzymatic reactions or mechanisms unless clearly indicated by the context.

“Compounds of the present disclosure” refers to compounds encompassed by generic formulae disclosed herein, any subgenus of those generic formulae, and any specific compounds within those generic or subgeneric formulae. The compounds of the present disclosure include a specific specie, a subgenus or a larger genus, each of which are identified either by the chemical structure and/or chemical name. Further, compounds of the present disclosure also include substitutions or modifications of any of such species, subgenuses or genuses, which are set forth herein.

When the chemical structure and chemical name conflict, the chemical structure is determinative of the identity of the compound. The compounds of the present disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Further, when partial structures of the compounds of the present disclosure are illustrated, asterisks indicate the point of attachment of the partial structure to the rest of the molecule. Enantiomeric and stereoisomeric mixtures can be resolved into the component enantiomers or stereoisomers using separation techniques or chiral synthesis techniques well known to the skilled artisan.

“Bond” refers to a covalent attachment between two atoms.

“Cyano” refers to the radical —CN.

“Cycloalkyl” refers to a saturated or unsaturated cyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In certain embodiments, the cycloalkyl group can be C₃₋₁₀ cycloalkyl, such as, for example, C₃₋₆ cycloalkyl. In certain embodiments, a cycloalkyl group includes a cycloalkyl group fused with one or more aryl or heteroaryl groups, as defined herein.

“Cycloalkylalkyl” refers to an acyclic alkyl group in which one of the hydrogen atoms bonded to a carbon atom, typically a terminal or Sp³ carbon atom, is replaced with an cycloalkyl group. In certain embodiments, a cycloalkylalkyl group can be C₆₋₃₀ cycloalkylalkyl, e.g., the alkanyl, alkenyl or alkynyl moiety of the cycloalkylalkyl group can be C₁₋₁₀ and the cycloalkyl moiety can be C₆₋₂₀.

“Dialkylamino” refers to a radical —NR′R″ where R′ and R″ independently chosen from hydrogen, alkyl, heteroalkyl, cycloalkyl, heterocycloalkyl, aryl, heteroaryl, cycloalkylalkyl, heterocycloalkylalkyl, arylalkyl or heteroarylalkyl as defined herein, or optionally R′ and R″ together with the nitrogen atom to which R′ and R″ are attached form one or more heterocyclic or substituted heterocyclic rings. Representative examples include, but are not limited to, dimethylamino, methylethylamino, di-(1-methylethyl)amino, (cyclohexyl)(methyl)amino, (cyclohexyl)(ethyl)amino, (cyclohexyl)(propyl)amino, and the like.

“Disease” refers to any disease, disorder, condition, symptom, or indication.

“Enzyme” refers to any naturally occurring or synthetic macromolecular substance composed wholly or largely of protein, that catalyzes, more or less specifically, one or more biochemical reactions. The substances upon which the enzyme acts are referred to “substrates,” for which the enzyme possesses a specific binding or “active site,” or “catalytic domain.” Enzymes can also act on macromolecular structures such as muscle fibers.

“Extended release” refers to dosage forms that provide for the delayed, slowed, over a period of time, continuous, discontinuous, or sustained release of the compounds of the present disclosure.

“Halogen” refers to a fluoro, chloro, bromo, or iodo group.

“Heteroalkyloxy” refers to an heteroalkyl group where heteroalkyl is as defined herein.

“Heteroalkyl, heteroalkanyl, heteroalkenyl, heteroalkynyl” refer to alkyl, alkanyl, alkenyl and alkynyl groups, respectively, in which one or more of the carbon atoms (and any associated hydrogen atoms) are each independently replaced with the same or different heteroatomic groups. Typical heteroatomic groups include, but are not limited to, —O—, —S—, —O—O—, —S—S—, —O—S—, —NR′—, ═N—N═, —N═N—, —N═N—NR′—, —PH—, —P(O)₂—, —O—P(O)₂—, —S(O)—, —S(O)₂—, —SnH₂— and the like, wherein R′ is chosen from hydrogen, alkyl, substituted alkyl, cycloalkyl, substituted cycloalkyl, aryl or substituted aryl.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group can be between 5 to 20 membered heteroaryl, such as, for example, a 5 to 10 membered heteroaryl. In certain embodiments, heteroaryl groups can be those derived from thiophene, pyrrole, benzothiophene, benzofuiran, indole, pyridine, quinoline, imidazole, oxazole, pyrazine, benzothiazole, isoxazole, thiadiaxole, and thiazole. In certain embodiments, a heteroaryl group includes a heteroaryl group fused with one or more cycloalkyl or heterocycloalkyl groups, as defined herein.

“Heteroarylalkyl” refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, and/or heteroarylalkynyl is used. In certain embodiments, the heteroarylalkyl group can be a 6 to 30 membered heteroarylalkyl, e.g., the alkanyl, alkenyl or alkynyl moiety of the heteroarylalkyl can be 1 to 10 membered and the heteroaryl moiety can be a 5 to 20-membered heteroaryl.

“Heterocycloalkyl” refers to a saturated or unsaturated cyclic alkyl group in which one or more carbon atoms (and any associated hydrogen atoms) are independently replaced with the same or different heteroatom. Typical heteroatoms to replace the carbon atom(s) include, but are not limited to, N, P, O, S, and Si. Where a specific level of saturation is intended, the nomenclature “heterocycloalkanyl” or “heterocycloalkenyl” is used. Typical heterocycloalkyl groups include, but are not limited to, groups derived from epoxides, imidazolidine, morpholine, piperazine, piperidine, pyrazolidine, pyrrolidine, quinuclidine, and the like. In certain embodiments, a heterocycloalkyl group includes one or more heterocycloalkyl groups fused with one or more aryl, or heteroaryl groups, as defined herein.

“Heterocycloalkylalkyl” refers to an acyclic alkyl group in which one of the hydrogen atoms bonded to a carbon atom, typically a terminal or sp³ carbon atom, is replaced with a heterocycloalkyl group. Where specific alkyl moieties are intended, the nomenclature heterocycloalkylalkanyl, heterocycloalkylalkenyl, and/or heterocycloalkylalkynyl is used. In certain embodiments, the heterocycloalkylalkyl group can be a 6 to 30 membered heterocycloalkyllalkyl, e.g., the alkanyl, alkenyl or alkynyl moiety of the heterocycloalkylalkyl can be 1 to 10 membered and the heterocycloalkyl moiety can be a 5 to 20-membered heterocycloalkyl.

“Heterocycloalkyloxycarbonyl” refers to a radical —C(O)—OR where R is heterocycloalkyl is as defined herein.

“Heteroaryloxycarbonyl” refers to a radical —C(O)—OR where R is heteroaryl as defined herein.

“Leaving group” refers to an atom or a group capable of being displaced by a nucleophile and includes halogen, such as chloro, bromo, fluoro, and iodo, alkoxycarbonyl (e.g., acetoxy), aryloxycarbonyl, mesyloxy, tosyloxy, trifluoromethanesulfonyloxy, aryloxy (e.g., 2,4-dinitrophenoxy), methoxy, N,O-dimethylhydroxylamino, and the like.

“Optional” or “optionally” means that the subsequently described event or circumstance may but need not occur, and that the description includes instances where the event or circumstance occurs and instances in which the event does not.

“Pharmaceutically acceptable” refers to approved or approvable by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Pharmaceutically acceptable excipient, carrier or adjuvant” refers to an excipient, carrier or adjuvant that can be administered to a subject, together with a compound of the present disclosure, and which does not destroy the pharmacological activity thereof and is nontoxic when administered in doses sufficient to deliver a therapeutic amount of the compound.

“Pharmaceutically acceptable vehicle” refers to a diluent, adjuvant, excipient or carrier with which a compound of the present disclosure is administered.

“Prodrug” refers to a derivative of a therapeutically effective compound that requires a transformation within the body to produce the therapeutically effective compound. Prodrugs can be pharmacologically inactive until converted to the parent compound.

“Promoiety” refers to a form of protecting group that when used to mask a functional group within a drug molecule converts the drug into a prodrug. For example, the promoiety can be attached to the drug via bond(s) that are cleaved by enzymatic or non-enzymatic means in vivo.

“Protecting group” refers to a grouping of atoms that when attached to a reactive group in a molecule masks, reduces or prevents that reactivity. Examples of protecting groups can be found in Green et al., “Protective Groups in Organic Chemistry,” (Wiley, 2^{nd} ed. 1991) and Harrison et al., “Compendium of Synthetic Organic Methods,” Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”), and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Protein kinase,” “kinase,” and “human protein kinase” refer to any enzyme that phosphorylates one or more hydroxyl or phenolic groups in proteins where ATP is the phosphoryl-group donor.

“Stereoisomer” refers to an isomer that differs in the arrangement of the constituent atoms in space. Stereoisomers that are mirror images of each other and optically active are termed “enantiomers,” and stereoisomers that are not mirror images of one another are termed “diastereoisomers.”

“Subject” includes mammals and humans. The terms “human” and “subject” are used interchangeably herein.

“Substituted” refers to a group in which one or more hydrogen atoms are each independently replaced with the same or different substituent(s). Typical substituents include, but are not limited to, —X, —R, —O⁻, ═O, —OR′, —SR′, —S⁻, ═S, —NR′R″, ═NR′, —CX₃, —CF₃, —CN, —OCN, —SCN, —NO, —NO₂, ═N₂, —N₃, C═N—OH, —S(O)₂O⁻, —S(O)₂OH, —S(O)₂R′, —OS(O₂)O⁻, —OS(O)₂R′, —P(O)(O⁻)₂, —P(O)(OR′)(O⁻), —OP(O)(OR′)(OR″), —C(O)R′, —C(S)R′, —C(O)OR′, —C(O)NR′R″, —C(O)O⁻, —C(S)OR′, —NR′″C(O)NR′R″, —NR′″C(S)NR′R″, —NR′″C(NR′)NR′R″. —C(NR′)NR′R″, —S(O)₂NR′R″, —NR′″S(O)₂R′, —NR′″C(O)R′, and —S(O)R′ where each X is independently a halogen; each R′ and R″ are independently hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, —NR′″R″″, —C(O)R′″ or —S(O)₂R″ or optionally R′ and R″ together with the atom to which R′ and R″ are attached form one or more heterocycloalkyl, substituted heterocycloalkyl, heteroaryl, or substituted heteroaryl rings; and R′″ and R″″ are independently hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl or substituted heteroarylalkyl, or optionally R′″ and R″″ together with the nitrogen atom to which R′″ and R″″ are attached form one or more heterocycloalkyl, substituted heterocycloalkyl, heteroaryl, or substituted heteroaryl rings. In certain embodiments, a tertiary amine or aromatic nitrogen may be substituted with one or more oxygen atoms to form the corresponding nitrogen oxide.

In certain embodiments, substituted cycloalkyl, substituted heterocycloalkyl, substituted aryl, and substituted heteroaryl include one or more of the following substituent groups: halogen, nitro, —OH, —CN, —COOH, —OCF₃, —N(CH₃)₂, ═O, ═S, —NH₂, —NHCOCH₃, C₁₋₈ alkyl, substituted C₁₋₈ alkyl, C₁₋₈ alkoxy, C₁₋₈ substituted alkoxy, C₁₋₈ heteroalkyl, C₁₋₈ substituted heteroalkyl, C₁₋₈ alkylsulfonyl, substituted C₁₋₈ alkylsulfonyl, C₅₋₁₀ arylsulfonyl, C₅₋₁₀ heteroarylsulfonyl, C₅₋₈ aryl, substituted C₅₋₈ as definded herein.

In certain embodiments, substituted cycloalkylalkyl, substituted heterocycloalkylalkyl, substituted arylalkyl, and substituted heteroarylalkyl include one or more of the following substitute groups: halogen, ═O, ═S, —C(O)—NH₂, nitro, —OH, ═NH, —NH₂, CF₃, C₁₋₈ alkyl, substituted C₁₋₈ alkyl, C₁₋₈ heteroalkyl, substituted C₁₋₆ heteroalkyl, C₁₋₈ alkoxy, substituted C₁₋₈ alkoxy, C₅₋₈ aryl, substituted C₄₋₈ aryl, _{C4-8} heteroaryl, and substituted C₄₋₈ heteroaryl, as defined herein.

In certain embodiments, substituted alkyl and substituted heteroalkyl includes one or more of the following substitute groups: halogen, —OH, and ═O, as defined herein.

“Sulfonyl” refers to a radical —S(O)₂ group.

“Thioalkoxy” refers to a radical —SR where R represents an alkyl, heteroalkyl, cycloalkyl, heterocycloalkyl, aryl, heteroaryl, cycloalkylalkyl, heterocycloalkylalkyl, arylalkyl or heteroarylalkyl group as defined herein.

“Thiocarbonyl” refers to a radical —C(S) group.

“Therapeutically effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Therapeutically effective dosage” refers to a dosage that provides effective treatment of a condition and/or disease in a subject. The therapeutically effective dosage can vary from compound to compound, and from subject to subject, and can depend upon factors such as the condition of the subject and the route of delivery. A therapeutically effective dosage can be determined in accordance with routine pharmacological procedures known to those skilled in the art.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, and inhibit at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

In the specification and the appended claims, the singular forms “a,” “an,” and “the” include plural reference unless the context clearly dictates otherwise.

Compounds

Certain embodiments of the present disclosure are directed to compounds of Formula (I): 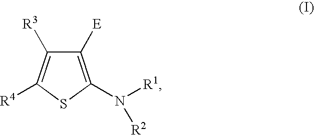
a stereoisomer thereof, a pharmaceutically acceptable salt thereof, a hydrate thereof, or a solvate of any of the foregoing, wherein:
E is chosen from —CN, halogen, —NO₂, and —C(═X)YR⁵; wherein
X is chosen from O, and S;
Y is chosen from —N(R¹⁰)—, O, S, and a direct bond; wherein
R¹⁰ is chosen H, alkyl, and substituted alkyl; and
R⁵ is chosen from H, alkyl, substituted alkyl, arylalkyl, substituted arylalkyl, cycloalkyl, substituted cycloalkyl, heterocycloalkyl, substituted heterocycloalkyl, heteroalkyl, substituted heteroalkyl, heteroarylalkyl, substituted heteroarylalkyl, and when Y is —N(R¹⁰)—, or a direct bond, then R⁵ is additionally chosen from aryl, substituted aryl, heteroaryl, substituted heteroaryl, —N(R⁷)₂, and —OR⁹; wherein
each R⁷ is independently chosen from alkyl, substituted alkyl, aryl, substituted aryl, and H; and
R⁹ is chosen from H, alkyl, and substituted alkyl;
or R⁵ and R¹⁰ together with the atoms to which R⁵ and R¹⁰ form a cycloalkyl, substituted cycloalkyl, heterocycloalkyl, or substituted heterocycloalkyl ring;
R¹ is chosen from H, alkyl, substituted alkyl, cycloalkyl, substituted cycloalkyl, heteroalkyl, and substituted heteroalkyl;
R is chosen from H, —CHO, alkyl, substituted alkyl, aryl, substituted aryl, cycloalkyl, substituted cycloalkyl, heteroaryl, substituted heteroaryl, heterocycloalkyl, substituted heterocycloalkyl, heteroarylalkyl, substituted heteroarylalkyl, heterocycloalkylalkyl, substituted heterocycloalkylalkyl, alkylsulfonyl, substituted alkylsulfonyl, heteroalkylsulfonyl, substituted heteroalkylsulfonyl, and -ZR⁶, wherein
Z is chosen from carbonyl, —C(O)O—, aminosulfonyl, aminothiocarbonyl, —C(═O)NR¹¹—, sulfonyl, and thiocarbonyl; wherein
R¹¹ is chosen from alkyl, substituted alkyl, cycloalkyl, substituted cycloalkyl, and H; and
R⁶ is chosen from H, —COOH, alkyl, substituted alkyl, aryl, substituted aryl, cycloalkyl, substituted cycloalkyl, heterocycloalkyl, substituted heterocycloalkyl, heteroalkyl, substituted heteroalkyl, heteroaryl, substituted heteroaryl, cycloalkylalkyl, substituted cycloalkylalkyl, heterocycloalkylalkyl, substituted heterocycloalkylalkyl, arylalkyl, substituted arylalkyl, heteroarylalkyl, substituted heteroarylalkyl, bicycloalkyl, substituted bicycloalkyl, bicycloheteroalkyl, and substituted bicycloheteroalkyl;
or R¹ and R², together with the atoms to which R¹ and R² are attached, form a heterocycloalkyl, or substituted heterocycloalkyl ring;
R³ is chosen from H, halogen, —NH₂, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, dialkylamino, and substituted dialkylamino; and
R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
or R³ and R⁴ together with the atoms to which R³ and R⁴ are attached form a cycloalkyl, substituted cycloalkyl, heterocycloalkyl, substituted heterocycloalkyl, bicycloalkyl, substituted bicycloalkyl, bicycloheteroalkyl, or substituted bicycloheteroalkyl ring;
with the provisos that
when E is —CO₂R⁵, then R³ is not H, 2-aminopyrimidine, substituted 2-aminopyrimidine 2-aminopyridine, substituted 2-aminopyridine, aminotriazine, or substituted aminotriazine; and R⁴ is not 2-aminopyrimidine, substituted 2-aminopyrimidine 2-aminopyridine, substituted 2-aminopyridine, aminotriazine, or substituted aminotriazine;
when E is —CN, then R³ is not H, 2-aminopyrimidine, substituted 2- aminopyrimidine 2-aminopyridine, substituted 2-aminopyridine, aminotriazine, or substituted aminotriazine; and R⁴ is not H, 2-aminopyrimidine, substituted 2-aminopyrimidine 2-aminopyridine, substituted 2-aminopyridine, aminotriazine, or substituted aminotriazine;
when E is —CN, and R² is —C(═X)NH₂, where X is O or S, then R³ is not unsubstituted phenyl, or a 5 to 7 member heteroaromatic ring containing 1 to 3 heteroatoms chosen from O, N or S; and R⁴ is not unsubstituted phenyl, or a 5 to 7 member heteroaromatic ring containing 1 to 3 heteroatoms chosen from O, N or S;
when E is —C(═O)NR⁵R¹⁰, and R is H, and R² is C(═O)NR¹²R¹¹, and R¹¹ is H, then R¹² is not alkyl or substituted alkyl; and
when E is —C(═O)NR⁵R¹⁰, and R¹ is H, and R⁵ is H, then R¹⁰ is not H
and wherein the compound of Formula (I), a stereoisomer thereof, a pharmaceutically acceptable salt thereof, a hydrate thereof, or a solvate of any of the foregoing, exhibits ATP-utilizing enzyme inhibitory activity.

In certain embodiments, compounds of the present disclosure are directed to compounds of Formula (II): 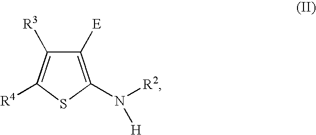
a stereoisomer thereof, a pharmaceutically acceptable salt thereof, a hydrate thereof, or a solvate of any of the foregoing, wherein:
E is chosen from —CN, halogen, —NO₂, and —C(═X)YR⁵; wherein
X is chosen from O, and S;
Y is chosen from —N(R¹⁰)—, O, S, and a direct bond; wherein
R¹⁰ is chosen H, alkyl, and substituted alkyl; and
R⁵ is chosen from H, alkyl, substituted alkyl, arylalkyl, substituted arylalkyl, cycloalkyl, substituted cycloalkyl, heterocycloalkyl, substituted heterocycloalkyl, heteroalkyl, substituted heteroalkyl, heteroarylalkyl, substituted heteroarylalkyl, and when Y is —N(R¹⁰)—, or a direct bond, then R⁵ is additionally chosen from aryl, substituted aryl, heteroaryl, substituted heteroaryl, —N(R⁷)₂, and —OR⁹; wherein
each R⁷is independently chosen from alkyl, substituted alkyl, aryl, substituted aryl, and H; and
R⁹ is chosen from H, alkyl, and substituted alkyl;
or R⁵ and R¹⁰ together with the atoms to which R⁵ and R¹⁰ form a cycloalkyl, substituted cycloalkyl, heterocycloalkyl, or substituted heterocycloalkyl ring;
R² is chosen from H, —CHO, alkyl, substituted alkyl, aryl, substituted aryl, cycloalkyl, substituted cycloalkyl, heteroaryl, substituted heteroaryl, heterocycloalkyl, substituted heterocycloalkyl, heteroarylalkyl, substituted heteroarylalkyl, heterocycloalkylalkyl, substituted heterocycloalkylalkyl, alkylsulfonyl, substituted alkylsulfonyl, heteroalkylsulfonyl, substituted heteroalkylsulfonyl, and -ZR⁶, wherein
Z is chosen from carbonyl, —C(O)O—, aminosulfonyl, aminothiocarbonyl, —C(═O)NR¹¹—, sulfonyl, and thiocarbonyl; wherein
R¹¹ is chosen from alkyl, substituted alkyl, cycloalkyl, substituted cycloalkyl, and H; and
R⁶ is chosen from H, —COOH, alkyl, substituted alkyl, aryl, substituted aryl, cycloalkyl, substituted cycloalkyl, heterocycloalkyl, substituted heterocycloalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, arylalkyl, substituted arylalkyl, heteroarylalkyl, substituted heteroarylalkyl, bicycloalkyl, substituted bicycloalkyl, bicycloheteroalkyl, and substituted bicycloheteroalkyl;
or R¹ and R², together with the atoms to which R¹ and R² are attached, form a heterocycloalkyl, or substituted heterocycloalkyl ring;
R³ is chosen from H, halogen, —NH₂, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, dialkylamino, and substituted dialkylamino; and
R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
or R³ and R⁴ together with the atoms to which R³ and R⁴ are attached form a cycloalkyl, substituted cycloalkyl, heterocycloalkyl, substituted heterocycloalkyl, bicycloalkyl, substituted bicycloalkyl, bicycloheteroalkyl, or substituted bicycloheteroalkyl ring;
with the provisos that
when E is —CO₂R⁵, then R³ is not H, 2-aminopyrimidine, substituted 2-aminopyrimidine, 2-aminopyridine, substituted 2-aminopyridine, aminotriazine or substituted aminotriazine; and R⁴ is not 2-aminopyrimidine, substituted 2-aminopyrimidine, 2-aminopyridine, substituted 2-aminopyridine, aminotriazine or substituted aminotriazine;
when E is —CN, then R³is not 2-aminopyrimidine, substituted 2-aminopyrimidine, 2-aminopyridine, substituted 2-aminopyridine, aminotriazine or substituted aminotriazine; and R⁴ is not 2-aminopyrimidine, substituted 2-aminopyrimidine, 2-aminopyridine, substituted 2-aminopyridine, aminotriazine or substituted aminotriazine;
when E is —CN, and R² is —(═X)NH₂, then R³is not unsubstituted phenyl or a 5 to 7 membered heteroaromatic ring containing 1 to 3 heteroatoms chosen from O, N or S; and R⁴ is not unsubstituted phenyl or a 5 to 7 membered heteroaromatic ring containing 1 to 3 heteroatoms chosen from O, N or S;
when E is C(═O)NR⁵R¹⁰, and R³is H, and R² is C(═O)NR¹²R¹¹, and R¹¹ is H, then R¹² is not alkyl, or substituted alkyl; and
when E is —C(═O)NR⁵R¹⁰, and R¹ is H, and R⁵ is H, then R¹⁰ is not H;
and wherein the compound of Formula (II), a stereoisomer thereof, a pharmaceutically acceptable salt thereof, a hydrate thereof, or a solvate of any of the foregoing, exhibits ATP-utilizing enzyme inhibitory activity.

In certain embodiments of compounds of Formula (II), Y is chosen from O, a direct bond, and —N(R¹⁰)— wherein R¹⁰ is H; and R⁵ is chosen from H, alkyl, substituted alkyl, aryl, substituted aryl, heteroaryl, substituted heteroaryl, arylalkyl, and substituted arylalkyl.

In certain embodiments of compounds of Formula (II), R⁵ is chosen from H, C₁₋₁₀ alkyl, substituted C₁₋₁₀ alkyl, C₃₋₁₂ aryl, substituted C₃₋₁₂ aryl, C₃₋₁₂ heteroaryl, substituted C₃₋₁₂ heteroaryl, C₄₋₁₈ arylalkyl, and substituted C₄₋₁₈ arylalkyl.

In certain embodiments of compounds of Formula (II), R² is chosen from H, and -ZR⁶, wherein Z is chosen from carbonyl, and —C(═O)NH—, and R⁶ is chosen from H, —COOH, C₁₋₁₀ alkyl, substituted C₁₋₁₀ alkyl, C₅₋₁₂ aryl, substituted C₅₋₁₂ aryl, C₃₋₁₂ cycloalkyl, substituted C₃₋₁₂ cycloalkyl, C₃₋₁₂ heterocycloalkyl, substituted C₃₋₁₂ heterocycloalkyl, C₁₋₁₀ heteroalkyl, substituted C₁₋₁₀ heteroalkyl, C₅₋₁₂ heteroaryl, substituted C₅₋₁₂ heteroaryl, C₆₋₁₈ heteroarylalkyl, substituted C₆₋₁₈ heteroarylalkyl, C₄₋₁₈ cycloalkylalkyl, substituted C₄₋₁₈ cycloalkylalkyl, C₄₋₁₈ heterocycloalkylalkyl, substituted C₄₋₁₈heterocycloalkylalkyl, C₆₋₁₈ arylalkyl, substituted C₆₋₁₈ garylalkyl, C₅₋₁₂ bicycloalkyl, substituted C₅₋₁₂ bicycloalkyl, C₅₋₁₂ bicycloheteroalkyl, and substituted C₅₋₁₂ bicycloheteroalkyl.

In certain embodiments of compounds of Formula (II), R³ is chosen from H, halogen, —NH₂, alkyl, substituted alkyl, acyl, substituted acyl, alkoxycarbonyl, substituted alkoxycarbonyl, aminocarbonyl, substituted aminocarbonyl, cycloalkyl, substituted cycloalkyl, heteroalkyl, substituted heteroalkyl, aryl, substituted aryl, heteroaryl, substituted heteroaryl, arylalkyl, substituted arylalkyl, heteroarylalkyl, substituted heteroarylalkyl, and dialkylamino.

In certain embodiments of compounds of Formula (II), R³ is chosen from H, halogen, —NH₂, C₁₋₁₀ alkyl, substituted C₁₋₁₀ alkyl, C₁₋₁₀ acyl, substituted C₁₋₁₀ acyl, C₁₋₁₀ alkoxycarbonyl, substituted C₁₋₁₀ alkoxycarbonyl, C₁₋₁₀ aminocarbonyl, substituted C₁₋₁₀ aminocarbonyl, C₃₋₁₂ cycloalkyl, substituted C₃₋₁₂ cycloalkyl, C₃₋₁₂ heteroalkyl, substituted C₃₋₁₂ heteroalkyl, C₅₋₁₂ aryl, substituted C₅₋₁₂ aryl, C₅₋₁₂ heteroaryl, substituted C₅₋₁₂ heteroaryl, C₆₋₁₈ arylalkyl, substituted C₆₋₁₈ arylalkyl, C₆₋₁₈ heteroarylalkyl, substituted C₆₋₁₈ heteroarylalkyl, and C₂₋₂₀ dialkylamino.

In certain embodiments of compounds of Formula (II), R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, and substituted arylalkyl, heteroaryl, substituted heteroaryl, heterocycloalkylalkyl, substituted heterocycloalkylalkyl, heteroarylalkyl, and substituted heteroarylalkyl.

In certain embodiments of compounds of Formula (II), R⁴ is chosen from H, halogen, C₁₋₁₀ acyl, substituted C₁₋₁₀ acyl, C₁₋₁₀ alkoxycarbonyl, substituted C₁₋₁₀ alkoxycarbonyl, C₁₋₁₀ alkyl, substituted C₁₋₁₀ alkyl, C₁₋₁₀ aminocarbonyl, substituted C₁₋₁₀ aminocarbonyl, C₅₋₁₂ aryl, substituted C₅₋₁₂ aryl, arylalkyl, and substituted arylalkyl, C₅₋₁₂ heteroaryl, substituted C₅₋₁₂ heteroaryl, C₄₋₁₈ heterocycloalkylalkyl, substituted C₄₋₁₈ heterocycloalkylalkyl, C₆₋₁₈ heteroarylalkyl, and substituted C₆₋₁₈ heteroarylalkyl.

In certain embodiments, compounds of the present disclosure are directed to compounds of Formula (III): 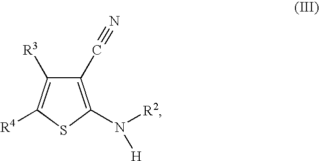
a stereoisomer thereof, a pharmaceutically acceptable salt thereof, a hydrate thereof, or a solvate of any of the foregoing, wherein:
R² is chosen from H, and -ZR⁶, wherein
Z is carbonyl; and
R⁶ is chosen from H, alkyl, substituted alkyl, heteroalkyl, substituted heteroalkyl, aryl, substituted aryl, heteroaryl, substituted heteroaryl, heterocycloalkyl, substituted heterocycloalkyl, heterocycloalkylalkyl, substituted heterocycloalkylalkyl, heteroarylalkyl, and substituted heteroarylalkyl;
R³ is chosen from H, —NH₂, alkyl, and substituted alkyl; and
R⁴ is chosen from H, halogen, alkyl, substituted alkyl, heteroalkyl, substituted heteroalkyl, arylalkyl, substituted arylalkyl, heterocycloalkylalkyl, substituted heterocycloalkylalkyl;
or R³ and R⁴ together with the atoms to which R³ and R⁴ are attached form a cycloalkyl, substituted cycloalkyl, heterocycloalkyl, or substituted heterocycloalkyl ring;
with the provisos that
R³ is not H, 2-aminopyrimidine, substituted 2-aminopyrimidine, 2-aminopyridine, substituted 2-aminopyridine, aminotriazine, or substituted aminotriazine;
R⁴ is not H, 2-aminopyrimidine, substituted 2-aminopyrimidine, 2-aminopyridine, substituted 2-aminopyridine, aminotriazine, or substituted aminotriazine; and
when R² is —C(═X)NH₂, where X is O or S, then R³ is not unsubstituted phenyl, or a 5 to 7 membered heteroaromatic ring containing 1 to 3 heteroatoms chosen from O, N or S; and R⁴ is not unsubstituted phenyl, or a 5 to 7 membered heteroaromatic ring containing 1 to 3 heteroatoms chosen from O, N or S;
and wherein the compound of Formula (II), a stereoisomer thereof, a pharmaceutically acceptable salt thereof, a hydrate thereof, or a solvate of any of the foregoing, exhibits ATP-utilizing enzyme inhibitory activity.

In certain embodiments of compounds of Formula (III), R⁴ is chosen from C₁₋₈ alkyl, substituted C₁₋₈ alkyl, C₁₋₈ heteroalkyl, substituted C₁₋₈ heteroalkyl, C₆₋₁₂ arylalkyl, substituted C₆₋₁₂ arylalkyl, C₆₋₁₂ heterocycloalkylalkyl, and substituted C₆₋₁₂ heterocycloalkylalkyl.

In certain embodiments of compounds of Formula (III), R³ and R⁴ together with the atoms to which R³ and R⁴ are attached form a C₅₋₁₀ cycloalkyl, substituted C₅₋₁₀ cycloalkyl, C₅₋₁₀ heterocycloalkyl, or substituted C₅₋₁₀ heterocycloalkyl ring.

In certain embodiments of compounds of Formula (III), wherein R³ and R⁴ together with the atoms to which R³ and R⁴ are attached form a C₅₋₁₀ cycloalkyl, substituted C₅₋₁₀ cycloalkyl, C₅₋₁₀ heterocycloalkyl, or substituted C₅₋₁₀ heterocycloalkyl ring, the at least one substituent group is chosen from halogen, C₁₋₆ alkyl, and ═O.

In certain embodiments of compounds of Formula (III), R⁴ is chosen from C₁₋₈ alkyl, substituted C₁₋₈ heteroalkyl, substituted C₅₋₁₀ arylalkyl, and substituted C₆₋₁₀ heterocycloalkylalkyl.

In certain embodiments of compounds of Formula (III), R³ is chosen from —NH₂, C₁₋₈ alkyl, and substituted C₁₋₈ alkyl.

In certain embodiments of compounds of Formula (III), R² is chosen from H, and —C(O)R⁶ wherein R⁶ is chosen from C₁₋₈ alkyl, substituted C₁₋₈ alkyl, C₁₋₈ heteroalkyl, substituted C₁₋₈ heteroalkyl, C₅₋₁₂ aryl, substituted C₅₋₁₂ aryl, C₅₋₁₂ heteroaryl, substituted C₅₋₁₂ heteroaryl, C₆₋₁₈ heterocycloalkyl, substituted C₆₋₁₈ heterocycloalkyl, C₆₋₁₈ heterocycloalkylalkyl, substituted C₆₋₁₈ heterocycloalkylalkyl, C₆₋₁₈ heteroarylalkyl, and substituted C₆₋₁₈ heteroarylalkyl.

In certain embodiments of compounds of Formula (III), wherein R² is chosen from H, and —C(O)R⁶ wherein R⁶ is chosen from C₁₋₈ alkyl, substituted C₁₋₈ alkyl, C₁₋₈ heteroalkyl, substituted C₁₋₈ heteroalkyl, C₅₋₁₂ aryl, substituted C₅₋₁₂ aryl, heteroaryl, substituted C₅₋₁₂ heteroaryl, C₆₋₁₈ heterocycloalkyl, substituted C₆₋₁₈ heterocycloalkyl, C₆₋₁₈ heterocycloalkylalkyl, substituted C₆₋₁₈ heterocycloalkylalkyl, C₆₋₁₈ heteroarylalkyl, and substituted C₆₋₁₈ heteroarylalkyl, the at least one substituent group is chosen from halogen, C₁₋₆ alkyl, C₁₋₆ alkoxy, C₅₋₈ aryl, substituted C₅₋₈ aryl, C₅₋₈ heteroaryl, substituted C₅₋₈ heteroaryl, ═O, ═S, —COOH, —CF₃, and —OH.

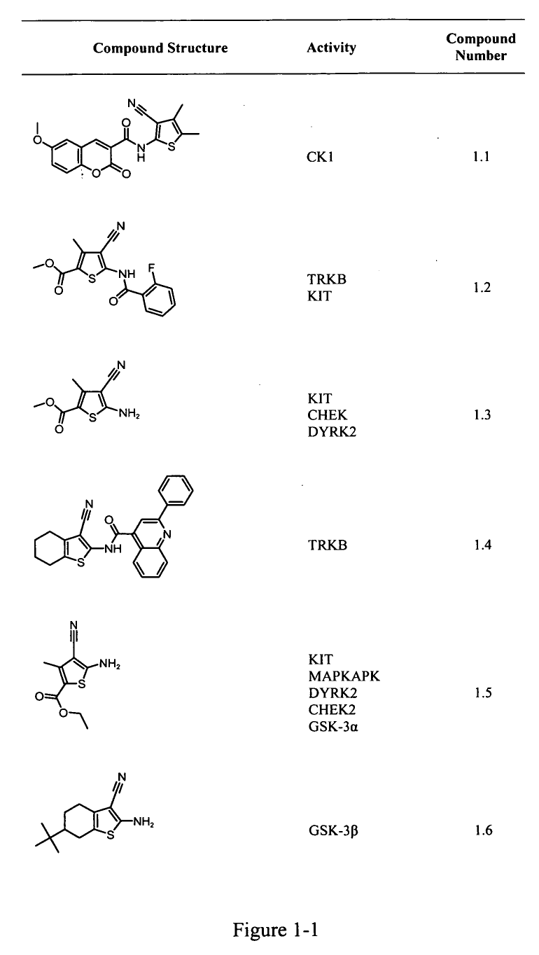

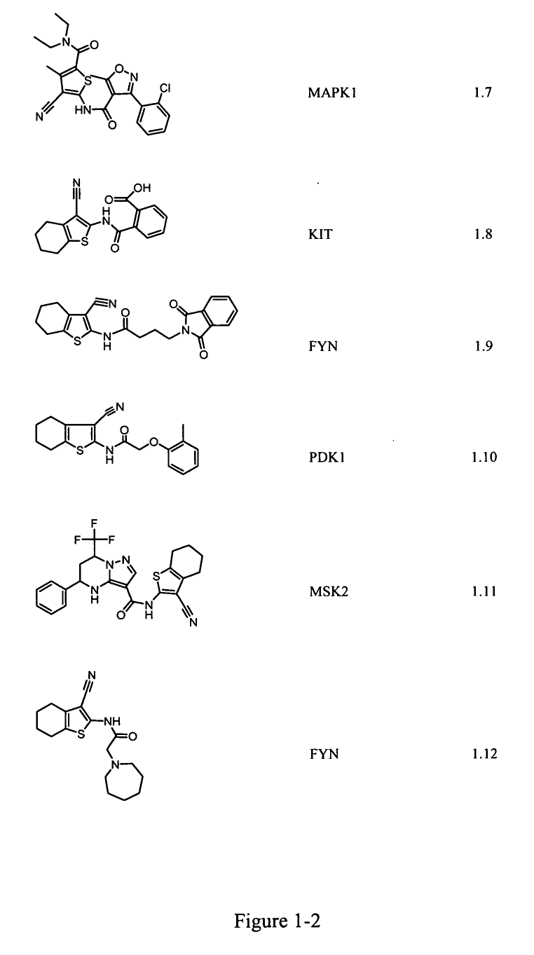

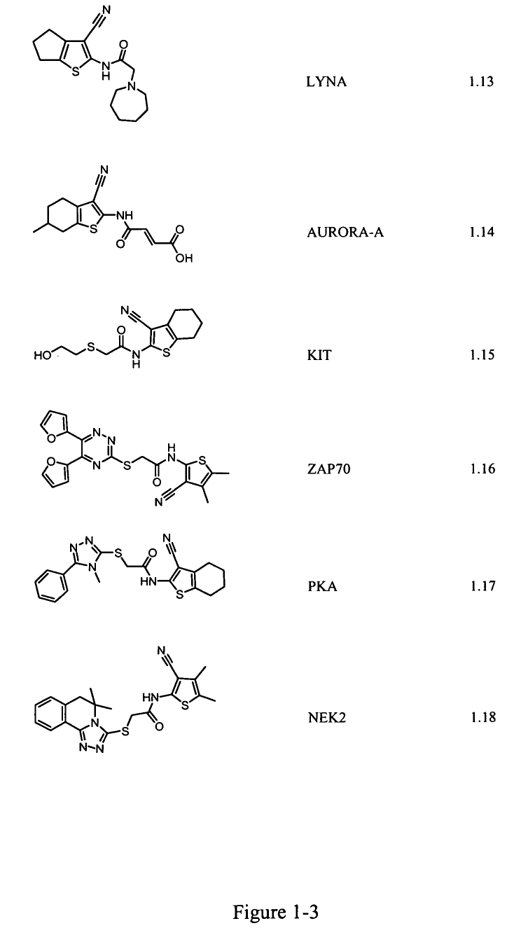

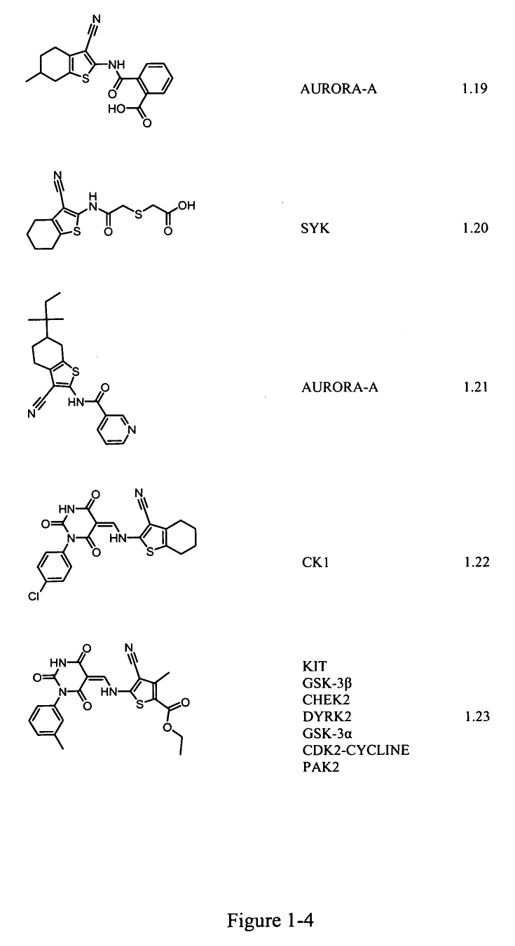

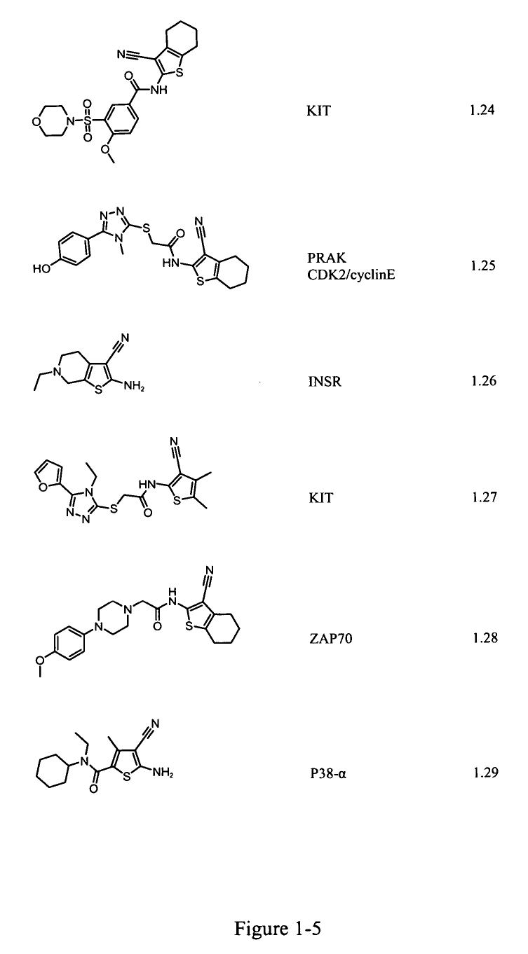

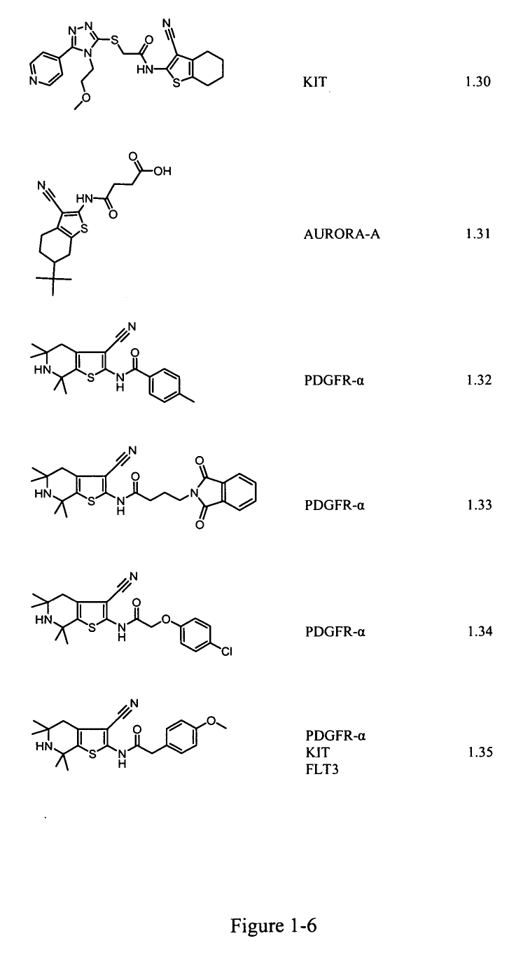

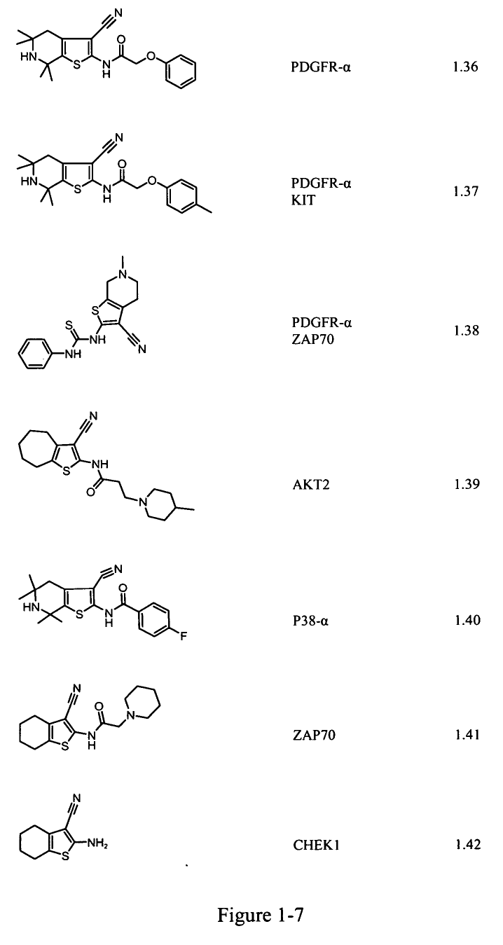

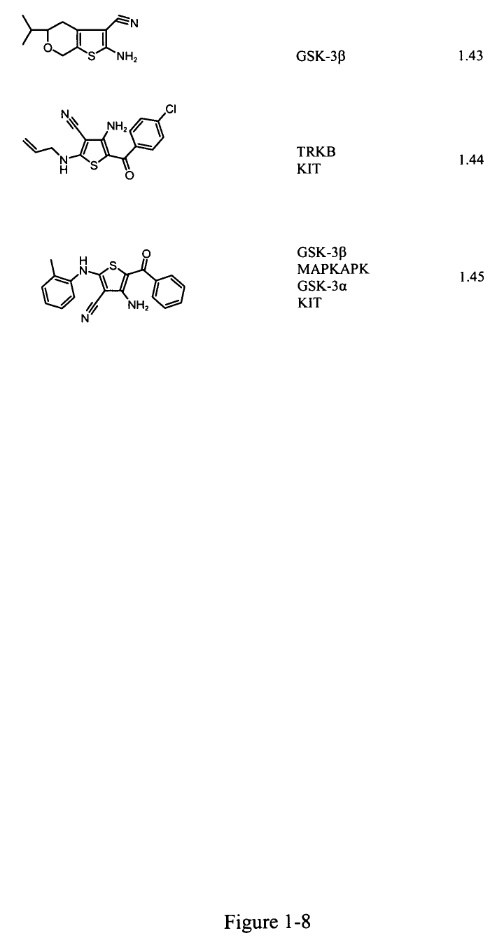

In certain embodiments of compounds of Formula (III), the at least one compound has the structure of any of compounds 1.1 to 1.45 listed in FIG. 1, a stereoisomer thereof, a pharmaceutically acceptable salt thereof, a hydrate thereof, or a solvate of any of the foregoing.

In certain embodiments, compounds of the present disclosure are directed to compounds of Formula (IV): 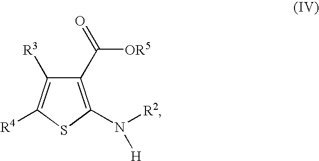
a stereoisomer thereof a pharmaceutically acceptable salt thereof a hydrate thereof, or a solvate of any of the foregoing, wherein:
R² is chosen from H, —CHO, heteroarylalkyl, substituted heteroarylalkyl, heterocycloalkylalkyl, substituted heterocycloalkylalkyl, alkylsulfonyl, substituted alkylsulfonyl, and -ZR⁶, wherein
Z is carbonyl; and
R⁶ is chosen from H, —COOH, alkyl, substituted alkyl, aryl, and substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, bicycloalkyl, substituted bicycloalkyl, bicycloheteroalkyl, and substituted bicycloheteroalkyl;
R³ is chosen from H, halogen, alkyl, substituted alkyl, aryl, substituted aryl, arylalkyl, substituted arylalkyl, heterocycloalkyl, substituted heterocycloalkyl, heteroaryl, substituted heteroaryl, heteroarylalkyl, substituted heteroarylalkyl, and dialkylamin;
R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, heteroarylalkyl, and substituted heteroarylalkyl;
or R³ and R⁴ together with the atoms to which R³ and R⁴ are attached form a cycloalkyl, substituted cycloalkyl, heterocycloalkyl, substituted heterocycloalkyl, bicycloalkyl, substituted bicycloalkyl, bicycloheteroalkyl, or substituted bicycloheteroalkyl ring; and
R⁵ is chosen from H, alkyl, substituted alkyl, arylalkyl, and substituted arylalkyl;
with the provisos that
R³ is not chosen from H, 2-aminopyrimidine, substituted 2-aminopyrimidine, 2-aminopyridine, substituted 2-aminopyridine, aminotriazine, or substituted aminotriazine; and R⁴ is not chosen from 2-aminopyrimidine, substituted 2-aminopyrimidine, 2-aminopyridine, substituted 2-aminopyridine, aminotriazine, or substituted aminotriazine;
and wherein the compound of Formula (IV), a stereoisomer thereof, a pharmaceutically acceptable salt thereof, a hydrate thereof, or a solvate of any of the foregoing, exhibits ATP-utilizing enzyme inhibitory activity.

In certain embodiments of compounds of Formula (IV), R³ is chosen from H, C₁₋₆alkyl, substituted C₁₋₆ alkyl, C₅₋₁₂ aryl, substituted C₅₋₁₂ aryl, C₅₋₁₂ heteroaryl, substituted C₅₋₁₂ heteroaryl, C₆₋₁₈ heterocycloalkyl, substituted C₆₋₁₈heterocycloalkyl, C₆₋₁₈ arylalkyl, substituted C₆₋₁₈ arylalkyl, C₂₋₆ dialkylamino, and substituted C₂₋₆ dialkylamino.

In certain embodiments of compounds of Formula (IV), wherein R³ is chosen from H, C₁₋₆ alkyl, substituted C₁₋₆ alkyl, C₅₋₁₂ aryl, substituted C₅₋₁₂ aryl, C₅₋₁₂ heteroaryl, substituted C₅₋₁₂ heteroaryl, C₆₋₁₈ heterocycloalkyl, substituted C₆₋₁₈ heterocycloalkyl, C₆₋₁₈ arylalkyl, substituted C₆₋₁₈ arylalkyl, C₂₋₆ dialkylamino, and substituted C₂₋₆ dialkylamino, the at least one substituent group is chosen from halogen, C₁₋₆ alkyl, C₁₋₆ alkoxy, C₁₋₆ alkylsulfonyl, C₅₋₁₂ aryl, substituted C₅₋₁₂ aryl NH₂, —CF₃, nitro, and —NHC(O)CH₃.

In certain embodiments of compounds of Formula (IV), R⁵ is chosen from H, C₁₋₆ alkyl, substituted C₁₋₆ alkyl, C₆₋₁₈ arylalkyl, and substituted C₆₋₁₈ arylalkyl.

In certain embodiments of compounds of Formula (IV), R⁵ is chosen from H, C₁₋₆ alkyl, and C₆₋₁₀ arylalkyl.

In certain embodiments of compounds of Formula (IV), R⁴ is chosen from H, C₁₋₆ alkyl, substituted C₁₋₆ alkyl, C₁₋₆ aminocarbonyl, substituted C₁₋₆ aminocarbonyl, C₁₋₆ carbonyl, substituted C₁₋₆ carbonyl, C₁₋₆ alkoxycarbonyl, substituted C₁₋₆ alkoxycarbonyl, C₅₋₁₂ aryl, substituted C₅₋₁₂ aryl, C₆₋₁₈ heteroarylalkyl, and substituted C₆₋₁₈heteroarylalkyl.

In certain embodiments of compounds of Formula (IV), wherein R⁴ is chosen from H, C₁₋₆ alkyl, substituted C₁₋₆ alkyl, C₁₋₆ aminocarbonyl, substituted C₁₋₆ aminocarbonyl, C₁₋₆ carbonyl, substituted C₁₋₆ carbonyl, C₁₋₆ alkoxycarbonyl, substituted C₁₋₆ alkoxycarbonyl, C₅₋₁₂ aryl, substituted C₅₋₁₂ aryl, C₆₋₁₈ heteroarylalkyl, and substituted C₆₋₁₈ heteroarylalkyl, the at least one substituent group is chosen from halogen, ═O, C₁₋₆ alkoxy, and C₁₋₆ alkyl.

In certain embodiments of compounds of Formula (IV), R³ and R⁴ together with the atoms to which R³ and R⁴ are attached form a C₅₋₁₂ cycloalkyl, substituted C₅₋₁₂ cycloalkyl, C₅₋₁₂ heterocycloalkyl, substituted C₅₋₁₂ heterocycloalkyl, C₅₋₁₂ bicycloalkyl, substituted C₅₋₁₂ bicycloalkyl, C₅₋₁₂ bicycloheteroalkyl, or substituted C₅₋₁₂ bicycloheteroalkyl ring.

In certain embodiments of compounds of Formula (IV), wherein R³ and R⁴ together with the atoms to which R³ and R⁴ are attached form a C₅₋₁₂ cycloalkyl, substituted C₅₋₁₂ cycloalkyl, C₅₋₁₂ heterocycloalkyl, substituted C₅₋₁₂ heterocycloalkyl, C₅₋₁₂ bicycloalkyl, substituted C₅₋₁₂ bicycloalkyl, C₅₋₁₂ bicycloheteroalkyl, or substituted C₅₋₁₂ bicycloheteroalkyl ring, the at least one substituent group is chosen from C₁₋₆ alkoxy, halogen, C₁₋₆ alkyl, C₅₋₁₂ aryl, substituted C₅₋₁₂ aryl, C₁₋₆ alkoxycarbonyl, substituted C₁₋₆ alkoxycarbonyl, C₆₋₁₂ arylalkyl, substituted C₆₋₁₂ arylalkyl, ═O, and ═N—OH.

In certain embodiments of compounds of Formula (IV), R² is chosen from H, —COOH, —CH═O, C₁₋₆ alkylsulfonyl, substituted C₁₋₆ alkylsulfonyl, C₆₋₁₂ heterocycloalkylalkyl, substituted C₆₋₁₂ heterocycloalkylalkyl, C₆₋₁₂ heteroarylalkyl, substituted C₆₋₁₂ heteroarylalkyl, and —COR⁶ wherein, R⁶ is chosen from C₁₋₁₀ alkyl, substituted C₁₋₁₀ alkyl, C₁₋₁₀ heteroalkyl, substituted C₁₋₁₀ heteroalkyl, C₃₋₁₂ cycloalkyl, substituted C₃₋₁₂ cycloalkyl, C₃₋₁₂ heterocycloalkyl, substituted C₃₋₁₂ heterocycloalkyl, C₅₋₁₃ aryl, substituted C₅₋₁₂ aryl, C₅₋₁₂ heteroaryl, substituted C₅₋₁₂ heteroaryl, C₆₋₁₈ cycloalkylalkyl, substituted C₆₋₁₈ cycloalkylalkyl, C₆₋₁₈ heterocycloalkylalkyl, substituted C₆₋₁₈ heterocycloalkylalkyl, C₆₋₁₈ arylalkyl, substituted C₆₋₁₈ arylalkyl, C₆₋₁₈ heteroarylalkyl, substituted C₆₋₁₈ sheteroarylalkyl, C₅₋₁₂ bicycloalkyl, substituted C₅₋₁₂ bicycloalkyl, C₅₋₁₂ bicycloheteroalkyl, and substituted C₅₋₁₂ bicycloheteroalkyl.

In certain embodiments of compounds of Formula (IV), wherein R² is chosen from H, —COOH, —CH═O, C₁₋₆ alkylsulfonyl, substituted C₁₋₆ alkylsulfonyl, C₆₋₁₂ heterocycloalkylalkyl, substituted C₆₋₁₂ heterocycloalkylalkyl, C₆₋₁₂ heteroarylalkyl, substituted C₆₋₁₂ heteroarylalkyl, and —COR⁶ wherein, R⁶ is chosen from C₁₋₁₀ alkyl, substituted C₁₋₁₀ alkyl, C₁₋₁₀ heteroalkyl, substituted C₁₋₁₀ heteroalkyl, C₃₋₁₂ cycloalkyl, substituted C₃₋₁₂ cycloalkyl, C₃₋₁₂ heterocycloalkyl, substituted C₃₋₁₂ heterocycloalkyl, C₅₋₁₂ aryl, substituted C₅₋₁₂ aryl, C₅₋₁₂ heteroaryl, substituted C₅₋₁₂ heteroaryl, C₆₋₁₈ cycloalkylalkyl, substituted C₆₋₁₈ cycloalkylalkyl, C₆₋₁₈ heterocycloalkylalkyl, substituted C₆₋₁₈ heterocycloalkylalkyl, C₆₋₁₈ arylalkyl, substituted C₆₋₁₈ arylalkyl, C₆₋₁₈ heteroarylalkyl, substituted C₆₋₁₈ heteroarylalkyl, C₅₋₁₂ bicycloalkyl, substituted C₅₋₁₂ bicycloalkyl, C₅₋₁₂ bicycloheteroalkyl, and substituted C₅₋₁₂ bicycloheteroalkyl, the at least one substituent group is chosen from C₁₋₆ alkyl, substituted C₁₋₆ alkyl, C₁₋₆heteroalkyl, substituted C₁₋₆ heteroalkyl, C₁₋₆ alkoxy, substituted C₁₋₆ alkoxy, C₅₋₈ aryl, substituted C₅₋₈ aryl, C₅₋₈ heteroaryl, substituted C₅₋₈ heteroaryl, C₅₋₈ cycloalkyl, substituted C₅₋₈ cycloalkyl, C₅₋₈ heterocycloalkyl, substituted C₅₋₈ heterocycloalkyl, C₆₋₁₀ arylalkyl, substituted C₆₋₁₀ arylalkyl, C₆₋₁₀ heteroarylalkyl, substituted C₆₋₁₀ heteroarylalkyl, C₆₋₁₀ cycloalkylalkyl, substituted C₆₋₁₀ cycloalkylalkyl, C₆₋₁₀ heterocycloalkylalkyl, substituted C₆₋₁₀ heterocycloalkylalkyl, C₁₋₆ alkylsulfonyl, substituted C₁₋₆ alkylsulfonyl, halogen, —OH, ═O, nitro, —COOH, —CF₃, ═NH, and —NH₂.

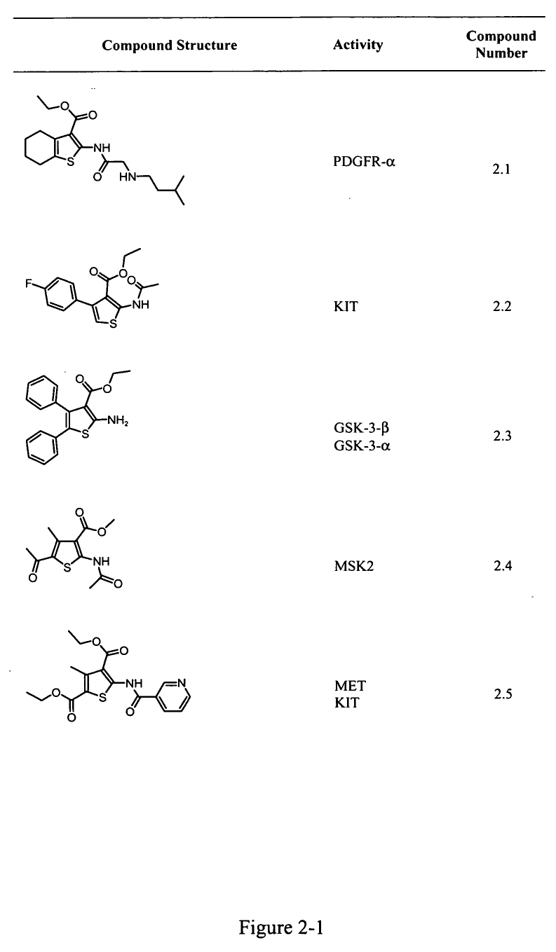

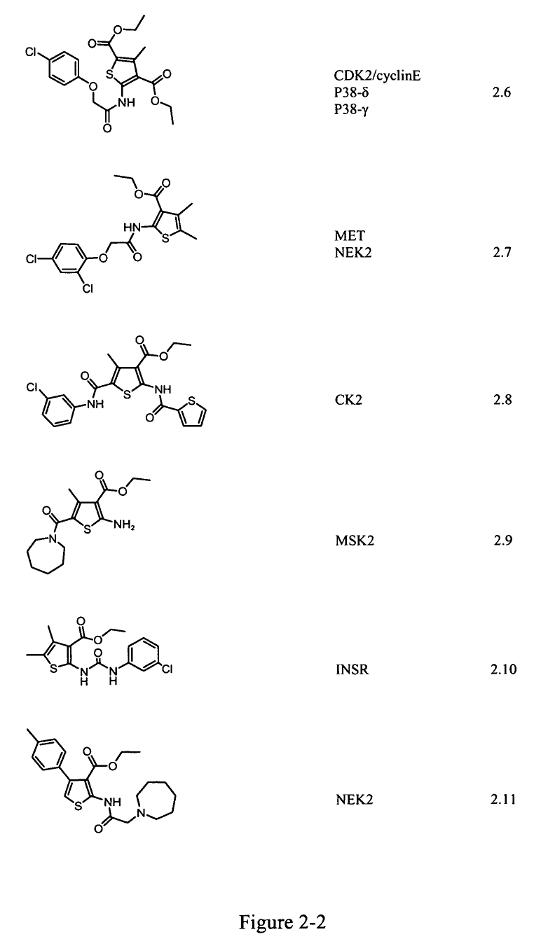

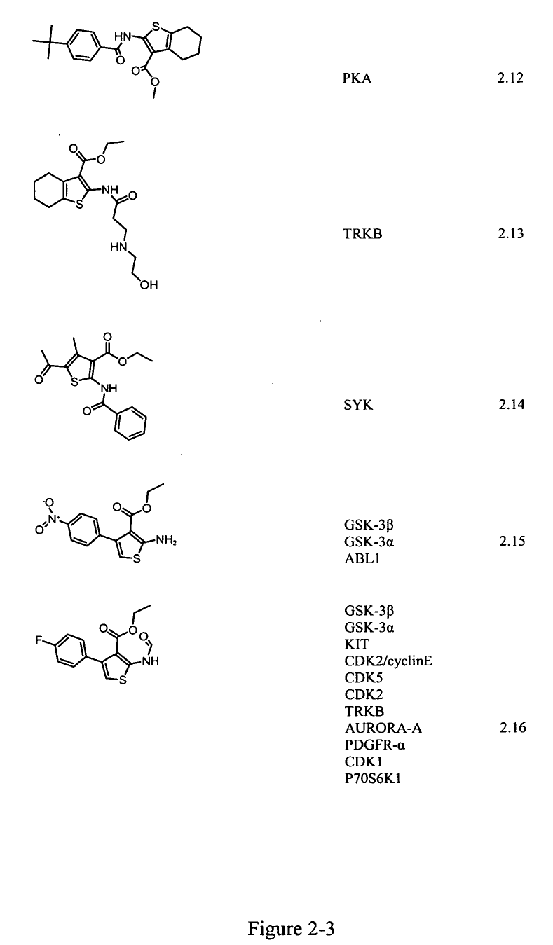

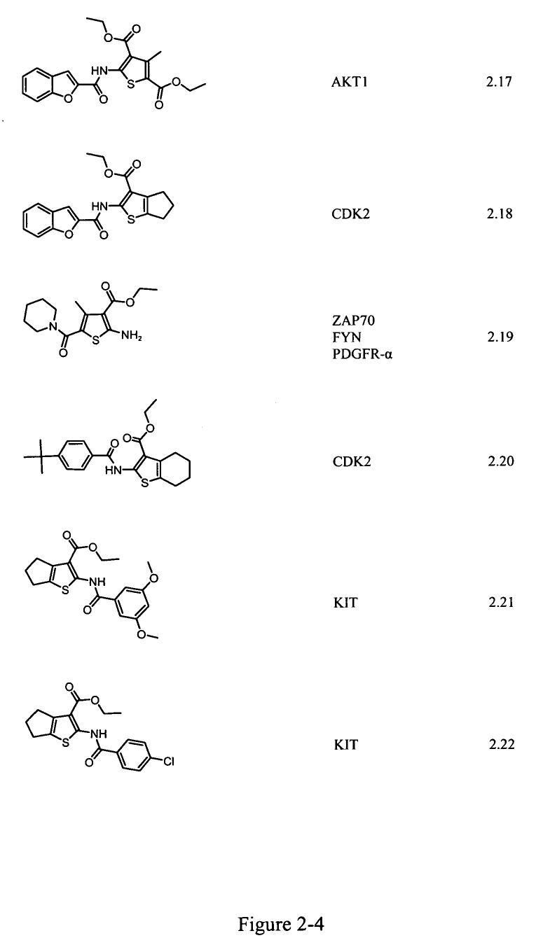

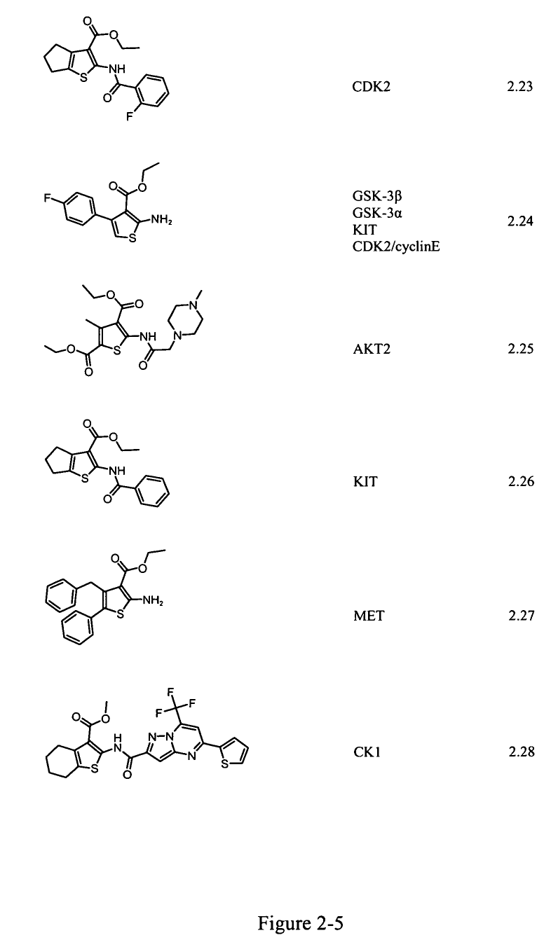

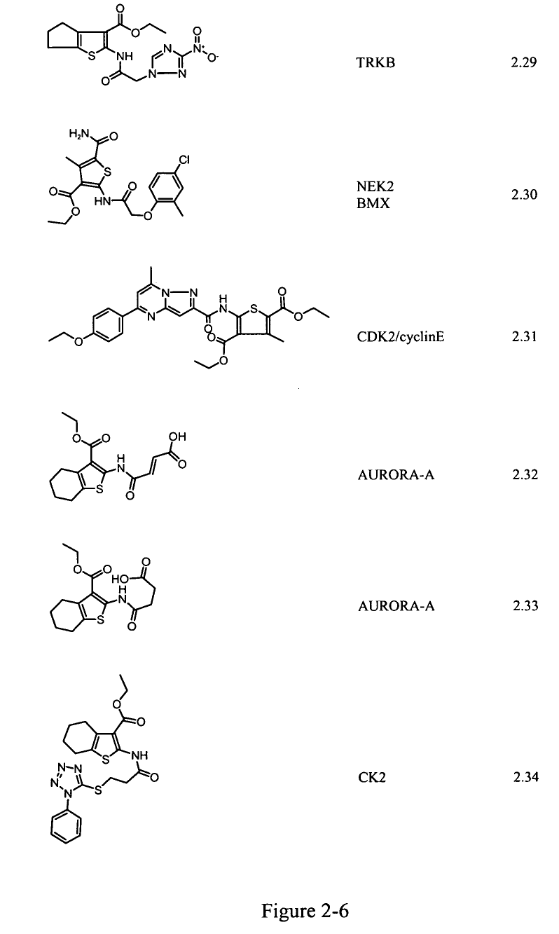

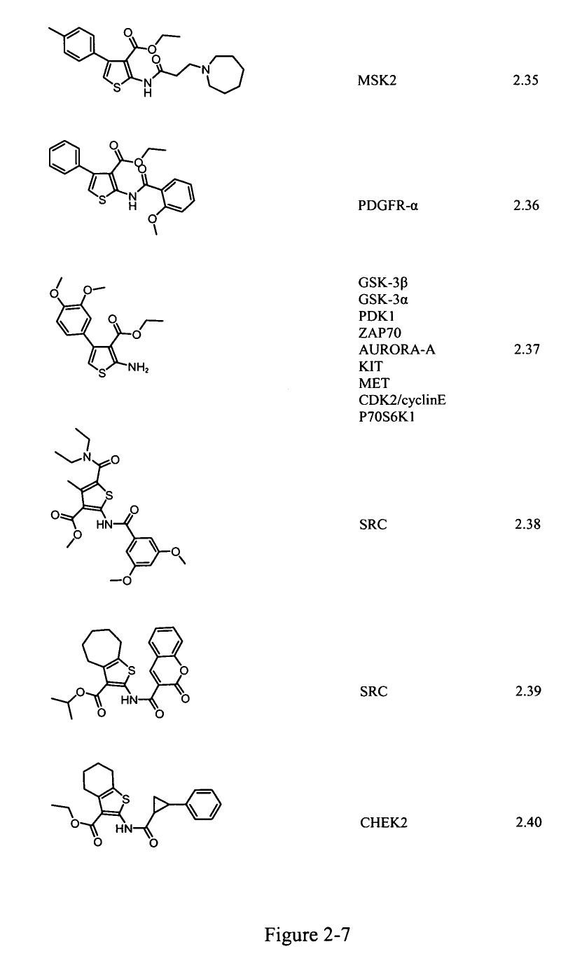

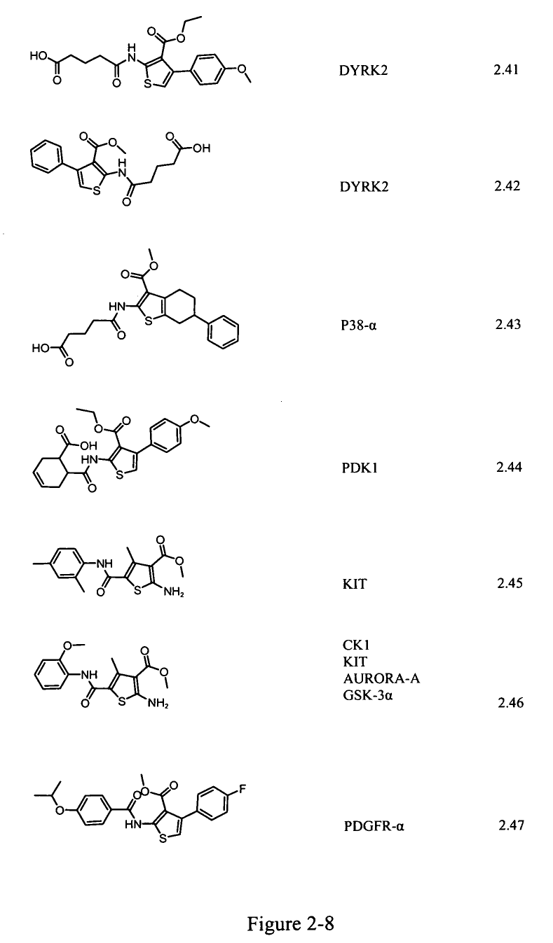

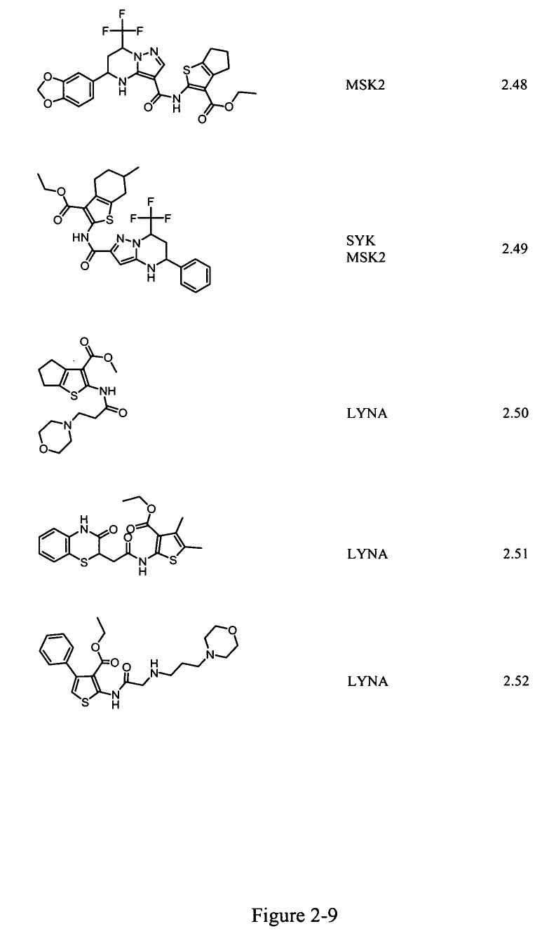

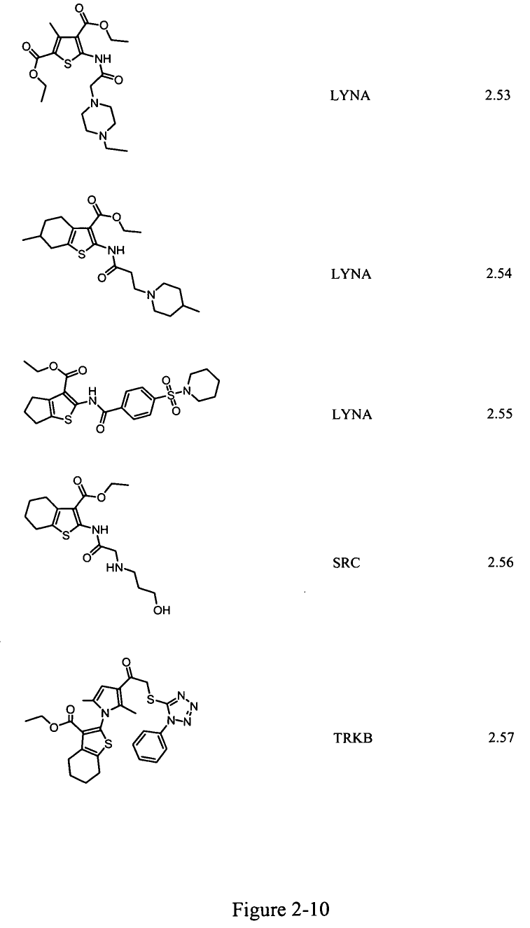

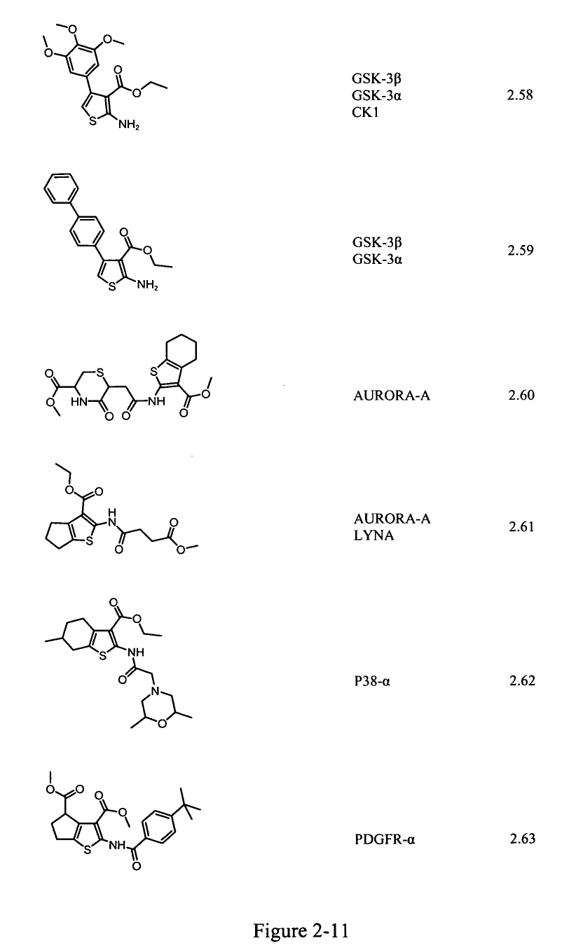

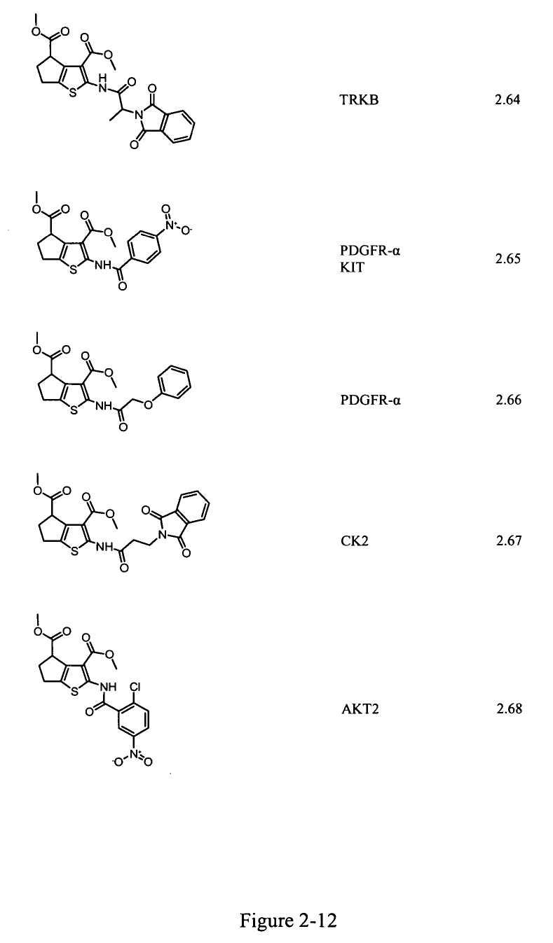

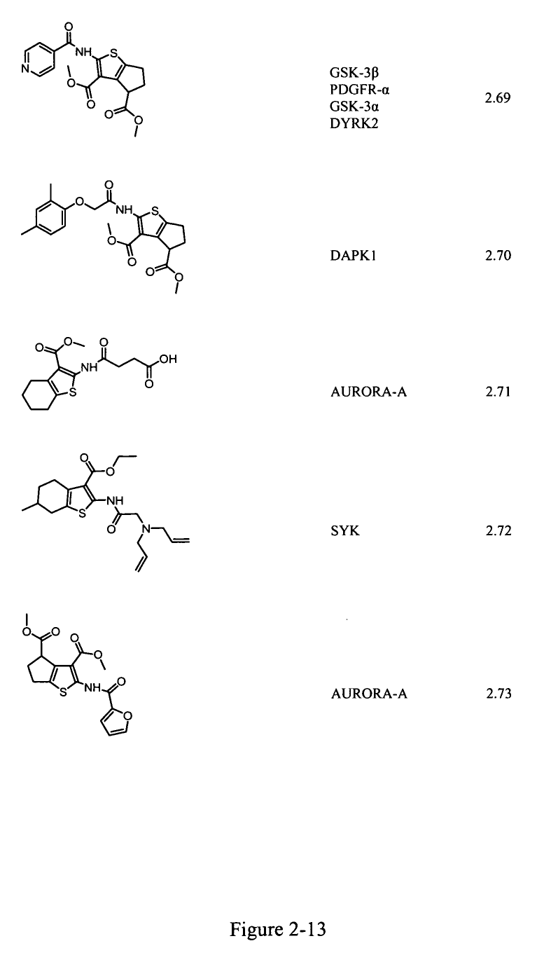

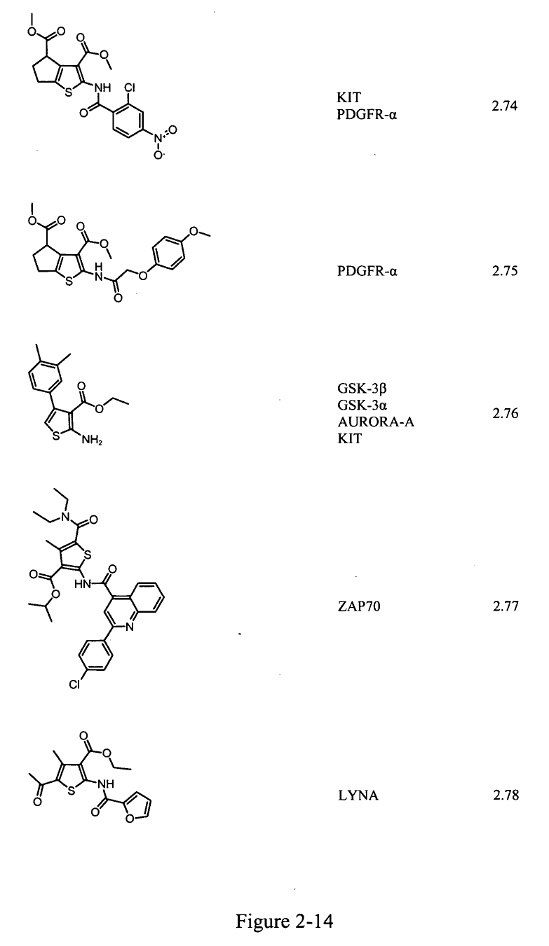

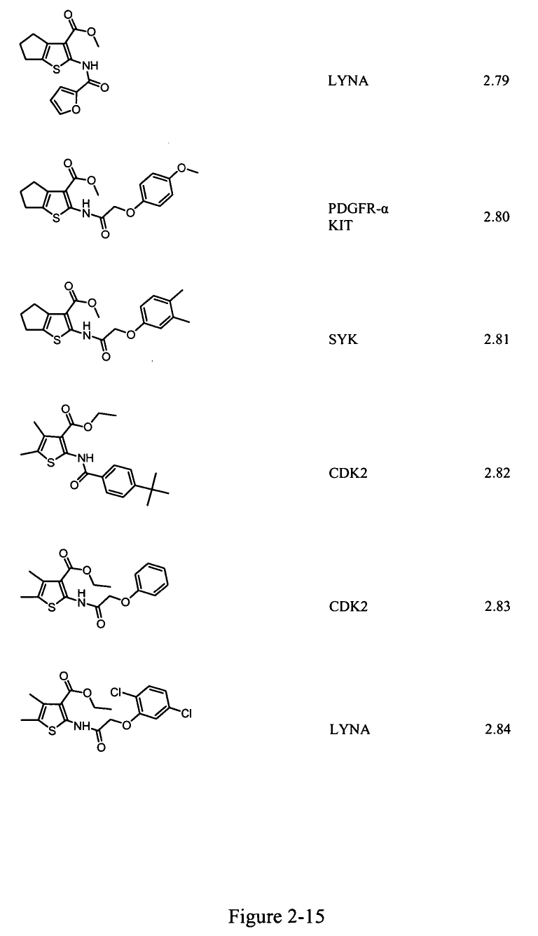

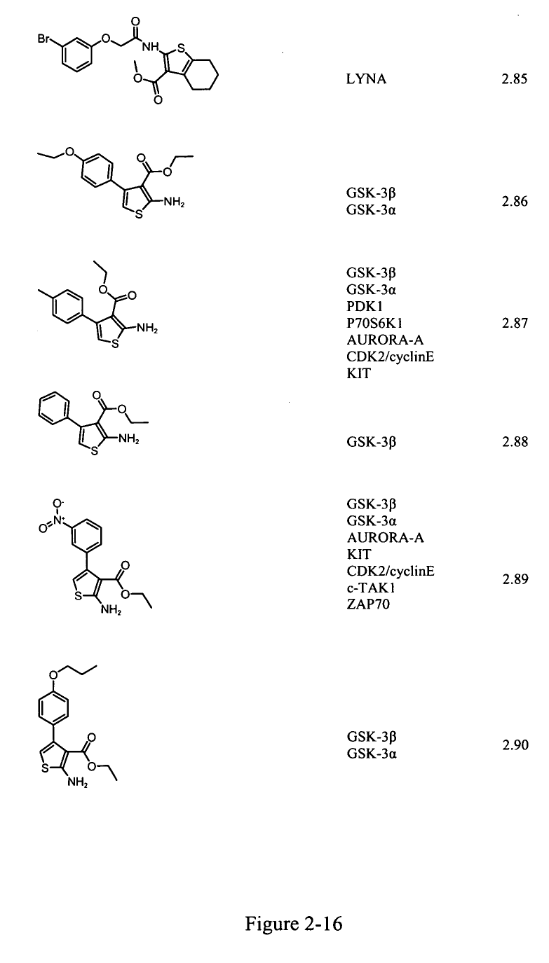

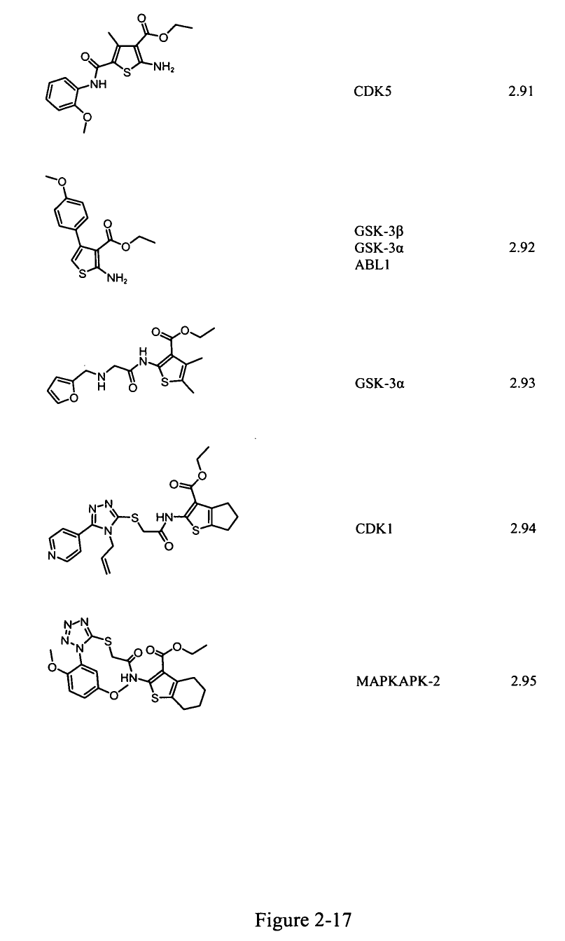

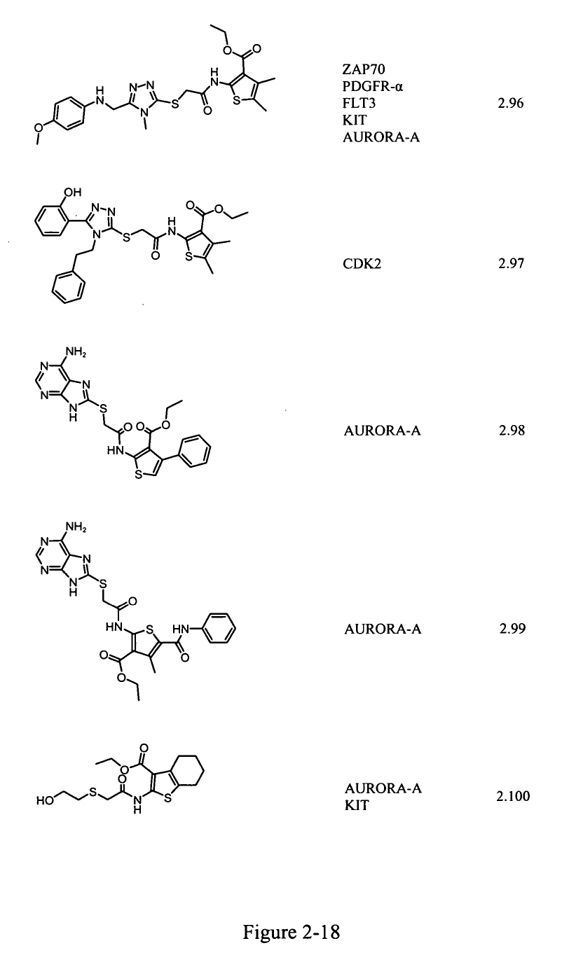

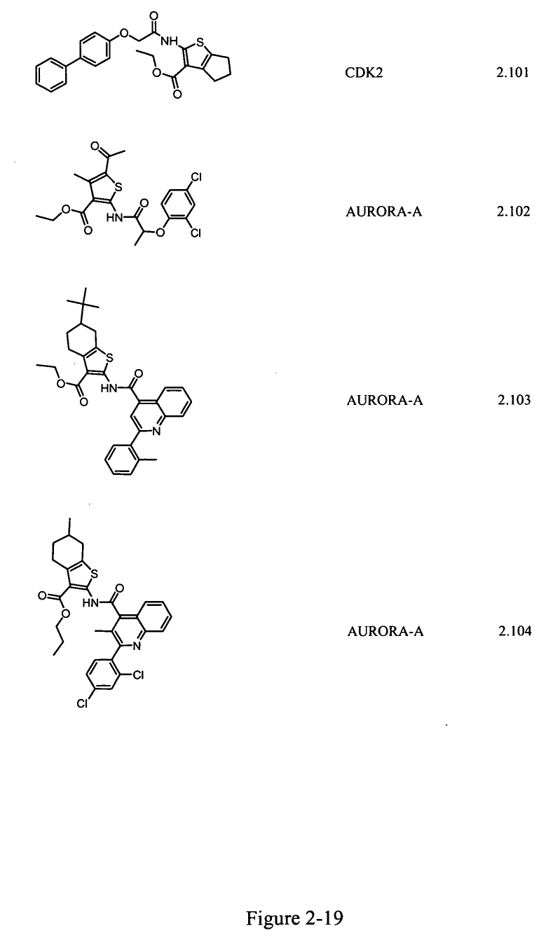

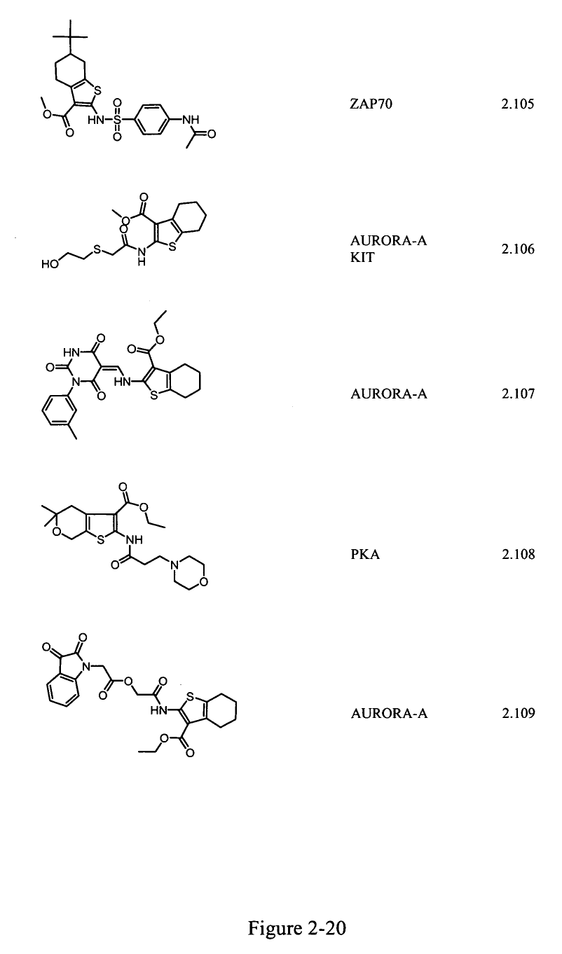

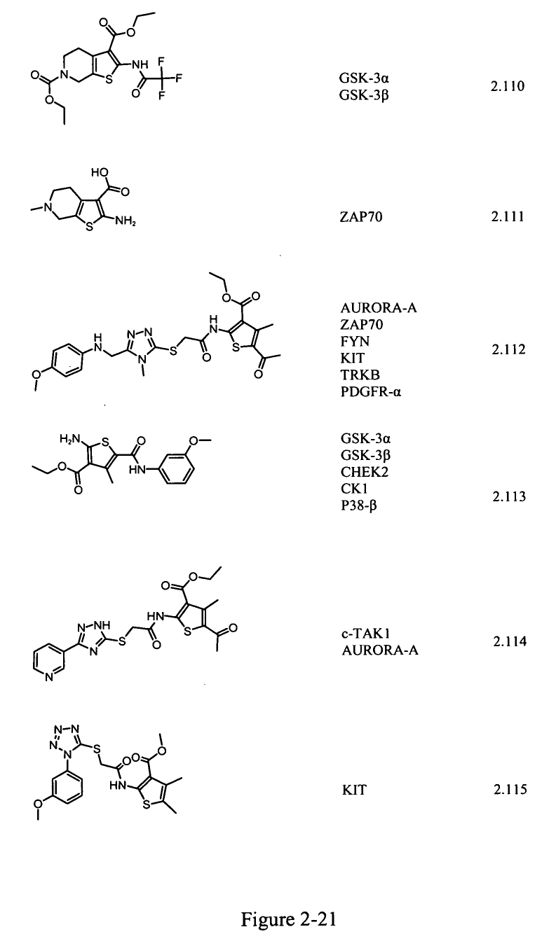

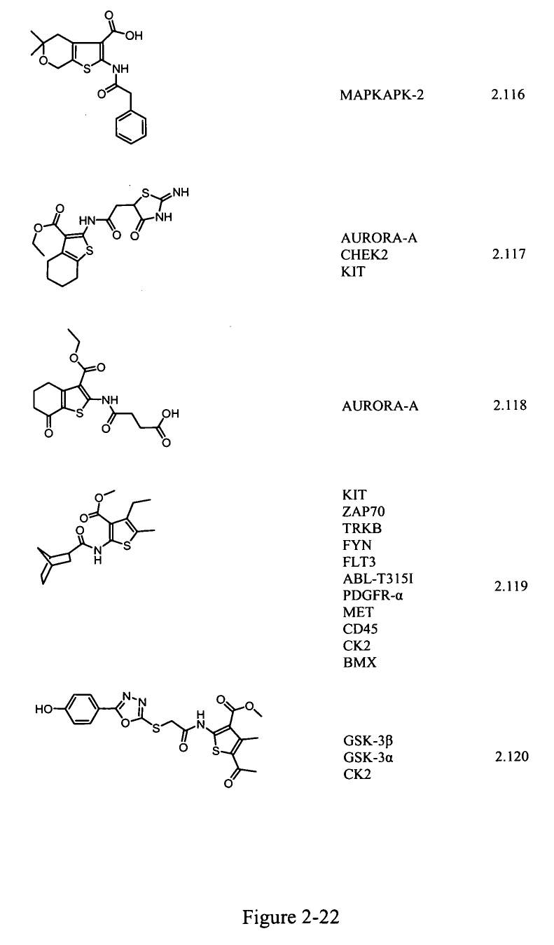

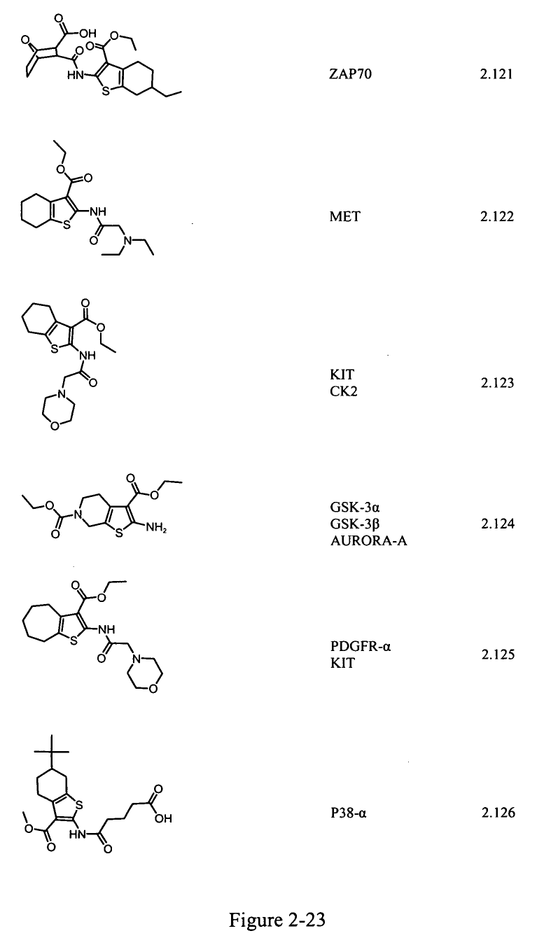

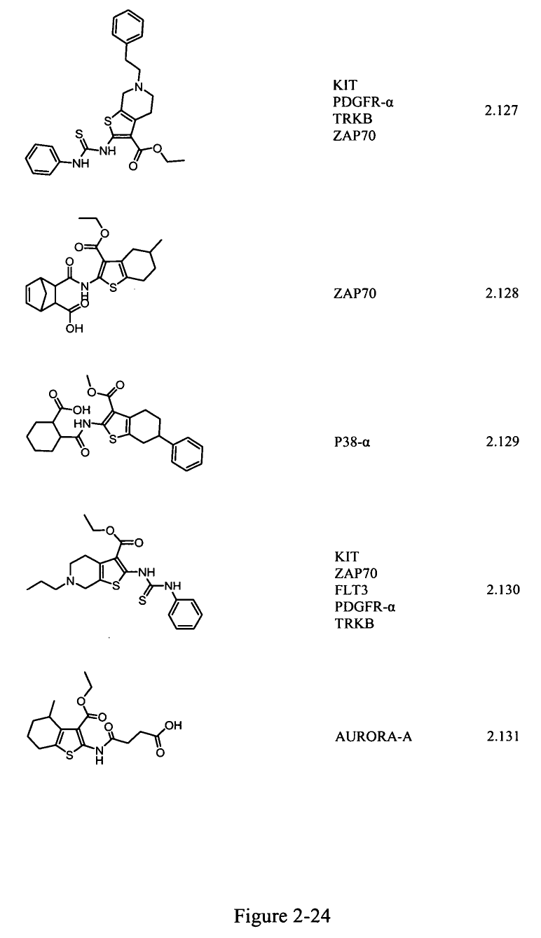

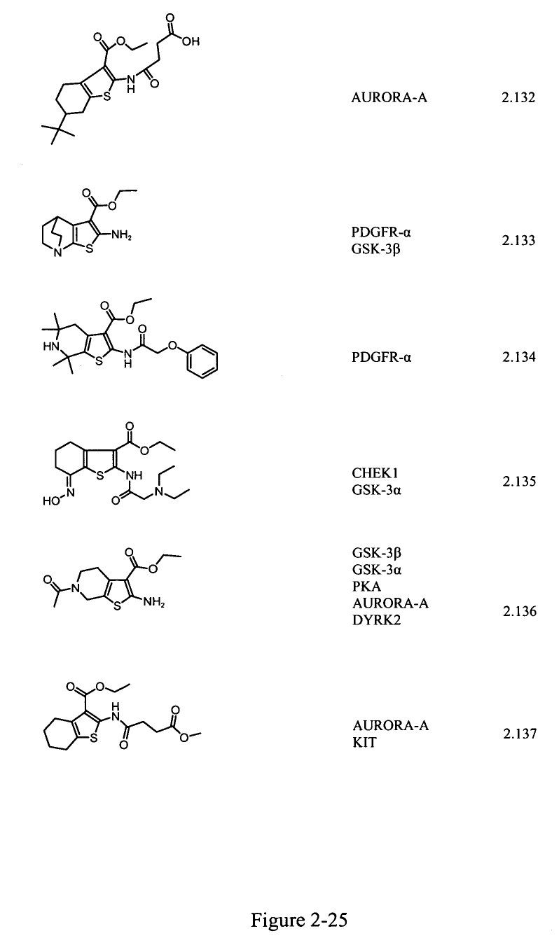

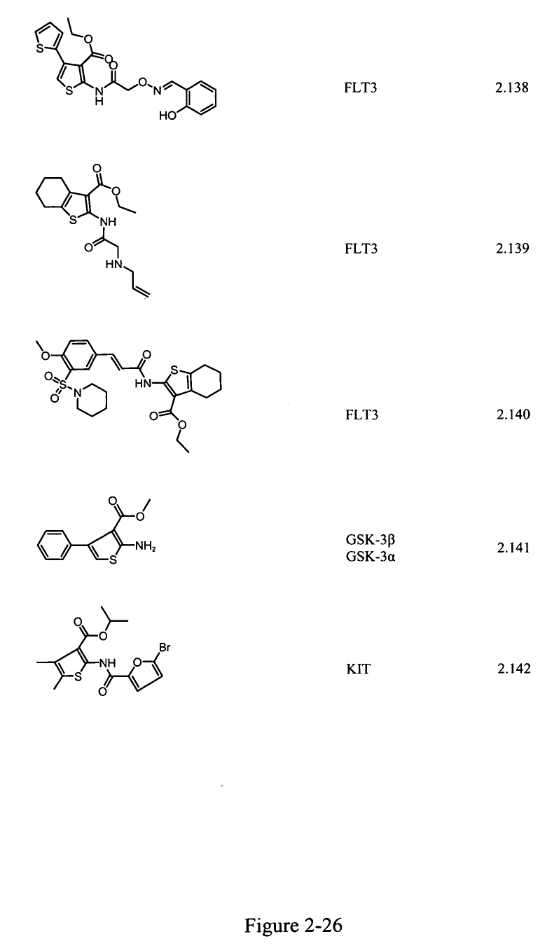

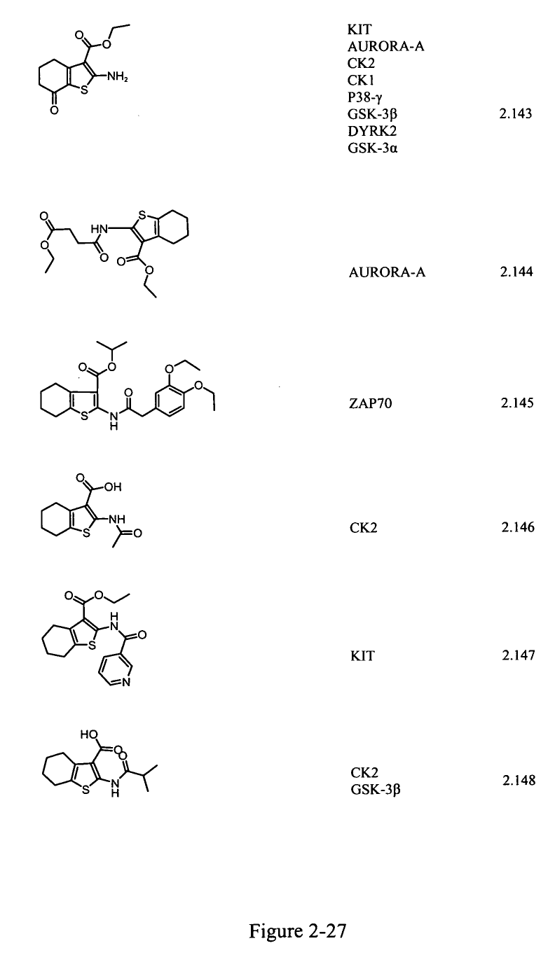

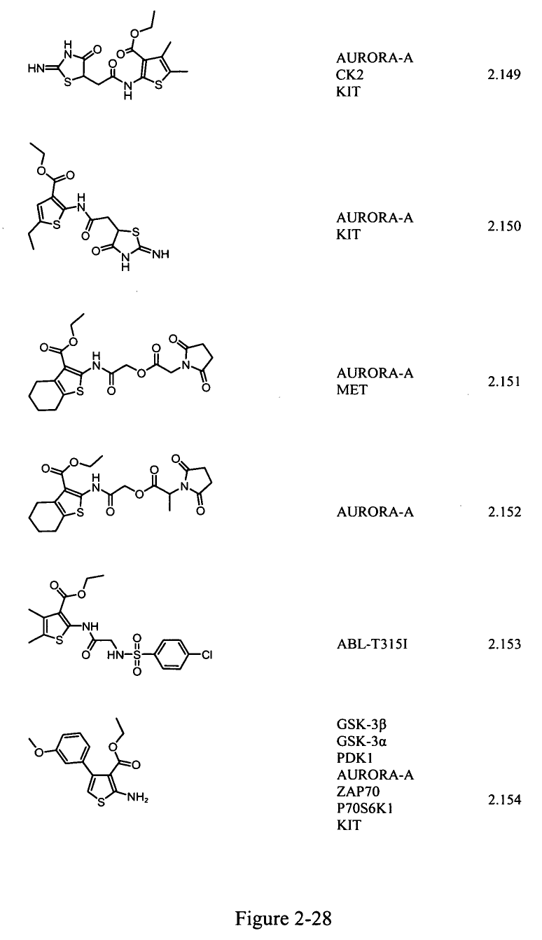

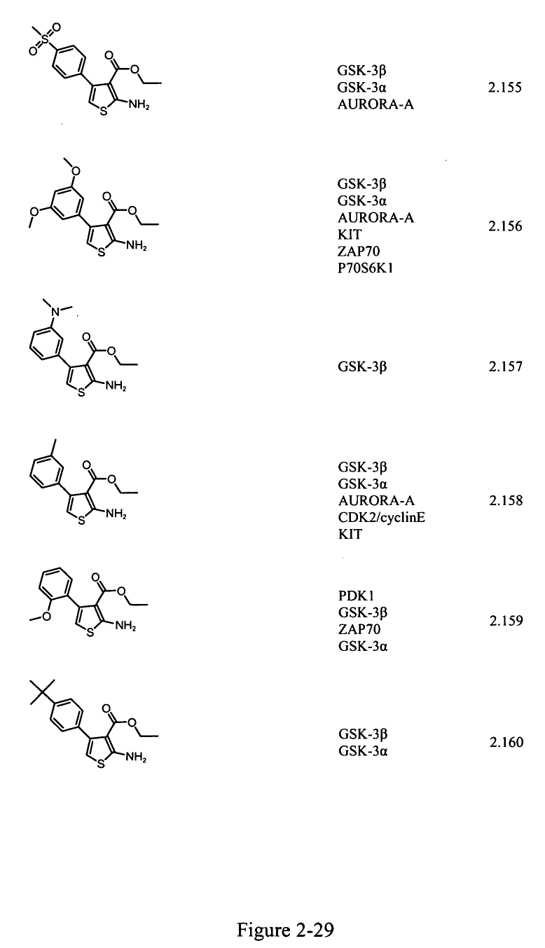

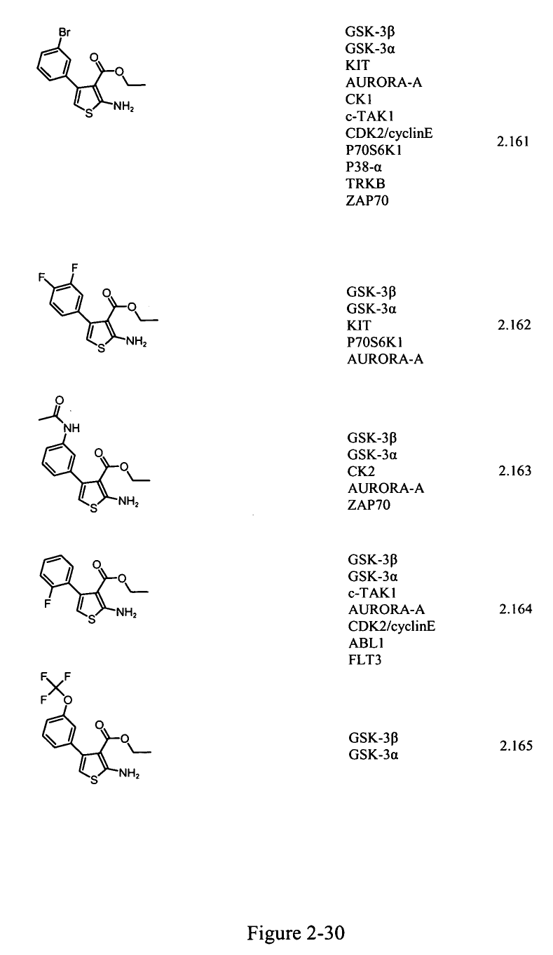

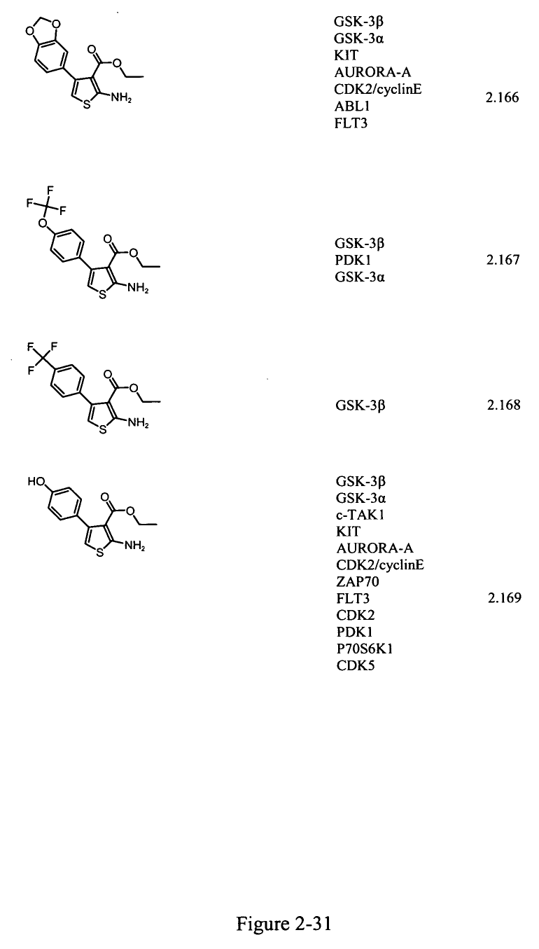

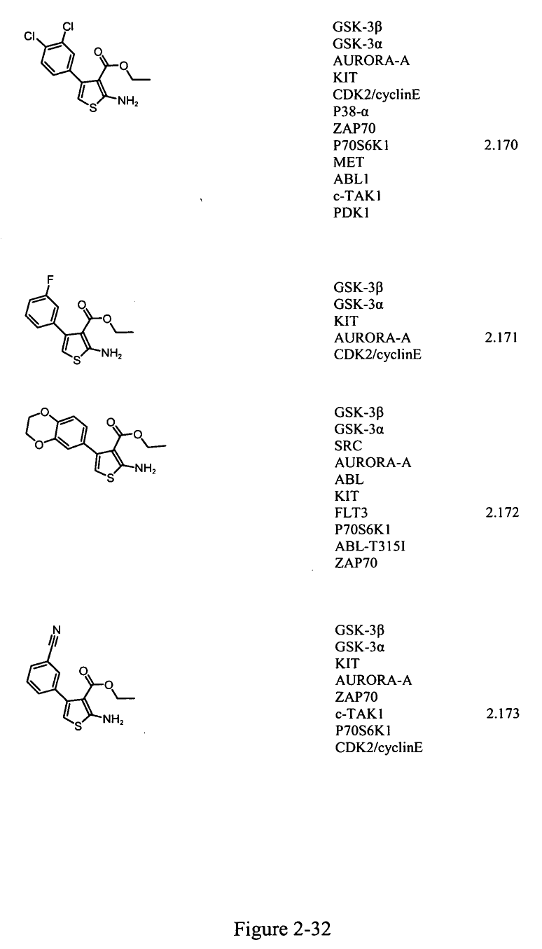

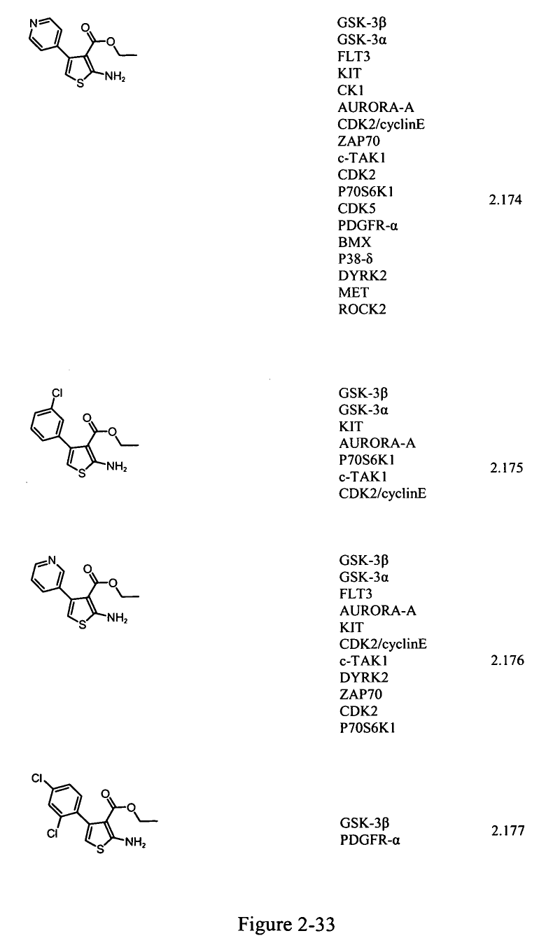

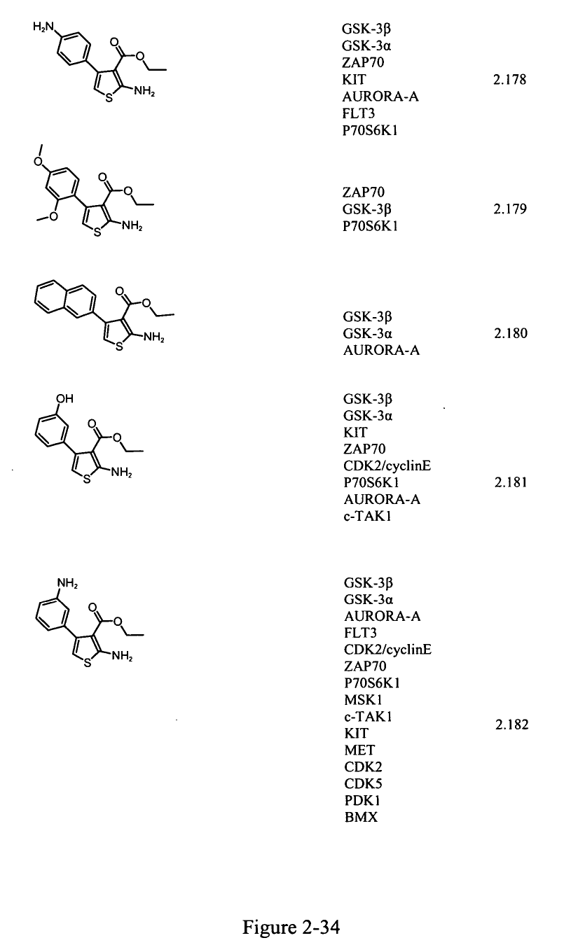

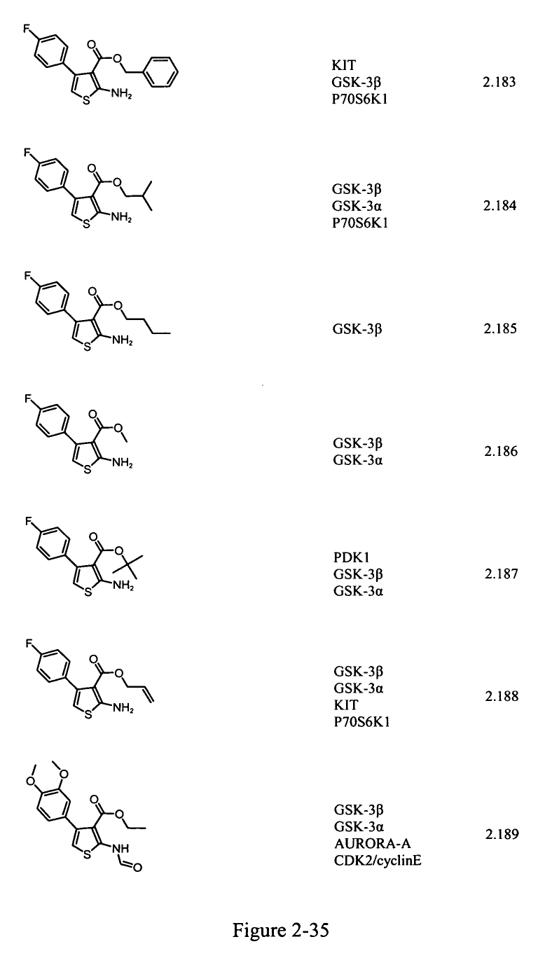

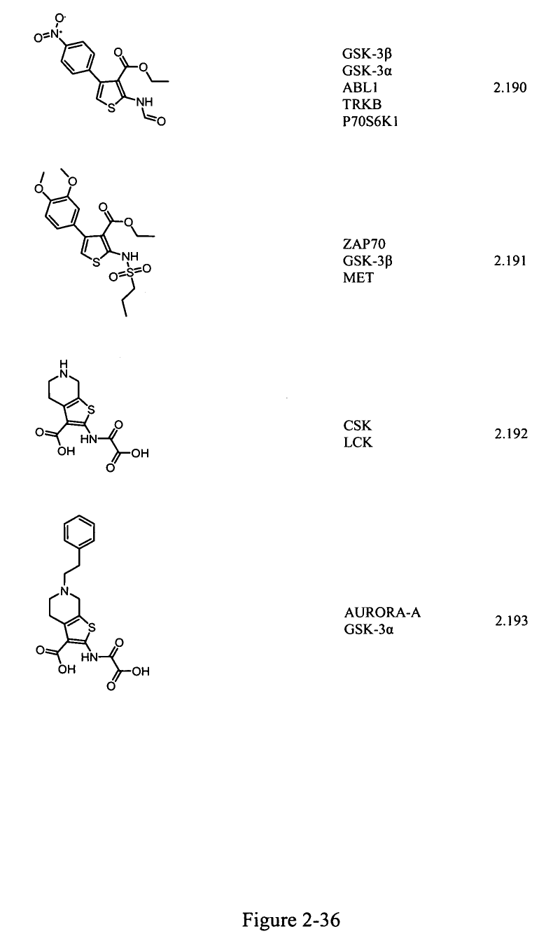

In certain embodiments of compounds of Formula (IV), the at least one compound has the structure of any of compounds 2.1 to 2.193 listed in FIG. 2, a stereoisomer thereof, a pharmaceutically acceptable salt thereof, a hydrate thereof, or a solvate of any of the foregoing.

In certain embodiments, compounds of the present disclosure are directed to compounds of Formula (V): 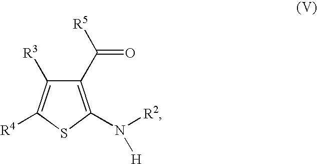
a stereoisomer thereof, a pharmaceutically acceptable salt thereof, a hydrate thereof, or a solvate of any of the foregoing, wherein:
R² is chosen from H, and -ZR⁶ wherein
Z is carbonyl; and
R⁶ is chosen from alkyl, substituted alkyl, heteroalkyl, substituted heteroalkyl, aryl, substituted aryl, heteroaryl, substituted heteroaryl, heterocycloalkylalkyl, and substituted heterocycloalkylalkyl;
R³ is chosen from H, halogen, alkyl, and substituted alkyl;
R⁴ is chosen from H, halogen, alkyl, and substituted alkyl;
or R³ and R⁴ together with the atoms to which R³ and R⁴ are attached form a cycloalkyl, substituted cycloalkyl ring;
R⁵ is chosen from H, aryl, substituted aryl, heteroaryl, and substituted heteroaryl;
and wherein the compound of Formula (V), a stereoisomer thereof, a pharmaceutically acceptable salt thereof, a hydrate thereof, or a solvate of any of the foregoing, exhibits ATP-utilizing enzyme inhibitory activity.

In certain embodiments of compounds of Formula (V), R⁴ is chosen from H, C₁₋₆alkyl, substituted C₁₋₆alkyl.

In certain embodiments of compounds of Formula (V), R³ is chosen from H, C₁₋₆alkyl, substituted C₁₋₆alkyl.

In certain embodiments of compounds of Formula (V), R³ and R⁴ together with the carbon atoms to which R³ and R⁴ are attached form a C₅₋₈ cycloalkyl or substituted C₅₋₈ cycloalkyl ring.

In certain embodiments of compounds of Formula (V), R⁵ is chosen from C₅₋₁₂ aryl, substituted C₅₋₁₂ aryl, C₅₋₁₂ heteroaryl, and substituted C₅₋₁₂ heteroaryl.

In certain embodiments of compounds of Formula (V), wherein R⁵ is chosen from C₅₋₁₂ aryl, substituted C₅₋₁₂ aryl, C₅₋₁₂ heteroaryl, and substituted C₅₋₁₂ heteroaryl, the at least one substituent group is chosen from halogen, C₁₋₆ alkyl, and C₁₋₆ alkoxy.

In certain embodiments of compounds of Formula (V), R⁵ is chosen from C₅₋₆ aryl, substituted C₅₋₆ aryl, C₅₋₆ heteroaryl, and substituted C₅₋₆ heteroaryl.

In certain embodiments of compounds of Formula (V), wherein R⁵ is chosen from C₅₋₆ aryl, substituted C₅₋₆ aryl, C₅₋₆ heteroaryl, and substituted C₅₋₆ heteroaryl, the at least one substituent group is chosen from halogen, C₁₋₆ alkyl, and C₁₋₆ alkoxy.

In certain embodiments of compounds of Formula (V), R² is chosen from H, and —C(O)R⁶ wherein R⁶ is chosen from C₁₋₁₀ alkyl, substituted C₁₋₁₀ alkyl, C₁₋₁₀ heteroalkyl, substituted C₁₋₁₀ heteroalkyl, C₅₋₁₂ aryl, substituted C₅₋₁₂ aryl, C₅₋₁₂ heteroaryl, substituted C₅₋₁₂ heteroaryl, C₆₋₁₈ heterocycloalkylalkyl, and substituted C₆₋₁₈ heterocycloalkylalkyl.

In certain embodiments of compounds of Formula (V), wherein R² is chosen from H, and —C(O)R⁶ wherein R⁶ is chosen from C₁₋₁₀ alkyl, substituted C₁₋₁₀ alkyl, C₁₋₁₀ heteroalkyl, substituted C₁₋₁₀ heteroalkyl, C₅₋₁₂ aryl, substituted C₅₋₁₂ aryl, C₅₋₁₂ heteroaryl, substituted C₅₋₁₂ heteroaryl, C₆₋₁₈ heterocycloalkylalkyl, and substituted C₆₋₁₈ heterocycloalkylalkyl, the at least one substituent group is chosen from halogen, —OH, and C₁₋₆ alkyl.

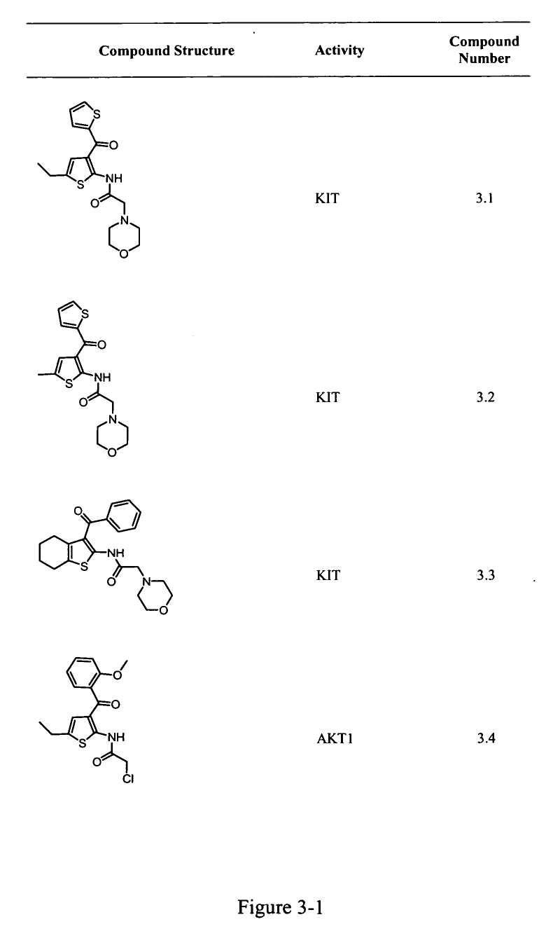

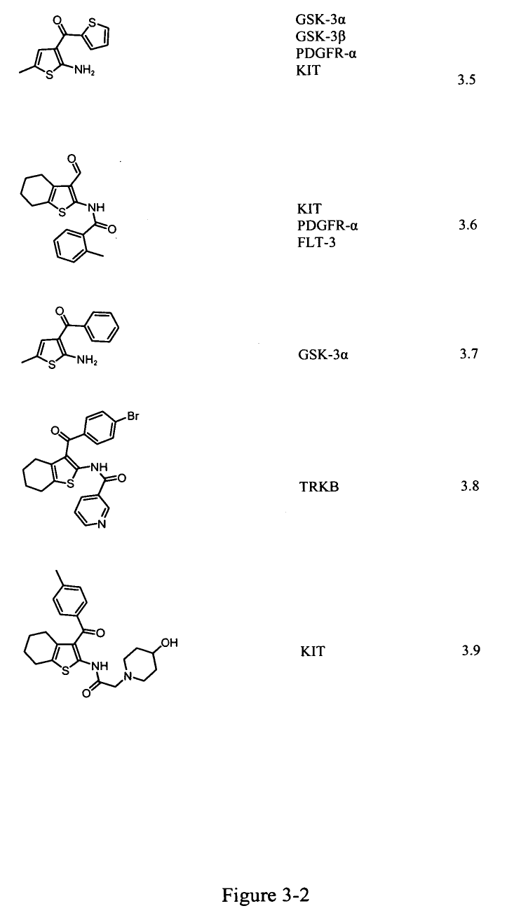

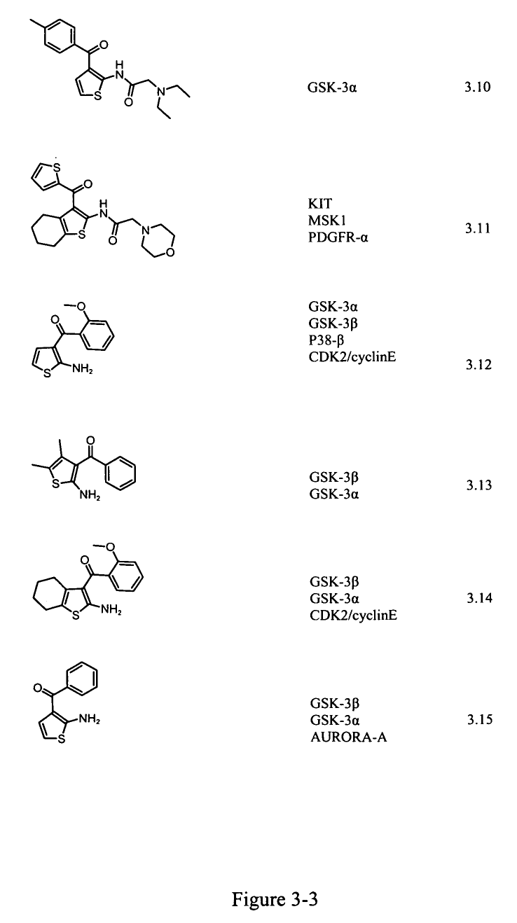

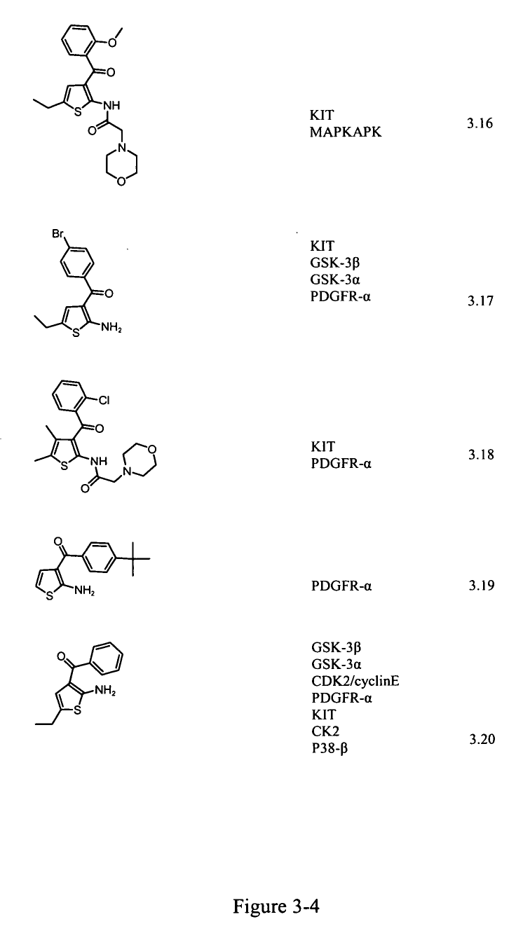

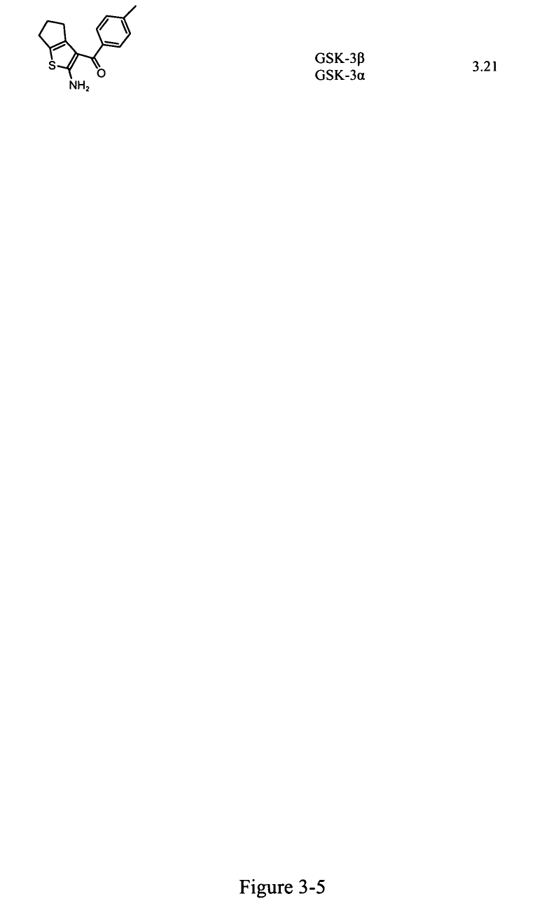

In certain embodiments of compounds of Formula (V), the at least one compound has the structure of any of compounds 3.1 to 3.21 listed in FIG. 3, a stereoisomer thereof, a pharmaceutically acceptable salt thereof, a hydrate thereof, or a solvate of any of the foregoing.

In certain embodiments, compounds of the present disclosure are directed to compounds of Formula (VI): 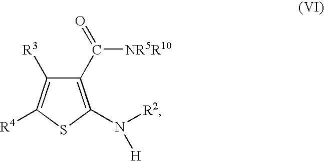
a stereoisomer thereof, a pharmaceutically acceptable salt thereof, a hydrate thereof, or a solvate of any of the foregoing, wherein:
R² is chosen from H, aryl, substituted aryl, heteroaryl, substituted heteroaryl, heterocycloalkyl, substituted heterocycloalkyl, alkylsulfonyl, substituted, alkylsulfonyl, heteroalkylsulfonyl, substituted heteroalkylsulfonyl, and -ZR⁶, wherein
Z is carbonyl; and
R⁶ is chosen from H, alkyl, substituted alkyl, aryl, and substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, arylalkyl, substituted arylalkyl, heteroarylalkyl, and substituted heteroalkyl;
R³ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
R⁴ is chosen from H, alkyl, substituted alkyl, aryl, substituted aryl, arylalkyl, substituted arylalkyl, heteroaryl, substituted heteroaryl, heteroarylalkyl, and substituted heteroarylalkyl;
or R³ and R⁴ together with the atoms to which R³ and R⁴ are attached form a cycloalkyl, substituted cycloalkyl, heterocycloalkyl, or substituted heterocycloalkyl ring;
R⁵ is chosen from H, alkyl, substituted alkyl;
R¹⁰ is chosen from H, alkyl, substituted alkyl, aryl, substituted aryl, arylalkyl, substituted arylalkyl, heteroalkyl, substituted heteroalkyl, heteroalkyl, and substituted heteroalkyl;
or, R⁵ and R¹⁰ together with the atoms to which R⁵ and R¹⁰ are attached form a cycloalkyl, substituted cycloalkyl, heterocycloalkyl, or substituted heterocycloalkyl ring; and
with the provisos that
when R³ is H, and R² is C(═O)NR¹²R¹¹, and R¹¹ is H, then R¹² is not alkyl or substituted alkyl; and
when R¹ is H, and R⁵ is H, then R¹⁰ is not H;
and wherein the compound of Formula (VI), a stereoisomer thereof, a pharmaceutically acceptable salt thereof, a hydrate thereof, or a solvate of any of the foregoing, is an inhibitor of at least one ATP-utilizing enzyme.

In certain embodiments of compounds of Formula (VI), R² is chosen from H, aryl, substituted aryl, heteroaryl, substituted heteroaryl, arylalkyl, substituted arylalkyl, heteroarylalkyl, substituted arylalkyl, cycloalkyl, substituted cycloalkyl, heterocycloalkyl, substituted heterocycloalkyl, alkylsulfonyl, substituted, alkylsulfonyl, heteroalkylsulfonyl, substituted heteroalkylsulfonyl, and -ZR⁶, wherein
Z is carbonyl, and
R⁶ is chosen from H, alkyl, substituted alkyl, aryl, and substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, arylalkyl, substituted arylalkyl, heteroarylalkyl, and substituted heteroarylalkyl;
R³is chosen from alkyl, substituted alkyl, aryl, substituted aryl, and H;
R⁴ is chosen from H, halogen, alkyl, substituted alkyl, aryl, substituted aryl, arylalkyl, and substituted arylalkyl; or
R³ and R⁴ together with the atoms to which R³ and R⁴ are attached, form a cycloalkyl, substituted cycloalkyl, heterocycloalkyl, or substituted heterocycloalkyl ring;
R⁵ is H; and
R¹⁰ is chosen from H, alkyl, substituted alkyl, aryl, substituted aryl, arylalkyl, and substituted arylalkyl;
or R⁵ and R¹⁰ together with the atoms to which R⁵ and R¹⁰ are attached form a heterocycloalkyl or substituted heterocycloalkyl ring.

In certain embodiments of compounds of Formula (VI), R³ is chosen from H, C₁₋₆ alkyl, substituted C₁₋₆ alkyl, C₅₋₁₀ aryl, and substituted C₅₋₁₀ aryl.

In certain embodiments of compounds of Formula (VI), R³ is chosen from H, methyl, and phenyl.

In certain embodiments of compounds of Formula (VI), R⁵ is chosen from H, and R¹⁰ is chosen from H, C₁₋₈ alkyl, substituted C₁₋₈ alkyl, C₁₋₁₂ heteroalkyl, substituted C₁₋₁₂ heteroalkyl, C₅₋₁₀ aryl, substituted C₅₋₁₀ aryl, C₆₋₁₂ arylalkyl, and substituted C₆₋₁₂ arylalkyl.

In certain embodiments of compounds of Formula (VI), wherein R⁵ is chosen from H, and R¹⁰ is chosen from H, C₁₋₈ alkyl, substituted C₁₋₈ alkyl, C₁₋₁₂ heteroalkyl, substituted C₁₋₁₂ heteroalkyl, C₅₋₁₀ aryl, substituted C₅₋₁₀ aryl, C₆₋₁₂ arylalkyl, and substituted C₆₋₁₂ arylalkyl, the at least one substituent group is chosen from halogen, C₁₋₆alkyl, C₁₋₆alkoxy, —OH, ═O, and —NH₂.

In certain embodiments of compounds of Formula (VI), R⁵ and R¹⁰ together with the atoms to which R⁵ and R¹⁰ are attached form a C₅₋₁₀ heterocycloalkyl or substituted C₅₋₁₀ heterocycloalkyl ring.

In certain embodiments of compounds of Formula (VI), R⁴ is chosen from H, C₁₋₆alkyl, substituted C₁₋₆ alkyl, C₅₋₁₀ aryl, substituted C₅₋₁₀aryl, C₆₋₁₂ arylalkyl, and substituted C₆₋₁₂ arylalkyl.

In certain embodiments of compounds of Formula (VI), R³ and R⁴ together with the atoms to which R³ and R⁴ are attached form a C₅₋₈ cycloalkyl, substituted C₅₋₈ cycloalkyl, C₅₋₈ heterocycloalkyl, or substituted C₅₋₈ heterocycloalkyl ring.

In certain embodiments of compounds of Formula (VI), wherein R³ and R⁴ together with the atoms to which R³ and R⁴ are attached form a C₅₋₈ cycloalkyl, substituted C₅₋₈ cycloalkyl, C₅₋₈ heterocycloalkyl, or substituted C₅₋₈ heterocycloalkyl ring, the at least one substituent group is chosen from halogen, C₁₋₆ alkyl, substituted C₁₋₆ alkyl, C₁₋₆heteroalkyl, substituted C₁₋₆heteroalkyl, C₆₋₁₀arylalkyl, substituted C₆₋₁₀ arylalkyl, and ═O.

In certain embodiments of compounds of Formula (VI), R² is chosen from H, C₅₋₈ aryl, substituted C₅₋₈ aryl, C₅₋₈ heteroaryl, substituted C₅₋₈ heteroaryl, C₆₋₁₀ heterocycloalkyl, substituted C₆₋₁₀ heterocycloalkyl, C₆₋₁₀ heteroarylalkyl, substituted C₆₋₁₀ heteroarylalkyl, C₁₋₁₀ alkylsulfonyl, substituted C₁₋₁₀ alkylsulfonyl, and —C(O)R⁶ wherein R⁶ is chosen from C₁₋₁₀ alkyl, substituted C₁₋₁₀ alkyl, C₁₋₁₀ heteroalkyl, substituted C₁₋₁₀heteroalkyl, C₃₋₁₀ cycloalkyl, substituted C₃₋₁₀ cycloalkyl, C₃₋₁₀ heterocycloalkyl, substituted C₃₋₁₀ heterocycloalkyl, C₅₋₁₀ aryl, substituted C₅₋₁₀ aryl, C₅₋₁₀ heteroaryl, substituted C₅₋₁₀ heteroaryl, C₆₋₁₈ cycloalkylalkyl, substituted C₆₋₁₈ cycloalkylalkyl, C₆₋₁₈ heterocycloalkylalkyl, substituted C₆₋₁₈ heterocycloalkylalkyl, C₆₋₁₈ arylalkyl, substituted C₆₋₁₈ arylalkyl, C₆₋₁₈ heteroarylalkyl, and substituted C₆₋₁₈ heteroarylalkyl.

In certain embodiments of compounds of Formula (VI), R² is chosen from —C(O)R⁶ and the at least one substituent group is chosen from halogen, C₁₋₆ alkyl, C₁₋₆ heteroalkyl, substituted C₁₋₆ heteroalkyl, C₁₋₆ alkoxy, substituted C₁₋₆ alkoxy, C₅₋₈ aryl, C₅₋₈ heterocycloalkyl, substituted C₅₋₈ heterocycloalkyl, C₅₋₈ heteroaryl, C₆₋₁₂ heterocycloalkylalkyl, substituted C₆₋₁₂ heterocycloalkylalkyl, C₆₋₁₂ heteroarylalkyl, substituted C₆₋₁₂ heteroarylalkyl, C₅₋₈ alkkylsulfonyl, ═O, ═S, —C(O)NH₂, —OH, —CF₃, nitro, —CN, —COOH, —OCF₃, and —N(CH₃)₂.

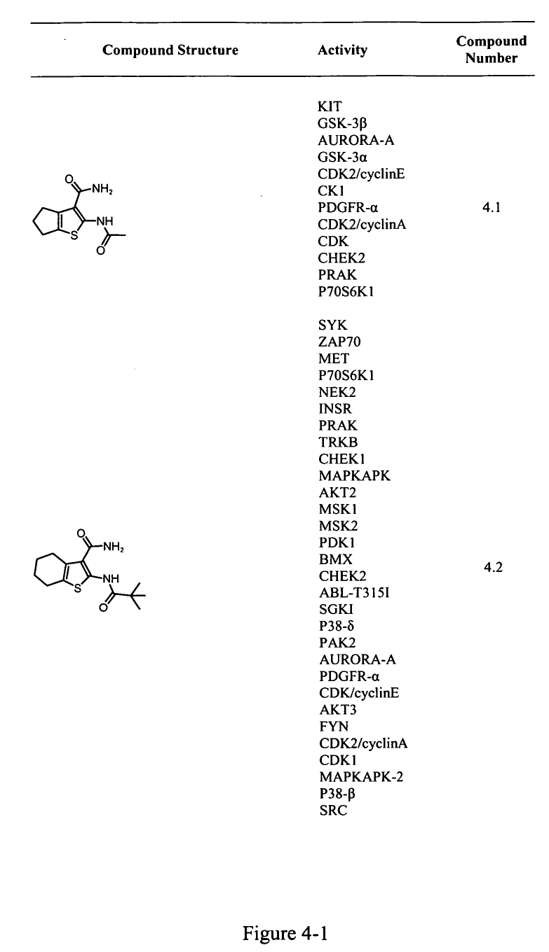

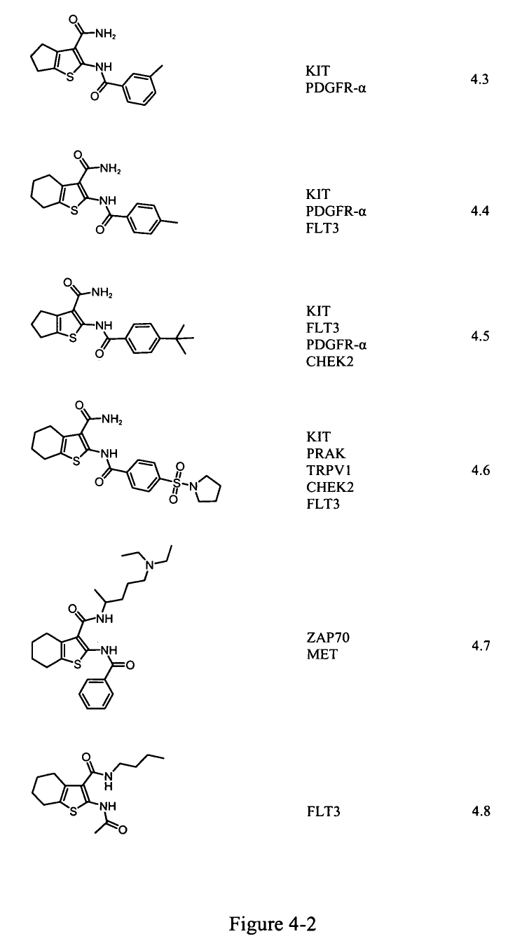

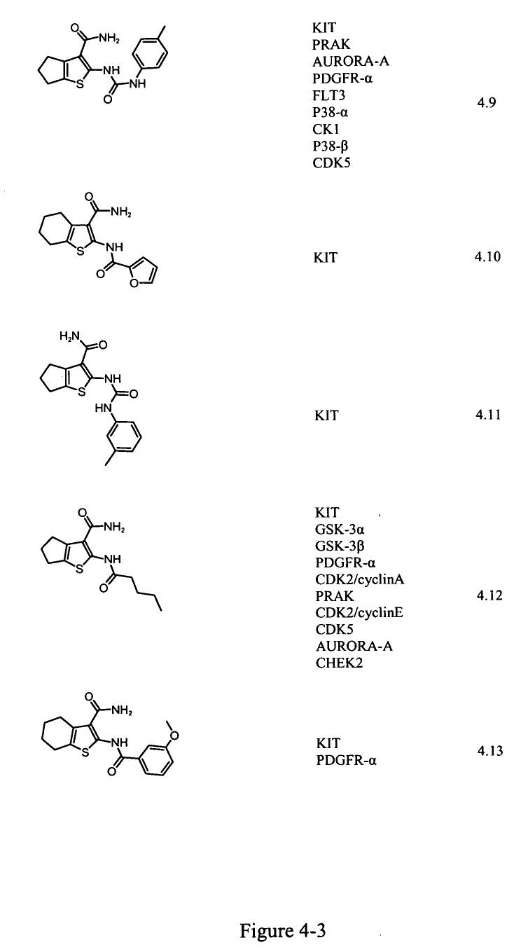

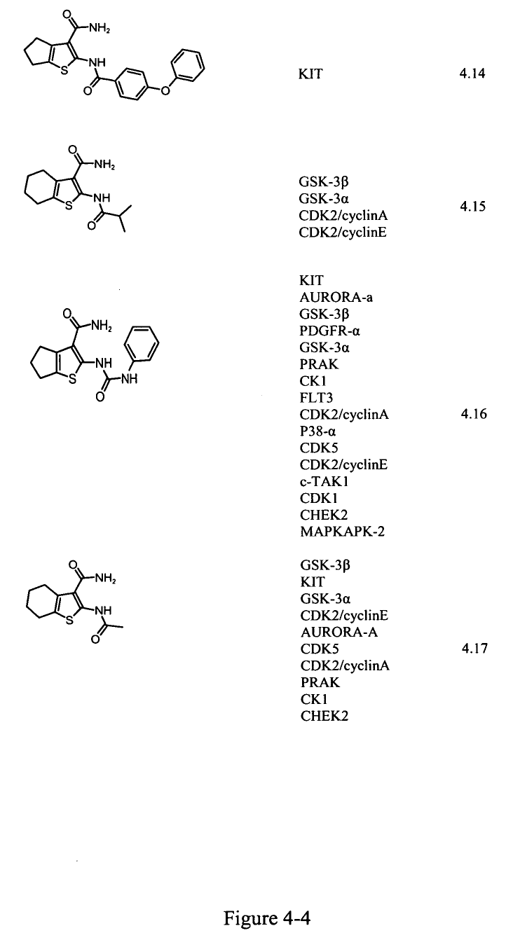

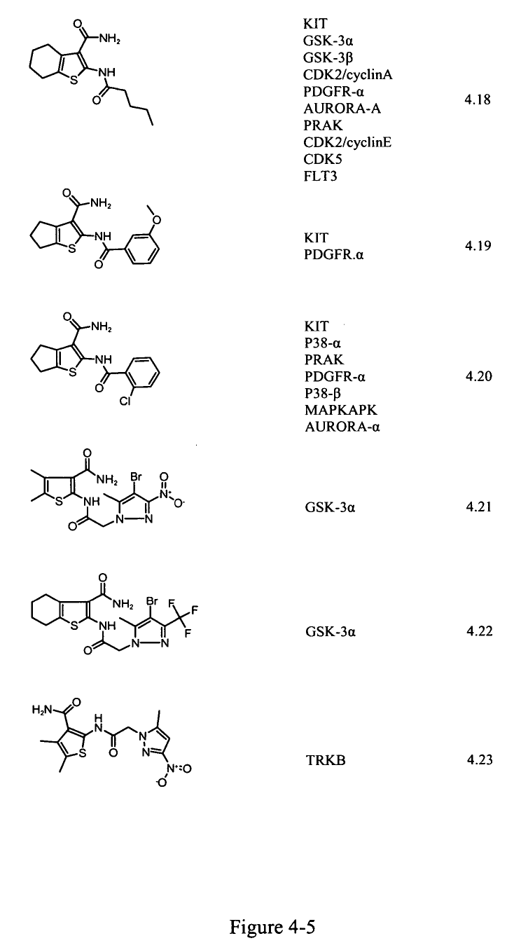

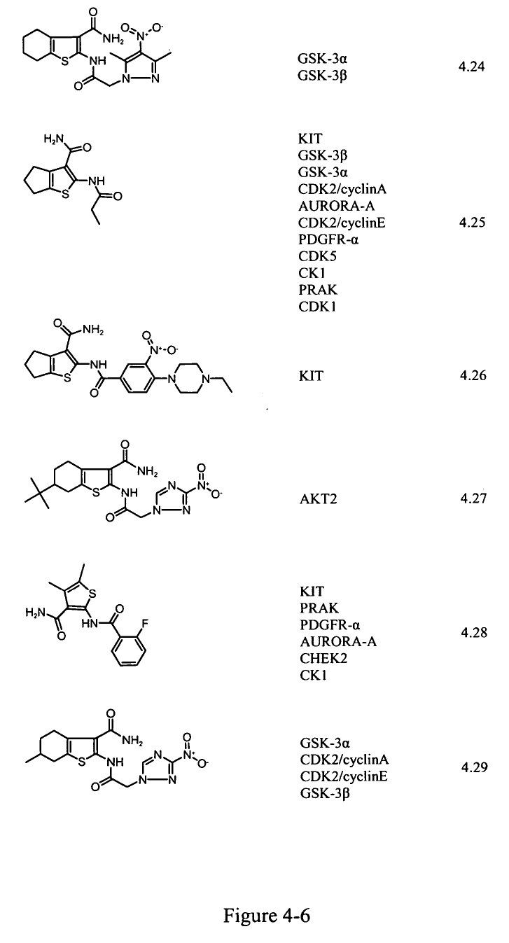

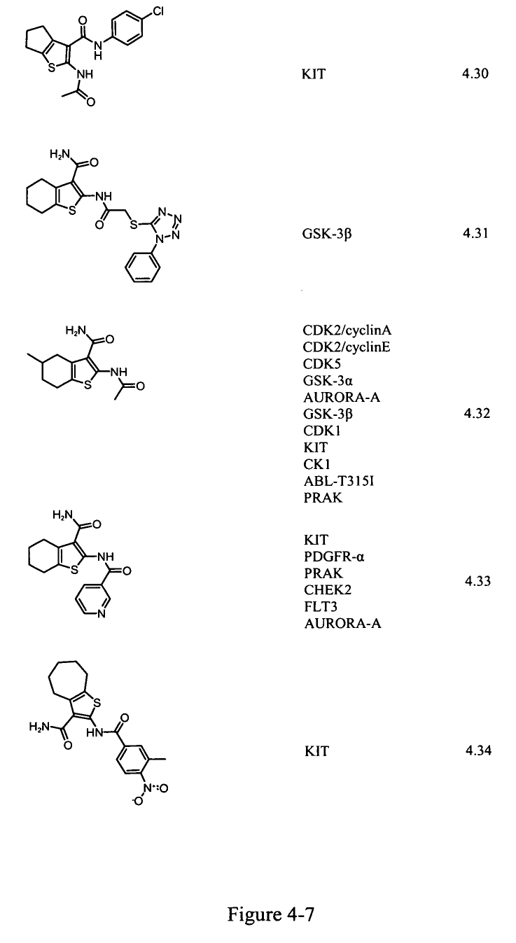

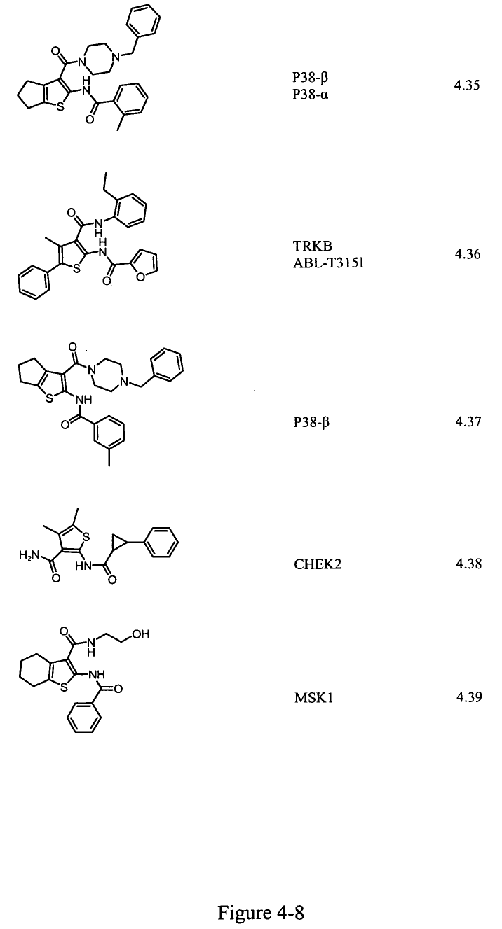

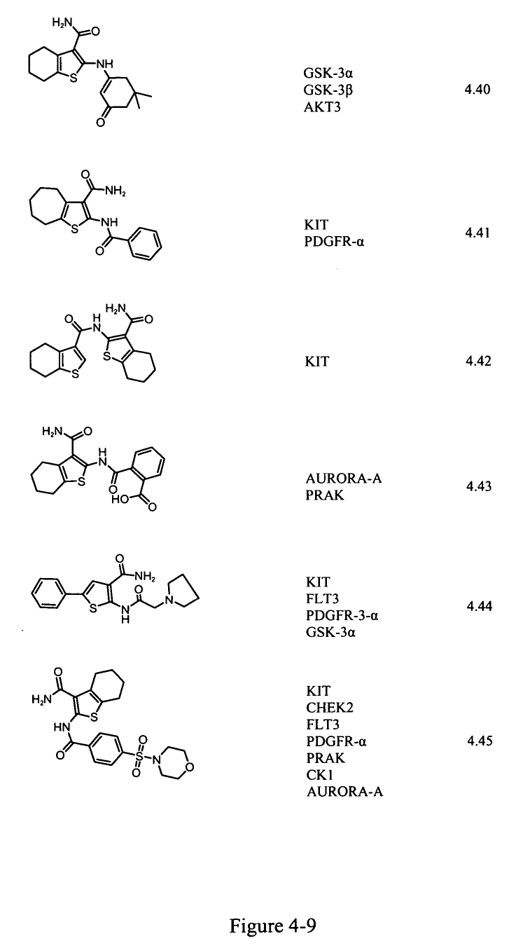

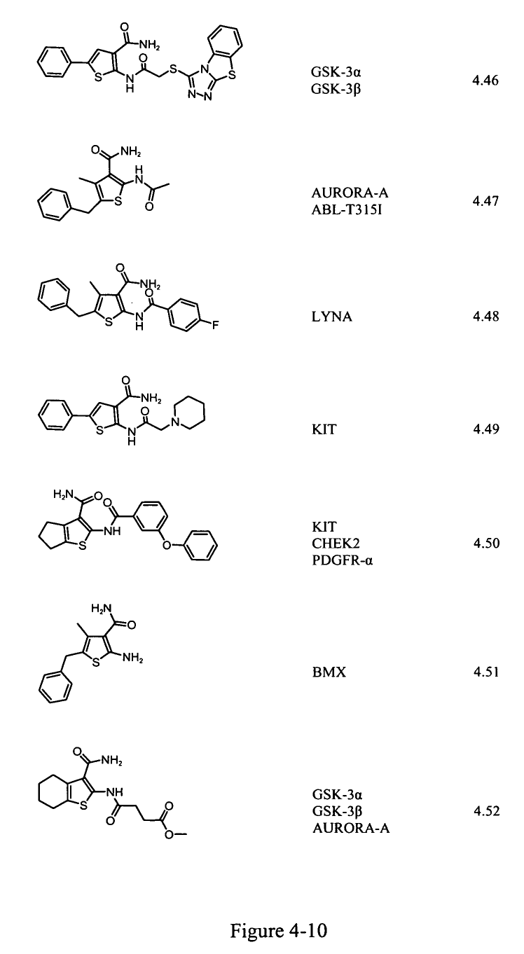

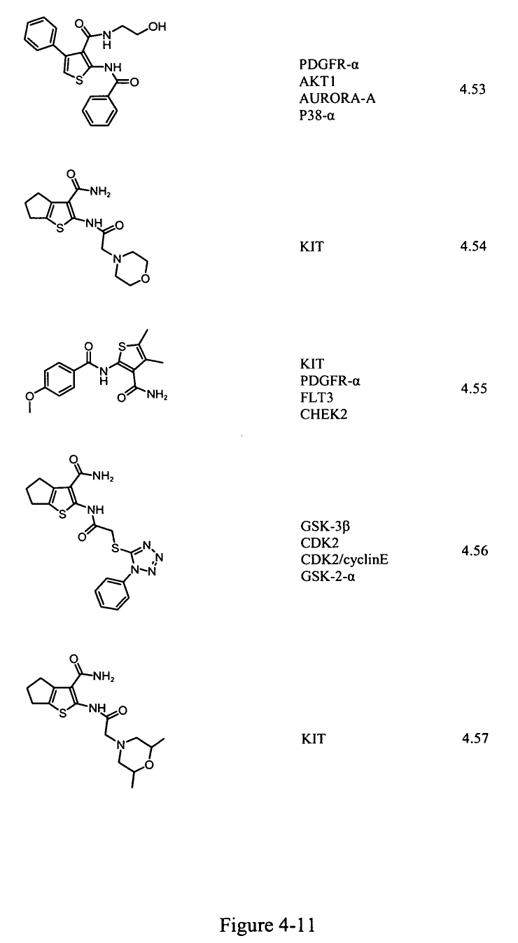

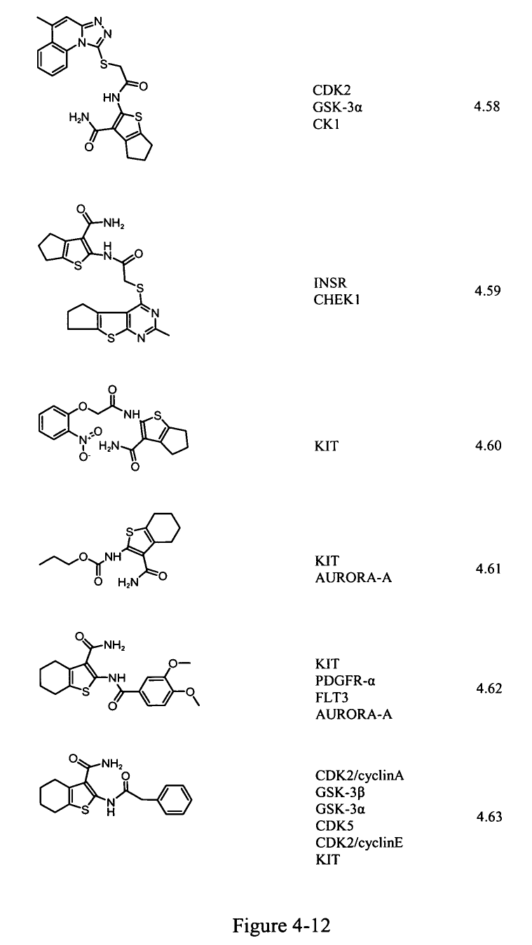

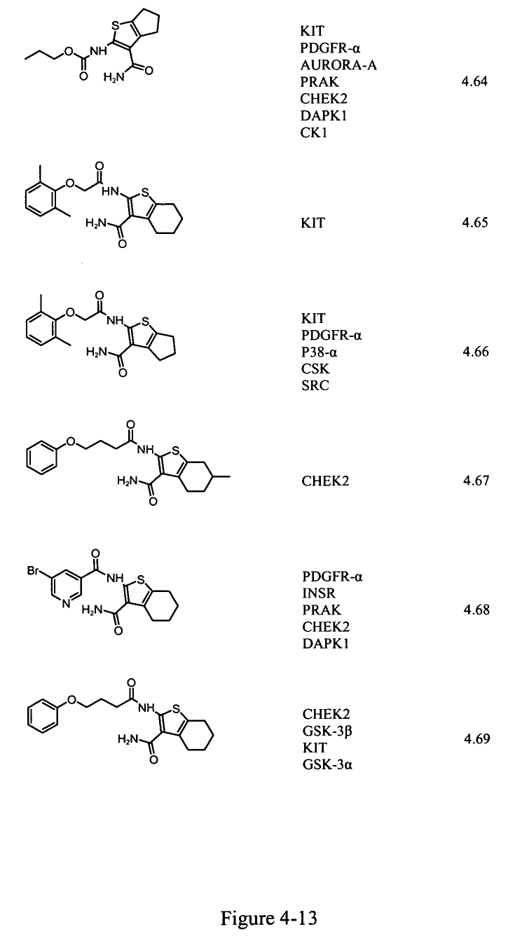

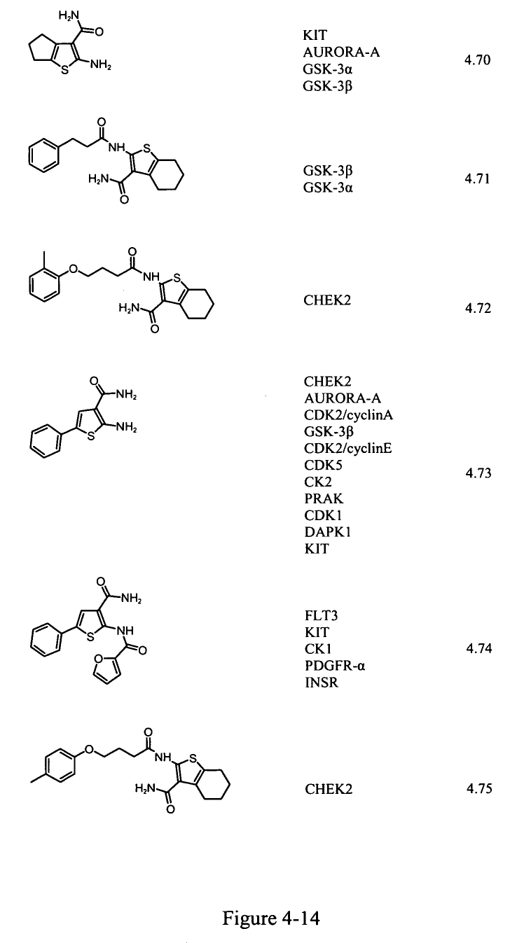

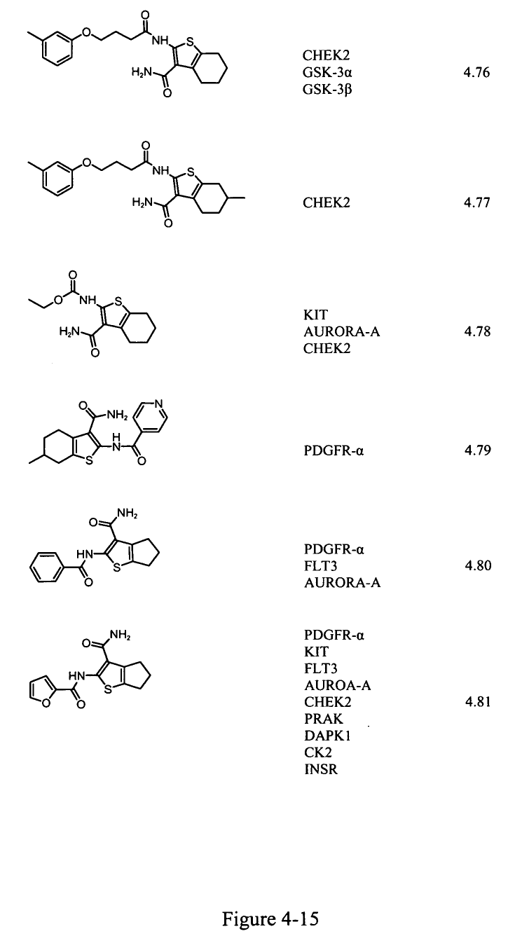

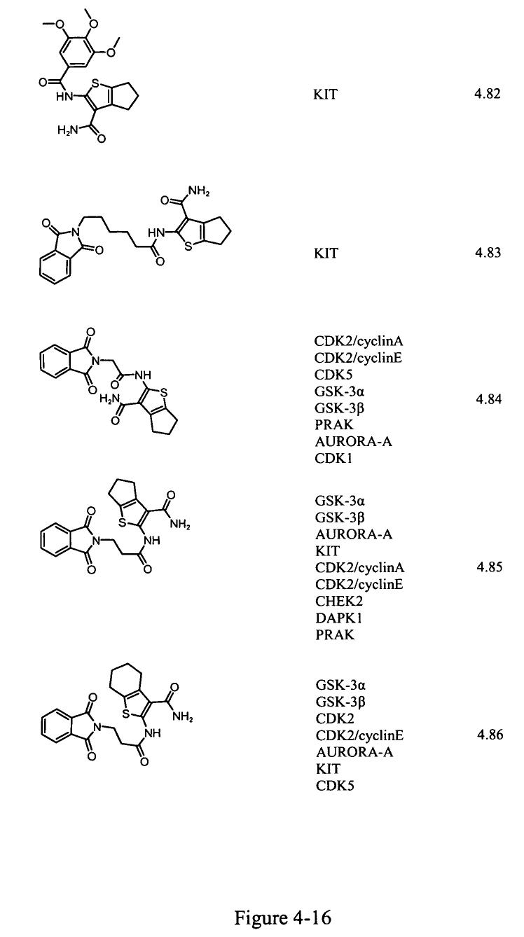

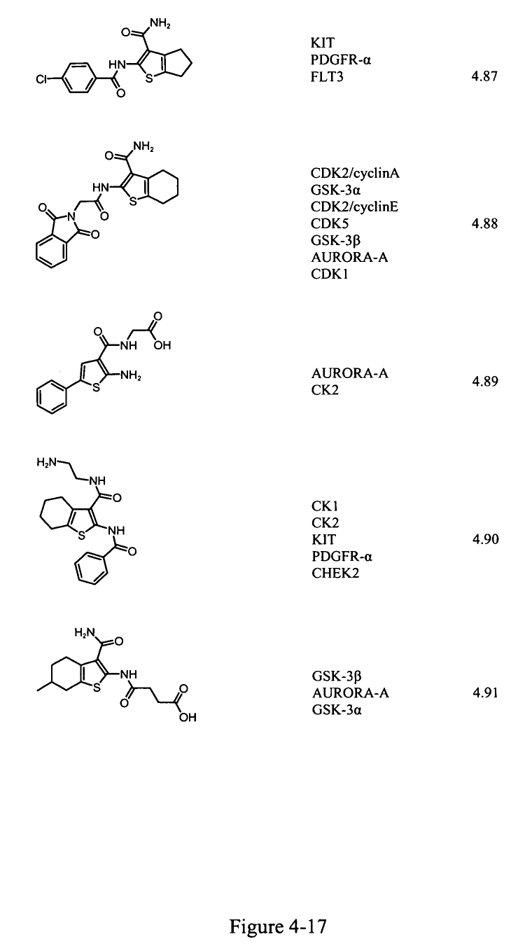

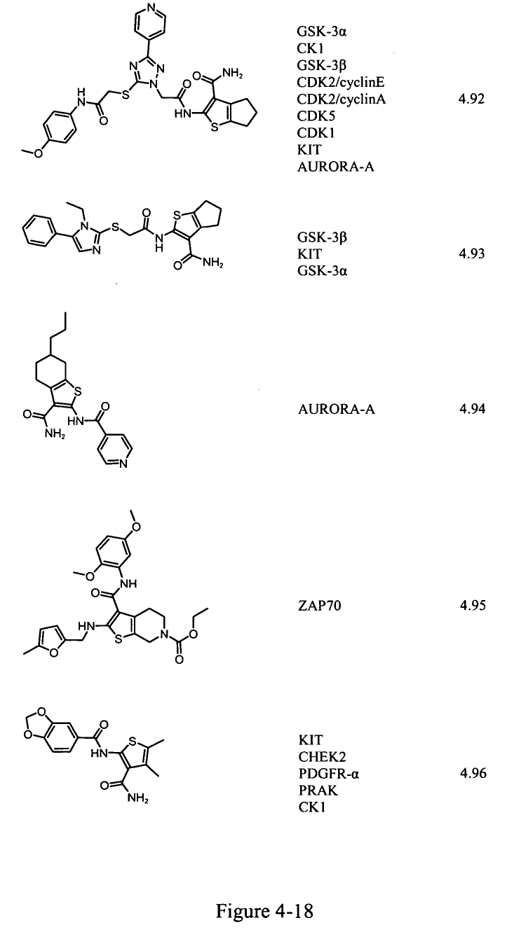

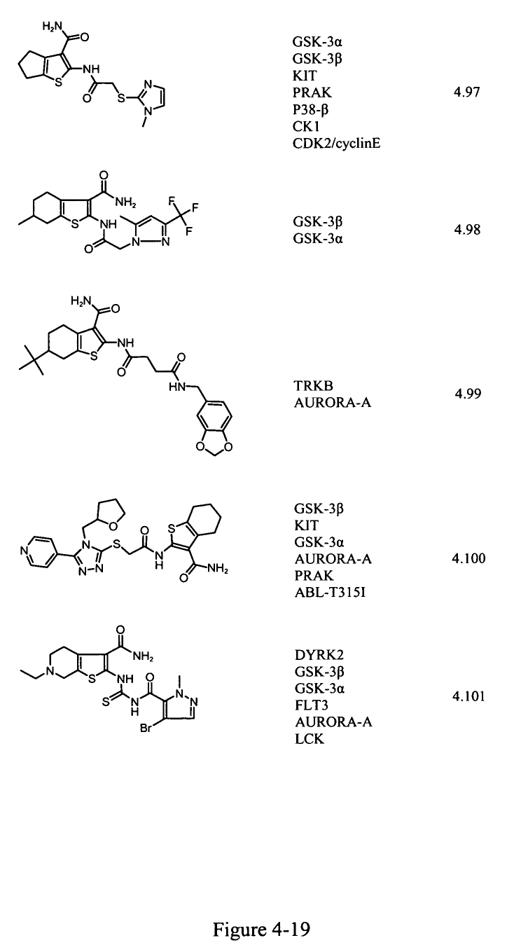

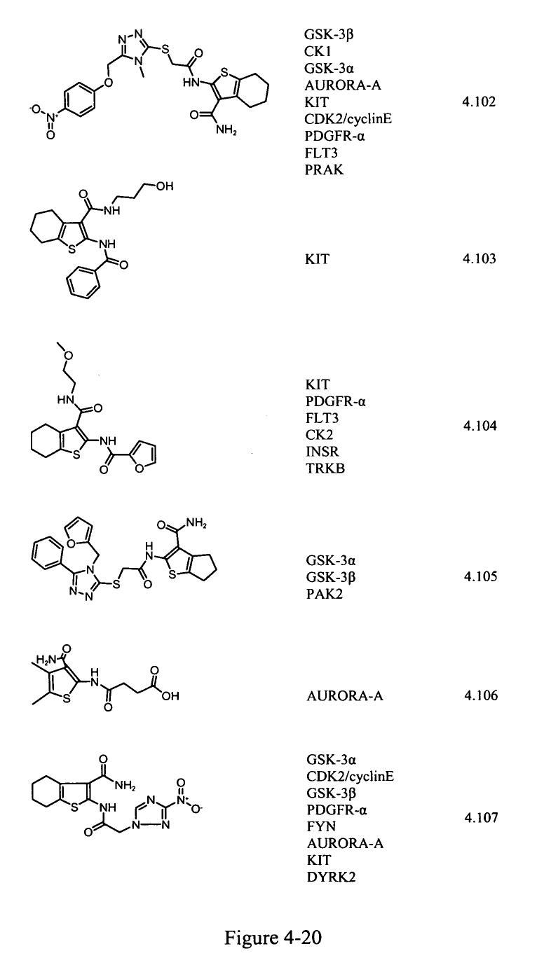

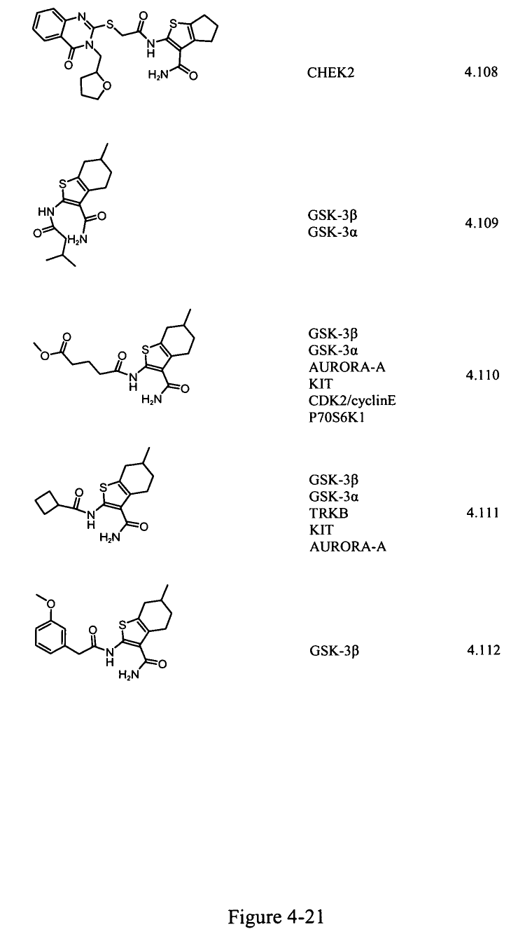

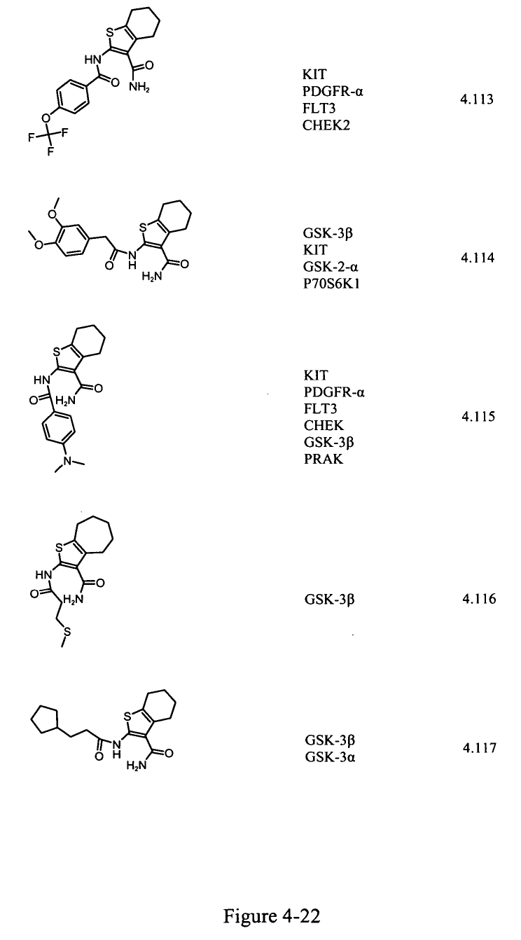

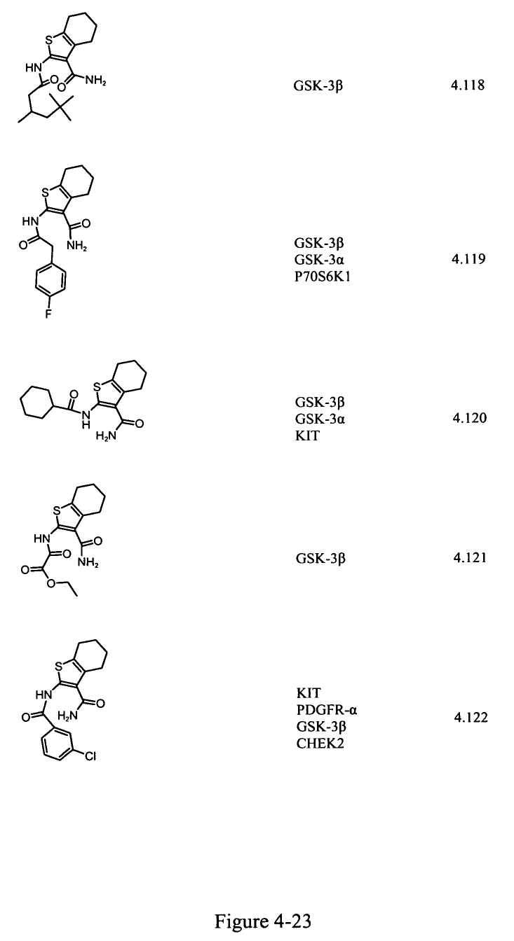

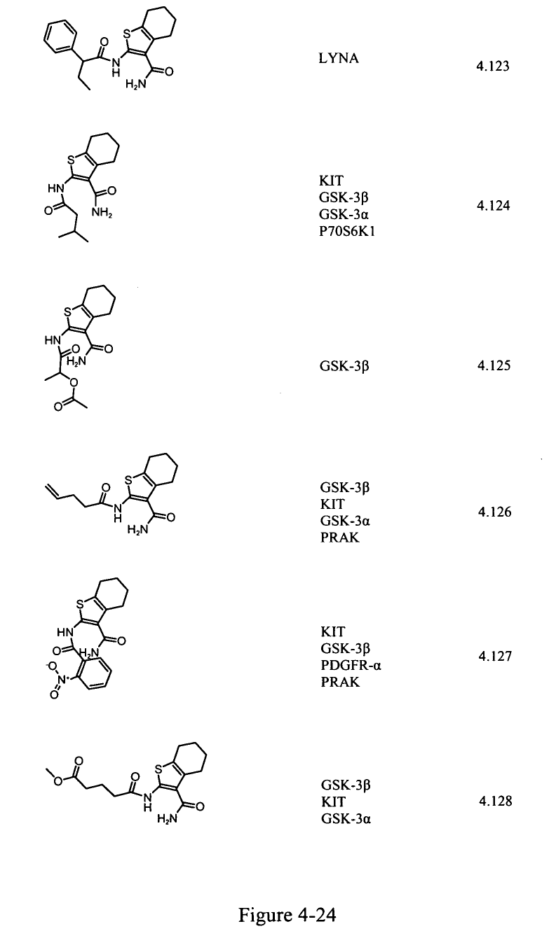

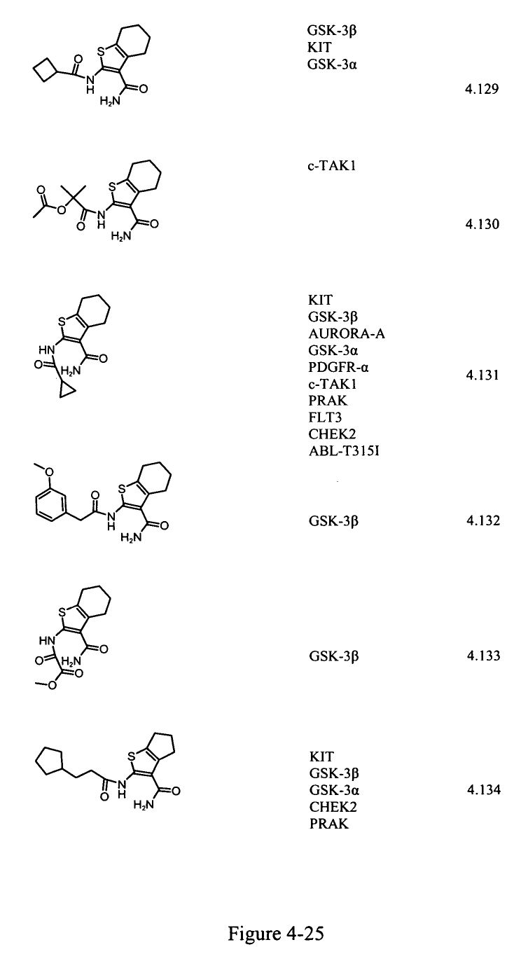

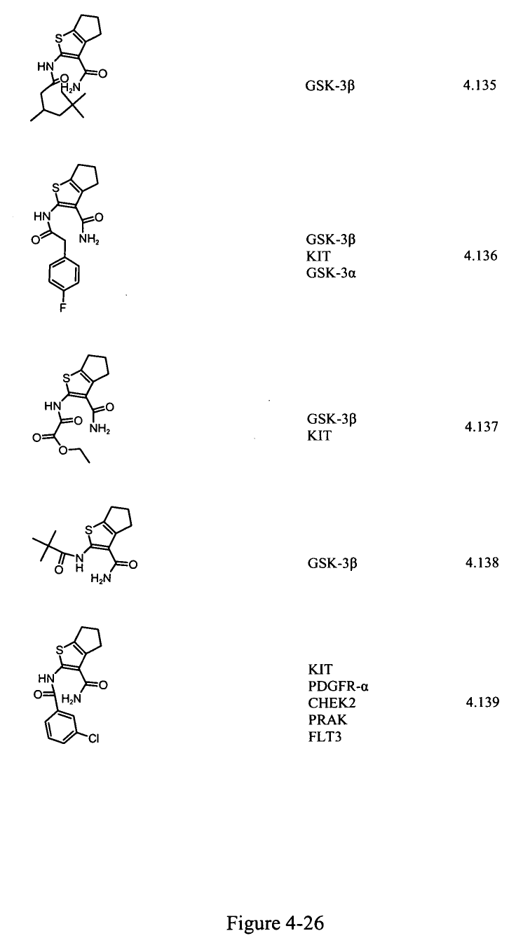

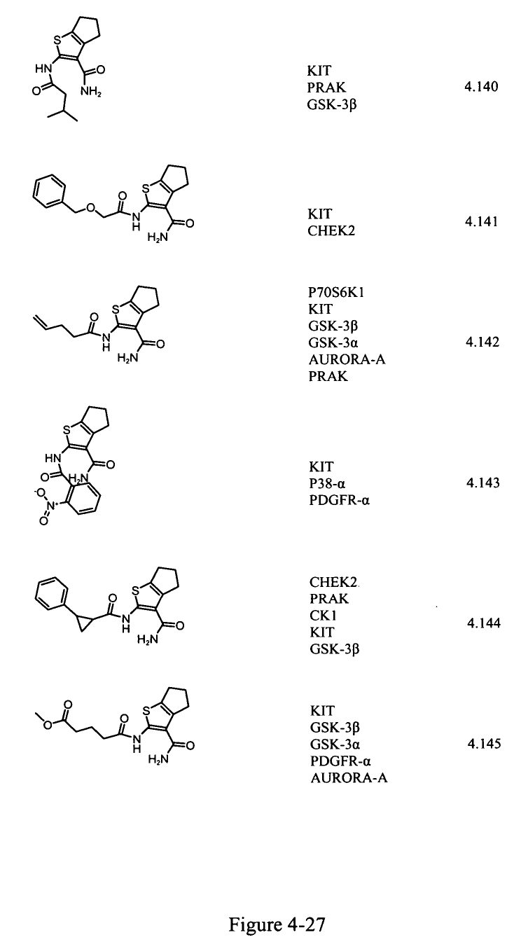

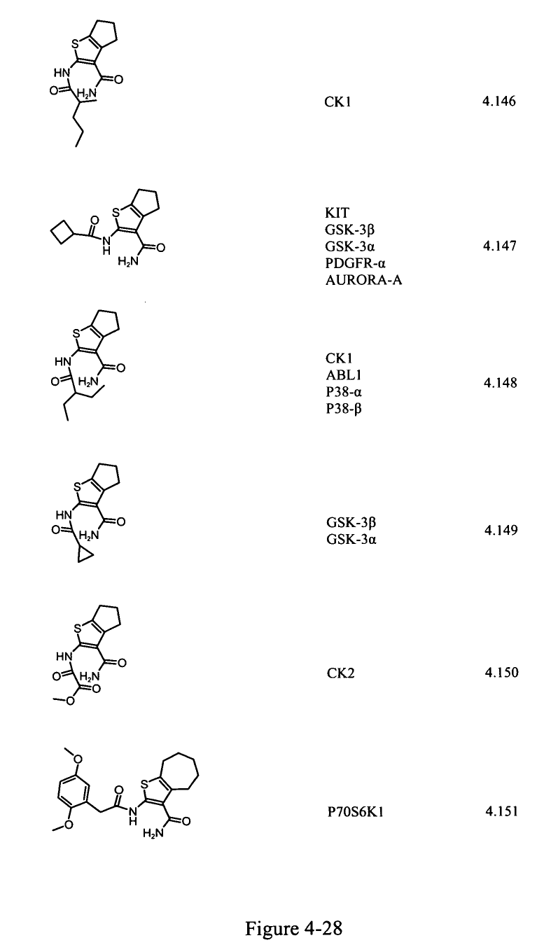

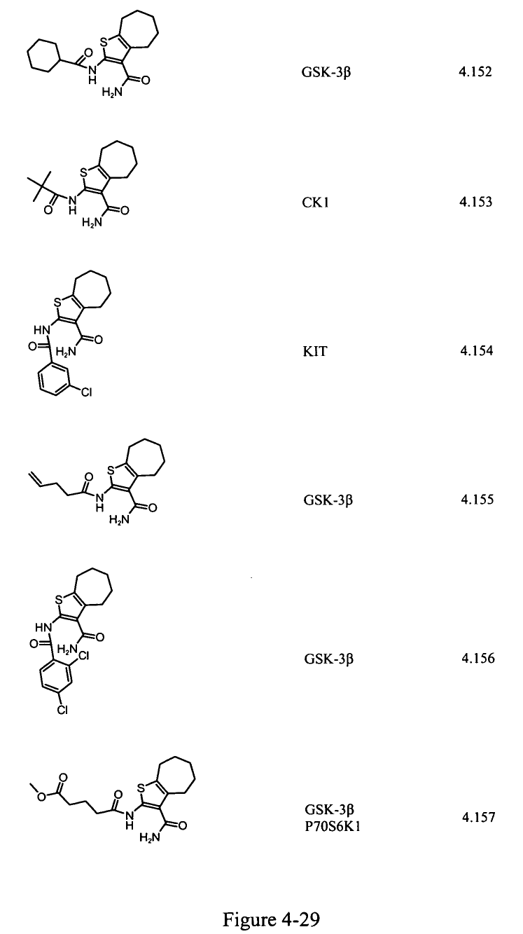

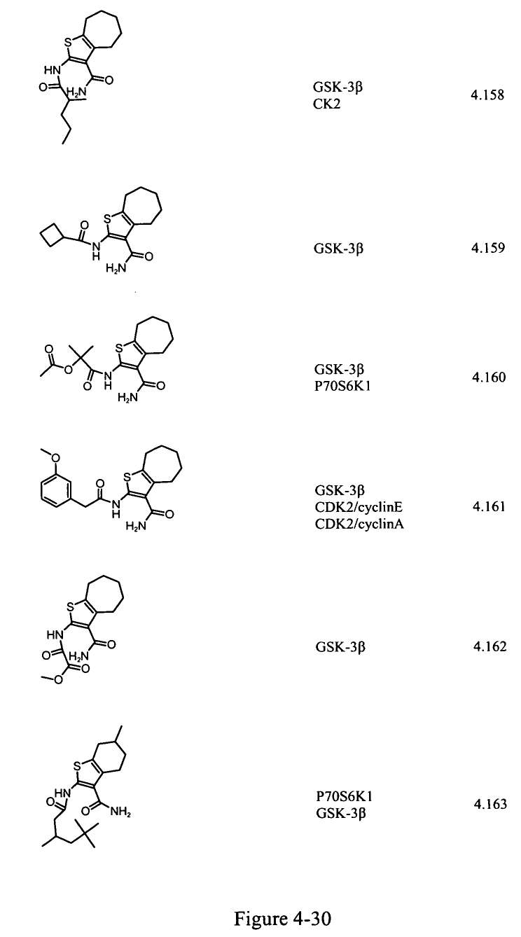

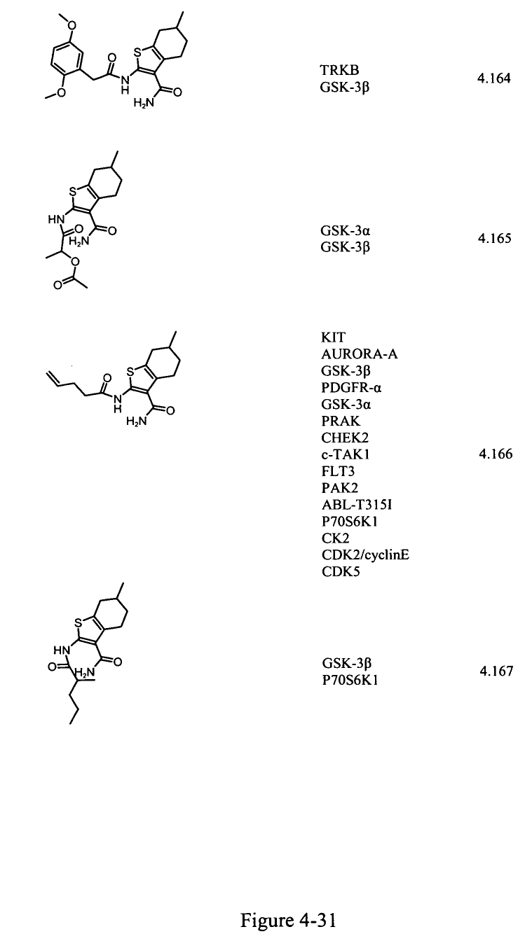

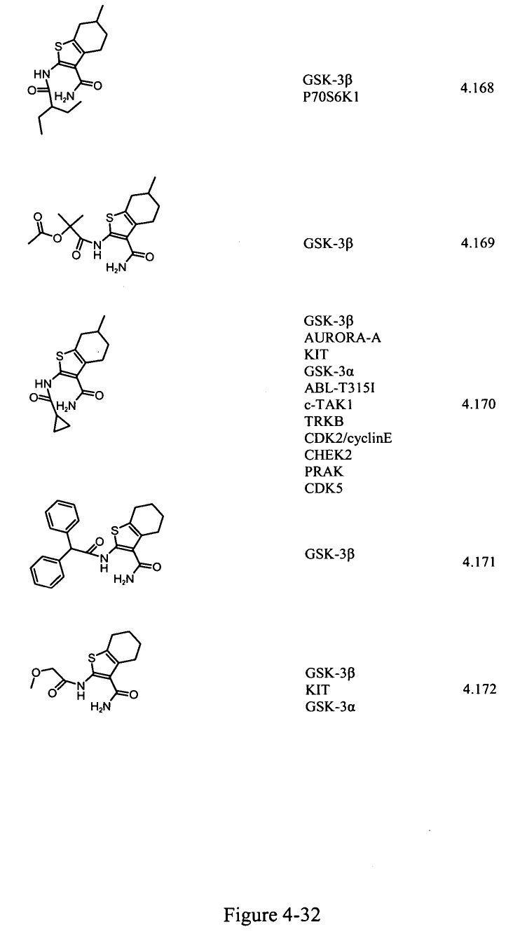

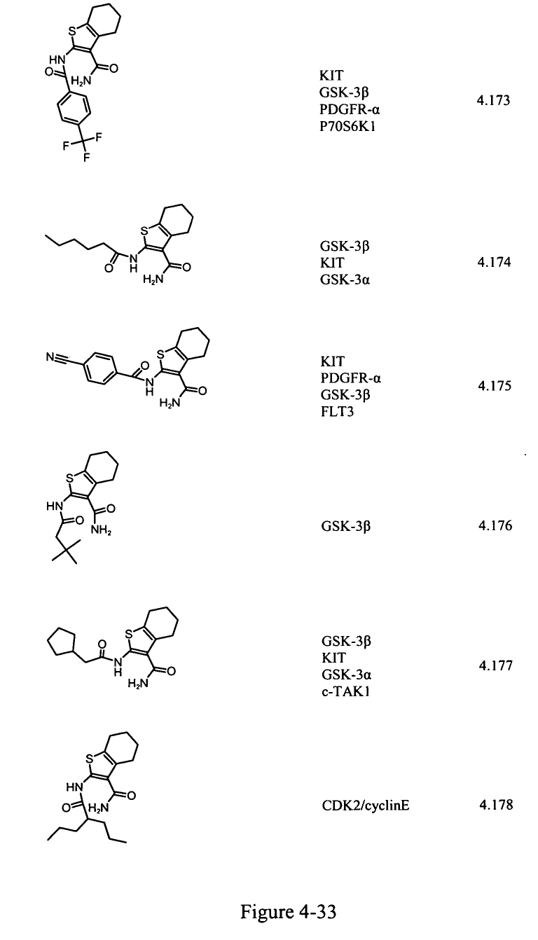

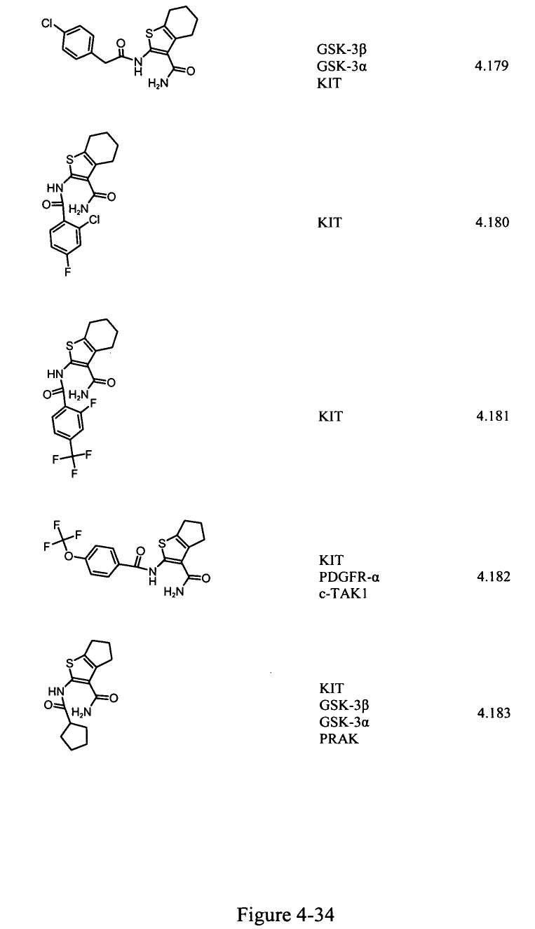

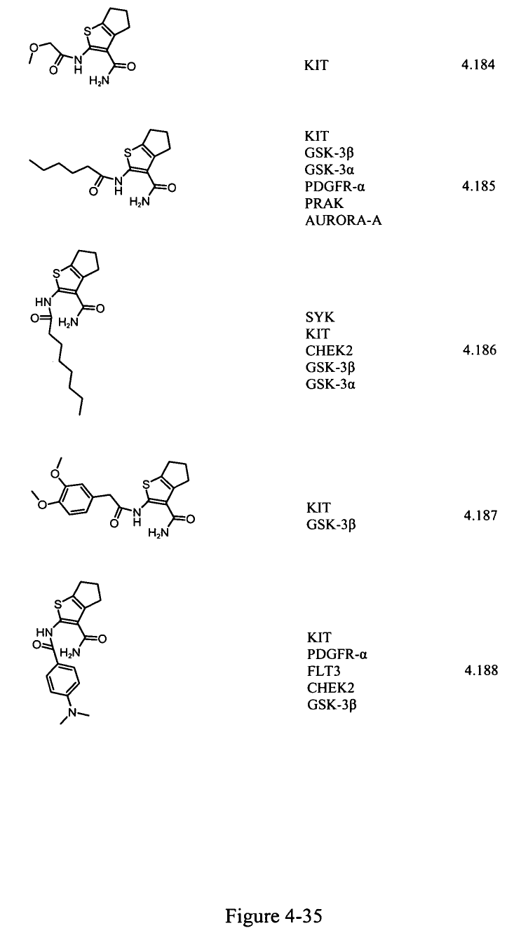

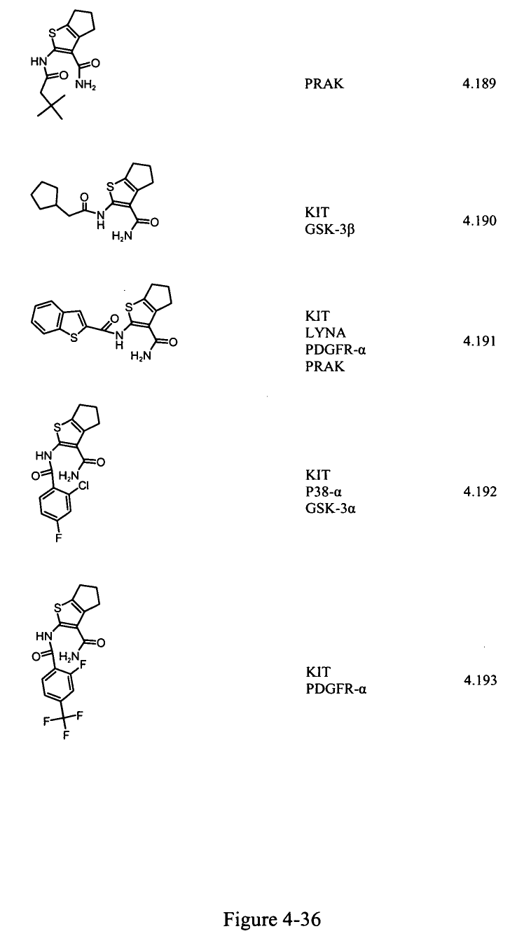

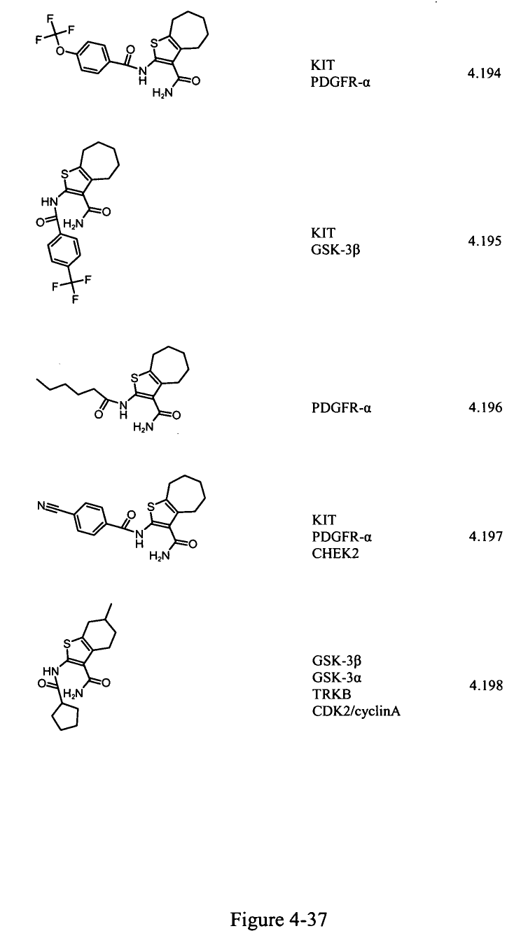

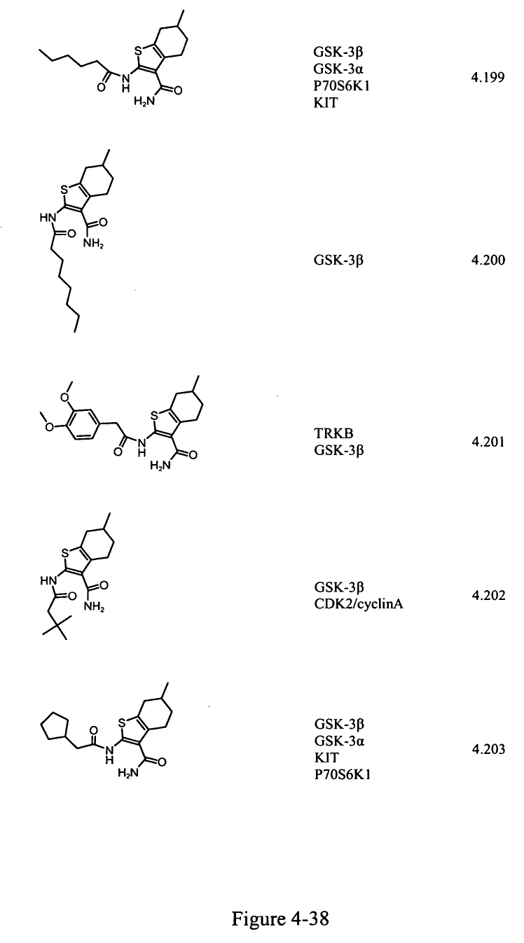

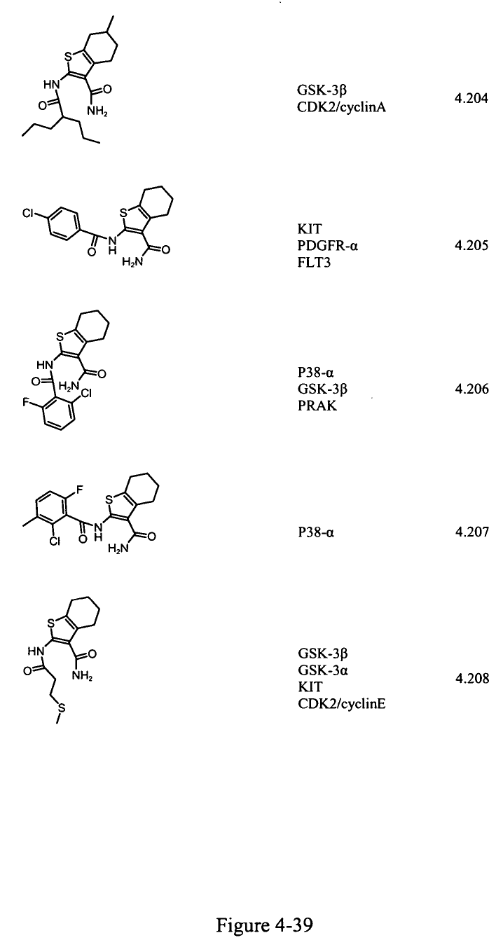

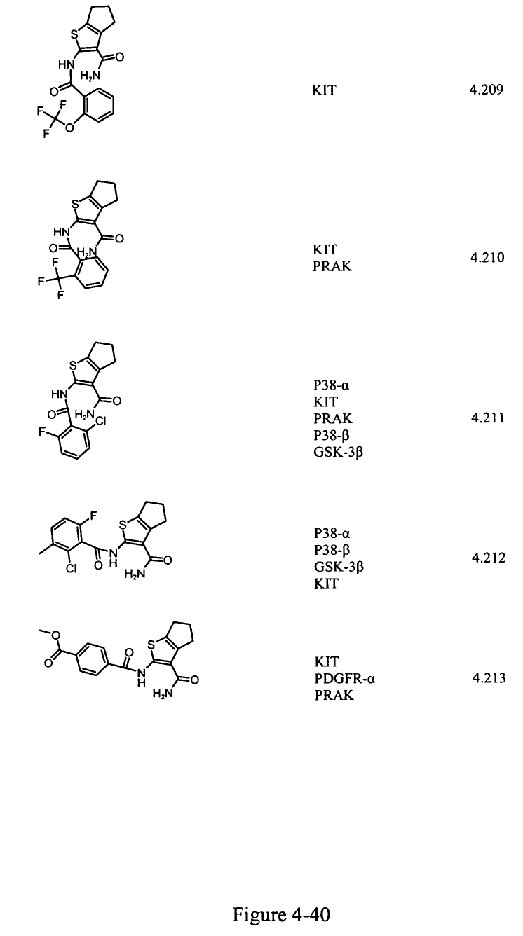

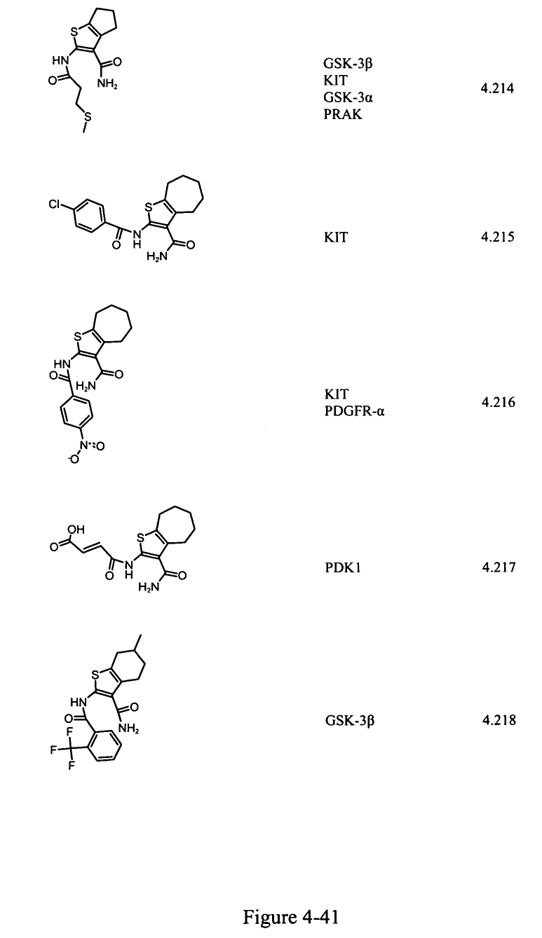

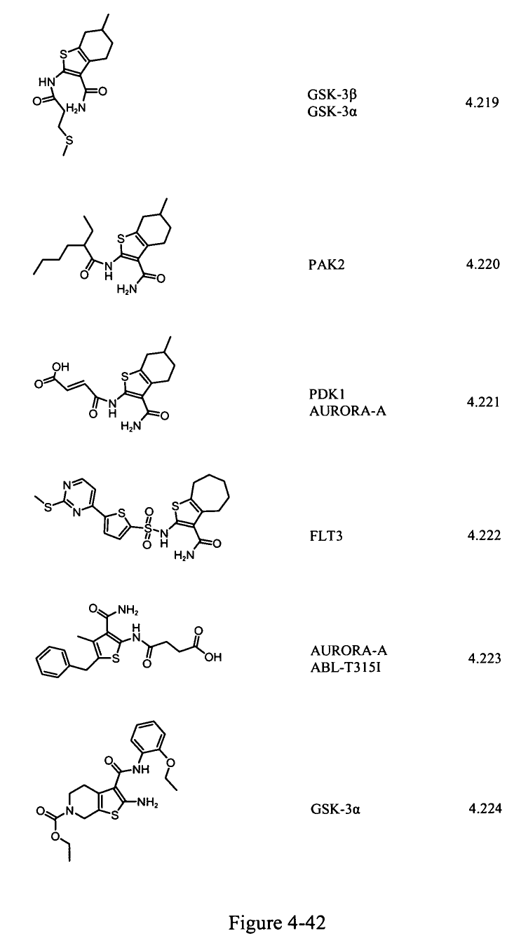

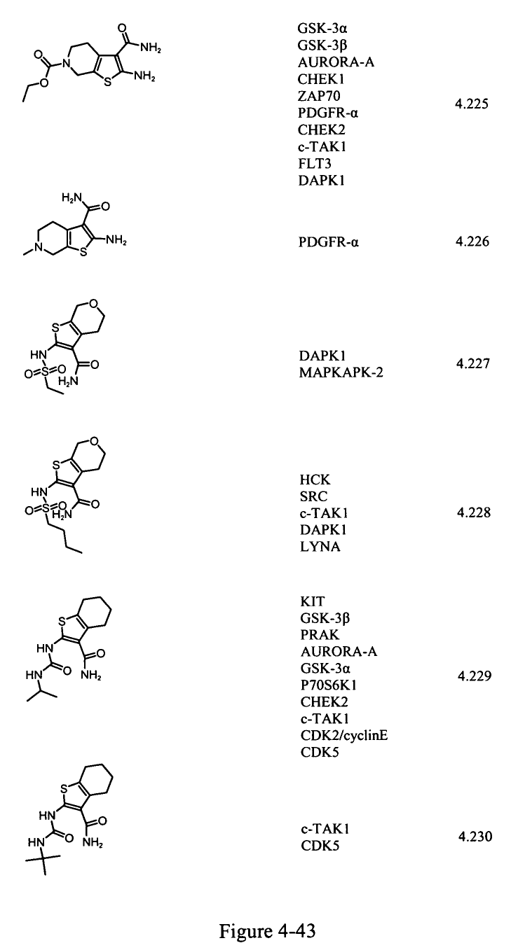

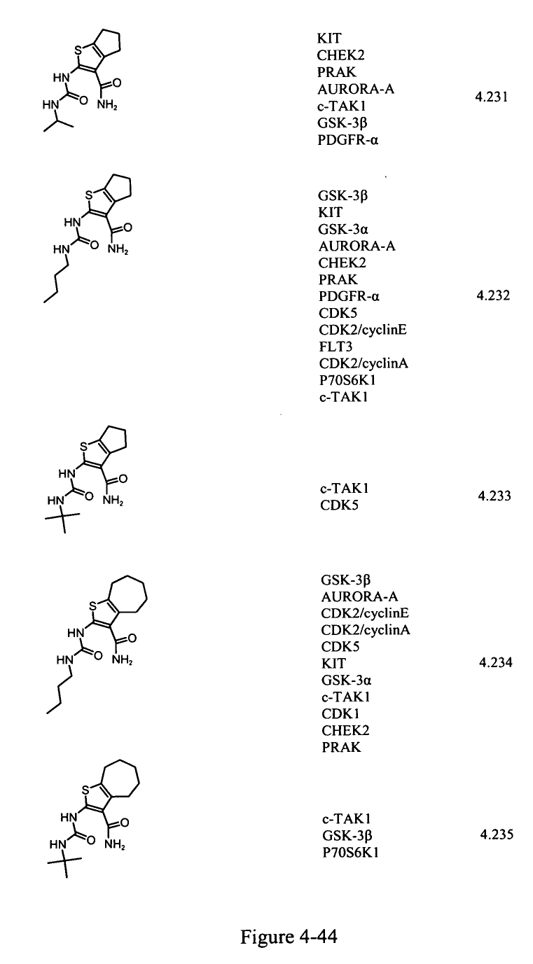

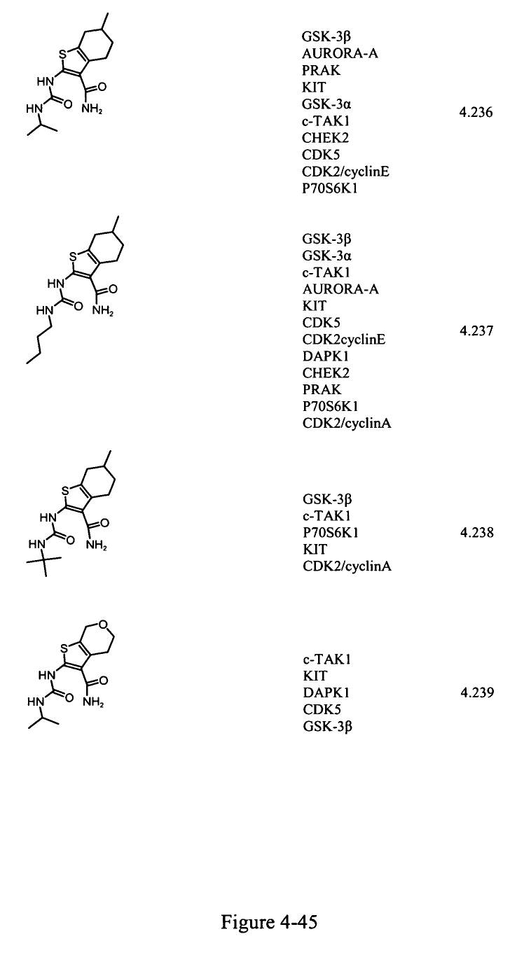

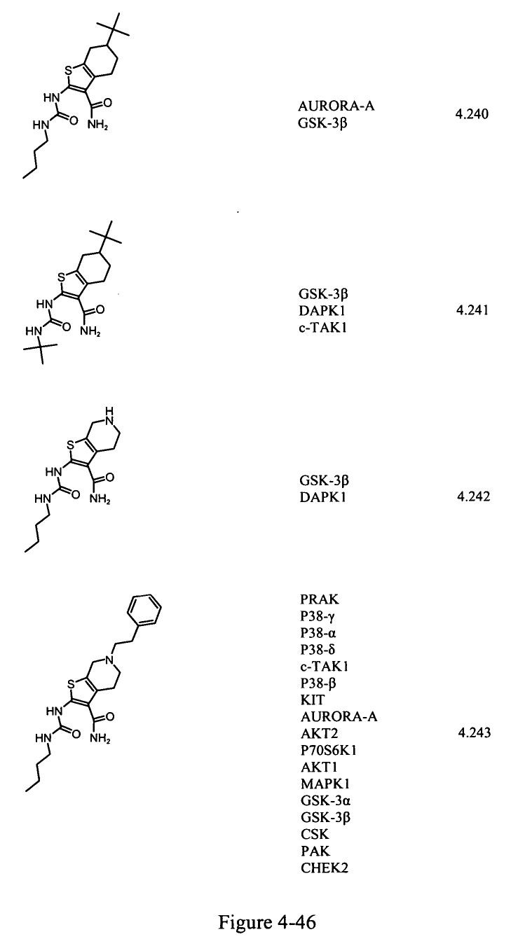

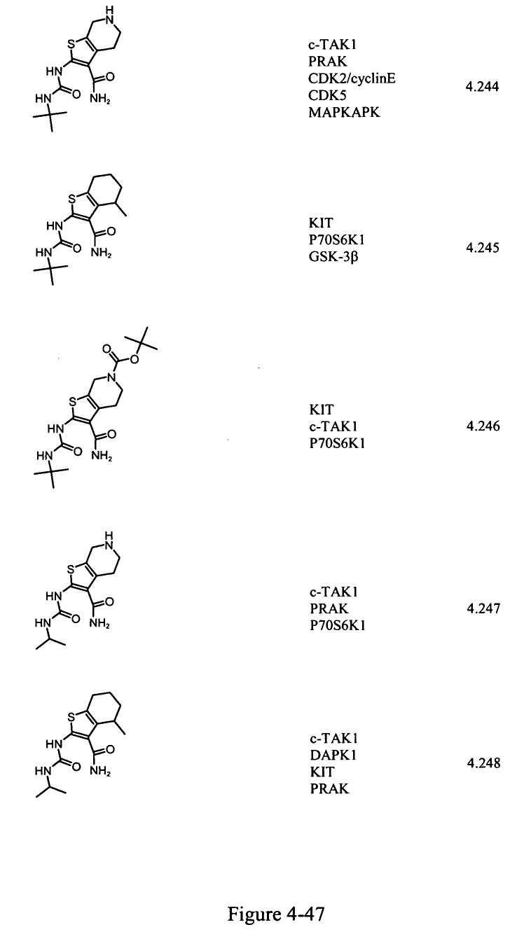

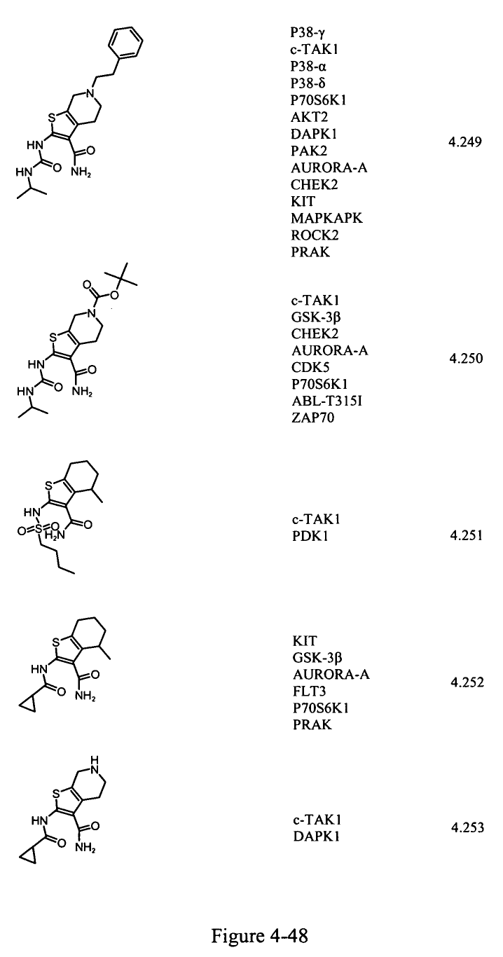

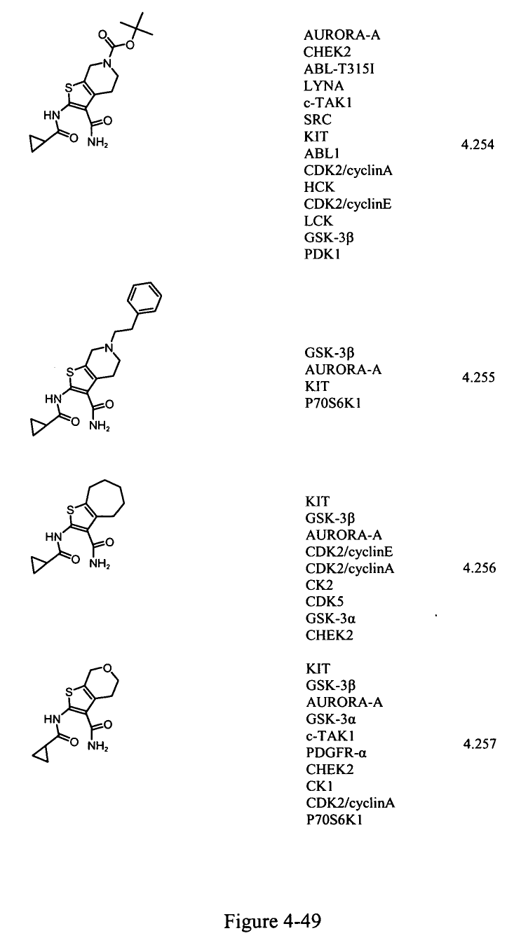

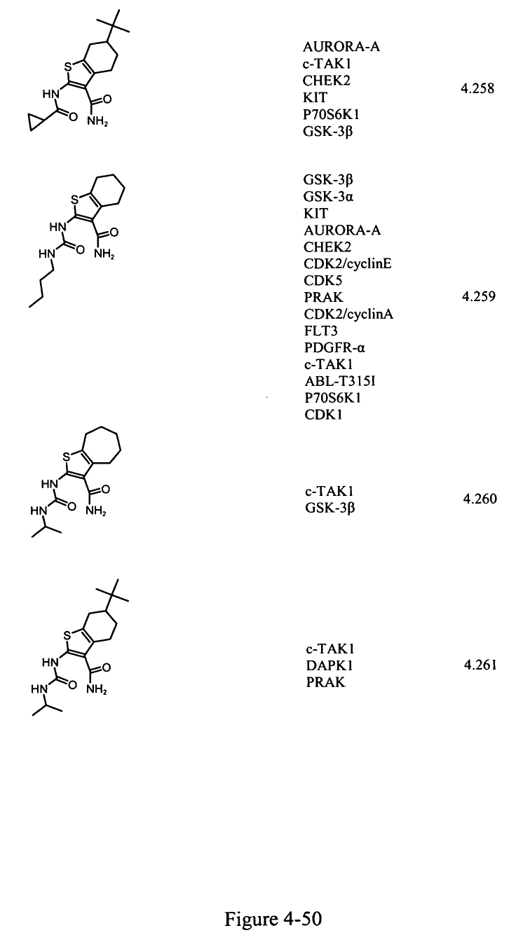

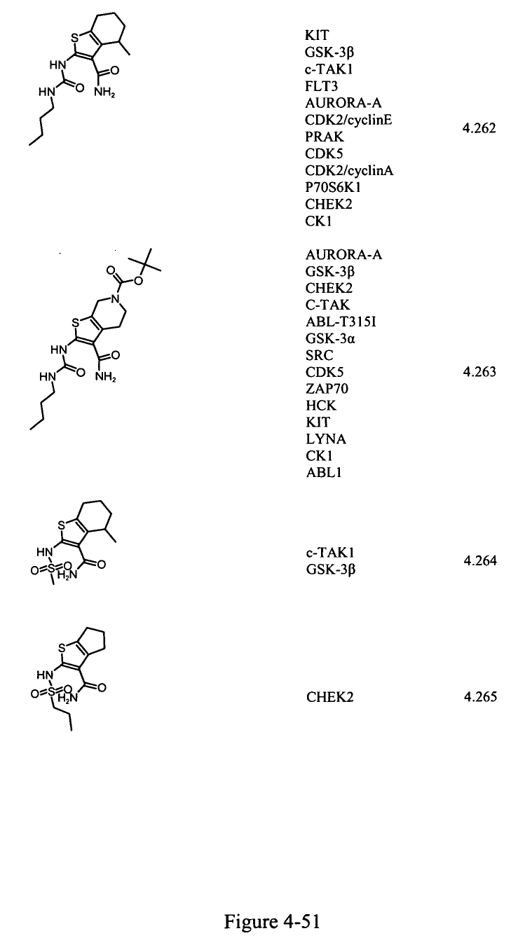

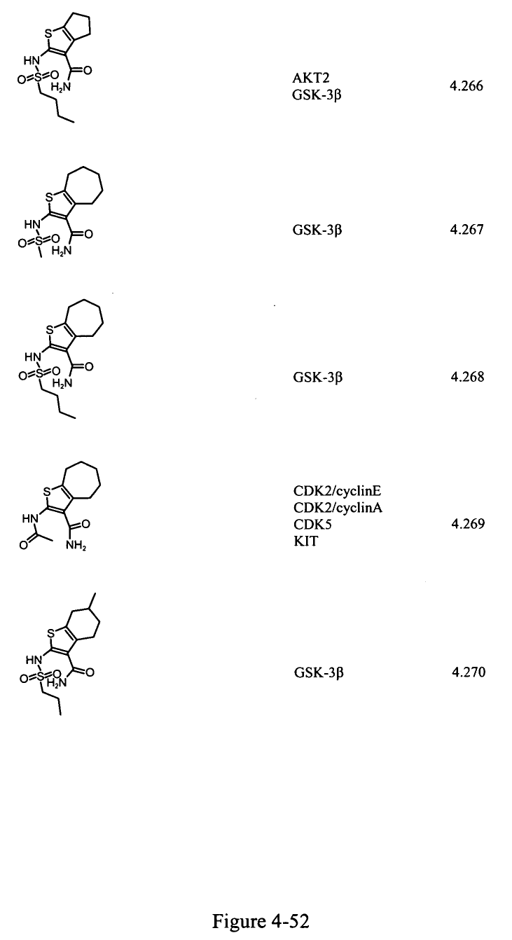

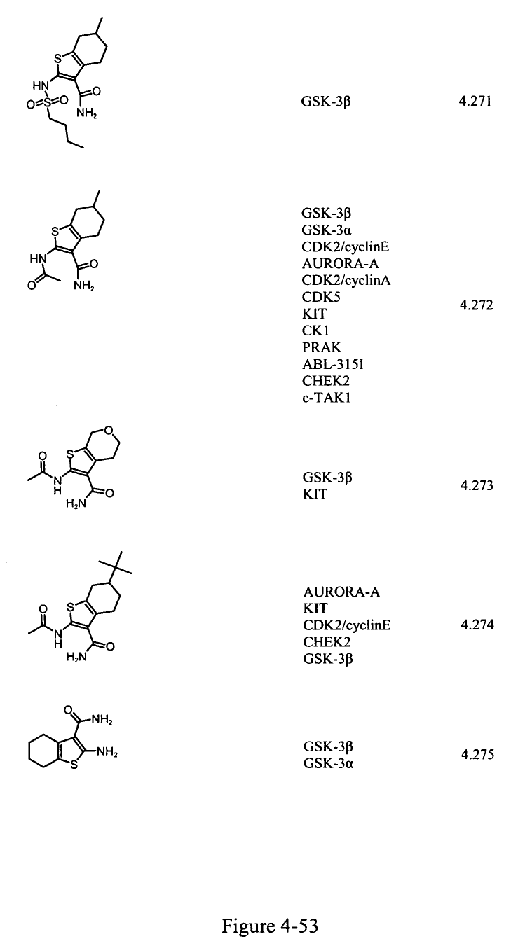

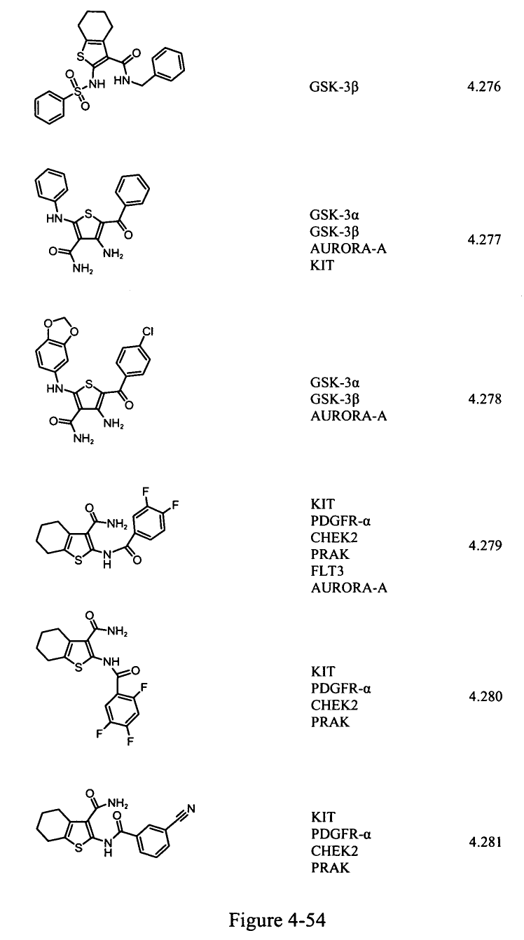

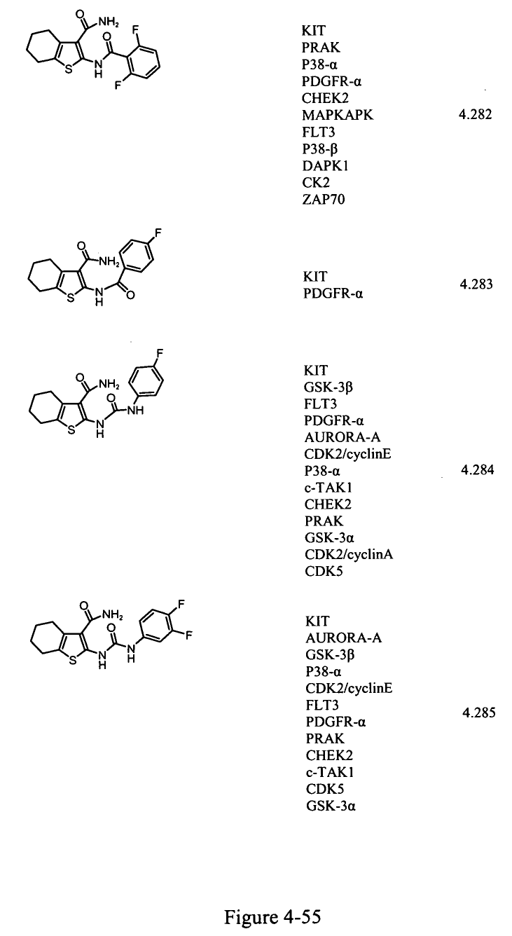

In certain embodiments of compounds of Formula (VI), the at least one compound has the structure of any of compounds 4.1 to 4.285 listed in FIG. 4, a stereoisomer thereof, a pharmaceutically acceptable salt thereof, a hydrate thereof, or a solvate of any of the foregoing.

In certain embodiments, compounds of the invention include stereoisomers thereof. The compounds may be purified and may include more than one stereoisomeric and/or enantiomeric form of a thiophene-based compound of the invention.

Examples of individual representative compounds of the present disclosure, and compounds comprised in compositions of the present disclosure, and used in methods of the present disclosure are listed in FIGS. 1 to 4. Each compound listed in FIGS. 1 to 4 was tested for protein kinase inhibitory activity according to the biological assays and definitions of protein kinase inhibitory activity as described herein. For each exemplary compound listed in FIGS. 1 to 4, the inhibitory activity for at least one protein kinase according to the biological assays and definitions of protein kinase inhibitory activity as described herein is indicated. The human protein kinase or kinases for which a compound exhibited selectivity as defined herein, is also presented in FIGS. 1 to 4.

As used herein, the compounds of the present disclosure, including the compounds of Formulae (I) to (VI), can include pharmaceutically acceptable derivatives or prodrugs thereof. A “pharmaceutically acceptable derivative or prodrug” refers to any appropriate pharmaceutically acceptable salt, ester, salt of an ester, hydrate, solvate, or other derivative of a compound of this present disclosure that, upon administration to a subject, is capable of providing, directly or indirectly, a compound of the present disclosure. Particularly favored derivatives and prodrugs include those that increase the bioavailability of the compounds of the present disclosure when such compounds are administered to a subject, for example by allowing an orally administered compound to be more readily absorbed into the blood, or which enhance delivery of the parent compound to a biological compartment, such as the brain or lymphatic system, relative to the parent species. Prodrugs can include derivatives where a group which enhances aqueous solubility or active transport through the gut membrane is appended to the structure of Formulae (I) to (VI). Other prodrugs can include a promoiety that modifies the ADME (absorption, distribution, metabolism and excretion) of the parent compound and thereby enhances the therapeutic effectiveness of the parent compound.

In certain embodiments, compounds of the present disclosure can be modified by appending appropriate functionalities to enhance selective biological properties. Such modifications are known in the art and include those which can increase biological penetration into a given biological compartment, such as blood, lymphatic system, central nervous system, to increase oral availability, increase solubility to allow administration by injection, alter metabolism, and alter the rate of excretion.

In some embodiments, compounds of the present disclosure can be modified to facilitate use in biological assay, screening, and analysis protocols. Such modifications can include, for example, derivatizing to effect or enhance binding to physical surfaces such as beads or arrays, or modifying to facilitate detection such as by radiolabeling, affinity labeling, or fluorescence labeling.

Compounds of the present disclosure possess inhibitory activity with at least one ATP-utilizing enzyme. An ATP-utilizing enzyme refers to an enzyme that catalyzes the transfer of a phosphate group from an ATP molecule to a biomolecule such as a protein or carbohydrate. Examples of ATP-utilizing enzymes include, but are not limited to, synthetases, ligases, synapsins, phosphatases, and kinases. The kinases can be animal kinases, including mammalian protein kinases, and human protein kinases.

Without being limited by theory, ATP-utilizing enzymes can be inhibited by compounds structurally similar to the phosphoryl-containing compounds that serve as the substrate for the phosphorylation reaction. For example, structurally similar compounds can bind to the active site or catalytic domain of an ATP-utilizing enzyme and thereby prevent substrate binding.

In certain embodiments, compounds of the present disclosure exhibited human protein kinase inhibitory activity.

Protein kinases are among the largest and most functionally diverse gene families. Most of the over 500 human protein kinases belong to a single superfamily of enzymes in which the catalytic domains are related in sequence and structure. Most human protein kinases can further be grouped into seven major groups based on the deoxyribonucleic acid (DNA) sequence homologies identified as CAMK (calcium/calmodulin-dependent protein kinases), AGC (including PKA (protein kinase A), PKG (protein kinase G), PKC (protein kinase C kinases), CK1 (casein kinases), CMGC (containing CDK (cyclin-dependent)), MAPK (mitogen activated), GSK3 (glycogen synthase) and CLK (CDC2-like kinases), STE (homologs of yeast Sterile 7, Sterile 11, and Sterile 20 kinases), TK (tyrosine kinases), and TKL (tyrosine-kinase like).

The AGC protein kinase family includes AKT1, AKT2, AKT3, AURORA-A, MSK1, MSK2, P70S6K, PAK1, PKA, and SGK1 protein kinases. The CMGC protein kinase family includes the CDK1, CDK2/cyclinA, CDK2/cyclinE, CDK5, DYRK2, GSK-3α, GSK-3β, P38-α, P38-β, P38-δ, and P38-γ, and MAPK1 protein kinases. The CAMK protein kinase family includes the DAPK1, MAPKAPK-2, MAPKAPK-3, CHEK1, CHEK2, PRAK, and c-TAK1 protein kinases. The TK protein kinase family includes the ABL1, CSK, FLT3, FYN, HCK, INSR, KIT, LCK, PDGFR-α, LYNA, SYK, and SRC protein kinases. The STE protein kinase family includes PAK2 protein kinase.

Certain compounds of the present disclosure exhibited selectivity for one or more protein kinases, where selectivity is as defined herein. Certain compounds of the present disclosure exhibited selective inhibitory activity for at least one of the following protein kinases: ABL, ABL-1, ABL-T315I, AKT1, AKT2, AKT3, AURORA-A, BMX, CDK, CDK1, CDK2/cyclinA, CDK2/cyclinE, CDK5, CHEK, CHEK1, CHEK2, CK1, CK2, CSK, c-TAK1, DAPK1, DYRK2, FLT-3, FYN, GSK-3α, GSK-3β, HCK, INSR, KIT, LCK, LYNA, MAPK1, MAPKAPK-2, MAPKAPK-3, MET, MSK1, MSK2, NEK2, P38-α, P38-β, P38-γ, P38-δ, P70S6K1, PAK2, PDGFR-α, PDK1, PKA, PRAK, ROCK2, SGK1, SRC, SYK, TRKB, and ZAP70.

In certain embodiments, compounds of Formula (III) exhibited selective inhibitory activity for at least one of the following human protein kinases: AKT2, AURORA-A, CDK2/cyclinE, CHEK1, CHEK2, CK1, DYRK2, FLT-3, FYN, GSK-3α, GSK-3β, INSR, KIT, LYNA, MAPK1, MAPKAPK-2, MAPKAPK-3, MSK2, NEK2, P38-α, PAK2, PDGFR-α, PDK1, PKA, PRAK, SYK, TRKB, and ZAP70.

In certain embodiments, compounds of Formula (IV) exhibited selective inhibitory activity for at least one of the following human protein kinases: ABL, ABL1, ABL-T315I, AKT1, AKT2, AURORA-A, BMX, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, INSR, KIT, LCK, LYNA, MAPKAPK-2, MET, MSK1, MSK2, NEK2, P38-α, P38-β, P38-γ, P38-δ, P70S6K1, PDGFR-α, PDK1, PKA, ROCK2, SRC, SYK, TRKB, and ZAP70.

In certain embodiments, compounds of Formula (V) exhibited selective inhibitory activity for at least one of the following human protein kinases: AURORA-A, CDK2/cyclinE, CK2, FLT-3, GSK-3α, GSK-3β, KIT, MSK1, P38-β, PDGFR-α, and TRKB.

In certain embodiments, compounds of Formula (VI) exhibited selective inhibitory activity for at least one of the following human protein kinases: ABL-1, ABL-T3151, AKT1, AKT2, AKT3, AURORA-A, BMX, CDK, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, HCK, INSR, KIT, LCK, LYNA, MAPKAPK-2, MET, MSK1, MSK2, NEK2, P38-α, P38-γ, P38-β, P38-δ, P70S6K1, PAK2, PDGFR-α, PDK1, PRAK, ROCK2, SGK1, SRC, SYK, TRKB, and ZAP70.

Synthesis of Certain Compounds

Compounds of the present disclosure can be prepared by methods well known in the art.

Compounds of the present disclosure can be prepared from readily available starting materials using the flowing general methods and procedures. It will be appreciated that where typical or preferred process conditions, such as, reaction temperatures, times, mole ratios of reactants, solvents, pressures, are given, other process conditions can also be used unless otherwise stated. Reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, 3^{rd} Edition, John Wiley & Sons, 1999, and references cited therein.

Furthermore, compounds of the present disclosure can contain one or more chiral centers. Accordingly, if desired, such compounds can be prepared or isolated as pure stereoisomers, i.e., as individual enantiomers or diastereomers, or as stereoisomer-enriched mixtures. All such stereoisomers, and enriched mixtures thereof, are included within the scope of the present disclosure, unless otherwise indicated. Pure stereoisomers, and enriched mixtures thereof, can be prepared using, for example, optically active starting materials or stereoselective reagents well-known in the art. Alternatively, racemic mixtures of such compounds can be separated using, for example, chiral column chromatography, chiral resolving agents and the like.

General synthetic schemes and specific reaction protocols used to prepare compounds of the present disclosure are presented in the reaction schemes and Examples provided herein.

Methods

In accordance with certain embodiments, compounds of the present disclosure exhibit ATP-utilizing enzyme inhibitory activity. Thus, one important use of the compounds of the present disclosure includes the administration of at least one compound of the present disclosure to a subject, such as a human. This administration can serve to arrest, ameliorate, reduce the risk of acquiring, reduce the development of or at least one of the clinical symptoms of, or reduce the risk of developing or at least one of the clinical symptoms of diseases or conditions regulated by ATP-utilizing enzymes, such as, protein kinases.

For example, unregulated or inappropriately high protein kinase activity has been implicated in many diseases resulting from abnormal cellular function. Unregulated or inappropriately high protein kinase activity can arise either directly or indirectly, for example, by failure of the proper control mechanisms of a protein kinase, related, for example, to mutation, over-expression or inappropriate activation of the enzyme; or by over- or under-production of cytokines or growth factors also participating in the transduction of signal upstream or downstream of the protein kinase. In all of these instances, selective inhibition of the action of a protein kinase can be expected to have a beneficial effect.

According to certain embodiments, the present disclosure relates to methods of treating a disease regulated by at least one ATP-utilizing enzyme in a subject. ATP-utilizing enzyme regulated diseases include, for example, those where the ATP-utilizing enzyme participates in the signaling, mediation, modulation, control or otherwise involved in the biochemical processes affecting the manifestation of a disease. In certain embodiments, the methods are useful in treating diseases regulated by protein kinase enzymes. Protein kinase regulated diseases include, for example, the following general disease classes: cancer, autoimmunological, metabolic, inflammatory, infection, diseases of the central nervous system, degenerative neural disease, allergy/asthma, angiogenesis, neovascularization, vasucolgenesis, cardiovascular, and the like. Without being limited by theory, specific examples of diseases that are known or believed to be regulated by protein kinase enzymes, include, transplant rejection, osteoarthritis, rheumatoid arthritis, multiple sclerosis, diabetes, diabetic retinopathy, asthma, inflammatory bowel disease such as Crohn's disease, and ulcerative colitis, renal disease cachexia, septic shock, lupus, diabetes mellitus, myasthenia gravis, psoriasis, dermatitis, eczema, seborrhea, Alzheimer's disease, Parkinson's disease, stem cell protection during chemotherapy, ex vivo selection or ex vivo purging for autologous or allogeneic bone marrow transplantation, leukemia including, but not limited to, acute myeloid leukemia, chronic myeloid leukemia, and acute lymphoblastic leukemia, cancer including but not limited to, breast cancer, lung cancer, colorectal cancer, ovary cancer, prostate cancer, renal cancer, squamous cell cancer, glioblastoma, melanoma, pancreatic cancer, and Kaposi's sarcoma, ocular disease, corneal disease, glaucoma, bacterial infections, viral infections, fungal infections, heart disease, stroke, and obesity.

Compounds of the present disclosure can be used in the treatment of diseases in which inappropriate protein kinase activity plays a role, including, for example, diabetes, inflammation, Alzheimer's disease, urodegeneration, stroke, obesity, and cancer.

Certain embodiments of the present disclosure are directed to methods of treating disease in a subject comprising the step of administering to a subject, in need of such treatment, a therapeutically effective dosage of at least one compound of the present disclosure. In some embodiments, a disease can be regulated by at least one ATP-utilizing enzyme such as a protein kinase. Certain diseases can be regulated by one or more ATP-utilizing enzymes. In such cases, treatment of the disease or disorder can include administering a therapeutically effective amount of at least one compound of the present disclosure that inhibits the activity of one or more ATP-utilizing enzymes, or more than one compound of the present disclosure, wherein each compound inhibits at least one different ATP-utilizing enzyme.

Other embodiments of the present disclosure are related to methods of inhibiting at least one ATP-utilizing enzyme, including for example, a protein kinase. In certain embodiments, the ATP-utilizing enzyme can be inhibited by the method of administering to a subject, at least one compound of any of the formulae described herein, or a composition comprising at least one compound of any of the formulae describe herein.

In certain embodiments, the present disclosure relates to methods of inhibiting ATP-utilizing enzyme activity by contacting at least one ATP-utilizing enzyme with at least one compound of the present disclosure. ATP-utilizing enzymes include phosphotransferase enzymes that catalyze the phosphorylation of a biological molecule by transferring a phosphate group from an ATP substrate. ATP-utilizing enzymes include for example, phosphatases, synthetases, ligases, synapsins, and kinases.

Certain methods of the present disclosure are useful in inhibiting protein kinase enzymes, including, for example, the following protein kinase enzymes: ABL, ABL-1, ABL-T315I, AKT1, AKT2, AKT3, AURORA-A, BMX, CDK, CDK1, CDK2/cyclinA, CDK2/cyclinE, CDK5, CHEK, CHEK1, CHEK2, CK1, CK2, CSK, c-TAK1, DAPK1, DYRK2, FLT-3, FYN, GSK-3α, GSK-3β, HCK, INSR, KIT, LCK, LYNA, MAPK1, MAPKAPK-2, MAPKAPK-3, MET, MSK1, MSK2, NEK2, P38-α, P38-β, P38-γ, P38-δ, P70S6K1, PAK2, PDGFR-α, PDK1, PKA, PRAK, ROCK2, SGK1, SRC, SYK, TRKB, and ZAP70.

Certain methods of the present disclosure using compounds of Formula (III) are useful in inhibiting protein kinase enzymes, including, for example, the following protein kinase enzymes: AKT2, AURORA-A, CDK2/cyclinE, CHEK1, CHEK2, CK1, DYRK2, FLT-3, FYN, GSK-3α, GSK-3β, INSR, KIT, LYNA, MAPK1, MAPKAPK-2, MAPKAPK-3, MSK2, NEK2, P38-α, PAK2, PDGFR-α, PDK1, PKA, PRAK, SYK, TRKB, and ZAP70.

Certain methods of the present disclosure using compounds of Formula (IV) are useful in inhibiting protein kinase enzymes, including, for example, the following protein kinase enzymes: ABL, ABL1, ABL-T315I, AKT1, AKT2, AURORA-A, BMX, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, INSR, KIT, LCK, LYNA, MAPKAPK-2, MET, MSK1, MSK2, NEK2, P38-α, P38-β, P38-γ, P38-δ, P70S6K1, PDGFR-α, PDK1, PKA, ROCK2, SRC, SYK, TRKB, and ZAP70.

Certain methods of the present disclosure using compounds of Formula (V) are useful in inhibiting protein kinase enzymes, including, for example, the following protein kinase enzymes: AURORA-A, CDK2/cyclinE, CK2, FLT-3, GSK-3α, GSK-3β, KIT, MSK1, P38-β, PDGFR-α, and TRKB.

Certain methods of the present disclosure using compounds of Formula (VI) are useful in inhibiting protein kinase enzymes, including, for example, the following protein kinase enzymes: ABL-1, ABL-T315I, AKT1, AKT2, AKT3, AURORA-A, BMX, CDK, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, HCK, INSR, KIT, LCK, LYNA, MAPKAPK-2, MET, MSK1, MSK2, NEK2, P38-α, P38-γ, P38-β, P38-δ, P70S6K1, PAK2, PDGFR-α, PDK1, PRAK, ROCK2, SGK1, SRC, SYK, TRKB, and ZAP70.

In certain embodiments, methods of the present disclosure can be used to inhibit ATP-utilizing enzymes that are present in a living organism, such as a mammal; contained in a biological sample such as a cell, cell culture, or extract thereof, biopsied material obtained from a mammal or extracts thereof, and blood, saliva, feces, semen, tears or other body fluids or extracts thereof; contained within a reagent, or bound to a physical support. In certain embodiments, an ATP-utilizing enzyme can regulate a disease or disorder and in other embodiments, the ATP-utilizing enzyme may not regulate a disease or disorder.

According to the methods of the present disclosure, at least one ATP-utilizing enzyme can be inhibited by contact with at least one compound of the present disclosure. In vivo ATP-utilizing enzymes can be inhibited by administration through routes and using compositions comprising at least one compound of the present disclosure previously described. For in vitro systems, contacting an ATP-utilizing enzyme with at least one compound of the present disclosure can include, for example, combining liquid reagents or combining a reagent and an ATP-utilizing enzyme and/or compound of the present disclosure attached to a solid support. The ATP-utilizing enzyme and compound of the present disclosure can be contacted in any appropriate device such as an affinity chromatography column, a microarray, a microfluidic device, assay plate, or other appropriate chemical or biotechnology apparatus used to perform biochemical analysis, assay, screening, and the like.

In certain embodiments, pharmaceutical compositions of the present disclosure may be administered orally, parenterally, by inhalation spray, topically, rectally, nasally, buccally, vaginally, via an implanted reservoir, or by any other appropriate route. Pharmaceutical compositions of the present disclosure can contain any conventional non-toxic pharmaceutically acceptable, excipients carriers, adjuvants and/or vehicles. In some embodiments, the pH of the formulation can be adjusted with pharmaceutically acceptable acids, bases or buffers to enhance the stability of the formulated compound or the delivery form. The term parenteral as used herein includes subcutaneous, intracutaneous, intravenous, intramuscular, intra-articular, intra-arterial, interasynovial, intrasternal, interathecal, intralesional, and intracranial injection or infusion techniques.

In certain embodiments, compounds disclosed herein can be delivered orally. Suitable dosage ranges for oral administration can depend on the potency of the compounds, but generally can range from 0.1 mg to 20 mg of a compound per kilogram of body weight. Appropriate dosages can be in the range of 25 to 500 mg/day and the dose of compounds administered can be adjusted to provide an equivalent molar quantity of compound in the plasma of a subject. Dosage ranges can be readily determined by methods known to those skilled in the art.

A dosage can be delivered in a composition by a single administration, by multiple applications, by sustained release or by controlled sustained release, or any other appropriate intervals and/or rates of release.

Compounds of the present disclosure can be assayed in vitro and in vivo, for the desired therapeutic or prophylactic activity prior to therapeutic use in mammals. For example, in vitro assays can be used to determine whether administration of a specific compound of the present disclosure or a combination of such compounds is effective for inhibiting the activity of certain ATP-utilizing enzymes or treating at least one disease. Compounds of the present disclosure can also be demonstrated to be effective and safe using animal model systems. A therapeutically effective dose of a compound of the present disclosure can, in certain embodiments, provide therapeutic benefit without causing substantial toxicity. Toxicity of compounds of the present disclosure can be determined using standard pharmaceutical procedures and can be readily ascertained by the skilled artisan. The dose ratio between toxic and therapeutic effect is the therapeutic index. Compounds of the present disclosure can exhibit high therapeutic indices in treating diseases and disorders. The dosage of a compound of the present disclosure can be within a range of circulating concentrations that include an effective dose with little or no toxicity.

Compositions

When employed as pharmaceuticals, compounds of the present disclosure can be administered in the form of pharmaceutical compositions. Such compositions can be prepared in a manner well known in the pharmaceutical art and can comprise at least one compound of the present disclosure.

Pharmaceutical compositions of the present disclosure can comprise a therapeutically effective amount of at least one compound of the present disclosure, and at least one pharmaceutically acceptable excipient, such as, for example, diluents, carriers, or adjuvants. Pharmaceutical compositions of the present disclosure can additionally comprise at least one compound that enhances the therapeutic efficacy of one or more compounds of the present disclosure. For example, such compounds can enhance the therapeutic efficacy of compounds of the present disclosure by effectively increasing the plasma concentration of the compounds. Without being limited by theory, certain compounds can decrease the degradation of the compounds of the present disclosure prior to administration or during transport to the plasma, or within the plasma. Certain compounds can increase the plasma concentration by increasing the absorption of compounds in the gastrointestinal tract. Pharmaceutical compositions of the present disclosure can also include additional therapeutic agents that are normally administered to treat a disease or disorder.

In certain embodiments, a pharmaceutical composition can include at least one compound of the present disclosure and at least one additional therapeutic agent appropriate for effecting combination therapy.

In some embodiments, compounds and compositions of the present disclosure can be administered by oral routes. The compositions can be prepared in a manner well known in the pharmaceutical art and can comprise at least one compound of the present disclosure. In some embodiments, compositions of the present disclosure contain a therapeutically effective amount of one or more thiophene-based compounds of the present disclosure, which can be in purified form, together with a therapeutically effective amount of at least one additional therapeutic agent, and a suitable amount of at least one pharmaceutically acceptable excipient, so as to provide the form for proper administration to a subject.

Some embodiments of the present disclosure are directed to compositions that contain, as the active ingredient, of one or more compounds of the present disclosure associated with pharmaceutically acceptable excipients. In making certain compositions of the present disclosure, the active ingredient can be mixed with an excipient, diluted by an excipient, or enclosed within such a carrier that can be in the form of a capsule, sachet, paper or other container. When the excipient serves as a diluent, the excipient can be a solid, semi-solid, or liquid material, which acts as a vehicle, carrier or medium for the active ingredient. Thus, for example, the compositions can be in the form of tablets, pills, powders, lozenges, sachets, cachets, elixirs, suspensions, emulsions, solutions, and syrups containing, for example, from 1% to 90% by weight of one or more compounds of the present disclosure using, for example, soft and hard gelatin capsules.

In preparing a composition, it can be necessary to mill the active compound to provide the appropriate particle size prior to combining with other ingredients. If the active compound is insoluble, the active component ordinarily can be milled to a particle size of less than 200 mesh. If the active compound is water soluble, the particle size can be adjusted by milling to provide a uniform distribution in the formulation, e.g. 40 mesh.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. Some compositions can additionally include, lubricating agents such as talc, magnesium stearate, and mineral oil, wetting agents, emulsifying and suspending agents, preserving agents such as methyl- and propylhydroxy-benzoates, sweetening agents, and flavoring agents. Compositions of the present disclosure can be formulated so as to provide quick, sustained or delayed release of the active ingredient after administration to the subject by employing procedures known in the art.

Some compositions of the present disclosure can be formulated in unit dosage form, each dosage containing, for example, 0.1 mg to 2 g of the active ingredient. As used herein, “unit dosage forms” refers to physically discrete units suitable as unitary dosages for human subjects and other mammals, each unit containing a predetermined quantity of active material calculated to produce the desired therapeutic effect, in association with a suitable pharmaceutical excipient, diluent, carrier and/or adjuvant. In certain embodiments, compositions of the present disclosure can be formulated in multiple dosage forms. The amount of the compounds of the present disclosure that can be combined with other materials and therapeutic agents to produce compositions of the present disclosure in a single dosage form will vary depending upon the subject and the particular mode of administration.

In the treatment of disease, compounds of the present disclosure can be administered in a therapeutically effective amount. It will be understood, however, that the amount of the compound administered will be determined by a physician, in the light of the relevant circumstances, including the condition to be treated, the chosen route of administration, the actual compound administered, the age, weight, and response of the individual subject, the severity of the subject's symptoms, and the like.

For preparing solid compositions such as tablets, the principal active ingredient can be mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills and capsules. The solid preformulation can then subdivided into unit dosage forms of the type described above containing from, for example, 0.1 mg to 2 g of the therapeutically effective compound of the present disclosure.

The tablets or pills comprising certain compositions of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials include a number of polymeric acids and mixtures of polymeric acids with such materials as shellac, cetyl alcohol, and cellulose acetate.

The liquid forms in which the compositions of the present disclosure may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

In addition to the compounds of this present disclosure, pharmaceutically acceptable derivatives or prodrugs of the compounds of this present disclosure may also be employed in pharmaceutical compositions to treat or prevent the above-identified disorders.

As used herein, a “pharmaceutically acceptable derivative or prodrug” refers to any pharmaceutically acceptable salt, ester, salt of an ester or other derivative of a compound of the present disclosure that, upon administration to a recipient, is capable of providing, either directly or indirectly, a compound of the present disclosure or an inhibitory active metabolite or residue thereof. Examples of such derivates or prodrugs include those that increase the bioavailability of the compounds of the present disclosure when such compounds are administered to a mammal, e.g., by allowing an orally administered compound to be more readily absorbed into the blood, or which enhance delivery of the parent compound to a biological compartment, e.g., the brain or lymphatic system, relative to the parent species.

In certain embodiments, acceptable formulation materials can be nontoxic to recipients at the dosages and concentrations employed.

In certain embodiments, a pharmaceutical composition of the present disclosure can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids such as glycine, glutamine, asparagine, arginine or lysine; antimicrobials; antioxidants such as ascorbic acid, sodium sulfite, or sodium hydrogen-sulfite; buffers such as borate, bicarbonate, Tris-HCl, citrates, phosphates or other organic acids; bulking agents such as mannitol or glycine; chelating agents such as ethylenediamine tetraacetic acid (EDTA); complexing agents such as caffeine, polyvinylpyrrolidone, beta-cyclodextrin or hydroxypropyl-beta-cyclodextrin; fillers; monosaccharides; disaccharides; and other carbohydrates such as glucose, mannose, or dextrins; proteins such as serum albumin, gelatin or immunoglobulins; coloring, flavoring and diluting agents; emulsifying agents; hydrophilic polymers such as polyvinylpyrrolidone; low molecular weight polypeptides; salt-forming counterions such as sodium; preservatives such as benzalkonium chloride, benzoic acid, salicylic acid, thimerosal, phenethyl alcohol, methylparaben, propylparaben, chlorhexidine, sorbic acid or hydrogen peroxide; solvents such as glycerin, propylene glycol or polyethylene glycol; sugar alcohols such as mannitol or sorbitol; suspending agents; surfactants or wetting agents such as pluronics, PEG, sorbitan esters, polysorbates such as polysorbate 20, polysorbate 80, triton, tromethamine, lecithin, cholesterol, tyloxapal; stability enhancing agents such as sucrose or sorbitol; tonicity enhancing agents such as alkali metal halides, such as sodium or potassium chloride, mannitol, sorbitol; delivery vehicles; diluents; excipients and/or pharmaceutical adjuvants (Remington's Pharmaceutical Sciences, 18^{th} Edition, A. R. Gennaro, ed., Mack Publishing Company (1990)).

In certain embodiments, the optimal pharmaceutical composition can be determined by one skilled in the art depending upon, for example the intended route of administration, delivery format, and desired dosage. See, for example, Remington's Pharmaceutical Sciences, supra. In certain embodiments, such compositions may influence the physical state, stability, rate of in vivo release, and rate of in vivo clearance of the antibodies of the present disclosure.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, neutral buffered saline or saline mixed with serum albumin are further exemplary vehicles. In certain embodiments, pharmaceutical compositions comprise Tris buffer of pH 7 to 8.5, or acetate buffer of pH 4 to 5.5, which can further comprise sorbitol or a suitable substitute thereof. In certain embodiments, buffers are used to maintain the composition at physiological pH or at a slightly lower pH, typically within a pH range of from 5 to 8.

In certain embodiments, pharmaceutical compositions of the present disclosure can be selected for parenteral delivery. In other embodiments, compositions can be selected for inhalation or for delivery through the digestive tract, such as orally. The preparation of such pharmaceutically acceptable compositions is within the skill of the art.

In certain embodiments, composition components can be present in concentrations that are acceptable to the site of administration. In certain embodiments, when parenteral administration is contemplated, a therapeutic composition can be in the form of a pyrogen-free, parenterally acceptable aqueous solution comprising at least one compound of the present disclosure, with or without additional therapeutic agents, in a pharmaceutically acceptable vehicle. In other embodiments, a vehicle for parenteral injection can be sterile distilled water in which at least one compound of the present disclosure, with or without at least one additional therapeutic agent, is formulated as a sterile, isotonic solution, properly preserved. In still other embodiments, the pharmaceutical composition can include encapsulation of at least one compound of the present disclosure with an agent, such as injectable microspheres, bio-erodible particles, polymeric compounds such as polyacetic acid or polyglycolic acid, beads or liposomes, that can provide the controlled or sustained release of the compound of the present disclosure which can then be delivered via a depot injection. In certain embodiments, implantable drug delivery devices can be used to introduce a compound of the present disclosure to the plasma of a subject, within a target organ, or to a specific site within the subject's body.

In certain embodiments, a pharmaceutical composition can be formulated for inhalation. In certain embodiments, a compound of the present disclosure, with or without at least one additional therapeutic agent, can be formulated as a dry powder for inhalation. In certain embodiments, an inhalation solution comprising a compound of the present disclosure with or without at least one additional therapeutic agent can be formulated with a propellant for aerosol delivery. In other embodiments, solutions can be nebulized. In still other embodiments, solutions, powders or dry films of compounds of the present disclosure can be aerosolized or vaporized for pulmonary delivery.

In certain embodiments, it is contemplated that formulations can be administered orally. In certain embodiments, a compound of the present disclosure, with or without at least one additional therapeutic agent that can be administered orally, can be formulated with or without carriers customarily used in the compounding of solid dosage forms such as tablets and capsules. In other embodiments, a capsule may be designed to release the active portion of the formulation in the region of the gastrointestinal tract where bioavailability can be maximized and pre-systemic degradation minimized. In still other embodiments, at least one additional agent can be included in the formulation to facilitate absorption of the compound of the present disclosure and/or any additional therapeutic agents into the systemic circulation. In certain embodiments, diluents, flavorings, low melting point waxes, vegetable oils, lubricants, suspending agents, tablet disintegrating agents, and binders can be employed.

In certain embodiments, a pharmaceutical composition of the present disclosure can include an effective quantity of compounds of the present disclosure, with or without at least one additional therapeutic agent, in a mixture with non-toxic excipients which are suitable for the manufacture of tablets. In certain embodiments, by dissolving the tablets in sterile water, or other appropriate vehicle, solutions can be prepared in unit-dose form. In certain embodiments, suitable excipients include inert diluents, such as calcium carbonate, sodium carbonate or bicarbonate, lactose, or calcium phosphate; or binding agents, such as starch, gelatin, or acacia; and lubricating agents such as magnesium stearate, stearic acid or talc.

In certain embodiments, the frequency of dosing will take into account the pharmacokinetic parameters of the compounds of the present disclosure and/or any additional therapeutic agents in the pharmaceutical composition used. In certain embodiments, a clinician can administer the composition until a dosage is reached that achieves the desired effect. The composition can be administered as a single dose, or as two or more doses, which may or may not contain the same amount of the therapeutically active compound time, or as a continuous infusion via an implantation device or catheter. Further refinement of an appropriate dosage can be routinely made by those of ordinary skill in the art. For example, therapeutically effective dosages and dosage regiments can be determined through use of appropriate dose-response data.

In certain embodiments, the route of administration of the pharmaceutical composition can be in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions can be administered by bolus injection or continuously by infusion, or by an implantation device.

In certain embodiments, the composition can be administered locally via implantation of a membrane, sponge or another appropriate material onto which the desired compound of the present disclosure has been absorbed or encapsulated. In certain embodiments, where an implantation device is used, the device can be implanted into any suitable tissue or organ, and delivery of the desired molecule via diffusion, timed-release bolus, or continuous administration.

In certain embodiments, it can be desirable to use a pharmaceutical composition comprising a compound of the present disclosure, with or without at least one additional therapeutic agent, in an ex vivo manner. For example, cells, tissues and/or organs that have been removed from a subject are exposed to a pharmaceutical composition comprising a compound of the present disclosure, with or without at least one additional therapeutic agent, after which the cells, tissues and/or organs are subsequently implanted back into the subject.

In certain embodiments, a compound of the present disclosure and/or any additional therapeutic agents can be delivered by implanting certain cells that have been genetically engineered, using methods known in the art, to express and secrete the compounds of the present disclosure. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials can be biocompatible, semi-permeable polymeric enclosures or membranes that enable the release of the protein product(s) while preventing the destruction of the cells by the subject's immune system or by other detrimental factors originating from the surrounding tissues.

Pharmaceutical compositions according to the present disclosure can take a form suitable for oral, buccal, parenteral, nasal, topical or rectal administration, or a form suitable for administration by inhalation or insufflation.

The compositions of the present disclosure can, if desired, be presented in a pack or dispenser device that can contain one or more unit dosage forms containing the active ingredient. The pack or dispensing device can be accompanied by instructions for administration.

The quantity of a compound of the present disclosure required for the treatment of a particular condition can vary depending on the compound, and the condition of the subject to be treated. In general, daily dosages can range from 100 ng/kg to 100 mg/kg, e.g., 0.01 mg/kg to 40 mg/kg body weight, for oral or buccal administration; from 10 ng/kg to 50 mg/kg body weight, e.g., 0.001 mg/kg to 20 mg/kg body weight, for parenteral administration; and from 0.05 mg to 1,000 mg for nasal administration or administration by inhalation or insufflation.

Certain compounds of the present disclosure and/or compositions of the present disclosure can be administered as sustained release systems. In certain embodiments, the compounds of the present disclosure can be delivered by oral sustained release administration. In this embodiment, the compounds of the present disclosure can be administered, for example, twice per day and, once per day.

The compounds of the present disclosure can be practiced with a number of different dosage forms, which can be adapted to provide sustained and/or extended release of a compound upon oral administration. Examples of sustained and/or extended release dosage forms include, but are not limited to, beads comprising a dissolution or diffusion release composition and/or structure, an oral sustained release pump, enteric-coated preparations, compound-releasing lipid matrices, compound releasing waxes, osmotic delivery systems, bioerodible polymer matrices, diffusible polymer matrices, a plurality of time-release pellets, and osmotic dosage forms.

Regardless of the specific form of sustained release oral dosage form used, the compounds and composition of the present disclosure can be released from the dosage form over an extended period of time. In certain embodiments, sustained release oral dosage forms can provide a therapeutically effective amount of a compound of the present disclosure over a period of at least several hours. In certain embodiments the extended release dosage form can provide a constant therapeutically effective concentration of a compound of the present disclosure in the plasma of a subject for a prolonged period of time, such as at least several hours. In other embodiments, the sustained release oral dosage form can provide a controlled and constant concentration of a therapeutically effective amount of a compound of the present disclosure in the plasma of a subject.

Dosage forms comprising compositions and compounds of the present disclosure can be administered at certain intervals such as, for example, twice per day or once per day.

Exemplary dosage ranges for oral administration are dependent on the potency of the compound of the present disclosure, but can range from 0.1 mg to 20 mg of the compound per kilogram of body weight. Dosage ranges may be readily determined by methods known to those skilled in the art.

Compounds of the present disclosure can be assayed in vitro and in vivo, to determine and optimize therapeutic or prophylactic activity prior to use in subjects. For example, in vitro assays can be used to determine whether administration of a specific compound of the present disclosure or a combination of such compounds exhibits therapeutic efficacy. Compounds of the present disclosure can also be demonstrated to be effective and safe using animal model systems.

It is desirable that a therapeutically effective dose of a compound of the present disclosure provide therapeutic benefit without causing substantial toxicity. Toxicity of compounds of the present disclosure can be determined using standard pharmaceutical procedures and can be readily ascertained by the skilled artisan. The dose ratio between toxic and therapeutic effect is the therapeutic index. In certain embodiments, compounds of the present disclosure can exhibit particularly high therapeutic indices in treating diseases and disorders. In certain embodiments, the dosage of a compound of the present disclosure can be within a range of circulating concentration that exhibits therapeutic efficacy with limited or no toxicity.

### EXAMPLES

Embodiments of the present disclosure can be further defined by reference to the following examples, which describe in detail preparation of compounds of the present disclosure and assays for using compounds of the present disclosure. It will be apparent to those skilled in the art that many modifications, both to materials and methods, may be practiced without departing from the scope of the present disclosure.

In the examples below, the following abbreviations have the following meanings. If an abbreviation is not defined, it has its generally accepted meaning.

| | |
|---|---|
| ATP = | adenosine triphosphate |
| BSA = | bovine serum albumin |
| Da = | Dalton |
| DMSO = | dimethylsulfoxide |
| DTT = | (R,R)-dithiothreitol |
| EDTA = | ethylenediaminetetraacetic acid |
| g = | gram |
| hr = | hour |
| L = | liter |
| HPLC = | high performance liquid chromatography |
| M = | molar |
| MS = | mass spectroscopy |
| min = | minute |
| mL = | milliliter |
| mm = | millimeter |
| mmol = | millimoles |
| mM = | millimolar |
| nM = | nanomolar |
| μL = | microliter |
| μM = | micromolar |
| psi = | pound per square inch |
| RT = | retention time |
| THF = | tetrahydrofuran |
| TFA = | trifluoroacetic acid |

### Example 1

### Synthesis of Certain Compounds of Formula (IV)

Certain compounds of the invention having the structure of Formula (IV) were prepared using the following general synthetic scheme:

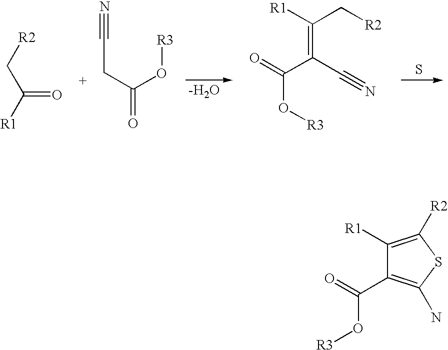

The synthesis was performed in a two-step process. The first step in the synthesis of certain compounds of the invention having the structure of Formula (IV) was the formation of the Schiff's base from the corresponding ketones and cyanoacetates according to the following general reaction scheme: 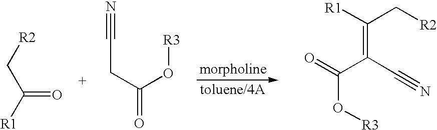
Five (5) mmol of each of the corresponding ketones and cyanoacetates were dissolved with gentle heating in 5 mL dry toluene followed by addition of 5 mmol dry morpholine. Activated molecular sieves 4A were then added to the reaction vessel. The reaction mixture was maintained at 80° C. for 12 hrs.

The second step involved a Gewald reaction according to the following general reaction scheme:

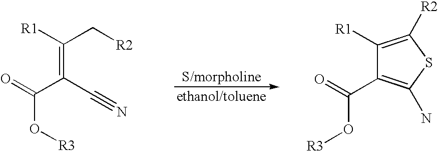

Five (5) mL of absolute ethanol and 0.16 g sulfur (5 mmol) were added to the reaction mixture from previous step. The reaction mixture was heated with mixing at 70° C. for 12 hrs. Residues were purified after evaporation of solvents by HPLC. Crude yields before purification were 60-90% based on HPLC analysis.

Following HPLC purification, compounds were characterized by HPLC/MS/JUV/ELSD. A Shimadzu reversed-phase high performance liquid chromatography (HPLC) system was interfaced to a Sciex API-100 electrospray single quadrupole mass spectrometer using a LEAP HTS Pal autosampler for sample introduction. The following HPLC conditions were used for characterizing the compounds:
Column: Chromolith SpeedRod RP-18e C18 analytical column (4.6 mm×50 mm) from Phenomenex (CA, USA)
Flow rate: 1.5 mL/min

Two mobile phases (phase A: 100% water, 0.1% trifluoroacetic acid (TFA); phase B: 100% acetonitrile, 0.12% TFA) were employed to run a gradient condition from 5% B to 100% B in 4.4 min, with a stay at 100% B for 1 min, and a re-equilibrate for 0.6 min. An injection volume of 10 μl was used.

Retention times (RT) for certain compounds of the invention with reference to FIG. 2 are provided in the following table.

| Compound | RT (min) |
|---|---|
| 2.15 | 3.19 |
| 2.16 | 3.78 |
| 2.24 | 3.55 |
| 2.37 | 3.09 |
| 2.59 | 4.08 |
| 2.76 | 3.85 |
| 2.86 | 3.71 |
| 2.89 | 3.45 |
| 2.154 | 3.42 |

### Example 2

### HTS ATP-Utilizing Enzyme Assays

The following procedures describe the reagent and plate preparation for a HTS of an ATP-utilizing enzyme, such as a protein kinase, run in an off-chip mobility-shift assay format. The following provides an HTS protocol for running a protein kinase HTS screen on a Caliper HTS 250 microfluidics system. The following parameters are dependent on the protein kinase used and can be determined by one skilled in the art as part of a typical assay development process. For example, the peptide substrate used can be identified from the current literature, by screening a peptide library of potential protein kinase substrates, or by other applicable means accepted in the field.

The following table provides typical screen assay parameters appropriate for a Caliper HTS 250 microfluidics system used to assay AKT1. Parameters used to assay other protein kinases can be determined by one skilled in the art.

| Reaction | | |
|---|---|---|
| Concentration | | |
| Inhibitor concentration | 10 | μM |
| Enzyme concentration | 18 | nM |
| Substrate/Peptide Conc. | 1 | μM |
| Reaction Properties | | |
| Inhibitor Volume | 5 | μL |
| Enzyme Volume | 10 | μL |
| Substrate Volume | 10 | μL |
| Termination Volume | 45 | μL |
| Reaction Time | 1-24 | hrs |
| Reaction Temperature | 20-25 | ° C. |
| Sipper Properties | | |
| Initial Delay | 18 | sec |
| Buffer | 18 | sec |
| Sample | 0.2 | sec |
| Final Delay | 120 | sec |
| Dye Well | | |
| Dye | 0.2 | sec |
| Pressure Driven Flow | | |
| Script Properties | | |
| Electrode 1 | −250 | Volts |
| Electrode 2 | −2250 | Volts |
| Electrode 3 | −2250 | Volts |
| Electrode 4 | −250 | Volts |
| Laser Properties | yes/no | |
| UV | no | |
| Blue | yes | |
| Red | no | |
| Data Collection | yes/no | |
| CCD1 | no | |
| CCD2 | yes | |
| CCD3 | no | |
| Inhibitor | | |
| Concentrations | | |
| Inhibitor: EDTA | | |
| 100% | 20 | mM |
| Inhibitor Staurosporine | | |
| 70% | 138 | nM |
| Pressure | −2 | psi |
| Base Pressure | −2 | psi |

The reagents and buffers listed in the following table are generally applicable for developing and running an HTS screen on a human protein kinase using the Caliper HTS 250 system.

| | REAGENT | |
|---|---|---|
| REAGENT | NAME | MANUFACTURER |
| 4 sipper | FS266 | Caliper Tech. Inc. |
| LABCHIP | | |
| Enzyme | Specific kinase | — |
| Substrate | Specific peptide | — |
| Control | Specific | LKT |
| Inhibitor | compound | |
| Buffer | HEPES (free | Calbiochem |
| Components | acid) | |
| | HEPES (Na Salt) | Calbiochem |
| | DMSO | Sigma |
| | Triton X-100 | Sigma |
| | BSA | Sigma |
| | DTT(Cleland's | Calbiochem |
| | Reagent) | |
| | EDTA (0.5M) | Sigma |
| | Coating Reagent 3 | Caliper Tech. Inc. |
| | 6N HCl | VWR |
| | ATP disodium | Sigma |
| | salt | |
| | Na₃VO₄ | Calbiochem |
| | B- | Calbiochem |
| | Glycerophosphate | |
| | MgCl₂.6H₂O | Sigma |

The following reagents were prepared using the previously described buffers.

A 2× Master Buffer solution was prepared by combining 200 mL of 1 M HEPES, 2 mL of 10% Triton X-100, 20 mL of 10% BSA, and 778 mL of H₂O.

A 2.5× Enzyme Buffer solution was prepared by combining 177.408 mL of 2× Master Buffer, 0.887 mL of 1 M DTT, 0.089 mL of 100 mM ATP, 8.870 mL of 1 M MgCl₂, 0.089 mL of 100 mM β-glycerophosphate, 0.089 mL of Na₃VO₄, 0.254 mL of 62.8 μM enzyme, and 167.13 mL H₂O.

A 2.5× Substrate Buffer solution was prepared by combining 177.408 mL of 2× Master Buffer, 0.887 mL of 1 mM peptide-3, and 176.521 mL of H₂O.

A 1.55× Termination Buffer solution was prepared by combining 762.05 mL of 2× Master Buffer, 95.1 mL of 0.5 M EDTA, and 666.94 MI of H₂O.

A TCB Buffer solution was prepared by combining 125 mL of 2× Master Buffer, 10 mL of 0.5 M EDTA, 6.25 mL of 4% coating reagent, 1.01 mL of 100% DMSO, and 107.74 mL H₂O.

A Dye Trough solution was prepared by combining 0.5 μL of peptide-X, and 2,999.5 μL of 1× Master Buffer.

A 0% Control solution was prepared by combining 6,804 μL of 2× Master Buffer, 770.21 μL of 100% DMSO, and 6,033.79 μL H₂O.

A 100% Inhibition solution was prepared by combining 2,268 mL of 2× Master Buffer, 907.2 μL of 500 mM EDTA, 256.74 μL of 500 mM DMSO, and 1,104.06 μL H₂O.

A 70% Inhibition Control solution was prepared by combining 4,536 μL of 2× Master Buffer, 6.26 μL of 1 mM of an inhibitor, 513.48 μL of 100% DMSO, and 4,016.27 μL of H₂O. Examples of inhibitors include, Staurosporine, GFF109203X, SB202190, H-89, AMPPNP, and K252a.

A 1.06× Assay Buffer solution was prepared by combining 205.15 mL of 2× Master Buffer, and 181.92 mL of H₂O.

Assays to determine the kinase inhibitory activity of compounds of the invention were performed using a Caliper HTS 250 microfluidics device, Greiner U-bottom assay plates, a Multidrop for transfer of reagents, and Biomek FX (AMNCBM03) software. Initially, 2.4 μL of a 100 μM solution of a test compound in 100% DMSO is added to a well of the Greiner U-bottom plate. A single Greiner U-bottom plate having 24×16 wells can include multiple test compounds. Next, 2.6 μL of 1.06× Assay Buffer was added to each well of the assay plate. Using the Multidrop, the 0% Control, 100% Control and the 70% Control reagents were added to certain wells. Again, using the Multidrop, 10 μL of 2.5× Enzyme Buffer, followed by 10 μL of 2.5× Substrate Buffer was added to each well of the assay plate. The total reaction volume in each well was 25 μL, and the concentration of the test compound was 10 μM. The assay plate was covered with foil and incubated for 2.5 hrs at 20° C. to 22° C. After the incubation period, 45 μL of 1.55× Termination Buffer was added to each well of the assay plate to stop the reaction. The inhibition of the ATP-utilizing enzyme, such as a kinase, was determined by measuring the ratio of the peptide substrate to phoshorylated product for each well of the assay plate using the Caliper HTS 250 system. Compounds exhibiting a ratio of at least 10% were determined to exhibit inhibitory activity for the particular ATP-utilizing enzyme assayed.

Assays to determine the kinase inhibitory activity of compounds of the present disclosure were performed using a Caliper HTS 250 microfluidics device, Greiner U-bottom assay plates, a Multidrop for transfer of reagents, and Biomek FX (AMNCBM03) software. Initially, 2.4 μL of a 1 mM solution of a test compound in 100% DMSO was added to a well of the Greiner U-bottom plate. A single Greiner U-bottom plate having 24×16 wells could include multiple test compounds. Next, 40 μL of 1.06× Assay Buffer was added to each well of the assay plate. Using the Biomek FX, 10 μL of 0.5 M EDTA was added by the span-8 to wells, indicated as 100% Control and 2.4 μL of 100% DMSO was added by the span-8 to wells, indicated as 0% Control. Using the Multidrop, 10 μL of 2.5× Enzyme Buffer, followed by 10 μL of 2.5× Substrate Buffer was added to each well of the assay plate. The total reaction volume in each well was 25 μL, and the concentration of the test compound was 10 μM. The assay plate was incubated for 2.5 hrs at 20° C. to 22° C. After the incubation period, using the Multidrop, 45 μL of 1.55× Termination Buffer was added to each well of the assay plate to stop the reaction. The inhibition of the ATP-utilizing enzyme, such as a particular protein kinase, was determined by measuring the ratio of the peptide substrate to phosphorylated product for each well of the assay plate using the Caliper HTS 250 system.

Compounds exhibiting a inhibitory activity for a particular target ATP-utilizing enzyme greater than three-sigma from the mean activity for the population of predominately inactive compounds for the same target ATP-utilizing enzyme were considered to be active inhibitors for a particular target ATP-utilizing enzyme. The use of three-sigma statistical limits represents a conservative method for declaring potential hits among targets. The three-sigma activity, as well as the mean population activity, can be different for each target enzyme. This method has an expected false positive rate, from an in-control measurement process, of one in one million. Compounds were considered to show selectivity between a primary target and one or more other targets if the activity (e.g. % inhibition, IC50, Ki, EC50, etc.) for that compound against the primary target was significantly different than that for the other target(s) within the error of the activity measurement.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. At least one compound of Formula (I): 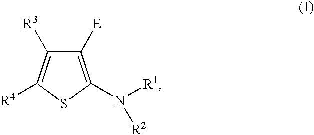
a stereoisomer thereof, a pharmaceutically acceptable salt thereof, a hydrate thereof, or a solvate of any of the foregoing, wherein:
E is chosen from CN, halogen, —NO₂, and —C(═X)YR⁵; wherein
X is chosen from O, and S;
Y is chosen from —N(R¹⁰)—, O, S, and a direct bond; wherein
R¹⁰ is chosen H, alkyl, and substituted alkyl; and
R⁵ is chosen from H, alkyl, substituted alkyl, arylalkyl, substituted arylalkyl, cycloalkyl, substituted cycloalkyl, heterocycloalkyl, substituted heterocycloalkyl, heteroalkyl, substituted heteroalkyl, heteroarylalkyl, substituted heteroarylalkyl, and when Y is —N(R¹⁰)—, or a direct bond, then R⁵ is additionally chosen from aryl, substituted aryl, heteroaryl, substituted heteroaryl, —N(R⁷)₂, and —OR⁹; wherein each R⁷ is independently chosen from alkyl, substituted alkyl, aryl, substituted aryl, and H; and
R⁹ is chosen from H, alkyl, and substituted alkyl;
or R⁵ and R¹⁰ together with the atoms to which R⁵ and R¹⁰ are attached form a cycloalkyl, substituted cycloalkyl, heterocycloalkyl, or substituted heterocycloalkyl ring;
R¹ is chosen from H, alkyl, substituted alkyl, cycloalkyl, substituted cycloalkyl, heteroalkyl, and substituted heteroalkyl;
R² is chosen from H, —CHO, alkyl, substituted alkyl, aryl, substituted aryl, cycloalkyl, substituted cycloalkyl, heteroaryl, substituted heteroaryl, heterocycloalkyl, substituted heterocycloalkyl, heteroarylalkyl, substituted heteroarylalkyl, heterocycloalkylalkyl, substituted heterocycloalkylalkyl, alkylsulfonyl, substituted alkylsulfonyl, heteroalkylsulfonyl, substituted heteroalkylsulfonyl, and -ZR⁶, wherein
Z is chosen from carbonyl, —C(O)O—, aminosulfonyl, aminothiocarbonyl, —C(═O)NR¹¹—, sulfonyl, and thiocarbonyl; wherein
R¹¹ is chosen from alkyl, substituted alkyl, cycloalkyl, substituted cycloalkyl, and H; and
R⁶ is chosen from H, —COOH, alkyl, substituted alkyl, aryl, substituted aryl, cycloalkyl, substituted cycloalkyl, heterocycloalkyl, substituted heterocycloalkyl, heteroalkyl, substituted heteroalkyl, heteroaryl, substituted heteroaryl, cycloalkylalkyl, substituted cycloalkylalkyl, heterocycloalkylalkyl, substituted heterocycloalkylalkyl, arylalkyl, substituted arylalkyl, heteroarylalkyl, substituted heteroarylalkyl, bicycloalkyl, substituted bicycloalkyl, bicycloheteroalkyl, and substituted bicycloheteroalkyl;
or R¹ and R², together with the atoms to which R¹ and R² are attached, form a heterocycloalkyl, or substituted heterocycloalkyl ring;
R³ is chosen from H, halogen, —NH₂, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, dialkylamino, and substituted dialkylamino; and
R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
or R³ and R⁴ together with the atoms to which R³ and R⁴ are attached form a cycloalkyl, substituted cycloalkyl, heterocycloalkyl, substituted heterocycloalkyl, bicycloalkyl, substituted bicycloalkyl, bicycloheteroalkyl, or substituted bicycloheteroalkyl ring;
with the provisos that
when E is —CO₂R⁵, then
R³ is not H, 2-aminopyrimidine, substituted 2-aminopyrimidine 2-aminopyridine, substituted 2-aminopyridine, aminotriazine, or substituted aminotriazine; and
R⁴ is not 2-aminopyrimidine, substituted 2-aminopyrimidine 2-aminopyridine, substituted 2-aminopyridine, aminotriazine, or substituted aminotriazine;
when E is —CN, then
R³ is not H, 2-aminopyrimidine, substituted 2-aminopyrimidine 2-aminopyridine, substituted 2-aminopyridine, aminotriazine, or substituted aminotriazine; and
R⁴ is not H, 2-aminopyrimidine, substituted 2-aminopyrimidine 2-aminopyridine, substituted 2-aminopyridine, aminotriazine, or substituted aminotriazine;
when E is —CN, and R² is —C(═X)NH₂, where X is O or S, then
R³ is not unsubstituted phenyl, or a 5 to 7 member heteroaromatic ring containing 1 to 3 heteroatoms chosen from O, N or S; and
R⁴ is not unsubstituted phenyl, or a 5 to 7 member heteroaromatic ring containing 1 to 3 heteroatoms chosen from O, N or S;
when E is —C(═O)NR⁵R¹⁰, and R³ is H, and R² is C(═O)NR¹²R¹¹, and R¹¹ is H, then R¹² is not alkyl or substituted alkyl; and
when E is —C(═O)NR⁵R¹⁰, and R¹ is H, and R⁵ is H, then R¹⁰ is not H;
and wherein the compound of Formula (I), a stereoisomer thereof, a pharmaceutically acceptable salt thereof, a hydrate thereof, or a solvate of any of the foregoing, exhibits ATP-utilizing enzyme inhibitory activity.

2. At least one compound of Formula (II): 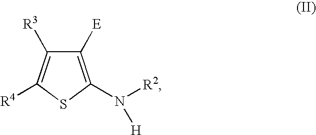
a stereoisomer thereof, a pharmaceutically acceptable salt thereof, a hydrate thereof, or a solvate of any of the foregoing, wherein:
E is chosen from —CN, halogen, —NO₂, and —C(═X)YR⁵; wherein
X is chosen from O, and S;
Y is chosen from —N(R¹⁰)—, O, S, and a direct bond; wherein
R¹⁰ is chosen H, alkyl, and substituted alkyl; and
R⁵ is chosen from H, alkyl, substituted alkyl, arylalkyl, substituted arylalkyl, cycloalkyl, substituted cycloalkyl, heterocycloalkyl, substituted heterocycloalkyl, heteroalkyl, substituted heteroalkyl, heteroarylalkyl, substituted heteroarylalkyl, and when Y is —N(R¹⁰)—, or a direct bond, then R⁵ is additionally chosen from aryl, substituted aryl, heteroaryl, substituted heteroaryl, —N(R⁷)₂, and —OR⁹; wherein
each R⁷ is independently chosen from alkyl, substituted alkyl, aryl, substituted aryl, and H; and
R⁹ is chosen from H, alkyl, and substituted alkyl;
or, R⁵ and R¹⁰ together with the atoms to which R⁵ and R¹⁰ form a cycloalkyl, substituted cycloalkyl, heterocycloalkyl, or substituted heterocycloalkyl ring;
R² is chosen from H, —CHO, alkyl, substituted alkyl, aryl, substituted aryl, cycloalkyl, substituted cycloalkyl, heteroaryl, substituted heteroaryl, heterocycloalkyl, substituted heterocycloalkyl, heteroarylalkyl, substituted heteroarylalkyl, heterocycloalkylalkyl, substituted heterocycloalkylalkyl, alkylsulfonyl, substituted alkylsulfonyl, heteroalkylsulfonyl, substituted heteroalkylsulfonyl, and -ZR⁶, wherein
Z is chosen from carbonyl, —C(O)O—, aminosulfonyl, aminothiocarbonyl, —C(═O)NR¹¹—, sulfonyl, and thiocarbonyl; wherein
R¹¹ is chosen from alkyl, substituted alkyl, cycloalkyl, substituted cycloalkyl, and H; and
R⁶ is chosen from H, —COOH, alkyl, substituted alkyl, aryl, substituted aryl, cycloalkyl, substituted cycloalkyl, heterocycloalkyl, substituted heterocycloalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, arylalkyl, substituted arylalkyl, heteroarylalkyl, substituted heteroarylalkyl, bicycloalkyl, substituted bicycloalkyl, bicycloheteroalkyl, and substituted bicycloheteroalkyl;
or R¹ and R², together with the atoms to which R¹ and R² are attached, form a cycloalkyl, substituted cycloalkyl, heterocycloalkyl, or substituted heterocycloalkyl ring;
R³ is chosen from H, halogen, —NH₂, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, dialkylamino, and substituted dialkylamino; and
R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
or R³ and R⁴ together with the atoms to which R³ and R⁴ are attached form a cycloalkyl, substituted cycloalkyl, heterocycloalkyl, substituted heterocycloalkyl, bicycloalkyl, substituted bicycloalkyl, bicycloheteroalkyl, or substituted bicycloheteroalkyl ring;
with the provisos that
when E is —CO₂R⁵, then
R³ is not H, 2-aminopyrimidine, substituted 2-aminopyrimidine, 2-aminopyridine, substituted 2-aminopyridine, aminotriazine or substituted aminotriazine; and
R⁴ is not 2-aminopyrimidine, substituted 2-aminopyrimidine, 2-aminopyridine, substituted 2-aminopyridine, aminotriazine or substituted aminotriazine;
when E is —CN, then
R³ is not 2-aminopyrimidine, substituted 2-aminopyrimidine, 2-aminopyridine, substituted 2-aminopyridine, aminotriazine or substituted aminotriazine; and
R⁴ is not 2-aminopyrimidine, substituted 2-aminopyrimidine, 2-aminopyridine, substituted 2-aminopyridine, aminotriazine or substituted aminotriazine;
when E is —CN, and R² is —C(═X)NH₂, then
R³ is not unsubstituted phenyl or a 5 to 7 membered heteroaromatic ring containing 1 to 3 heteroatoms chosen from O, N or S; and
R⁴ is not unsubstituted phenyl or a 5 to 7 membered heteroaromatic ring containing 1 to 3 heteroatoms chosen from O, N or S;
when E is —C(═O)NR⁵R¹⁰, and R³ is H, and R² is —C(═O)NR¹²R¹¹, and R¹¹ is H, then R¹² is not alkyl, or substituted alkyl; and
when E is —C(═O)NR⁵R¹⁰, and R¹ is H, and R⁵ is H, then R¹⁰ is not H;
and wherein the compound of Formula (II), a stereoisomer thereof, a pharmaceutically acceptable salt thereof, a hydrate thereof, or a solvate of any of the foregoing, exhibits ATP-utilizing enzyme inhibitory activity.

3. The compound of claim 2, wherein
Y is chosen from O, a direct bond, and —N(R¹⁰)— wherein R¹⁰ is H; and
R⁵ is chosen from H, alkyl, substituted alkyl, aryl, substituted aryl, heteroaryl, substituted heteroaryl, arylalkyl, and substituted arylalkyl.

4. The compound of claim 3, wherein R⁵ is chosen from H, C₁₋₁₀ alkyl, substituted C₁₋₁₀ alkyl, C₃₋₁₂ aryl, substituted C₃₋₁₂ aryl, C₃₋₁₂ heteroaryl, substituted C₃₋₁₂ heteroaryl, C₄₋₁₈ arylalkyl, and substituted C₄₋₁₈ arylalkyl.

5. The compound of claim 2, wherein R² is chosen from H, and -ZR⁶, wherein
Z is chosen from carbonyl, and —C(═O)NH—, and
R⁶ is chosen from H, —COOH, C₁₋₁₀ alkyl, substituted C₁₋₁₀ alkyl, C₅₋₁₂ aryl, substituted C₅₋₁₂ aryl, C₃₋₁₂ cycloalkyl, substituted C₃₋₁₂ cycloalkyl, C₃₋₁₂ heterocycloalkyl, substituted C₃₋₁₂heterocycloalkyl, C₁₋₁₀ heteroalkyl, substituted C₁₋₁₀ heteroalkyl, C₅₋₁₂ heteroaryl, substituted C₅₋₁₂ heteroaryl, C₆₋₁₈ heteroarylalkyl, substituted C₆₋₁₈ heteroarylalkyl, C₄₋₁₈cycloalkylalkyl, substituted C₄₋₁₈ cycloalkylalkyl, C₄₋₁₈ heterocycloalkylalkyl, substituted C₄₋₁₈ heterocycloalkylalkyl, C₆₋₁₈ arylalkyl, substituted C₆₋₁₈ arylalkyl, C₅₋₁₂ bicycloalkyl, substituted C₅₋₁₂ bicycloalkyl, C₅₋₁₂ bicycloheteroalkyl, and substituted C₅₋₁₂ bicycloheteroalkyl.

6. The compound of claim 2, wherein R³ is chosen from H, halogen, —NH₂, alkyl, substituted alkyl, acyl, substituted acyl, alkoxycarbonyl, substituted alkoxycarbonyl, aminocarbonyl, substituted aminocarbonyl, cycloalkyl, substituted cycloalkyl, heteroalkyl, substituted heteroalkyl, aryl, substituted aryl, heteroaryl, substituted heteroaryl, arylalkyl, substituted arylalkyl, heteroarylalkyl, substituted heteroarylalkyl, and dialkylamino.

7. The compound of claim 2, wherein R³ is chosen from H, halogen, —NH₂, C₁₋₁₀ alkyl, substituted C₁₋₁₀ alkyl, C₁₋₁₀ acyl, substituted C₁₋₁₀ acyl, C₁₋₁₀ alkoxycarbonyl, substituted C₁₋₁₀ alkoxycarbonyl, C₁₋₁₀ aminocarbonyl, substituted C₁₋₁₀ aminocarbonyl, C₃₋₁₂ cycloalkyl, substituted C₃₋₁₂ cycloalkyl, C₃₋₁₂ heteroalkyl, substituted C₃₋₁₂ heteroalkyl, C₅₋₁₂ aryl, substituted C₅₋₁₂ aryl, C₅₋₁₂ heteroaryl, substituted C₅₋₁₂ heteroaryl, C₆₋₁₈ arylalkyl, substituted C₆₋₁₈ arylalkyl, C₆₋₁₈ heteroarylalkyl, substituted C₆₋₁₈ heteroarylalkyl, and C₂₋₂₀ dialkylamino

8. The compound of claim 2, wherein R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, and substituted arylalkyl, heteroaryl, substituted heteroaryl, heterocycloalkylalkyl, substituted heterocycloalkylalkyl, heteroarylalkyl, and substituted heteroarylalkyl.

9. The compound of claim 2, wherein R⁴ is chosen from H, halogen, C₁₋₁₀ acyl, substituted C₁₋₁₀ acyl, C₁₋₁₀ alkoxycarbonyl, substituted C₁₋₁₀ alkoxycarbonyl, C₁₋₁₀ alkyl, substituted C₁₋₁₀ alkyl, C₁₋₁₀ aminocarbonyl, substituted C₁₋₁₀ aminocarbonyl, C₅₋₁₂ aryl, substituted C₅₋₁₂ aryl, arylalkyl, and substituted arylalkyl, C₅₋₁₂ heteroaryl, substituted C₅₋₁₂ heteroaryl, C₄₋₁₈ heterocycloalkylalkyl, substituted C₄₋₁₈ heterocycloalkylalkyl, C₆₋₁₈ heteroarylalkyl, and substituted C₆₋₁₈ heteroarylalkyl.

10. At least one compound of Formula (III): 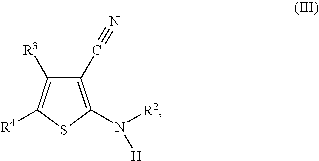
a stereoisomer thereof, a pharmaceutically acceptable salt thereof, a hydrate thereof, or a solvate of any of the foregoing, wherein:
R² is chosen from H, and -ZR⁶, wherein
Z is carbonyl; and
R⁶ is chosen from H, alkyl, substituted alkyl, heteroalkyl, substituted heteroalkyl, aryl, substituted aryl, heteroaryl, substituted heteroaryl, heterocycloalkyl, substituted heterocycloalkyl, heterocycloalkylalkyl, substituted heterocycloalkylalkyl, heteroarylalkyl, and substituted heteroarylalkyl;
R³ is chosen from H, —NH₂, alkyl, and substituted alkyl; and
R⁴ is chosen from H, halogen, alkyl, substituted alkyl, heteroalkyl, substituted heteroalkyl, arylalkyl, substituted arylalkyl, heterocycloalkylalkyl, substituted heterocycloalkylalkyl;
or R³ and R⁴ together with the atoms to which R³ and R⁴ are attached form a cycloalkyl, substituted cycloalkyl, heterocycloalkyl, or substituted heterocycloalkyl ring;
with the provisos that
R³ is not H, 2-aminopyrimidine, substituted 2-aminopyrimidine, 2-aminopyridine, substituted 2-aminopyridine, aminotriazine, or substituted aminotriazine;
R⁴ is not H, 2-aminopyrimidine, substituted 2-aminopyrimidine, 2-aminopyridine, substituted 2-aminopyridine, aminotriazine, or substituted aminotriazine; and
when R² is —C(═X)NH₂, where X is O or S, then
R³ is not unsubstituted phenyl, or a 5 to 7 membered heteroaromatic ring containing 1 to 3 heteroatoms chosen from O, N or S; and
R⁴ is not unsubstituted phenyl, or a 5 to 7 membered heteroaromatic ring containing 1 to 3 heteroatoms chosen from O, N or S;
and wherein the compound of Formula (III), a stereoisomer thereof, a pharmaceutically acceptable salt thereof, a hydrate thereof, or a solvate of any of the foregoing, exhibits ATP-utilizing enzyme inhibitory activity.

11. The compound of claim 10, wherein R⁴ is chosen from C₁₋₈ alkyl, substituted C₁₋₈ alkyl, C₁₋₈ heteroalkyl, substituted C₁₋₈ heteroalkyl, C₆₋₁₂ arylalkyl, substituted C₆₋₁₂ arylalkyl, C₆₋₁₂ heterocycloalkylalkyl, and substituted C₆₋₁₂ heterocycloalkylalkyl.

12. The compound of claim 10, wherein R³ and R⁴ together with the atoms to which R³ and R⁴ are attached form a C₅₋₁₀ cycloalkyl, substituted C₅₋₁₀ cycloalkyl, C₅₋₁₀ heterocycloalkyl, or substituted C₅₋₁₀ heterocycloalkyl ring.

13. The compound of claim 12, wherein the at least one substituent group is chosen from halogen, C₁₋₆ alkyl, and ═O.

14. The compound of claim 10, wherein R⁴ is chosen from C₁₋₈ alkyl, substituted C₁₋₈ heteroalkyl, substituted C₅₋₁₀ arylalkyl, and substituted C₆₋₁₀ heterocycloalkylalkyl.

15. The compound of claim 10, wherein R³ is chosen from —NH₂, C₁₋₈ alkyl, and substituted C₁₋₈ alkyl.

16. The compound of claim 10, wherein R is chosen from H, and —C(O)R⁶ wherein
R⁶ is chosen from C₁₋₈ alkyl, substituted C₁₋₈ alkyl, C₁₋₈ heteroalkyl, substituted C₁₋₈ heteroalkyl, C₅₋₁₂ aryl, substituted C₅₋₁₂ aryl, C₅₋₁₂ heteroaryl, substituted C₅₋₁₂ heteroaryl, C₆₋₁₈ heterocycloalkyl, substituted C₆₋₁₈ heterocycloalkyl, C₆₋₁₈ heterocycloalkylalkyl, substituted C₆₋₁₈ heterocycloalkylalkyl, C₆₋₁₈ heteroarylalkyl, and substituted C₆₋₁₈ heteroarylalkyl.

17. The compound of claim 16, wherein the at least one substituent group is chosen from halogen, C₁₋₆ alkyl, C₁₋₆ alkoxy, C₅₋₈ aryl, substituted C₅₋₈ aryl, C₅₋₈ heteroaryl, substituted C₅₋₈ heteroaryl, ═O, ═S, —COOH, —CF₃, and —OH.

18. The compound of claim 10, wherein the at least one compound has the structure of any of compounds 1.1 to 1.45: 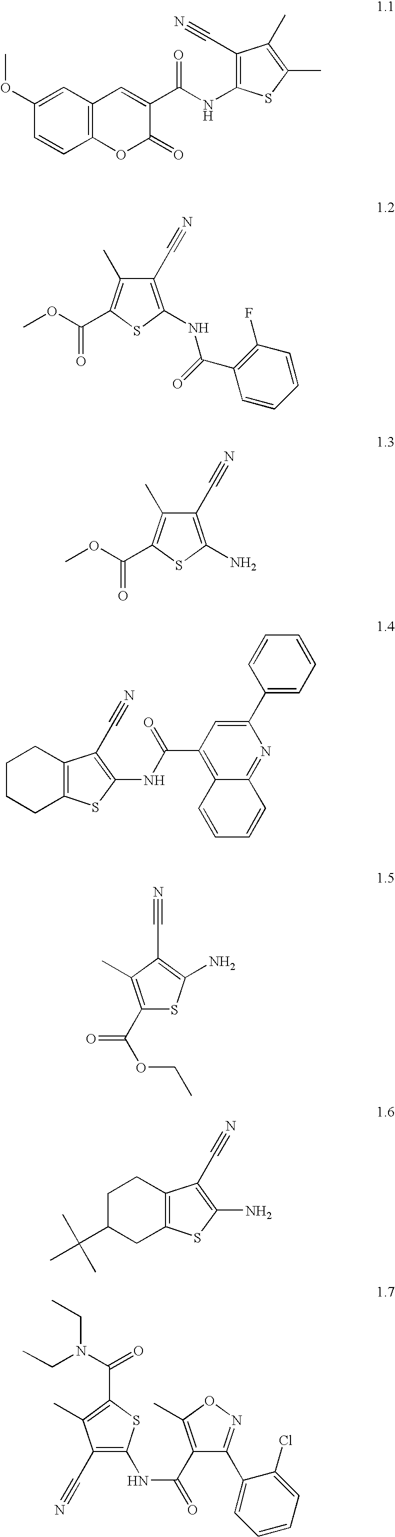 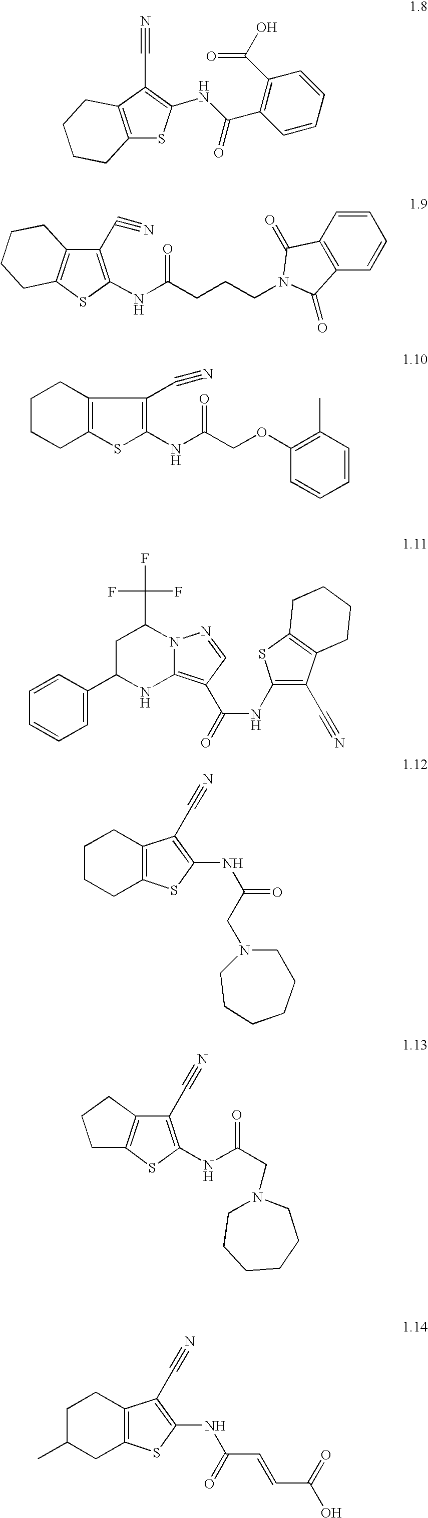 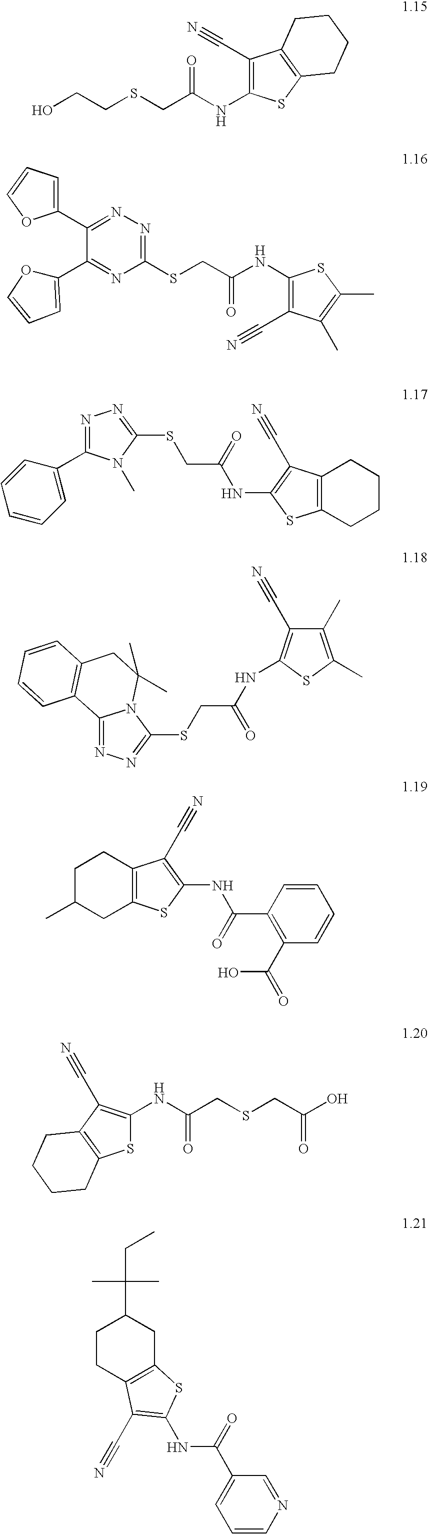 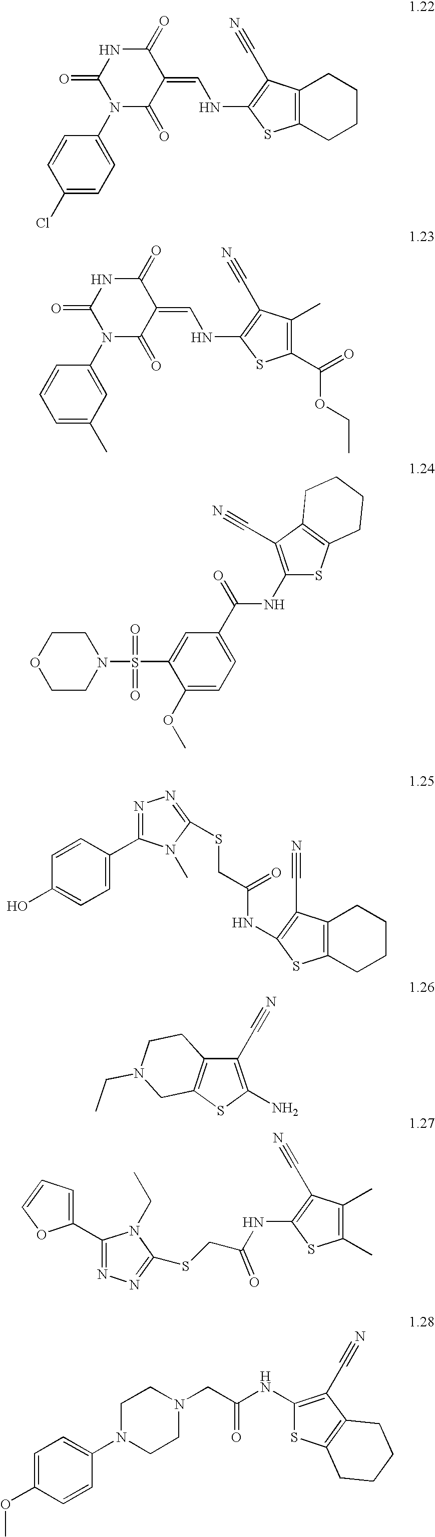 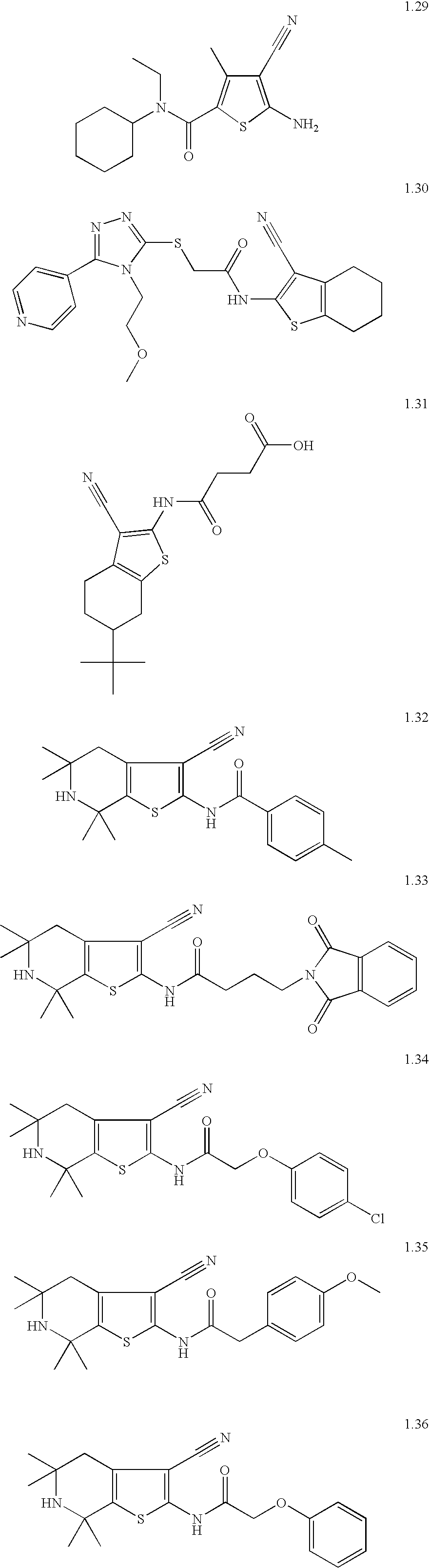 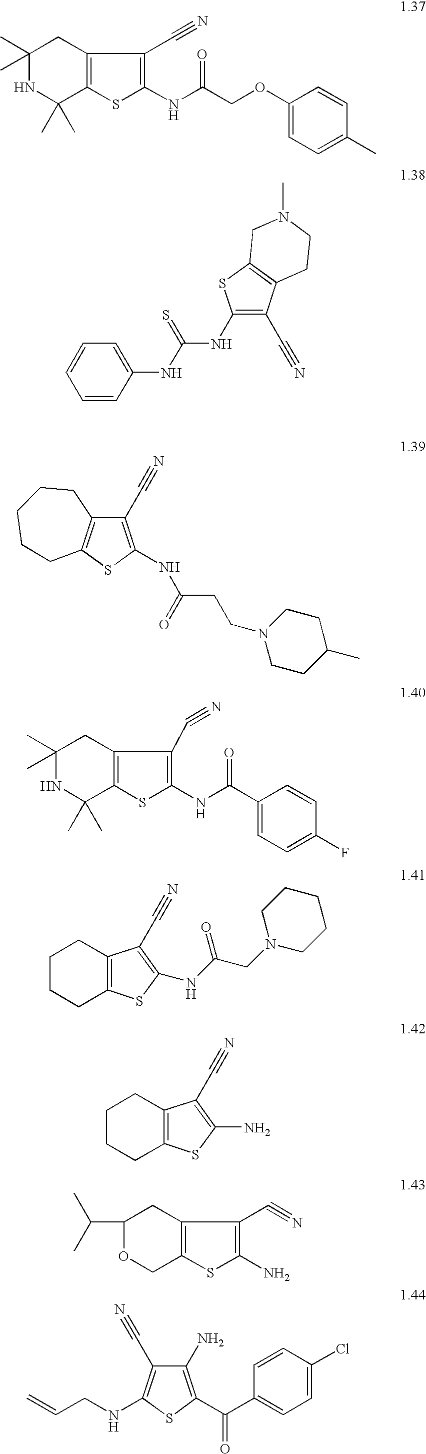 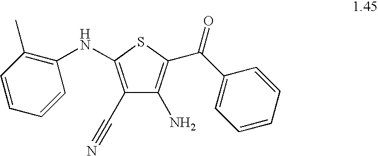
a stereoisomer thereof, a pharmaceutically acceptable salt thereof, a hydrate thereof, or a solvate of any of the foregoing.

19. The compound of claim 10, wherein the at least one ATP-utilizing enzyme is chosen from a human protein kinase.

20. The compound of claim 19, wherein the human protein kinase is chosen from AKT2, AURORA-A, CDK2/cyclinE, CHEK1, CHEK2, CK1, DYRK2, FLT-3, FYN, GSK-3α, GSK-3β, INSR, KIT, LYNA, MAPK1, MAPKAPK-2, MAPKAPK-3, MSK2, NEK2, P38-α, PAK2, PDGFR-α, PDK1, PKA, PRAK, SYK, TRKB, and ZAP70.

21. A pharmaceutical composition comprising at least one pharmaceutically acceptable excipient, and a therapeutically effective amount of at least one compound according to claim 10.

22. A pharmaceutical composition comprising at least one pharmaceutically acceptable excipient, and a therapeutically effective amount of at least one compound according to claim 18.

23. The pharmaceutical composition of claim 21, wherein the at least one compound is present in an amount effective for the treatment in a patient of at least one disease chosen from Alzheimer's disease, stroke, diabetes, obesity, inflammation, and cancer.

24. The pharmaceutical composition of claim 23, wherein cancer is chosen from at least one of glioblastoma, ovarian, breast, endometrial, hepatocellular carcinoma, melanoma, digestive tract, lung, renal-cell carcinoma, thyroid, lymphoid, prostate, and pancreatic cancer.

25. A method of treating a disease in a patient in need of such treatment comprising administering to the patient a therapeutically effective amount of at least one compound according to claim 10.

26. A method of treating a disease in a patient in need of such treatment comprising administering to the patient a therapeutically effective amount of at least one compound according to claim 18.

27. The method of claim 25, wherein the at least one disease is chosen from Alzheimer's disease, stroke, diabetes, obesity, inflammation, and cancer.

28. The method of claim 27, wherein cancer is chosen from at least one of glioblastoma, ovarian, breast, endometrial, hepatocellular carcinoma, melanoma, digestive tract, lung, renal-cell carcinoma, thyroid, lymphoid, prostate, and pancreatic cancer.

29. A method of inhibiting at least one ATP-utilizing enzyme in a subject comprising administering to the subject at least one compound according to claim 10.

30. A method of inhibiting at least one ATP-utilizing enzyme in a subject comprising administering to the subject at least one compound according to claim 18.

31. The method of claim 29, wherein the ATP-utilizing enzyme is chosen from a human protein kinase.

32. The method of claim 31, wherein the human protein kinase is chosen from AKT2, AURORA-A, CDK2/cyclinE, CHEK1, CHEK2, CK1, DYRK2, FLT-3, FYN, GSK-3α, GSK-3β, INSR, KIT, LYNA, MAPK1, MAPKAPK-2, MAPKAPK-3, MSK2, NEK2, P38-α, PAK2, PDGFR-α, PDK1, PKA, PRAK, SYK, TRKB, and ZAP70.

33. A method of inhibiting at least one ATP-utilizing enzyme comprising contacting the ATP-utilizing enzyme with at least one compound according to claim 10.

34. A method of inhibiting at least one ATP-utilizing enzyme comprising contacting the ATP-utilizing enzyme with at least one compound according to claim 18.

35. The method of claim 33, wherein the ATP-utilizing enzyme is chosen from a human protein kinase.

36. The method of claim 35, wherein human protein kinase is chosen from AKT2, AURORA-A, CDK2/cyclinE, CHEK1, CHEK2, CK1, DYRK2, FLT-3, FYN, GSK-3α, GSK-3β, INSR, KIT, LYNA, MAPK1, MAPKAPK-2, MAPKAPK-3, MSK2, NEK2, P38-α, PAK2, PDGFR-α, PDK1, PKA, PRAK, SYK, TRKB, and ZAP70.

37. A method of treating a disease regulated by at least one ATP-utilizing enzyme in a subject in need of such treatment comprising administering to the subject a therapeutically effective amount of at least one compound according to claim 10.

38. A method of treating a disease regulated by at least one ATP-utilizing enzyme in a subject in need of such treatment comprising administering to the subject a therapeutically effective amount of at least one compound according to claim 18.

39. The method of claim 37, wherein the ATP-utilizing enzyme is chosen from a human protein kinase.

40. The method of claim 39, wherein the protein kinase is chosen from AKT2, AURORA-A, CDK2/cyclinE, CHEK1, CHEK2, CK1, DYRK2, FLT-3, FYN, GSK-3α, GSK-3β, INSR, KIT, LYNA, MAPK1, MAPKAPK-2, MAPKAPK-3, MSK2, NEK2, P38-α, PAK2, PDGFR-α, PDK1, PKA, PRAK, SYK, TRKB, and ZAP70.

41. At least one compound of Formula (IV): 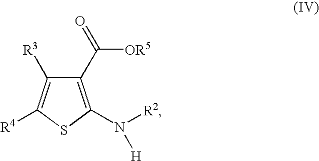
a stereoisomer thereof, a pharmaceutically acceptable salt thereof, a hydrate thereof, or a solvate of any of the foregoing, wherein:
R² is chosen from H, —CHO, heteroarylalkyl, substituted heteroarylalkyl, heterocycloalkylalkyl, substituted heterocycloalkylalkyl, alkylsulfonyl, substituted alkylsulfonyl, and -ZR⁶, wherein
Z is carbonyl; and
R⁶ is chosen from H, —COOH, alkyl, substituted alkyl, aryl, and substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, bicycloalkyl, substituted bicycloalkyl, bicycloheteroalkyl, and substituted bicycloheteroalkyl;
R³ is chosen from H, halogen, alkyl, substituted alkyl, aryl, substituted aryl, arylalkyl, substituted arylalkyl, heterocycloalkyl, substituted heterocycloalkyl, heteroaryl, substituted heteroaryl, heteroarylalkyl, substituted heteroarylalkyl, and dialkylamino;
R⁴ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, heteroarylalkyl, and substituted heteroarylalkyl;
or R³ and R⁴ together with the atoms to which R³ and R⁴ are attached form a cycloalkyl, substituted cycloalkyl, heterocycloalkyl, substituted heterocycloalkyl, bicycloalkyl, substituted bicycloalkyl, bicycloheteroalkyl, or substituted bicycloheteroalkyl ring; and
R⁵ is chosen from H, alkyl, substituted alkyl, arylalkyl, and substituted arylalkyl;
with the provisos that
R³ is not chosen from H, 2-aminopyrimidine, substituted 2-aminopyrimidine, 2-aminopyridine, substituted 2-aminopyridine, aminotriazine, or substituted aminotriazine; and
R⁴ is not chosen from 2-aminopyrimidine, substituted 2-aminopyrimidine, 2-aminopyridine, substituted 2-aminopyridine, aminotriazine, or substituted aminotriazine;
and wherein the compound of Formula (IV), a stereoisomer thereof, a pharmaceutically acceptable salt thereof, a hydrate thereof, or a solvate of any of the foregoing, exhibits ATP-utilizing enzyme inhibitory activity.

42. The compound of claim 41, wherein R³ is chosen from H, C₁₋₆ alkyl, substituted C₁₋₆ alkyl, C₅₋₁₂ aryl, substituted C₅₋₁₂ aryl, C₅₋₁₂ heteroaryl, substituted C₅₋₁₂ heteroaryl, C₆₋₁₈ heterocycloalkyl, substituted C₆₋₁₈ heterocycloalkyl, C₆₋₁₈ arylalkyl, substituted C₆₋₁₈ arylalkyl, C₂₋₆ dialkylamino, and substituted C₂₋₆ dialkylamino.

43. The compound of claim 42, wherein the at least one substituent group is chosen from halogen, C₁₋₆ alkyl, C₁₋₆ alkoxy, C₁₋₆ alkylsulfonyl, C₅₋₁₂ aryl, substituted C₅₋₁₂ aryl, —OH, —CN, —NH₂, —CF₃, nitro, and —NHC(O)CH₃.

44. The compound of claim 41, wherein R⁵ is chosen from H, C₁₋₆ alkyl, substituted C₁₋₆ alkyl, C₆₋₁₈ arylalkyl, and substituted C₆₋₁₈ arylalkyl.

45. The compound of claim 41, wherein R⁵ is chosen from H, C₁₋₆ alkyl, and C₆₋₁₀ arylalkyl.

46. The compound of claim 41, wherein R⁴ is chosen from H, C₁₋₆alkyl, substituted C₁₋₆ alkyl, C₁₋₆ aminocarbonyl, substituted C₁₋₆ aminocarbonyl, C₁₋₆ carbonyl, substituted C₁₋₆ carbonyl, C₁₋₆ alkoxycarbonyl, substituted C₁₋₆alkoxycarbonyl, C₅₋₁₂ aryl, substituted C₅₋₁₂ aryl, C₆₋₁₈ heteroarylalkyl, and substituted C₆₋₁₈ heteroarylalkyl.

47. The compound of claim 46, wherein the at least one substituent group is chosen from halogen, ═O, C₁₋₆ alkoxy, and C₁₋₆ alkyl.

48. The compound of claim 41, wherein R³ and R⁴ together with the atoms to which R³ and R⁴ are attached form a C₅₋₁₂ cycloalkyl, substituted C₅₋₁₂ cycloalkyl, C₅₋₁₂ heterocycloalkyl, substituted C₅₋₁₂ heterocycloalkyl, C₅₋₁₂ bicycloalkyl, substituted C₅₋₁₂ bicycloalkyl, C₅₋₁₂ bicycloheteroalkyl, or substituted C₅₋₁₂ bicycloheteroalkyl ring.

49. The compound of claim 48, wherein the at least one substituent group is chosen from C₁₋₆ alkoxy, halogen, C₁₋₆ alkyl, C₅₋₁₂ aryl, substituted C₅₋₁₂ aryl, C₁₋₆ alkoxycarbonyl, substituted C₁₋₆ alkoxycarbonyl, C₆₋₁₂ arylalkyl, substituted C₆₋₁₂ arylalkyl, ═O, and ═N—OH.

50. The compound of claim 41, wherein R² is chosen from H, —COOH, —CH═O, C₁₋₆ alkylsulfonyl, substituted C₁₋₆ alkylsulfonyl, C₆₋₁₂ heterocycloalkylalkyl, substituted C₆₋₁₂ heterocycloalkylalkyl, C₆₋₁₂ heteroarylalkyl, substituted C₆₋₁₂ heteroarylalkyl, and —COR⁶ wherein,
R⁶ is chosen from C₁₋₁₀ alkyl, substituted C₁₋₁₀ alkyl, C₁₋₁₀ heteroalkyl, substituted C₁₋₁₀ heteroalkyl, C₃₋₁₂ cycloalkyl, substituted C₃₋₁₂ cycloalkyl, C₃₋₁₂ heterocycloalkyl, substituted C₃₋₁₂ heterocycloalkyl, C₅₋₁₂ aryl, substituted C₅₋₁₂ aryl, C₅₋₁₂ heteroaryl, substituted C₅₋₁₂ heteroaryl, C₆₋₁₈ cycloalkylalkyl, substituted C₆₋₁₈ cycloalkylalkyl, C₆₋₁₈ heterocycloalkylalkyl, substituted C₆₋₁₈ heterocycloalkylalkyl, C₆₋₁₈ arylalkyl, substituted C₆₋₁₈ arylalkyl, C₆₋₁₈ heteroarylalkyl, substituted C₆₋₁₈ heteroarylalkyl, C₅₋₁₂ bicycloalkyl, substituted C₅₋₁₂ bicycloalkyl, C₅₋₁₂ bicycloheteroalkyl, and substituted C₅₋₁₂ bicycloheteroalkyl.

51. The compound of claim 50, wherein the at least one substituent group is chosen from C₁₋₆ alkyl, substituted C₁₋₆ alkyl, C₁₋₆ heteroalkyl, substituted C₁₋₆ heteroalkyl, C₁₋₆ substituted C₁₋₆ alkoxy, C₅₋₈ aryl, substituted C₅₋₈ aryl, C₅-8 heteroaryl, substituted C₅₋₈ heteroaryl, C₅₋₈ cycloalkyl, substituted C₅₋₈ cycloalkyl, C₅₋₈ heterocycloalkyl, substituted C₅₋₈ heterocycloalkyl, C₆₋₁₀ arylalkyl, substituted C₆₋₁₀ arylalkyl, C₆₋₁₀ heteroarylalkyl, substituted C₆₋₁₀ heteroarylalkyl, C₆₋₁₀ cycloalkylalkyl, substituted C₆₋₁₀ cycloalkylalkyl, C₆₋₁₀ heterocycloalkylalkyl, substituted C₆₋₁₀ heterocycloalkylalkyl, C₁₋₆alkylsulfonyl, substituted C₁₋₆ alkylsulfonyl, halogen, —OH, ═O, nitro, —COOH, —CF₃, ═NH, and —NH₂.

52. The compound of claim 41, wherein the at least one compound has the structure of any of compounds 2.1 to 2.193: 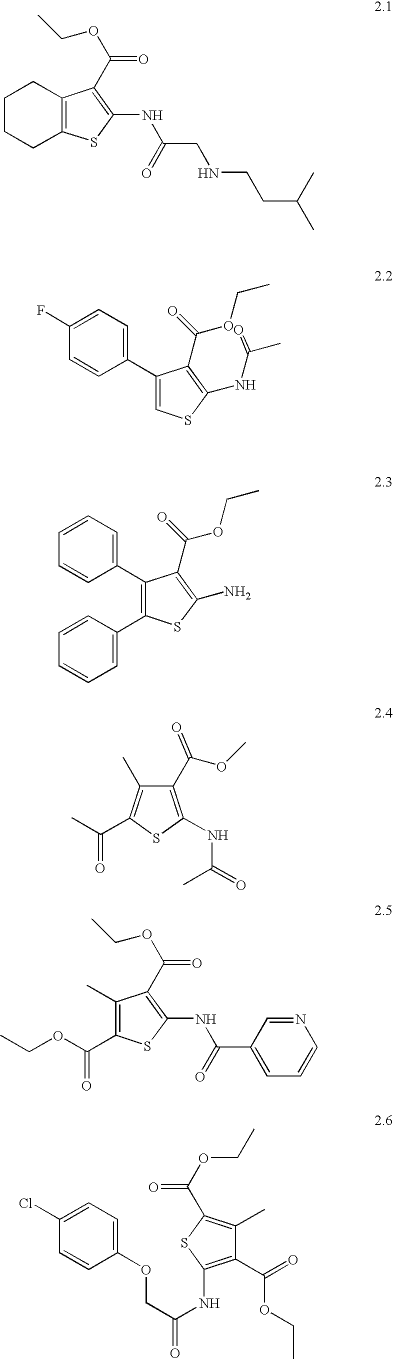 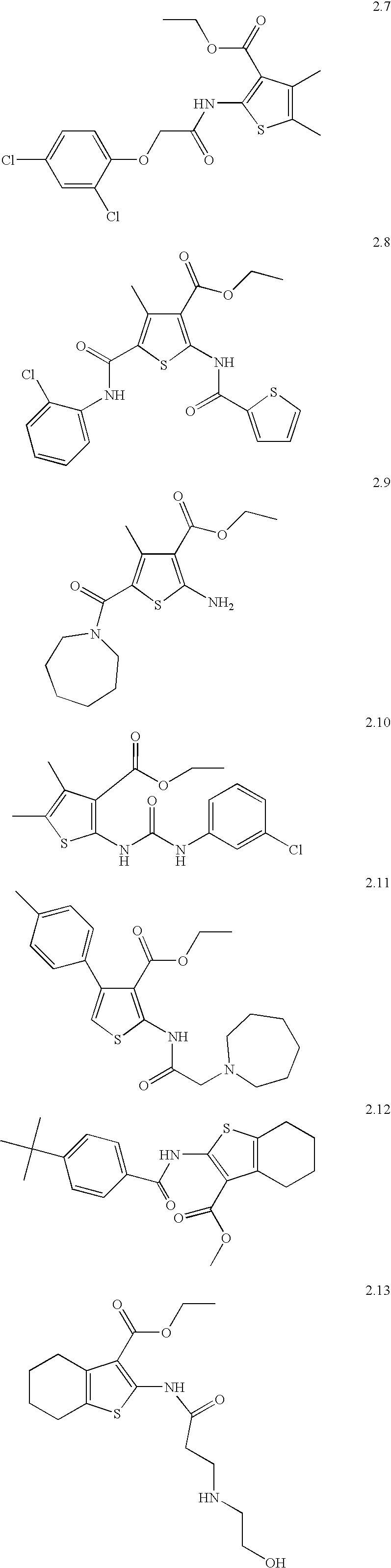 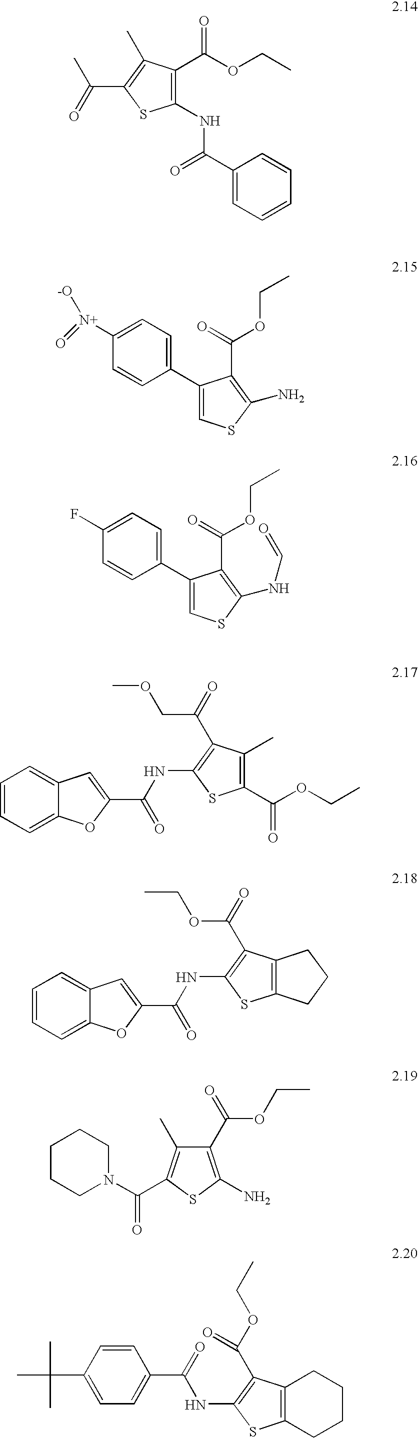 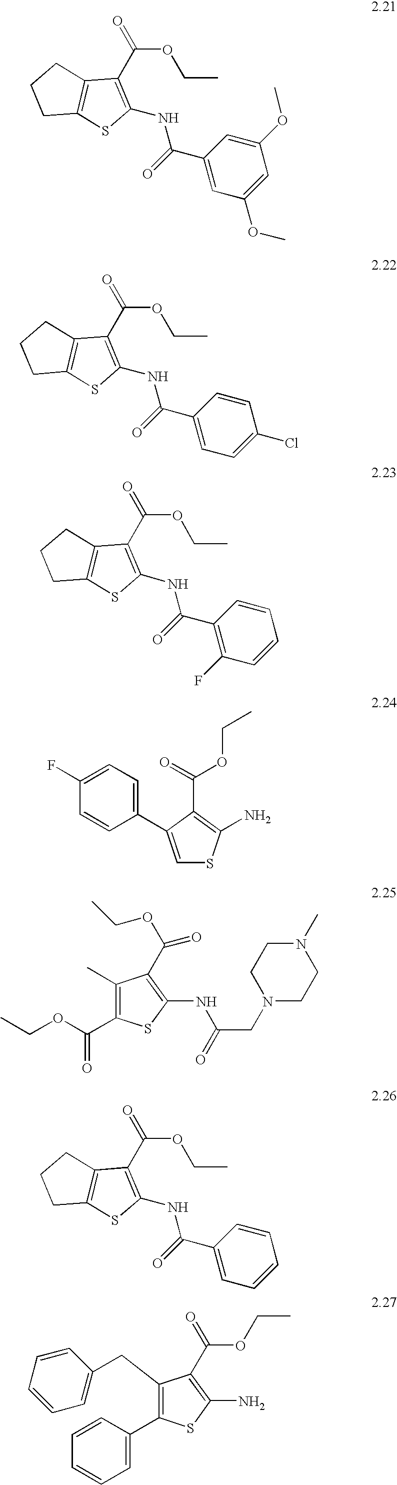 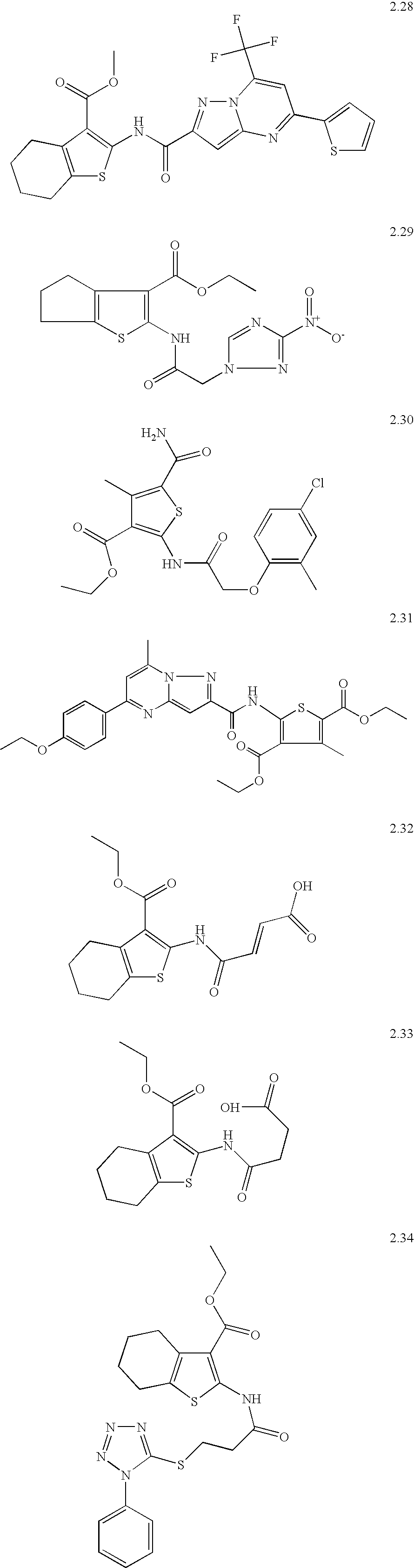 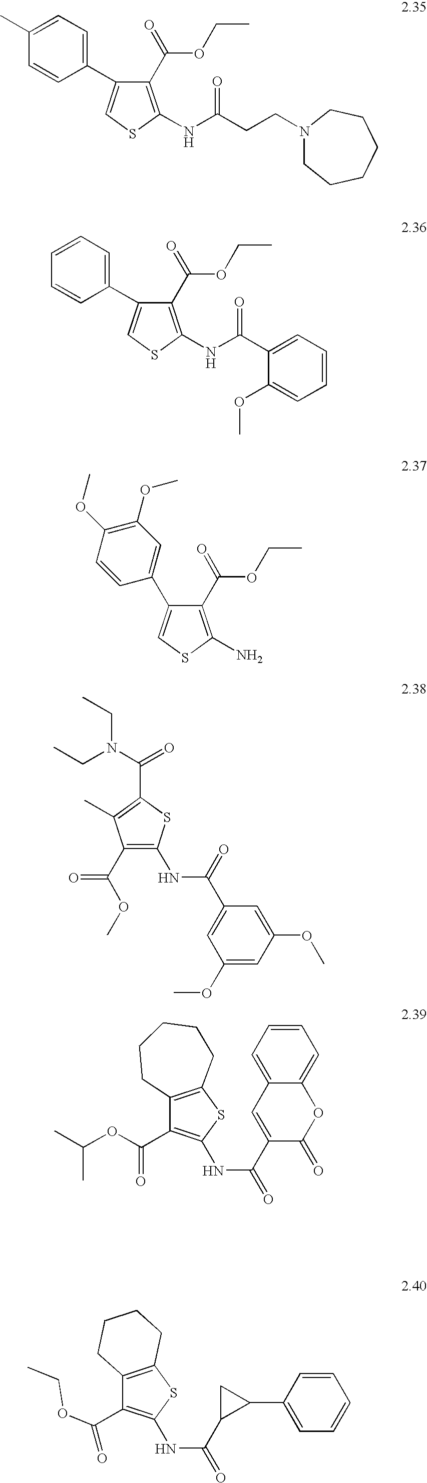 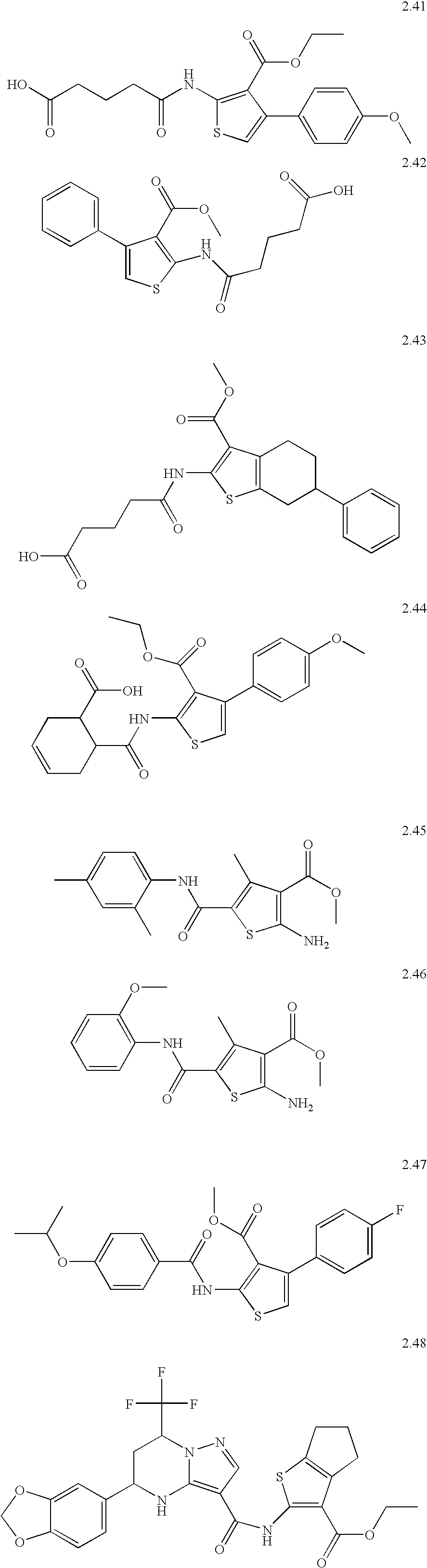 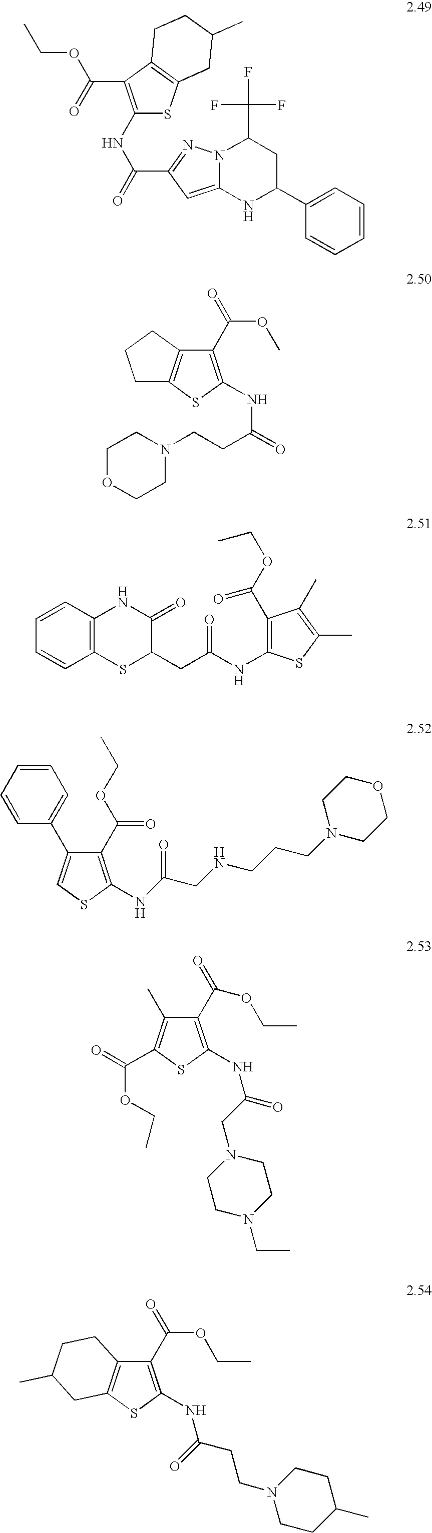 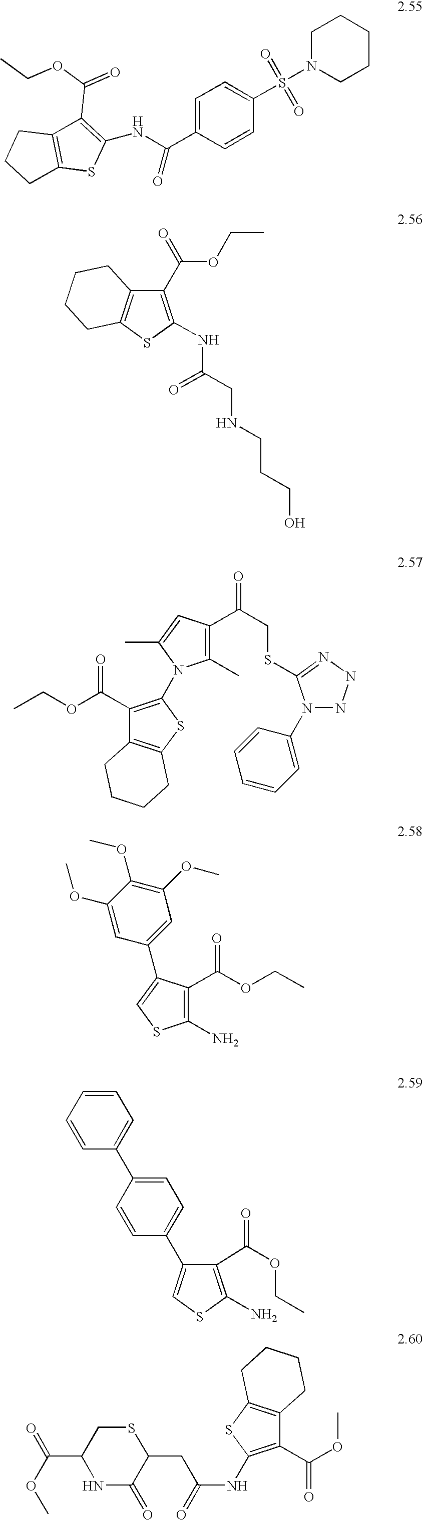 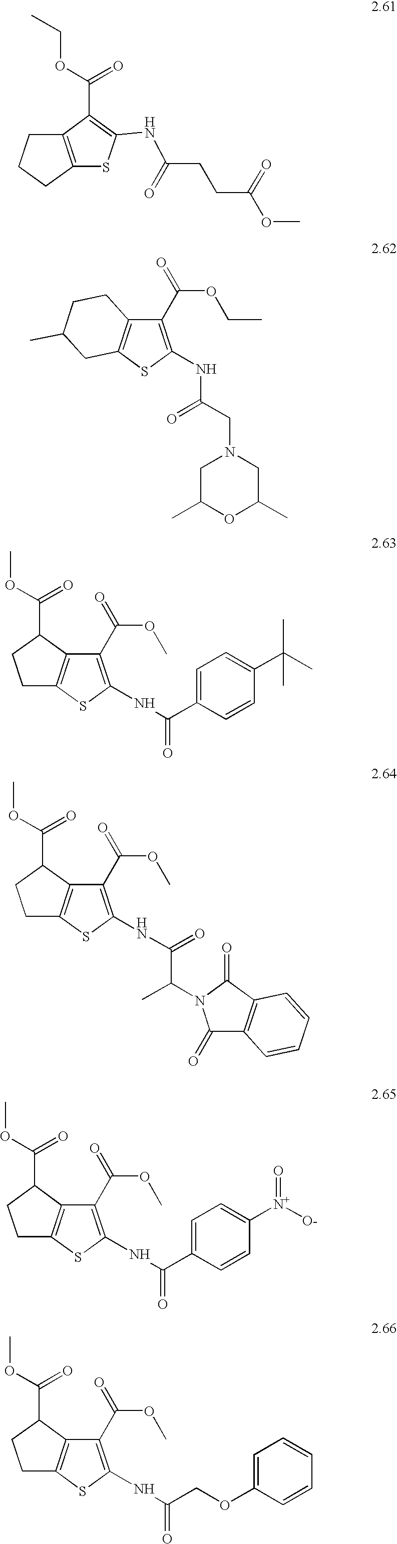 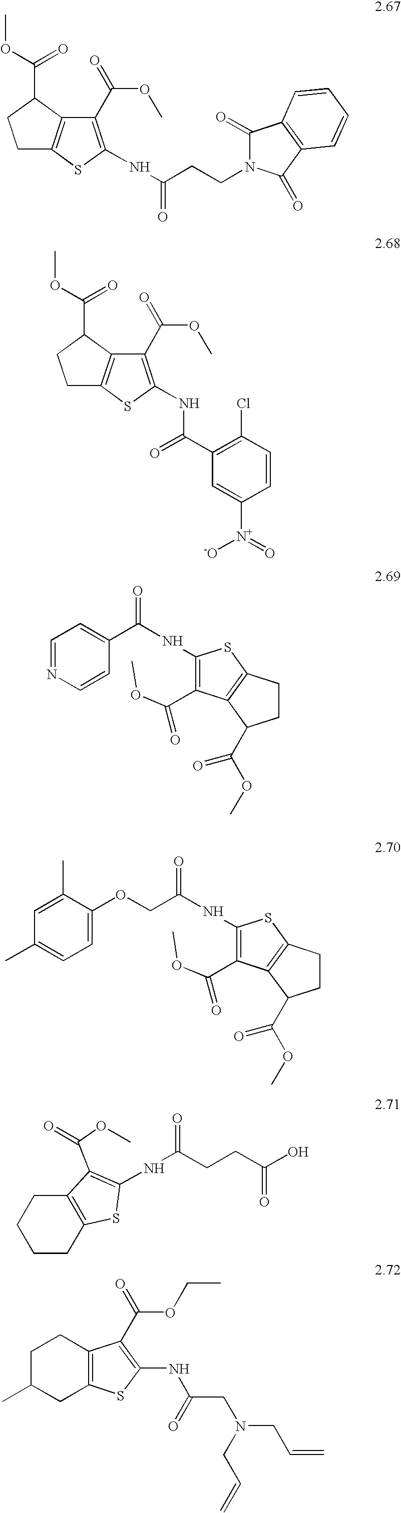 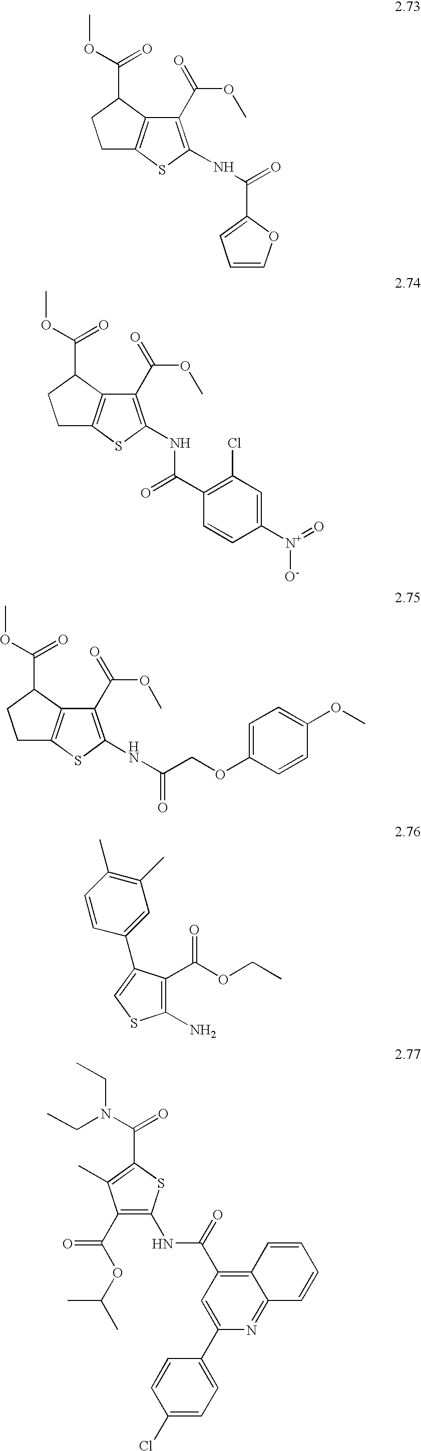 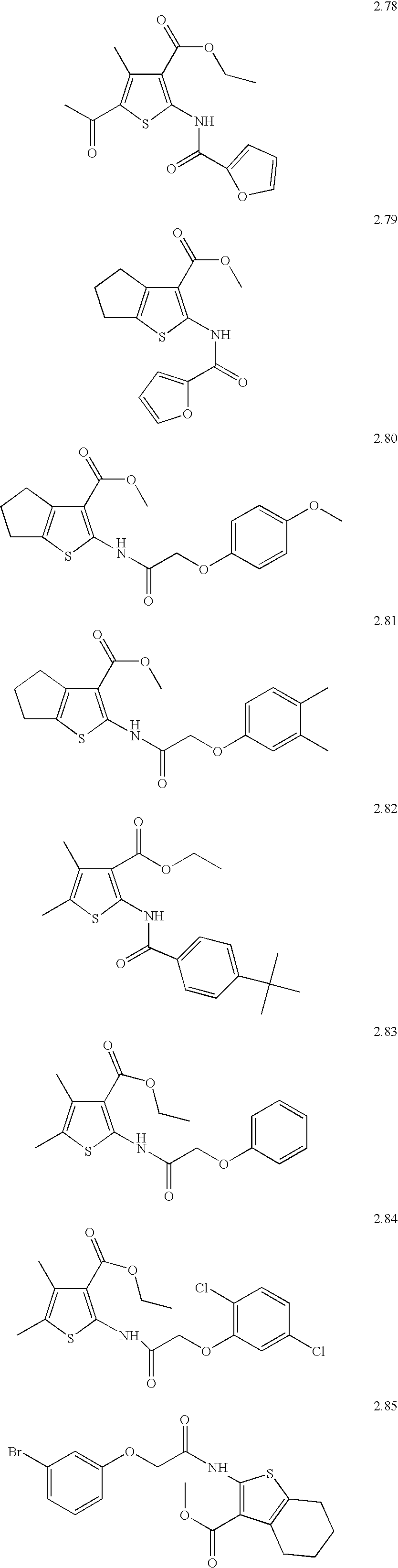 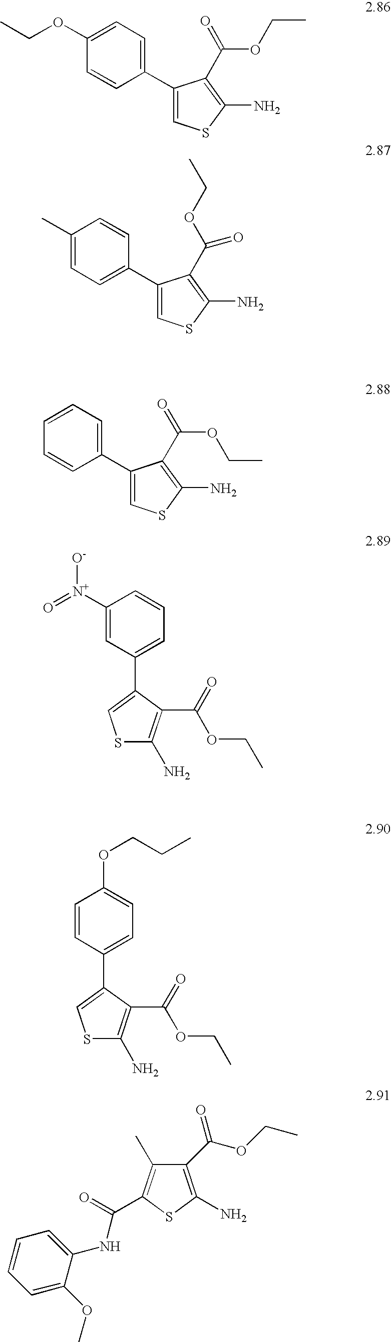 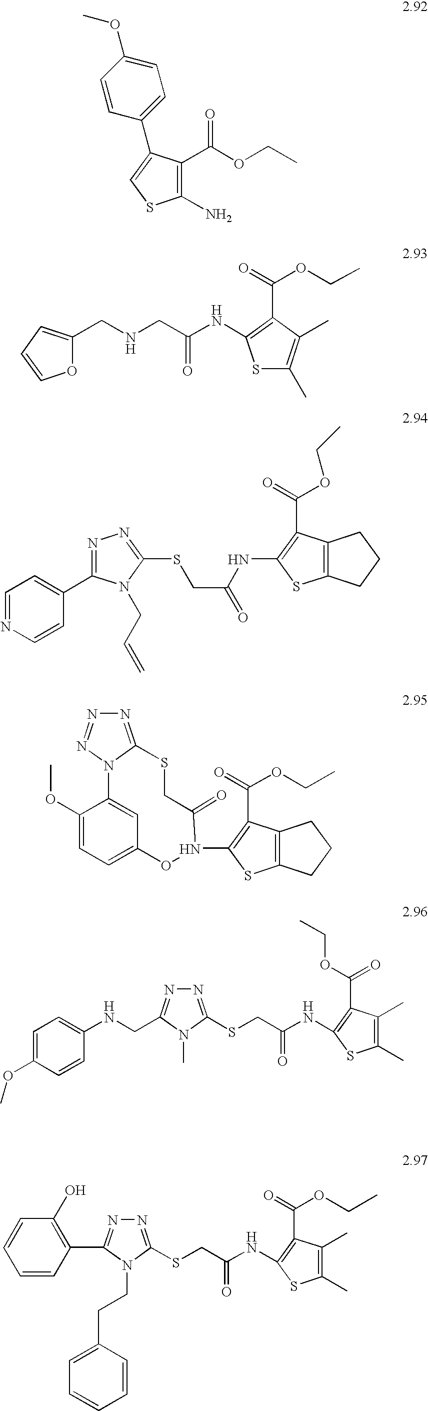 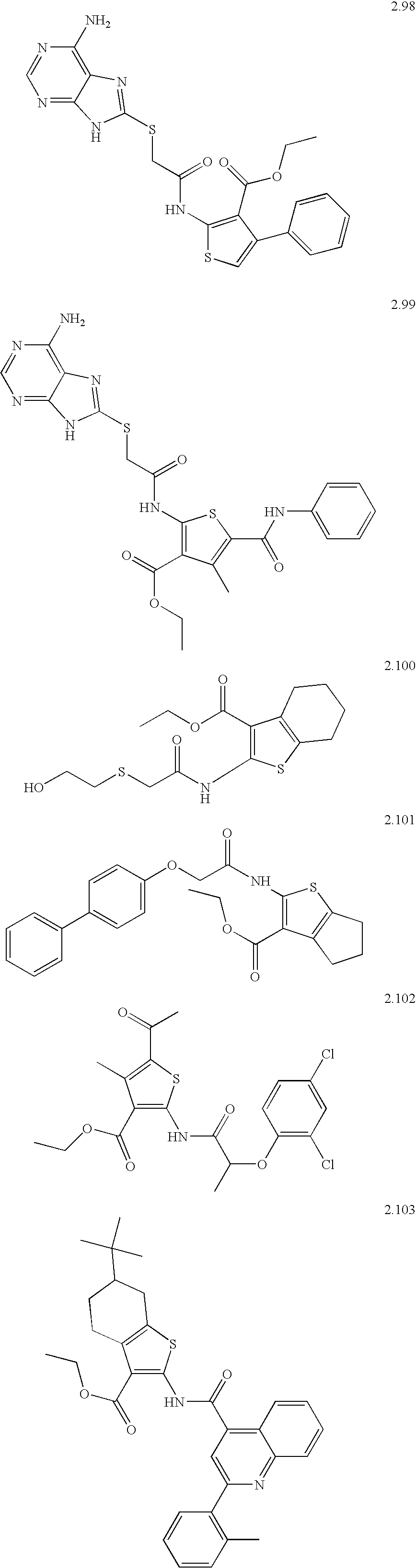 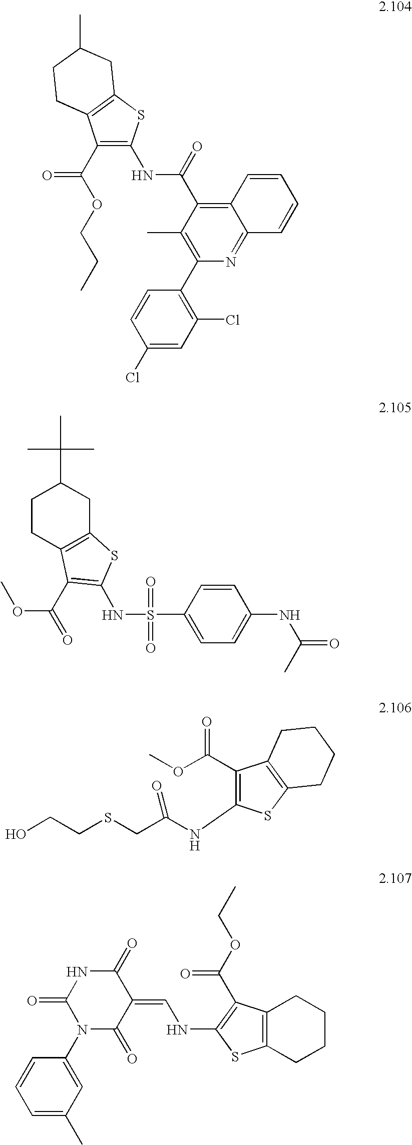 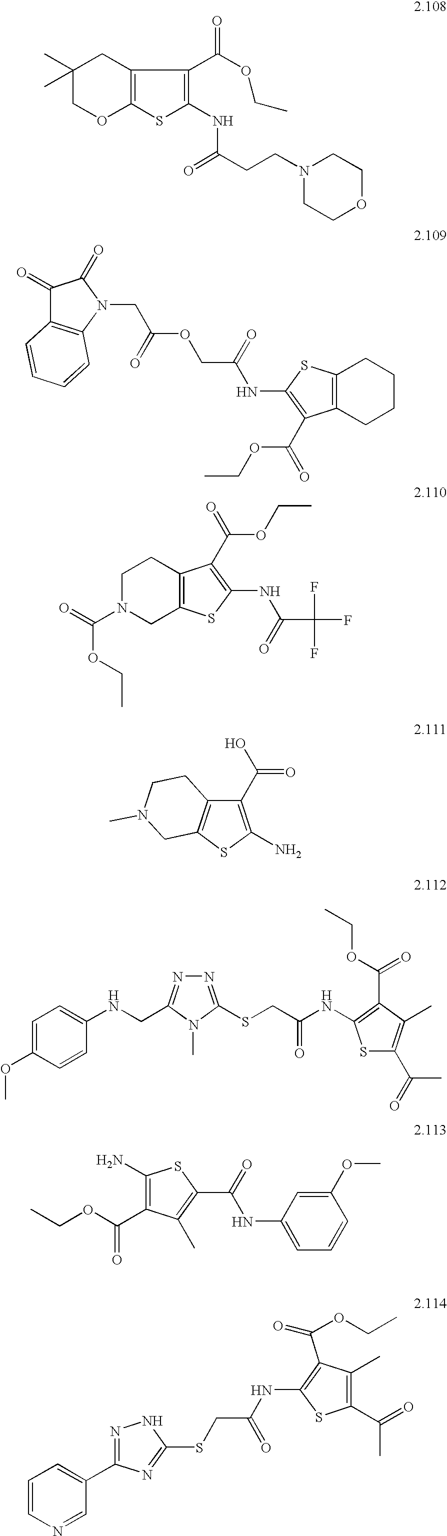 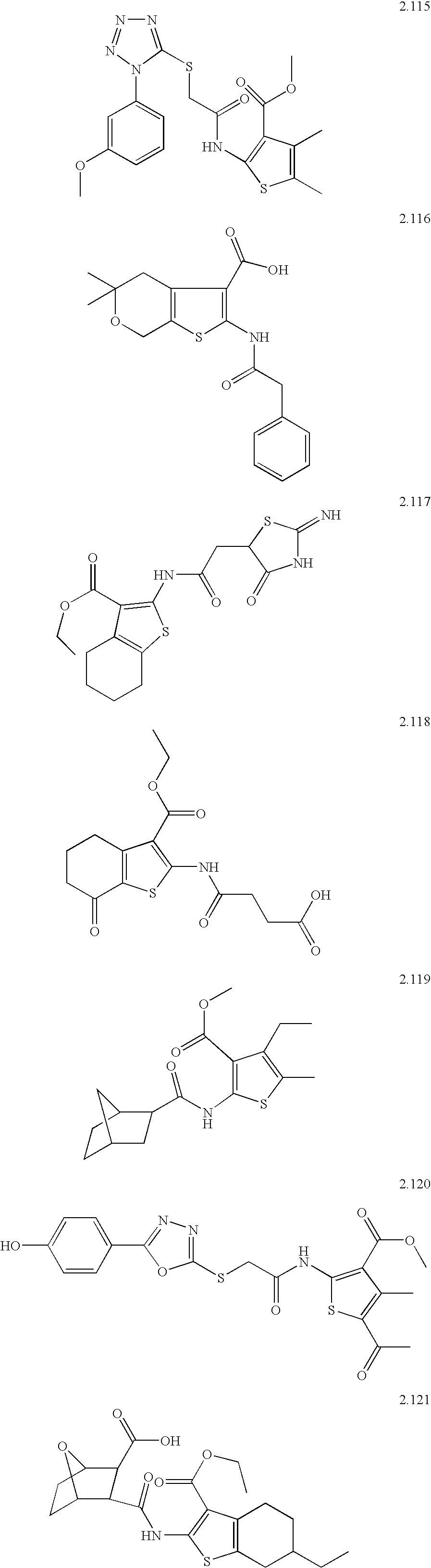 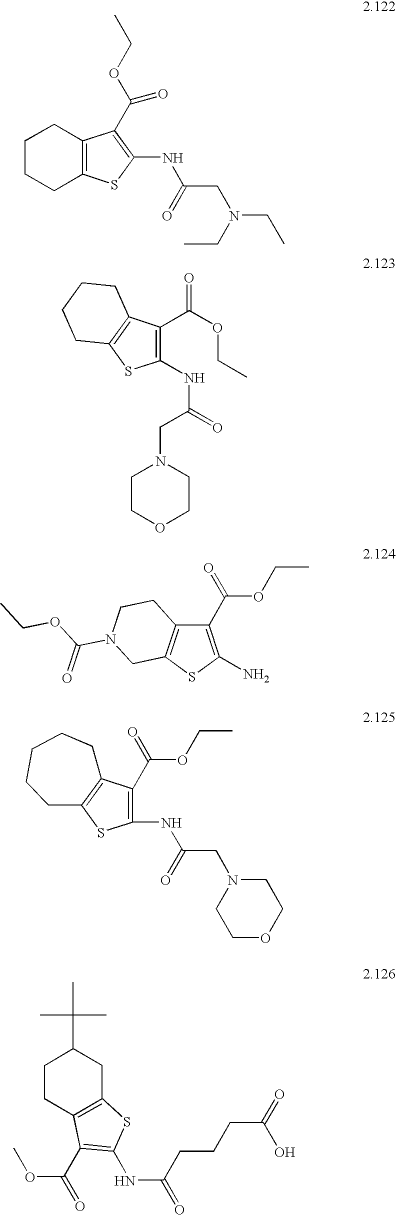 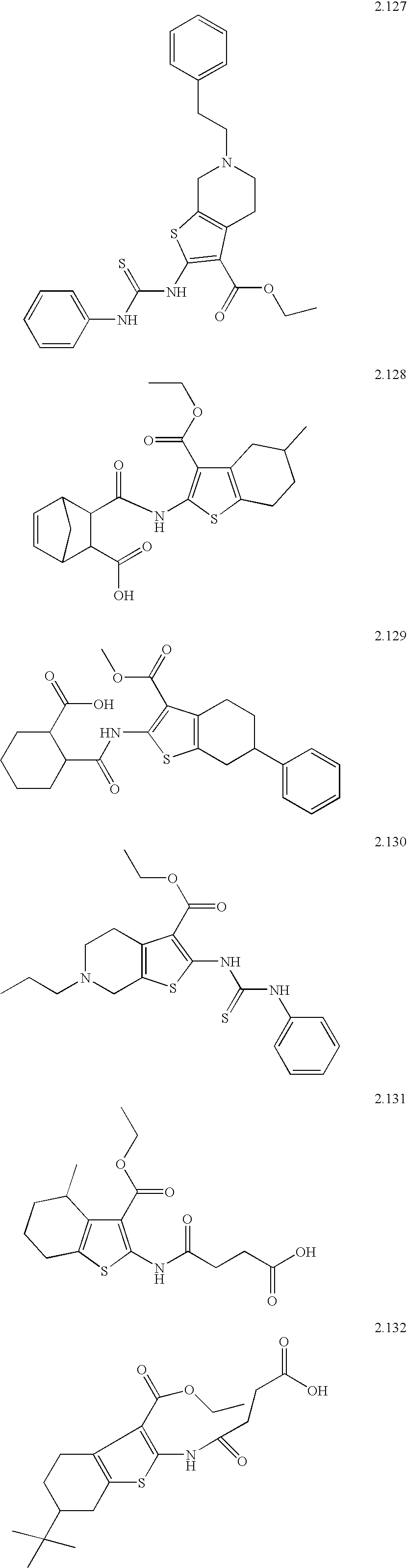 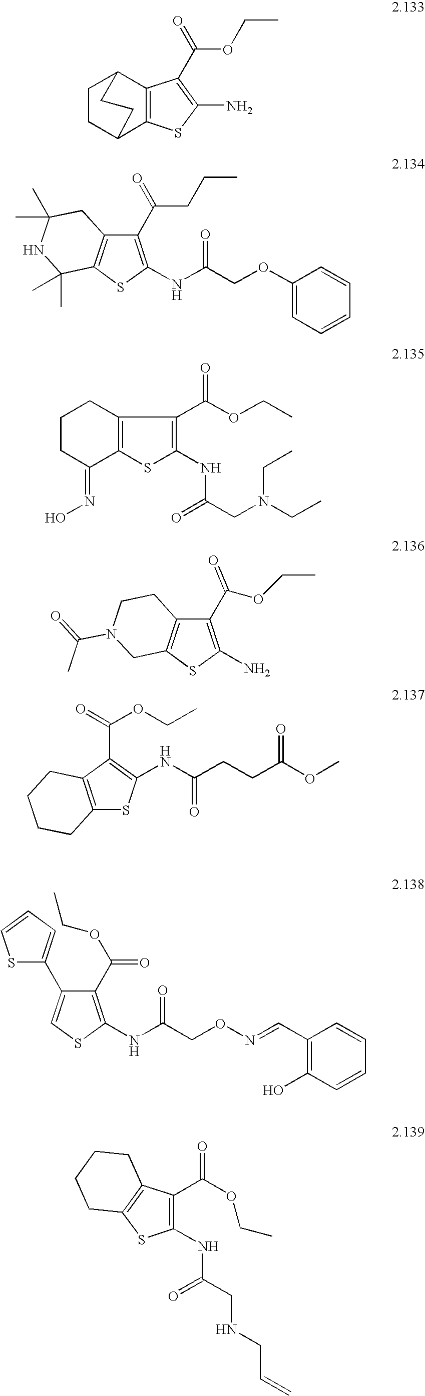 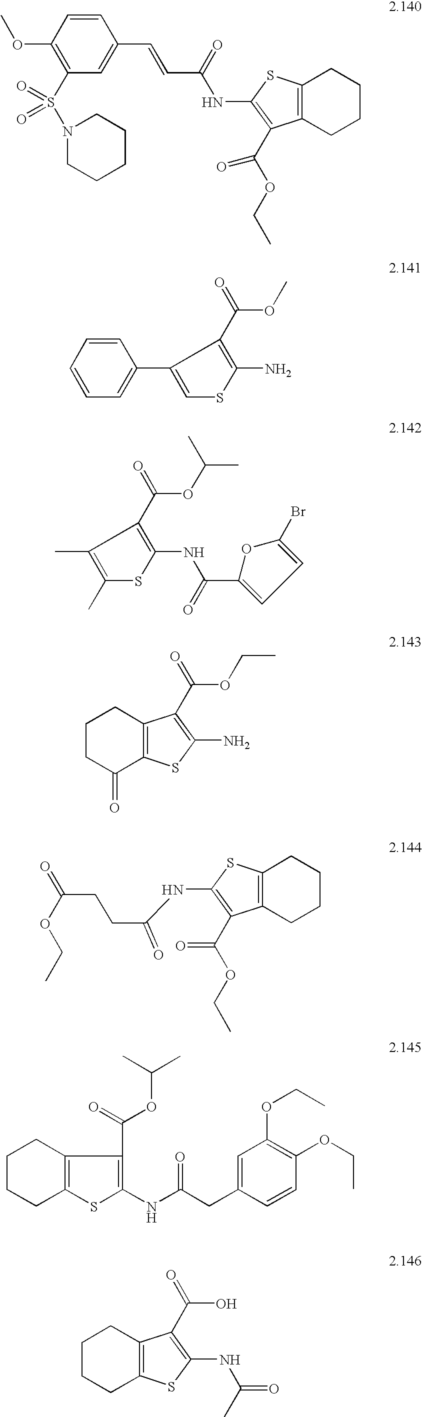 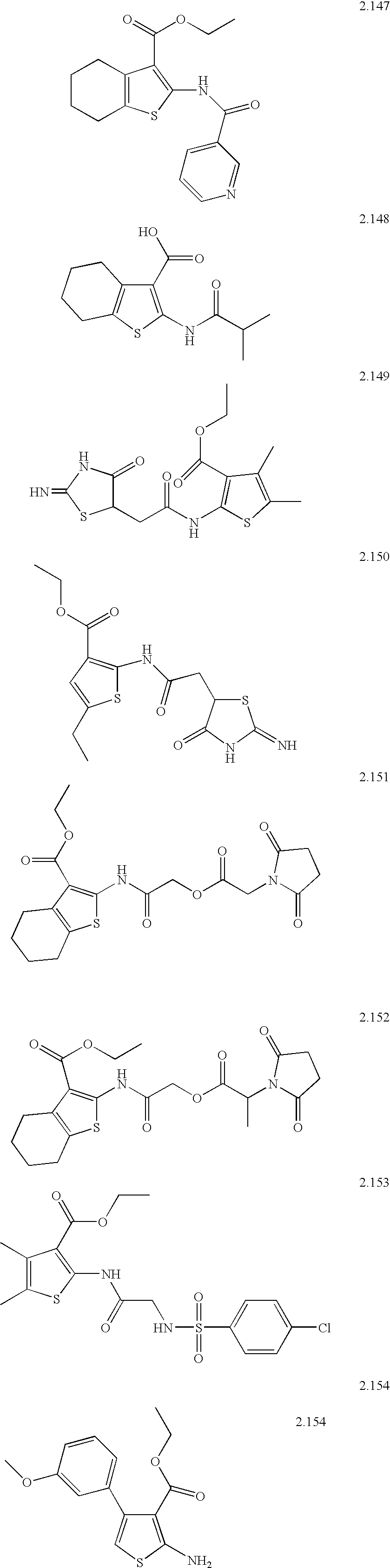 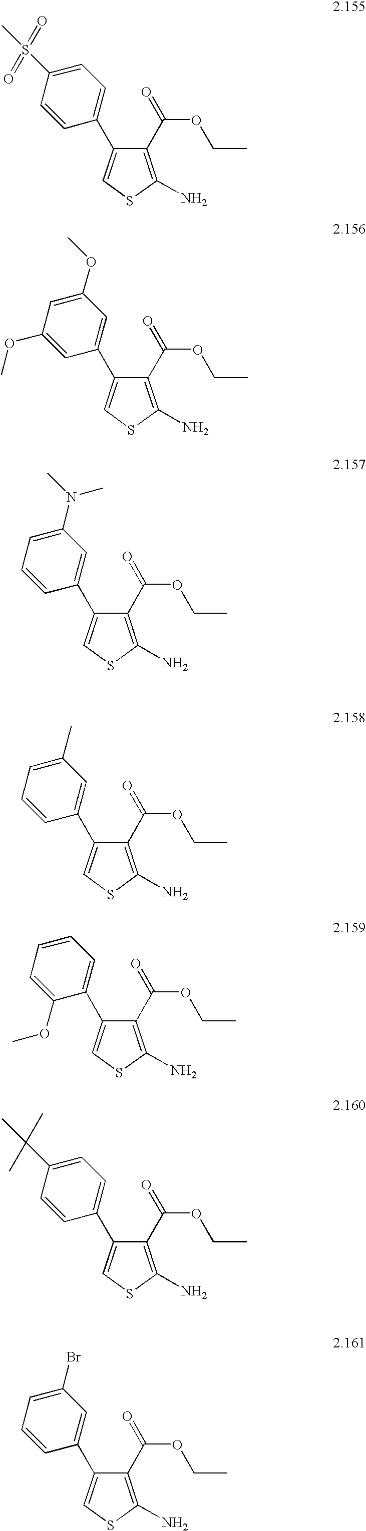 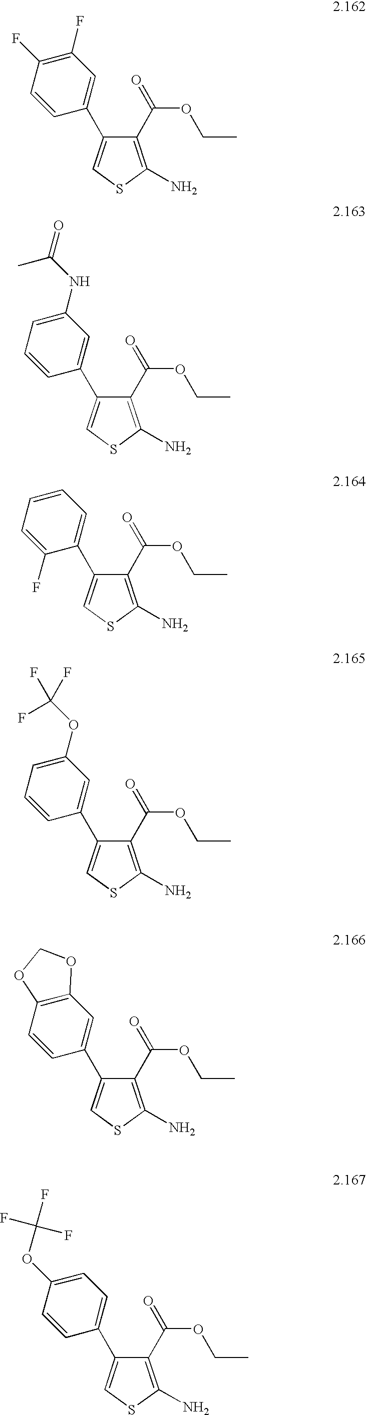 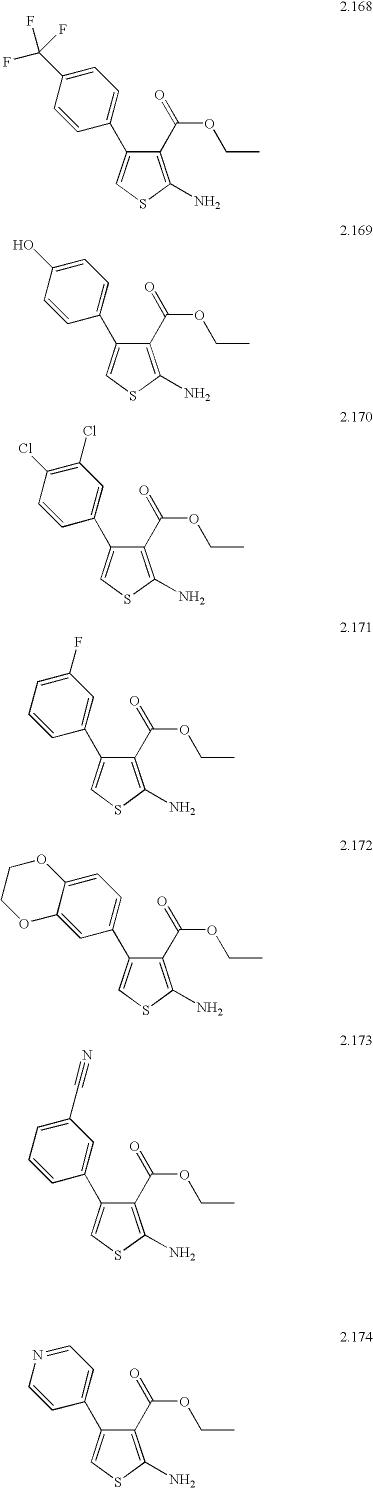 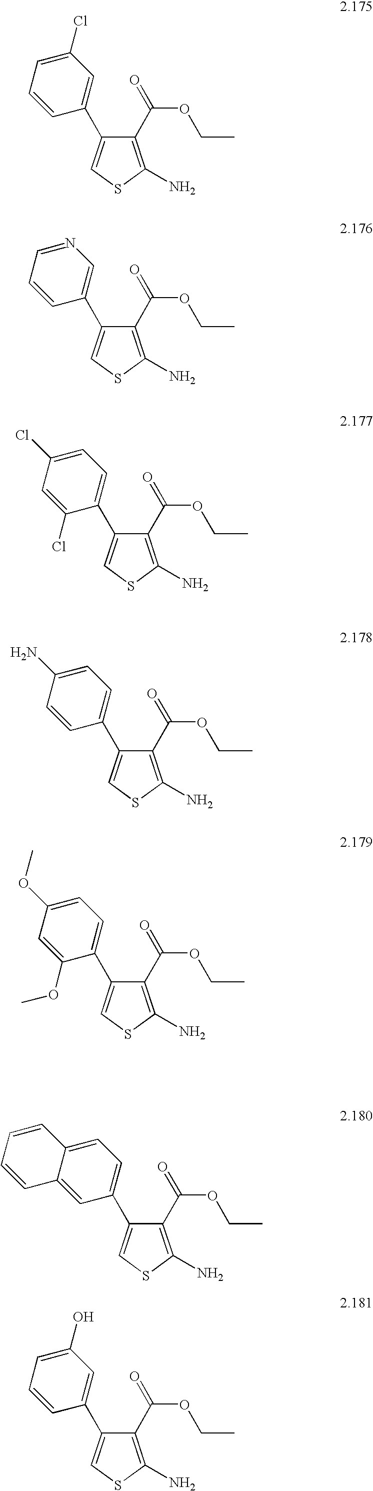 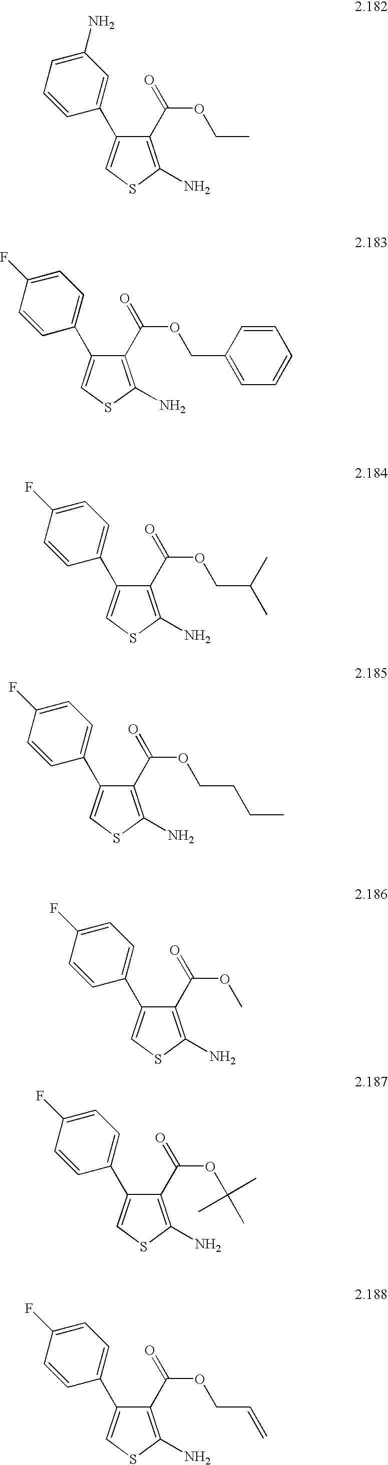 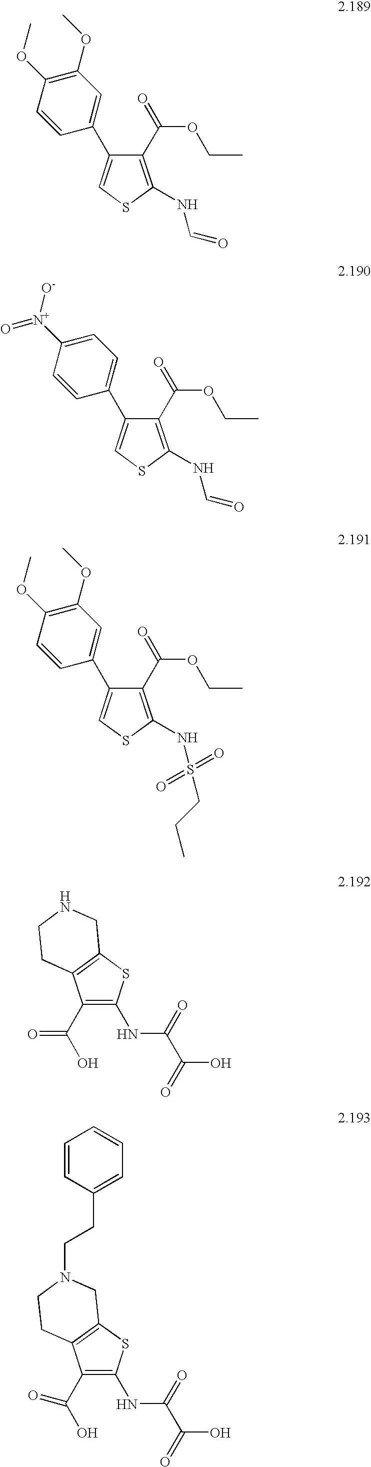
a stereoisomer thereof, a pharmaceutically acceptable salt thereof, a hydrate thereof, or a solvate of any of the foregoing.

53. The compound of claim 41, wherein the at least one ATP-utilizing enzyme is chosen from a human protein kinase.

54. The compound of claim 53, wherein the human protein kinase is chosen from ABL, ABL1, ABL-T315I, AKT1, AKT2, AURORA-A, BMX, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, INSR, KIT, LCK, LYNA, MAPKAPK-2, MET, MSK1, MSK2, NEK2, P38-α, P38-β, P38-γ, P38-δ, P70S6K1, PDGFR-Q, PDK1, PKA, ROCK2, SRC, SYK, TRKB, and ZAP70.

55. A pharmaceutical composition comprising at least one pharmaceutically acceptable excipient, and a therapeutically effective amount of at least one compound according to claim 41.

56. A pharmaceutical composition comprising at least one pharmaceutically acceptable excipient, and a therapeutically effective amount of at least one compound according to claim 52.

57. The pharmaceutical composition of claim 55, wherein the at least one compound is present in an amount effective for the treatment in a patient of at least one disease chosen from Alzheimer's disease, stroke, diabetes, obesity, inflammation, and cancer.

58. The pharmaceutical composition of claim 57, wherein cancer is chosen from at least one of glioblastoma, ovarian, breast, endometrial, hepatocellular carcinoma, melanoma, digestive tract, lung, renal-cell carcinoma, thyroid, lymphoid, prostate, and pancreatic cancer.

59. A method of treating a disease in a patient in need of such treatment comprising administering to the patient a therapeutically effective amount of at least one compound according to claim 41.

60. A method of treating a disease in a patient in need of such treatment comprising administering to the patient a therapeutically effective amount of at least one compound according to claim 52.

61. The method of claim 59, wherein the at least one disease is chosen from Alzheimer's disease, stroke, diabetes, obesity, inflammation, and cancer.

62. The method of claim 61, wherein cancer is chosen from at least one of glioblastoma, ovarian, breast, endometrial, hepatocellular carcinoma, melanoma, digestive tract, lung, renal-cell carcinoma, thyroid, lymphoid, prostate, and pancreatic cancer.

63. A method of inhibiting at least one ATP-utilizing enzyme in a subject comprising administering to the subject at least one compound according to claim 41.

64. A method of inhibiting at least one ATP-utilizing enzyme in a subject comprising administering to the subject at least one compound according to claim 52.

65. The method of claim 63, wherein the ATP-utilizing enzyme is chosen from a human protein kinase.

66. The method of claim 65, wherein the human protein kinase is chosen from ABL, ABL1, ABL-T315I, AKT1, AKT2, AURORA-A, BMX, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, INSR, KIT, LCK, LYNA, MAPKAPK-2, MET, MSK1, MSK2, NEK2, P38-α, P38-β, P38-γ, P38-δ, P70S6K1, PDGFR-α, PDK1, PKA, ROCK2, SRC, SYK, TRKB, and ZAP70.

67. A method of inhibiting at least one ATP-utilizing enzyme comprising contacting the ATP-utilizing enzyme with at least one compound according to claim 41.

68. A method of inhibiting at least one ATP-utilizing enzyme comprising contacting the ATP-utilizing enzyme with at least one compound according to claim 52.

69. The method of claim 67, wherein the ATP-utilizing enzyme is chosen from a human protein kinase.

70. The method of claim 69, wherein human protein kinase is chosen from ABL, ABL1, ABL-T315I, AKT1, AKT2, AURORA-A, BMX, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, INSR, KIT, LCK, LYNA, MAPKAPK-2, MET, MSK1, MSK2, NEK2, P38-α, P38-β, P38-γ, P38-δ, P70S6K1, PDGFR-α, PDK1, PKA, ROCK2, SRC, SYK, TRKB, and ZAP70.

71. A method of treating a disease regulated by at least one ATP-utilizing enzyme in a subject in need of such treatment comprising administering to the subject a therapeutically effective amount of at least one compound according to claim 41.

72. A method of treating a disease regulated by at least one ATP-utilizing enzyme in a subject in need of such treatment comprising administering to the subject a therapeutically effective amount of at least one compound according to claim 52.

73. The method of claim 71, wherein the ATP-utilizing enzyme is chosen from a human protein kinase.

74. The method of claim 73, wherein the protein kinase is chosen from ABL, ABL1, ABL-T315I, AKT1, AKT2, AURORA-A, BMX, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, INSR, KIT, LCK, LYNA, MAPKAPK-2, MET, MSK1, MSK2, NEK2, P38-α, P38-β, P38-γ, P38-δ, P70S6K1, PDGFR-α, PDK1, PKA, ROCK2, SRC, SYK, TRKB, and ZAP70.

75. At least one compound of Formula (V): 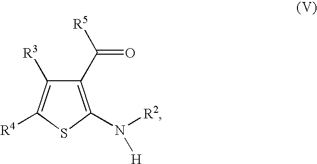
a stereoisomer thereof, a pharmaceutically acceptable salt thereof, a hydrate thereof, or a solvate of any of the foregoing, wherein:
R² is chosen from H, and -ZR⁶ wherein
Z is carbonyl; and
R⁶ is chosen from alkyl, substituted alkyl, heteroalkyl, substituted heteroalkyl, aryl, substituted aryl, heteroaryl, substituted heteroaryl, heterocycloalkylalkyl, and substituted heterocycloalkylalkyl;
R³ is chosen from H, halogen, alkyl, and substituted alkyl;
R⁴ is chosen from H, halogen, alkyl, and substituted alkyl;
or R³ and R⁴ together with the atoms to which R³ and R⁴ are attached form a cycloalkyl, substituted cycloalkyl ring;
R⁵ is chosen from H, aryl, substituted aryl, heteroaryl, and substituted heteroaryl;
and wherein the compound of Formula (V), a stereoisomer thereof, a pharmaceutically acceptable salt thereof, a hydrate thereof, or a solvate of any of the foregoing, exhibits ATP-utilizing enzyme inhibitory activity.

76. The compound of claim 75, wherein R⁴ is chosen from H, C₁₋₆ alkyl, substituted C₁₋₆ alkyl.

77. The compound of claim 75, wherein R³ is chosen from H, C₁₋₆ alkyl, substituted C₁₋₆ alkyl.

78. The compound of claim 75, wherein R³ and R⁴ together with the carbon atoms to which R³ and R⁴ are attached form a C₅₋₈ cycloalkyl or substituted C₅₋₈ cycloalkyl ring.

79. The compound of claim 75, wherein R⁵ is chosen from C₅₋₁₂ aryl, substituted C₅₋₁₂ aryl, C₅₋₁₂ heteroaryl, and substituted C₅₋₁₂ heteroaryl.

80. The compound of claim 79, where the at least one substituent group is chosen from halogen, C₁₋₆ alkyl, and C₁₋₆ alkoxy.

81. The compound of claim 75, wherein R⁵ is chosen from C₅₋₆ aryl, substituted C₅₋₆ aryl, C₅₋₆ heteroaryl, and substituted C₅₋₆ heteroaryl.

82. The compound of claim 81, where the at least one substituent group is chosen from halogen, C₁₋₆ alkyl, and C₁₋₆ alkoxy.

83. The compound of claim 75, wherein R² is chosen from H, and —C(O)R⁶ wherein R⁶ is chosen from C₁₋₁₀ alkyl, substituted C₁₋₁₀ alkyl, C₁₋₁₀ heteroalkyl, substituted C₁₋₁₀ heteroalkyl, C₅₋₁₂ aryl, substituted C₅₋₁₂ aryl, C₅₋₁₂ heteroaryl, substituted C₅₋₁₂ heteroaryl, C₆₋₁₈ heterocycloalkylalkyl, and substituted C₆₋₁₈ heterocycloalkylalkyl.

84. The compound of claim 83, wherein the at least one substituent group is chosen from halogen, —OH, and C₁₋₆ alkyl.

85. The compound of claim 75, wherein the at least one compound has the structure of any of compounds 3.1 to 3.21: 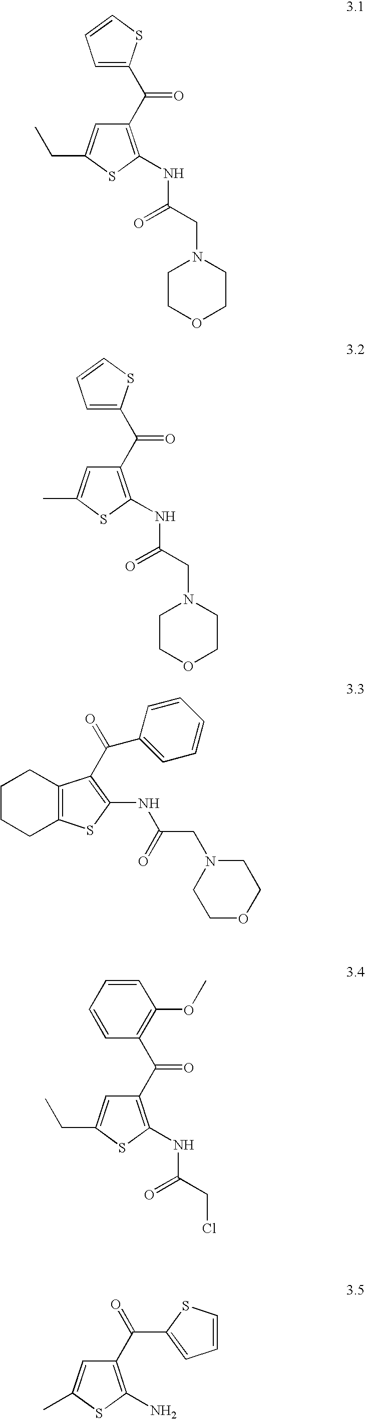 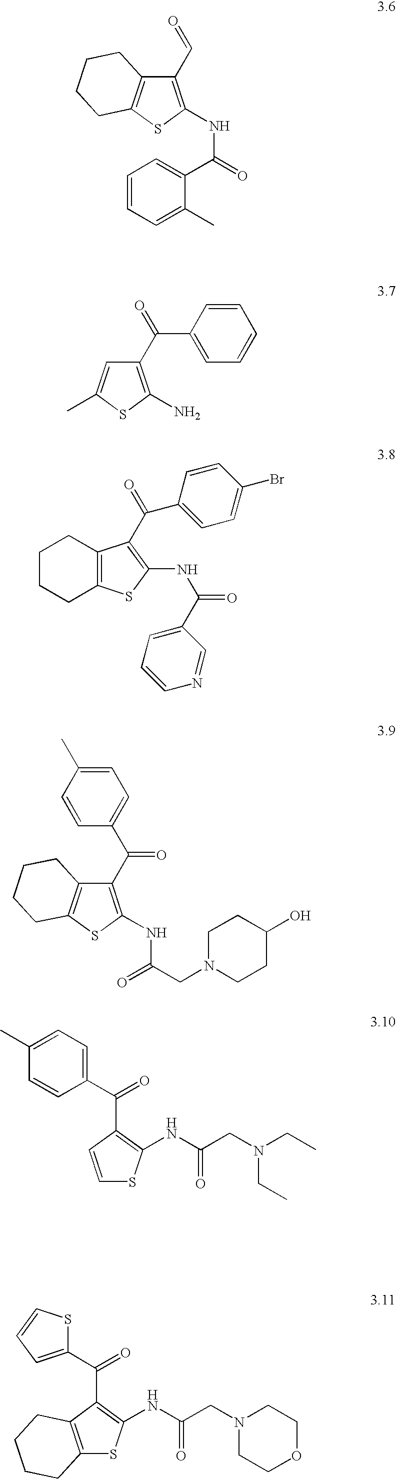 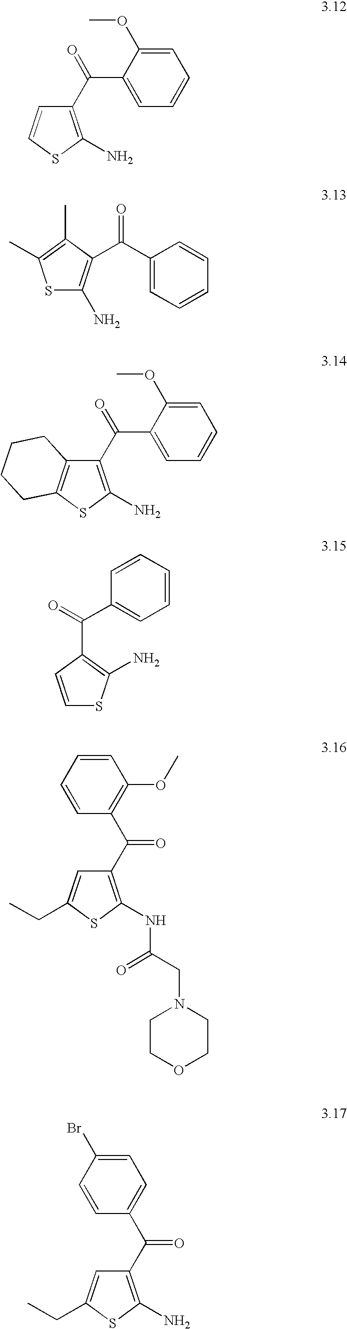 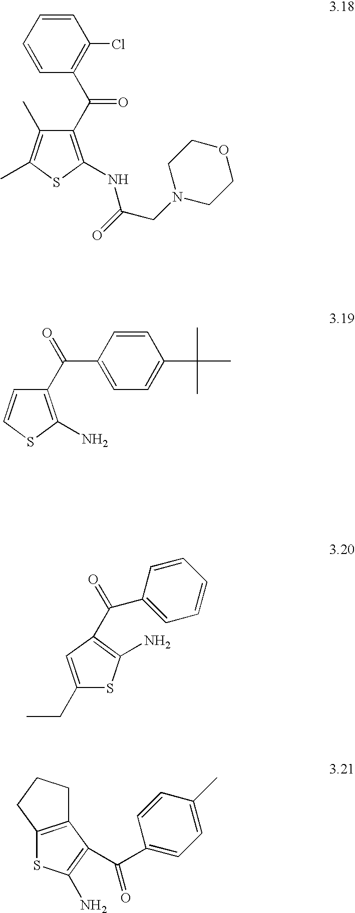
a stereoisomer thereof, a pharmaceutically acceptable salt thereof, a hydrate thereof, or a solvate of any of the foregoing.

86. The compound of claim 75, wherein the at least one ATP-utilizing enzyme is chosen from a human protein kinase.

87. The compound of claim 86, wherein the human protein kinase is chosen from AURORA-A, CDK2/cyclinE, CK2, FLT-3, GSK-3α, GSK-3β, KIT, MAPKAPK-2, MSK1, P38-β, PDGFR-α, and TRKB.

88. A pharmaceutical composition comprising at least one pharmaceutically acceptable excipient, and a therapeutically effective amount of at least one compound according to claim 75.

89. A pharmaceutical composition comprising at least one pharmaceutically acceptable excipient, and a therapeutically effective amount of at least one compound according to claim 85.

90. The pharmaceutical composition of claim 88, wherein the at least one compound is present in an amount effective for the treatment in a patient of at least one disease chosen from Alzheimer's disease, stroke, diabetes, obesity, inflammation, and cancer.

91. The pharmaceutical composition of claim 88, wherein cancer is chosen from at least one of glioblastoma, ovarian, breast, endometrial, hepatocellular carcinoma, melanoma, digestive tract, lung, renal-cell carcinoma, thyroid, lymphoid, prostate, and pancreatic cancer.

92. A method of treating a disease in a patient in need of such treatment comprising administering to the patient a therapeutically effective amount of at least one compound according to claim 75.

93. A method of treating a disease in a patient in need of such treatment comprising administering to the patient a therapeutically effective amount of at least one compound according to claim 85.

94. The method of claim 92, wherein the at least one disease is chosen from Alzheimer's disease, stroke, diabetes, obesity, inflammation, and cancer.

95. The method of claim 94, wherein cancer is chosen from at least one of glioblastoma, ovarian, breast, endometrial, hepatocellular carcinoma, melanoma, digestive tract, lung, renal-cell carcinoma, thyroid, lymphoid, prostate, and pancreatic cancer.

96. A method of inhibiting at least one ATP-utilizing enzyme in a subject comprising administering to the subject at least one compound according to claim 75.

97. A method of inhibiting at least one ATP-utilizing enzyme in a subject comprising administering to the subject at least one compound according to claim 85.

98. The method of claim 96, wherein the ATP-utilizing enzyme is chosen from a human protein kinase.

99. The method of claim 98, wherein the human protein kinase is chosen from AURORA-A, CDK2/cyclinE, CK2, FLT-3, GSK-3α, GSK-3β, KIT, MAPKAPK-2, MSK1, P38-β, PDGFR-α, and TRKB.

100. A method of inhibiting at least one ATP-utilizing enzyme comprising contacting the ATP-utilizing enzyme with at least one compound according to claim 75.

101. A method of inhibiting at least one ATP-utilizing enzyme comprising contacting the ATP-utilizing enzyme with at least one compound according to claim 85.

102. The method of claim 100, wherein the ATP-utilizing enzyme is chosen from a human protein kinase.

103. The method of claim 102, wherein human protein kinase is chosen from AURORA-A, CDK2/cyclinE, CK2, FLT-3, GSK-3α, GSK-3β, KIT, MAPKAPK-2MSK1, P38-β, PDGFR-α, and TRKB.

104. A method of treating a disease regulated by at least one ATP-utilizing enzyme in a subject in need of such treatment comprising administering to the subject a therapeutically effective amount of at least one compound according to claim 75.

105. A method of treating a disease regulated by at least one ATP-utilizing enzyme in a subject in need of such treatment comprising administering to the subject a therapeutically effective amount of at least one compound according to claim 85.

106. The method of claim 104, wherein the ATP-utilizing enzyme is chosen from a human protein kinase.

107. The method of claim 106, wherein the protein kinase is chosen from AURORA-A, CDK2/cyclinE, CK2, FLT-3, GSK-3α, GSK-3β, KIT, MAPKAPK-2, MSK1, P38-β, PDGFR-α, and TRKB.

108. At least one compound of Formula (VI): 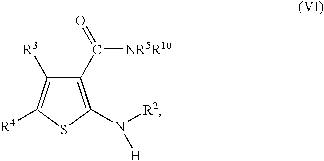
a stereoisomer thereof, a pharmaceutically acceptable salt thereof, a hydrate thereof, or a solvate of any of the foregoing, wherein:
R² is chosen from H, aryl, substituted aryl, heteroaryl, substituted heteroaryl, heterocycloalkyl, substituted heterocycloalkyl, alkylsulfonyl, substituted, alkylsulfonyl, heteroalkylsulfonyl, substituted heteroalkylsulfonyl, and -ZR⁶, wherein
Z is carbonyl; and
R⁶ is chosen from H, alkyl, substituted alkyl, aryl, and substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, arylalkyl, substituted arylalkyl, heteroarylalkyl, and substituted heteroalkyl;
R³ is chosen from H, halogen, acyl, substituted acyl, alkoxycarbonyl, substituted alkoxycarbonyl, alkyl, substituted alkyl, aminocarbonyl, substituted aminocarbonyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
R⁴ is chosen from H, alkyl, substituted alkyl, aryl, substituted aryl, arylalkyl, substituted arylalkyl, heteroaryl, substituted heteroaryl, heteroarylalkyl, and substituted heteroarylalkyl;
or R³ and R⁴ together with the atoms to which R³ and R⁴ are attached form a cycloalkyl, substituted cycloalkyl, heterocycloalkyl, or substituted heterocycloalkyl ring;
R⁵ is chosen from H, alkyl, substituted alkyl;
R¹⁰ is chosen from H, alkyl, substituted alkyl, aryl, substituted aryl, arylalkyl, substituted arylalkyl, heteroalkyl, substituted heteroalkyl, heteroalkyl, and substituted heteroalkyl;
or, R⁵ and R¹⁰ together with the atoms to which R⁵ and R¹⁰ are attached form a cycloalkyl, substituted cycloalkyl, heterocycloalkyl, or substituted heterocycloalkyl ring; and
with the provisos that
when R³ is H, and R² is —C(═O)NR¹²R¹¹, and R¹¹ is H, then R¹² is not alkyl or substituted alkyl; and
when R¹ is H, and R⁵ is H, then R¹⁰ is not H;
and wherein the compound of Formula (VI), a stereoisomer thereof, a pharmaceutically acceptable salt thereof, a hydrate thereof, or a solvate of any of the foregoing, is an inhibitor of at least one ATP-utilizing enzyme.

109. The compound of claim 108, wherein
R² is chosen from H, aryl, substituted aryl, heteroaryl, substituted heteroaryl, arylalkyl, substituted arylalkyl, heteroarylalkyl, substituted arylalkyl, cycloalkyl, substituted cycloalkyl, heterocycloalkyl, substituted heterocycloalkyl, alkylsulfonyl, substituted, alkylsulfonyl, heteroalkylsulfonyl, substituted heteroalkylsulfonyl, and -ZR⁶, wherein
Z is carbonyl, and
R⁶ is chosen from H, alkyl, substituted alkyl, aryl, and substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, cycloalkylalkyl, substituted cycloalkylalkyl, heterocycloalkylalkyl, substituted heterocycloalkylalkyl, arylalkyl, substituted arylalkyl, heteroarylalkyl, and substituted heteroarylalkyl;
R³ is chosen from alkyl, substituted alkyl, aryl, substituted aryl, and H;
R⁴ is chosen from H, halogen, alkyl, substituted alkyl, aryl, substituted aryl, arylalkyl, and substituted arylalkyl; or
R³ and R⁴ together with the atoms to which R³ and R⁴ are attached, form a cycloalkyl, substituted cycloalkyl, heterocycloalkyl, or substituted heterocycloalkyl ring;
R⁵ is H; and
R¹⁰ is chosen from H, alkyl, substituted alkyl, aryl, substituted aryl, arylalkyl, and substituted arylalkyl;
or R⁵ and R¹⁰ together with the atoms to which R⁵ and R¹⁰ are attached form a heterocycloalkyl or substituted heterocycloalkyl ring.

110. The compound of claim 108, wherein R³ is chosen from H, C₁₋₆ alkyl, substituted C₁₋₆ alkyl, C₅₋₁₀ aryl, and substituted C₅₋₁₀ aryl.

111. The compound of claim 108, wherein R³ is chosen from H, methyl, and phenyl.

112. The compound of claim 108, wherein R⁵ is chosen from H, and R¹⁰ is chosen from H, C₁₋₈ alkyl, substituted C₁₋₈ alkyl, C₁₋₁₂ heteroalkyl, substituted C₁₋₁₂ heteroalkyl, C₅₋₁₀ aryl, substituted C₅₋₁₀ aryl, C₆₋₁₂ arylalkyl, and substituted C₆₋₁₂ arylalkyl.

113. The compound of claim 112, wherein the at least one substituent group is chosen from halogen, C₁₋₆ alkyl, C₁₋₆ alkoxy, —OH, ═O, and —NH₂.

114. The compound of claim 108, wherein R⁵ and R¹⁰ together with the atoms to which R⁵ and R¹⁰ are attached form a C₅₋₁₀ heterocycloalkyl or substituted C₅₋₁₀ heterocycloalkyl ring.

115. The compound of claim 108, wherein R⁴ is chosen from H, C₁₋₆ alkyl, substituted C₁₋₆ alkyl, C₅₋₁₀ aryl, substituted C₅₋₁₀ aryl, C₆₋₁₂ arylalkyl, and substituted C₆₋₁₂ arylalkyl.

116. The compound of claim 108, wherein R³ and R⁴ together with the atoms to which R³ and R⁴ are attached form a C₅₋₈ cycloalkyl, substituted C₅₋₈ cycloalkyl, C₅₋₈ heterocycloalkyl, or substituted C₅₋₈ heterocycloalkyl ring.

117. The compound of claim 116, wherein the at least one substituent group is chosen from halogen, C₁₋₆ alkyl, substituted C₁₋₆ alkyl, C₁₋₆ heteroalkyl, substituted C₁₋₆ heteroalkyl, C₆₋₁₀ arylalkyl, substituted C₆₋₁₀ arylalkyl, and ═O.

118. The compound of claim 108, wherein R² is chosen from H, C₅-8 aryl, substituted C₅₋₈ aryl, C₅₋₈ heteroaryl, substituted C₅₋₈ heteroaryl, C₆₋₁₀ heterocycloalkyl, substituted C₆₋₁₀ heterocycloalkyl, C₆₋₁₀ heteroarylalkyl, substituted C₆₋₁₀ heteroarylalkyl, C₁₋₁₀ alkylsulfonyl, substituted C₁₋₁₀ alkylsulfonyl, and —C(O)R⁶ wherein
R⁶ is chosen from C₁₋₁₀ alkyl, substituted C₁₋₁₀ alkyl, C₁₋₁₀ heteroalkyl, substituted C₁₋₁₀ heteroalkyl, C₃₋₁₀ cycloalkyl, substituted C₃₋₁₀ cycloalkyl, C₃₋₁₀ heterocycloalkyl, substituted C₃₋₁₀ heterocycloalkyl, C₅₋₁₀ aryl, substituted C₅₋₁₀ aryl, C₅₋₁₀ heteroaryl, substituted C₅₋₁₁₀ heteroaryl, C₆₋₁₈ cycloalkylalkyl, substituted C₆₋₁₈ cycloalkylalkyl, C₆₋₁₈ heterocycloalkylalkyl, substituted C₆₋₁₈ heterocycloalkylalkyl, C₆₋₁₈ arylalkyl, substituted C₆₋₁₈ arylalkyl, C₆₋₁₈ heteroarylalkyl, and substituted C₆₋₁₈ heteroarylalkyl.

119. The compound of claim 118, wherein R² is chosen from —C(O)R⁶ and the at least one substituent group is chosen from halogen, C₁₋₆ alkyl, C₁₋₆ heteroalkyl, substituted C₁₋₆ heteroalkyl, C₁₋₆ alkoxy, substituted C₁₋₆ alkoxy, C₅₋₈ aryl, C₅₋₈ heterocycloalkyl, substituted C₅₋₈ heterocycloalkyl, C₅₋₈ heteroaryl, C₆₋₁₂ heterocycloalkylalkyl, substituted C₆₋₁₂ heterocycloalkylalkyl, C₆₋₁₂ heteroarylalkyl, substituted C₆₋₁₂ heteroarylalkyl, C₅₋₈ alkkylsulfonyl, ═O, ═S, —C(O)NH₂, —OH, —CF₃, nitro, —CN, —COOH, —OCF₃, and —N(CH₃)₂.

120. The compound of claim 108, wherein the at least one compound has the structure of any of compounds 4.1 to 4.285: 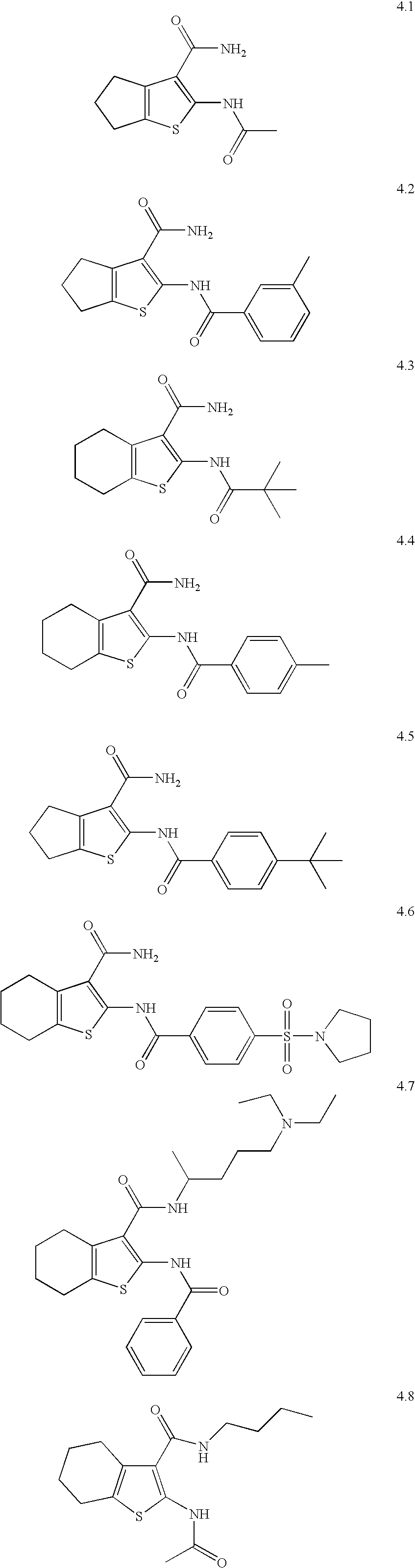 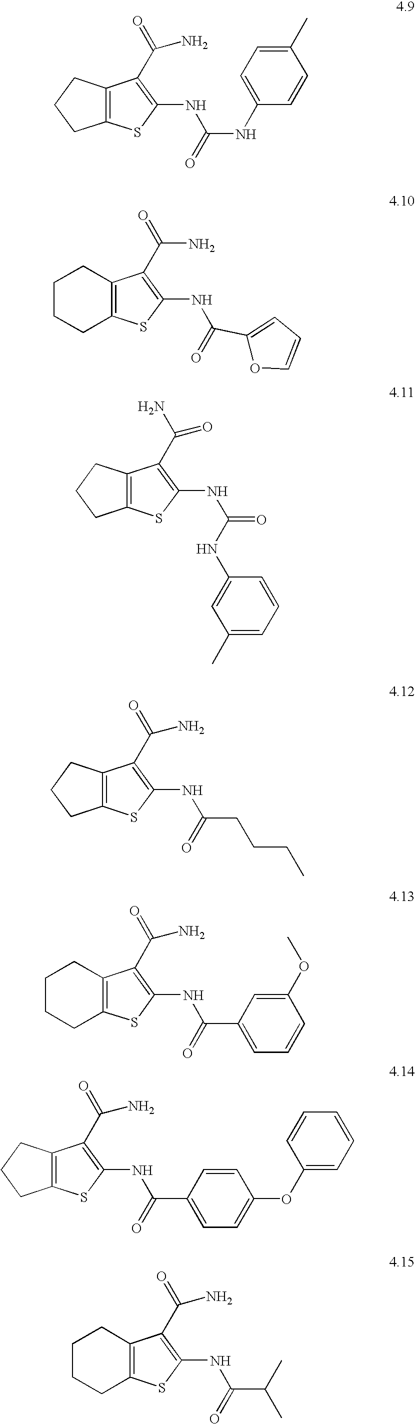 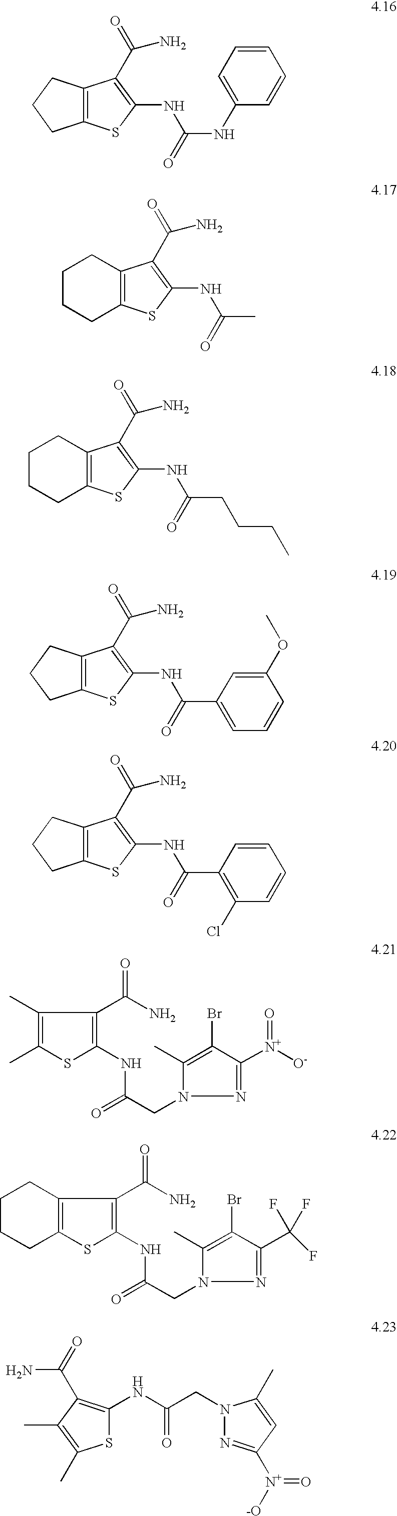 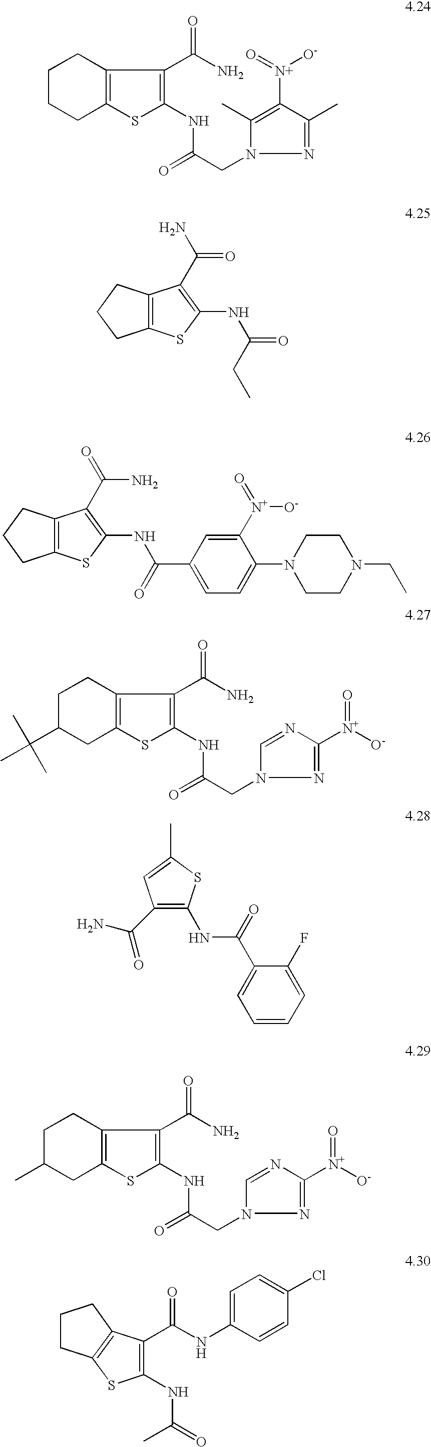 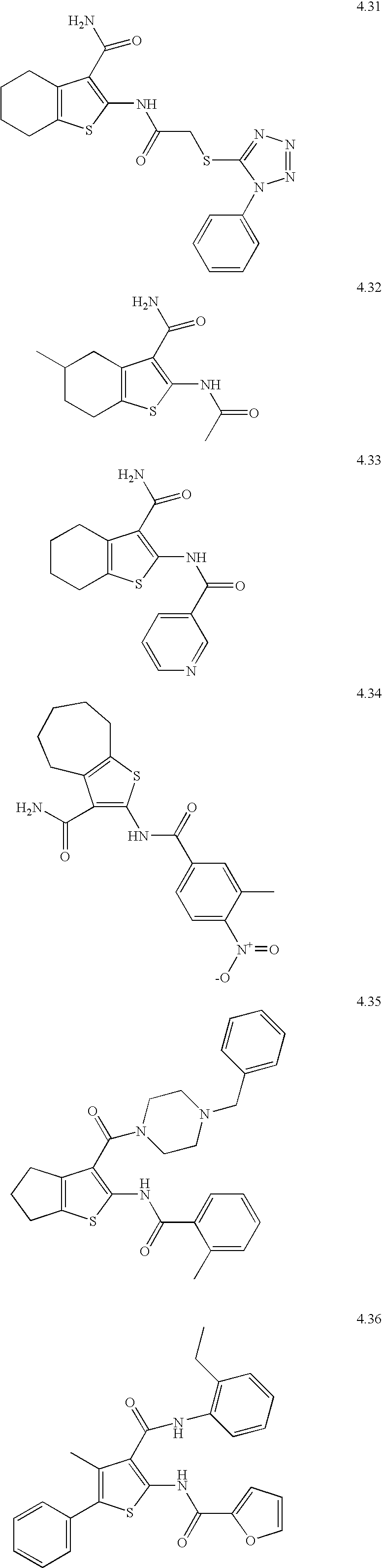 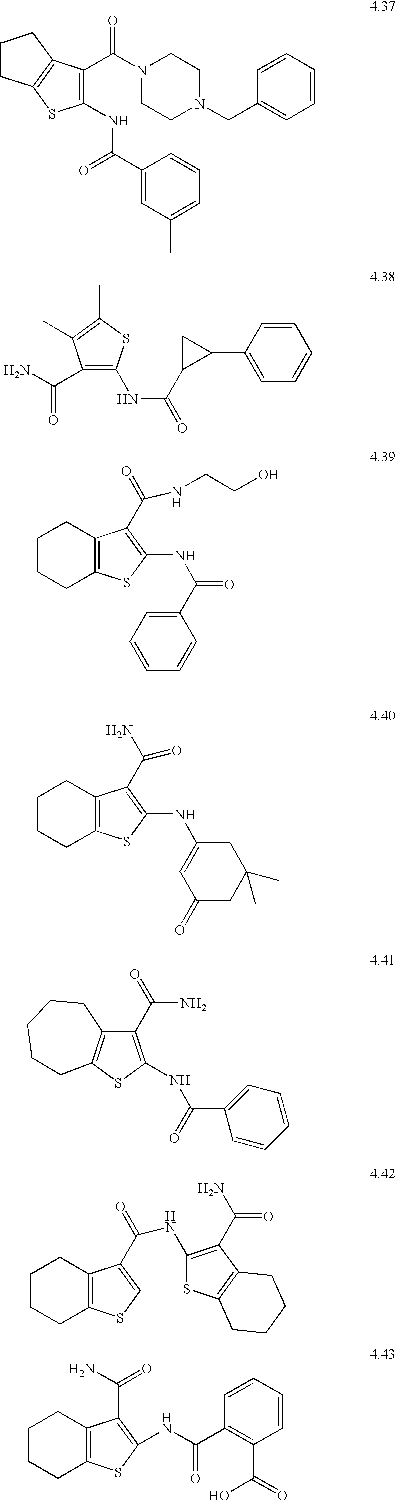 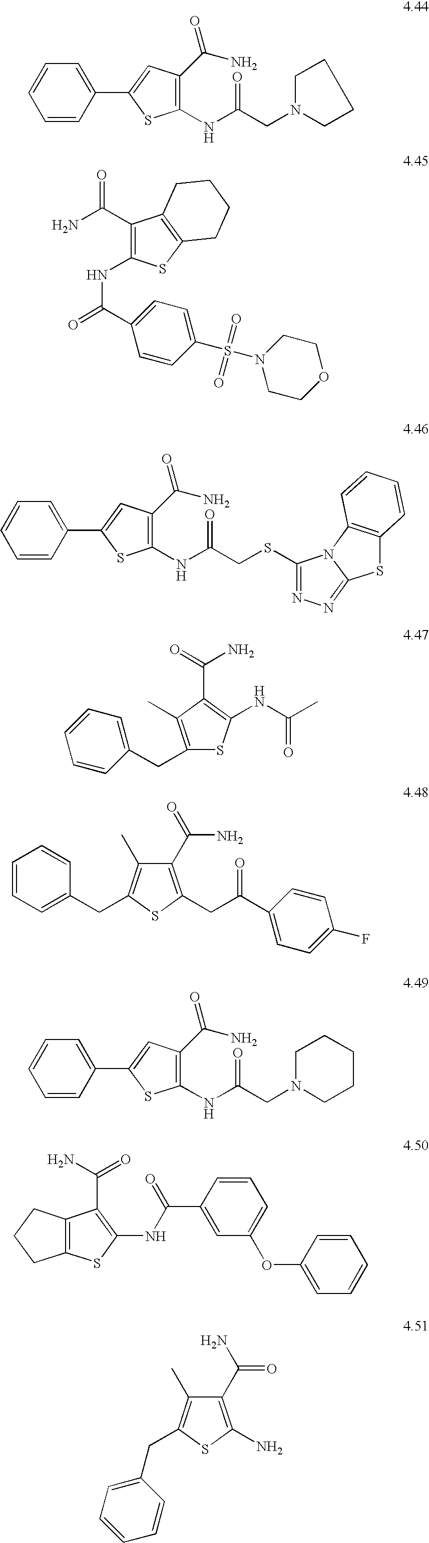 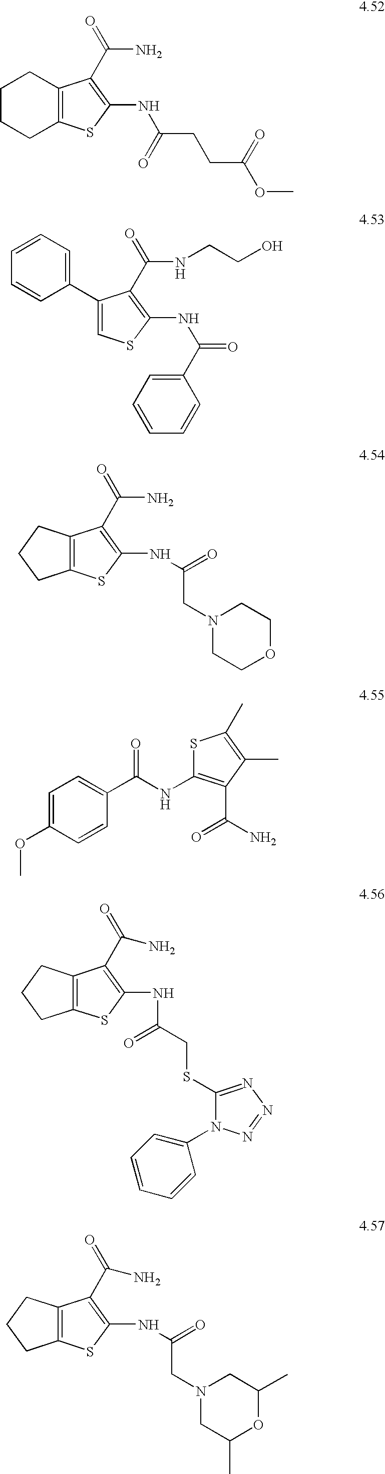 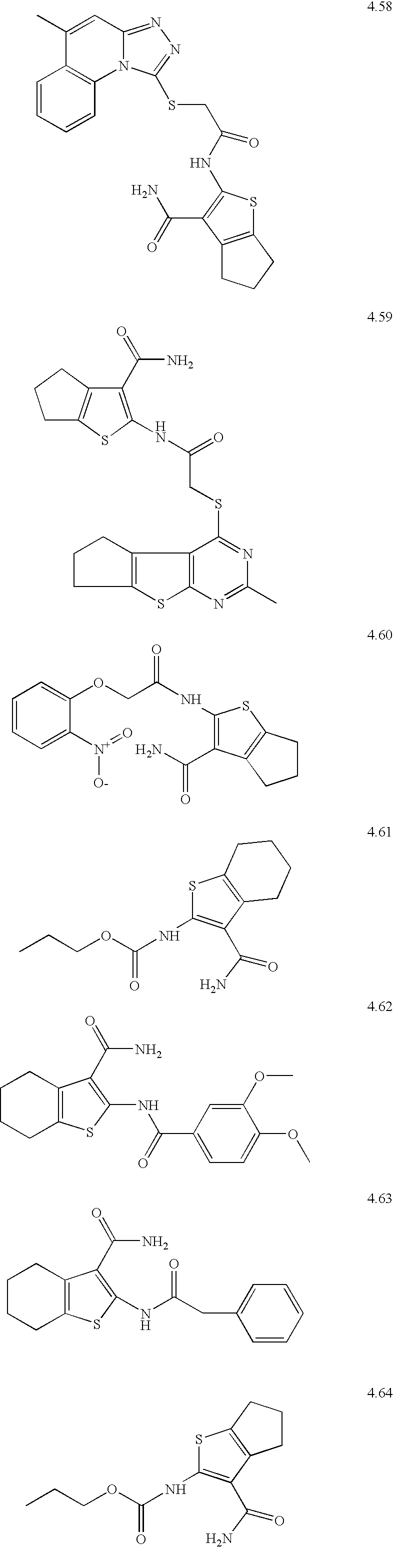 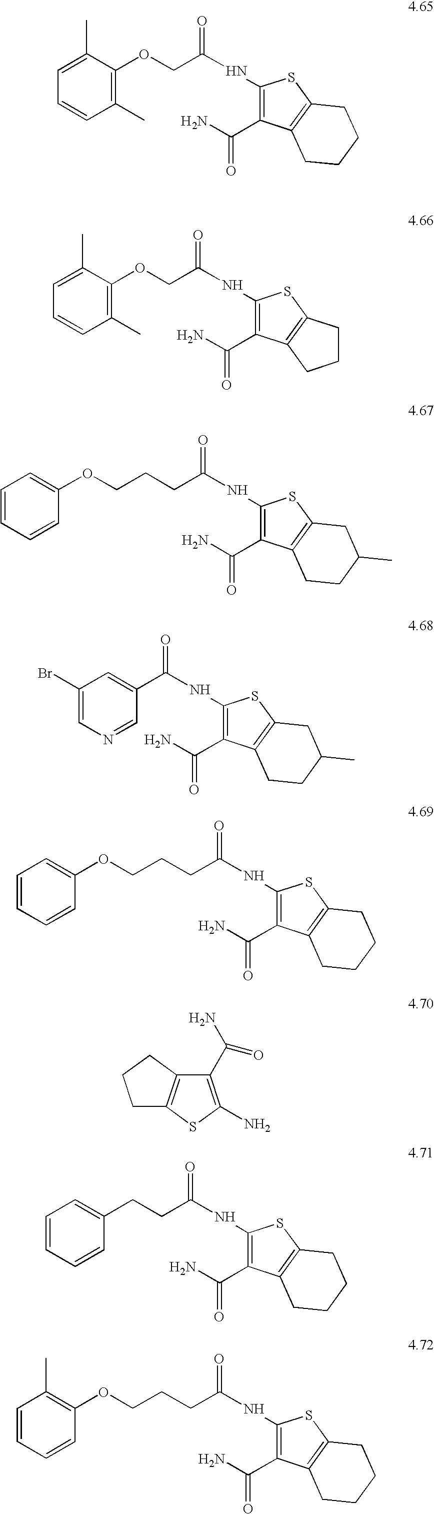 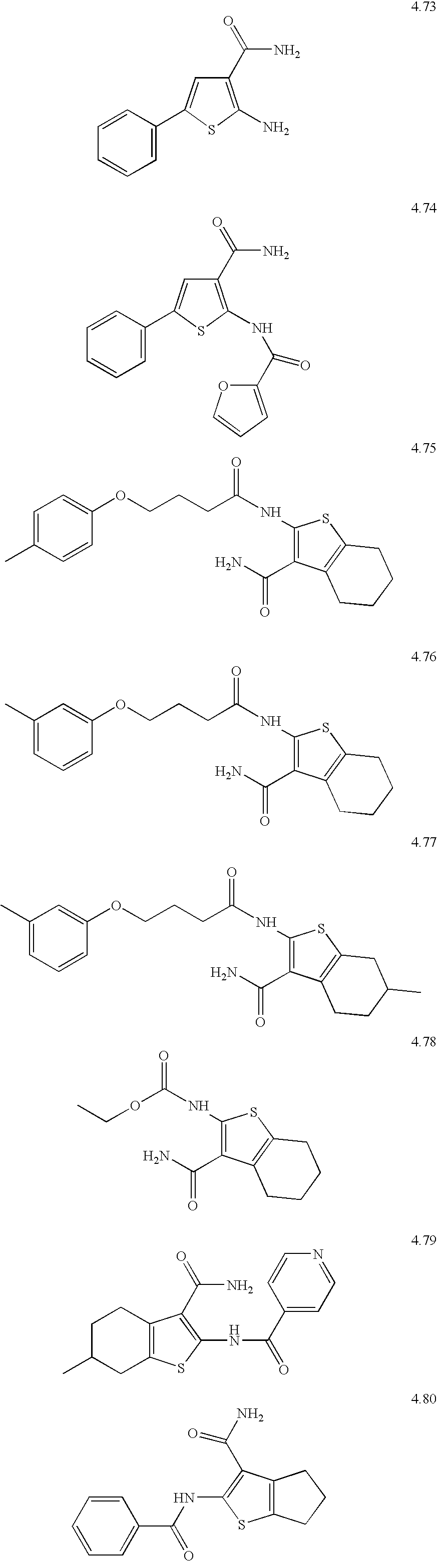 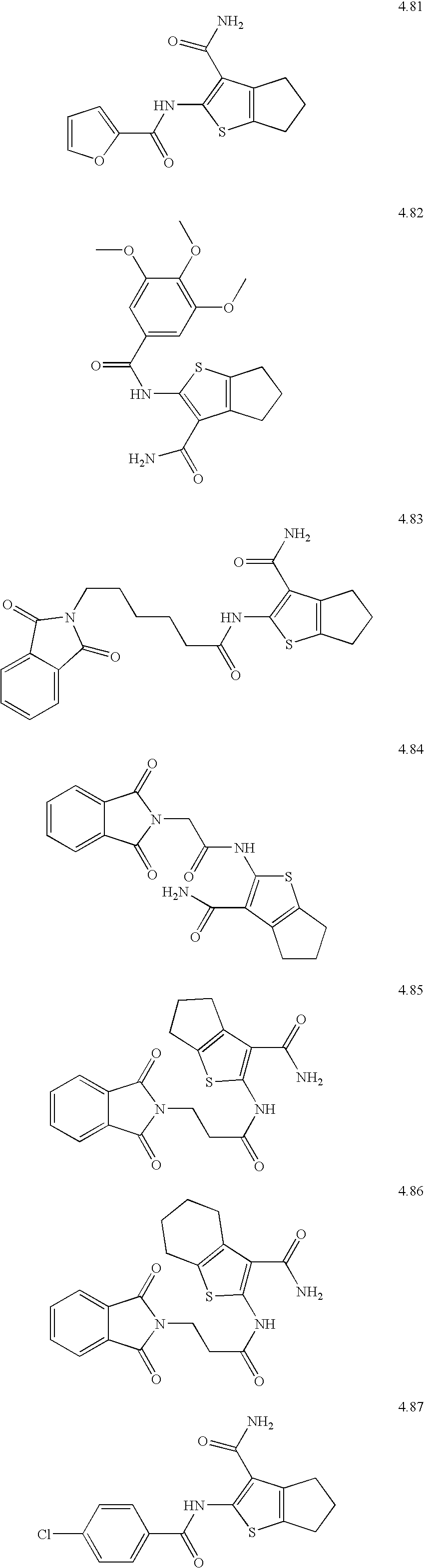 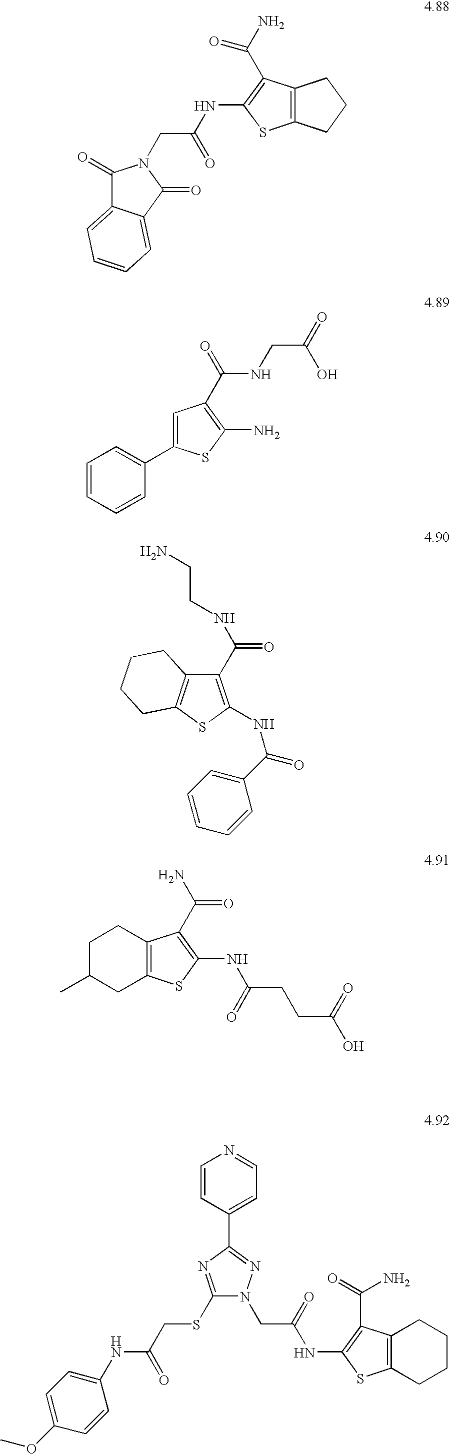 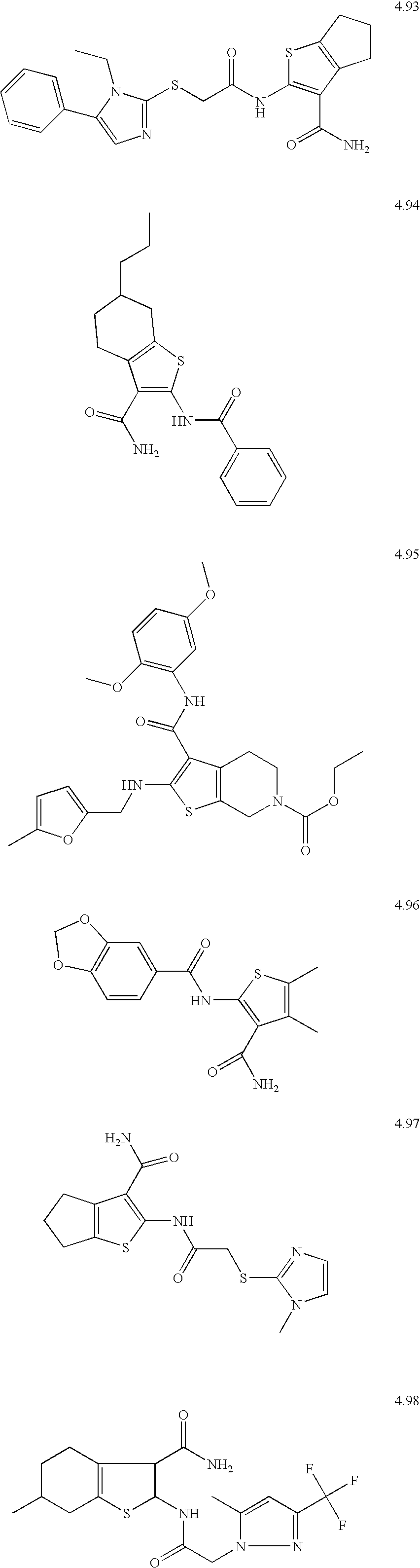 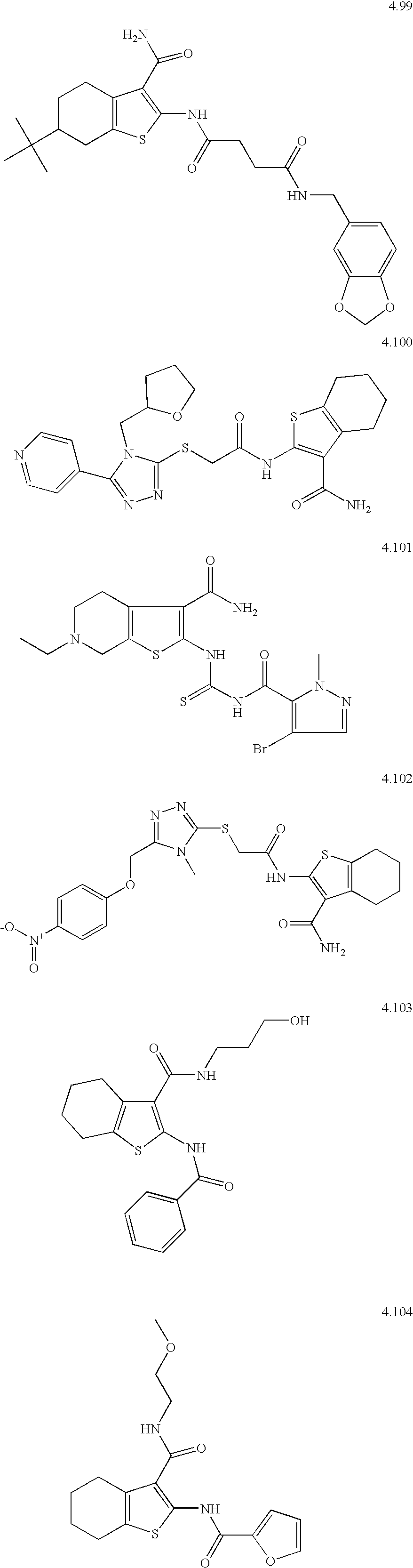 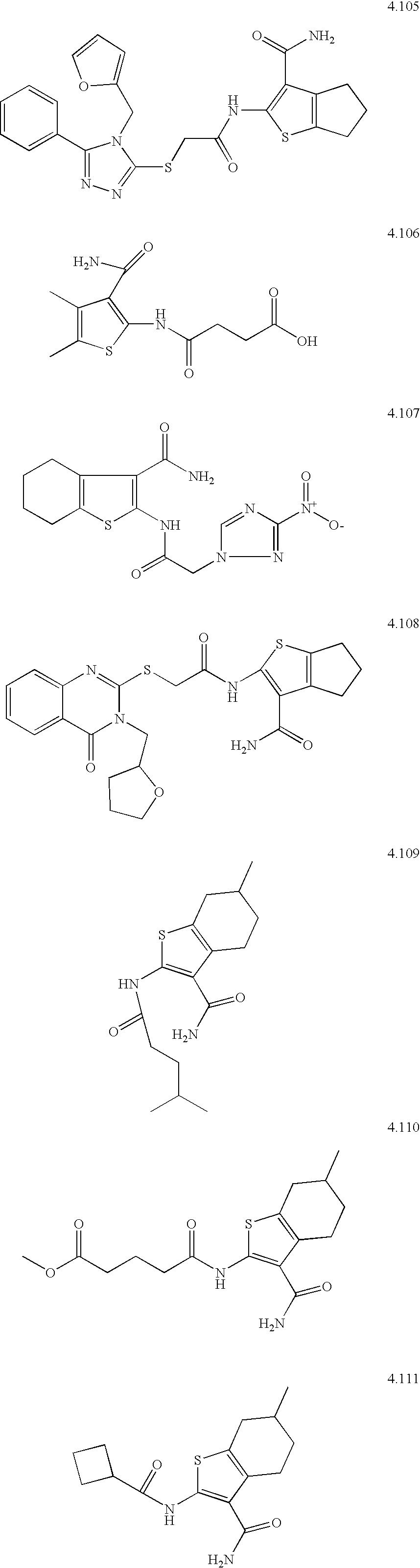 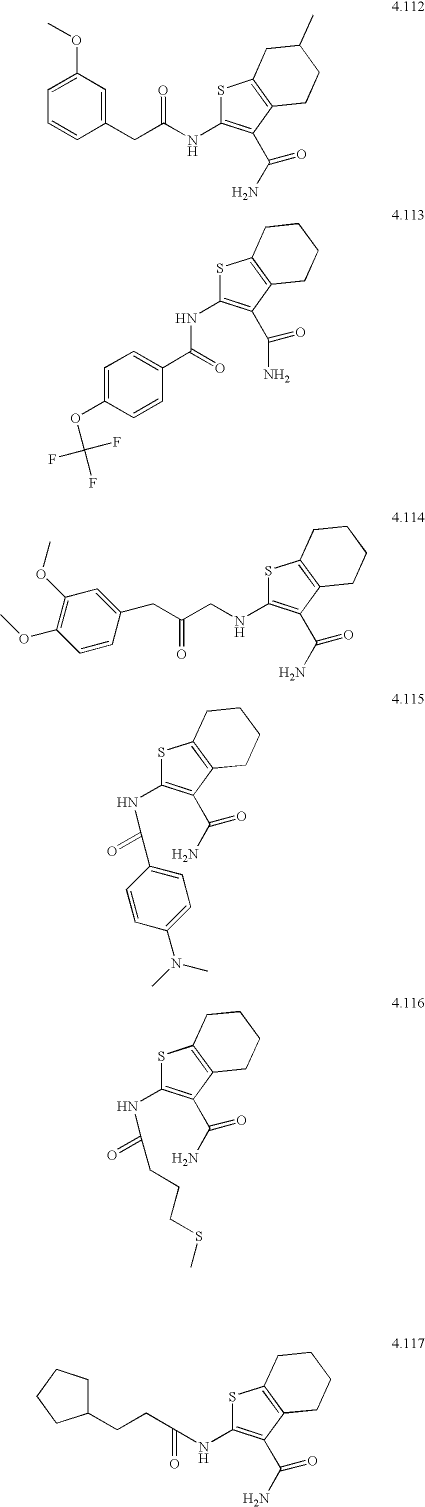 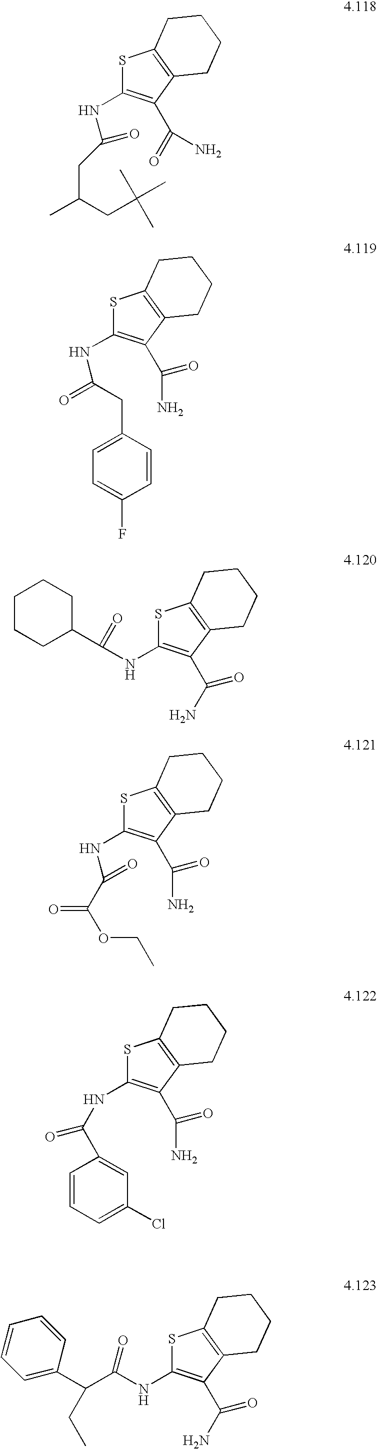 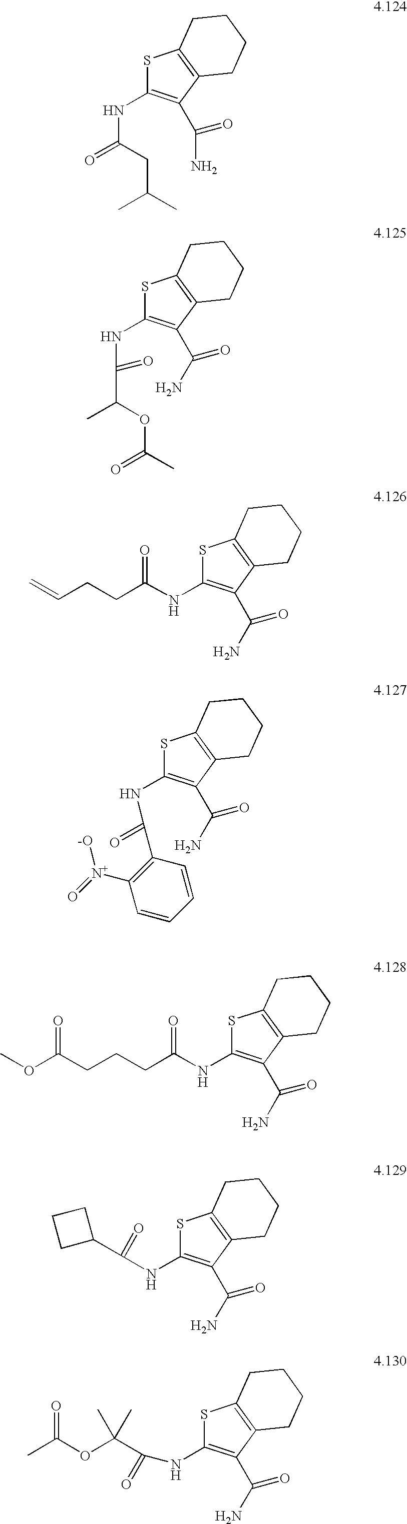 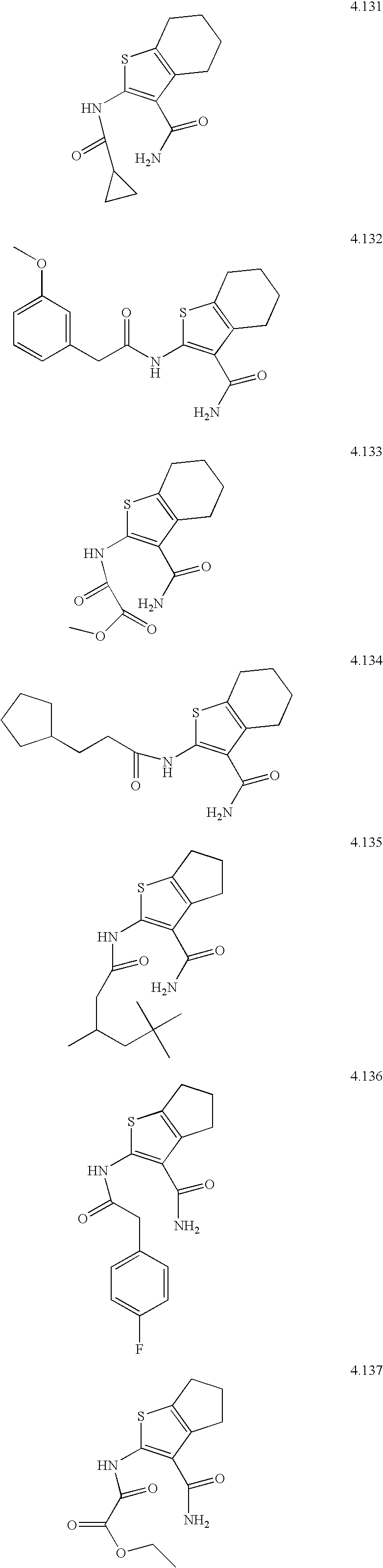 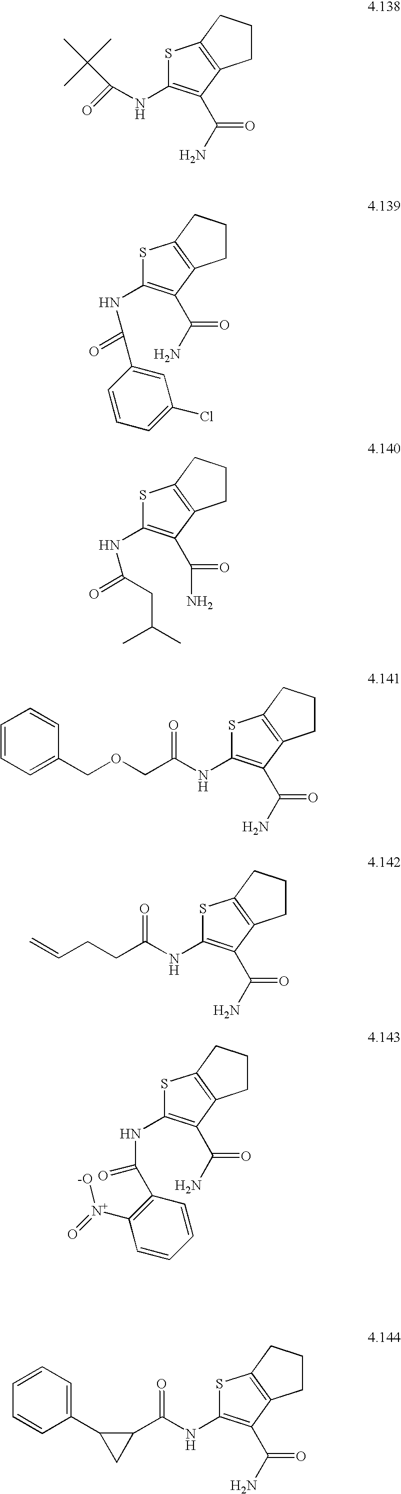 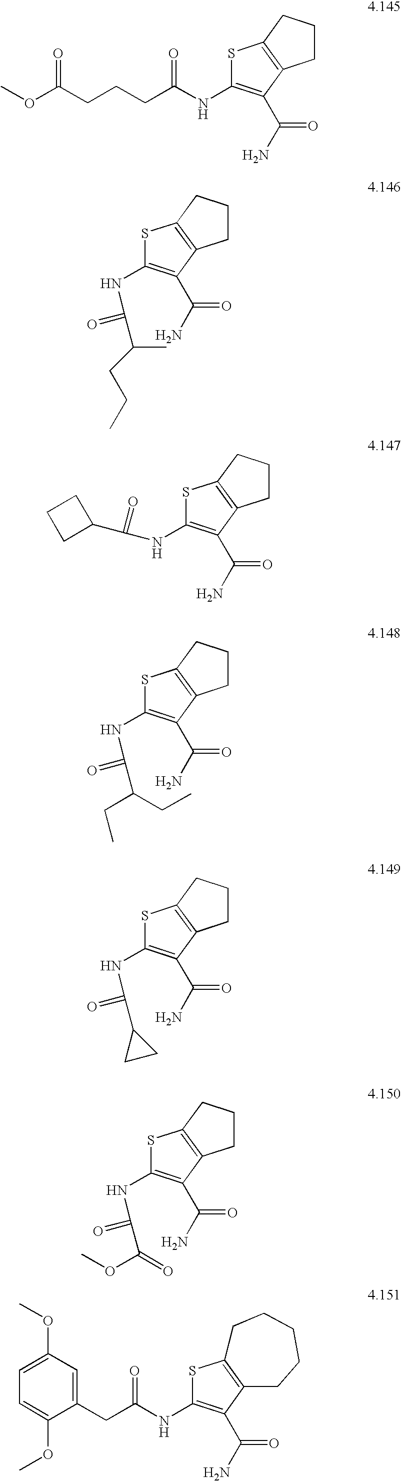 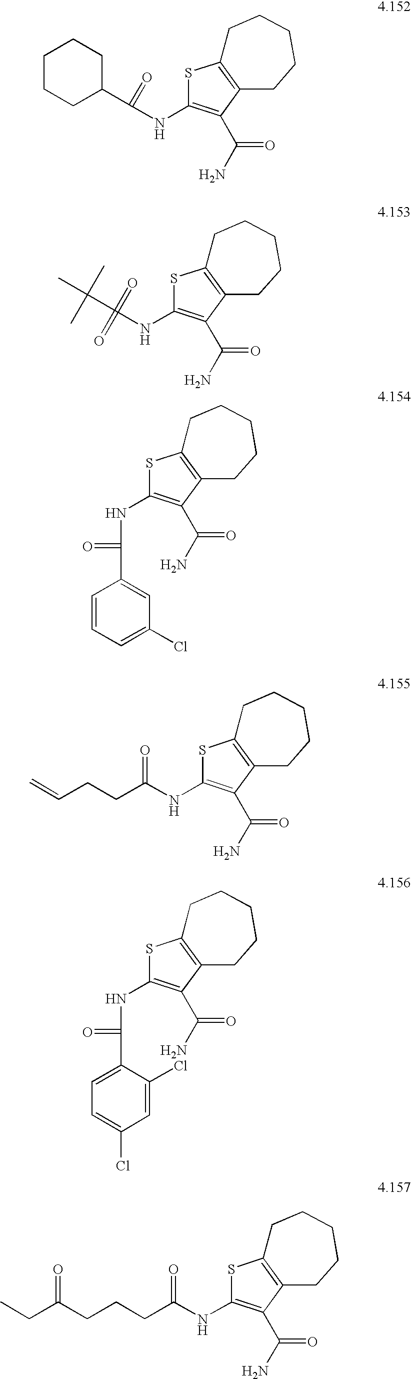 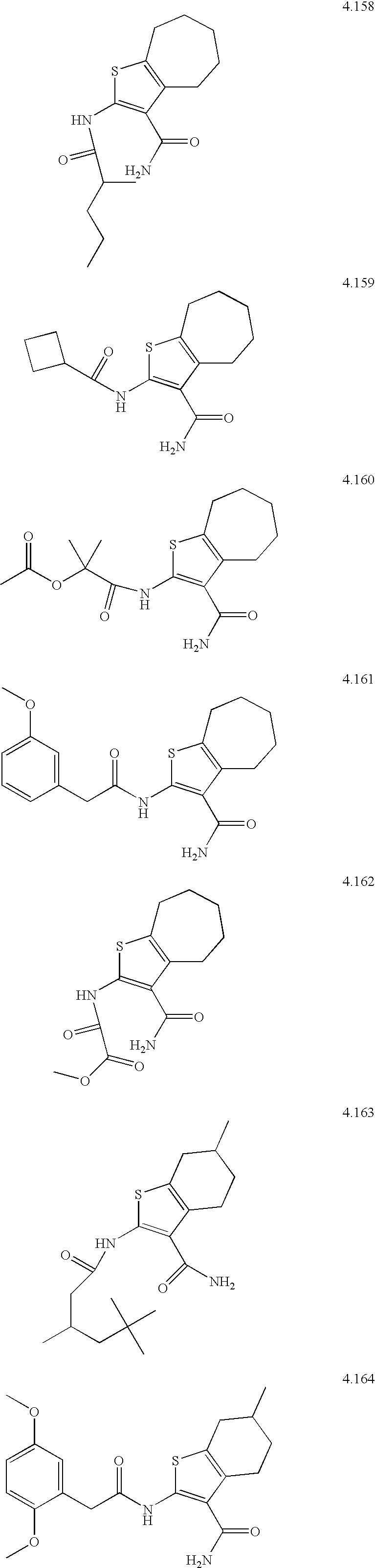 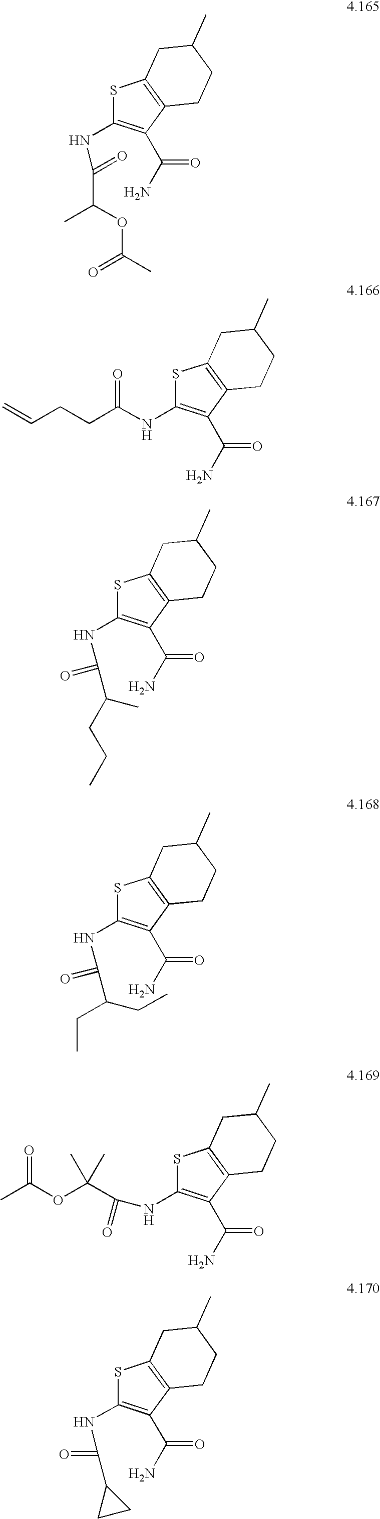 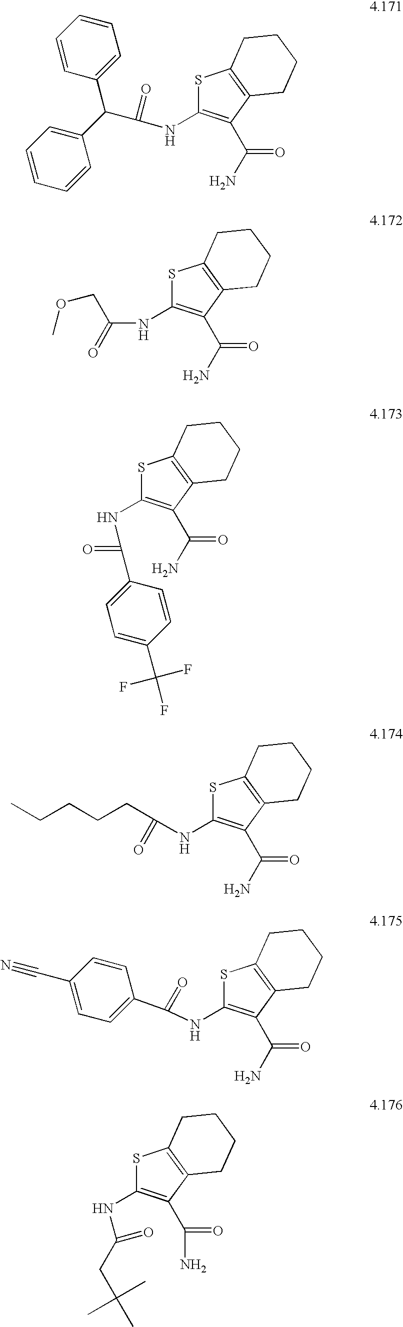 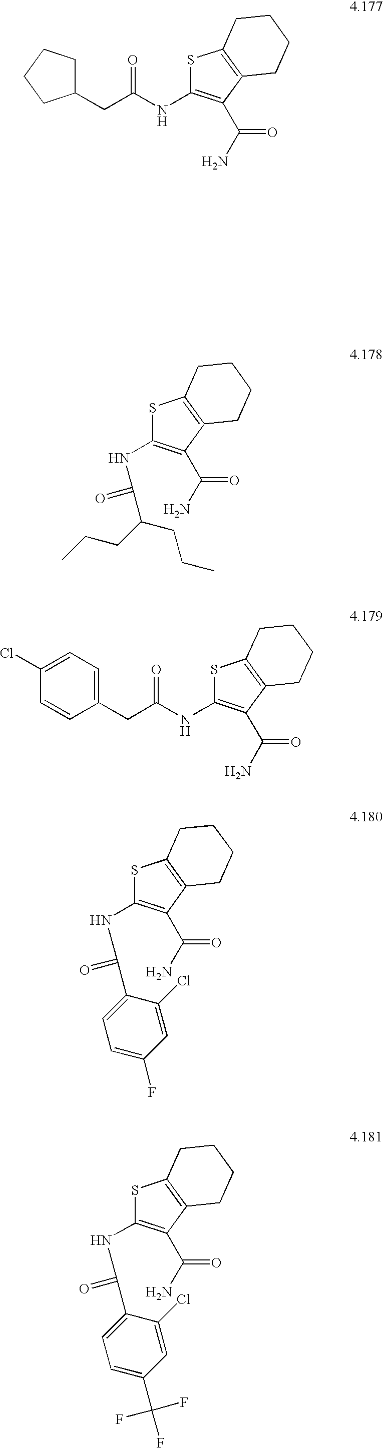 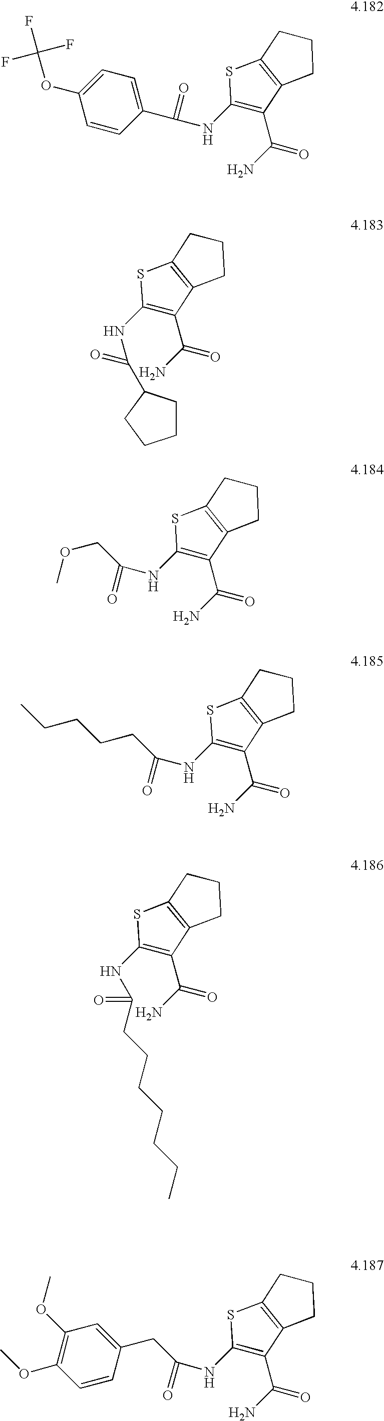 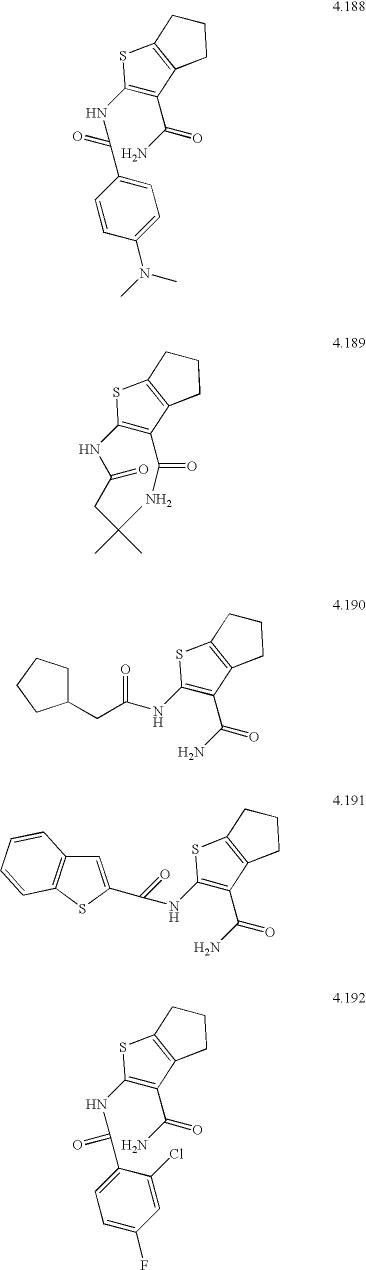 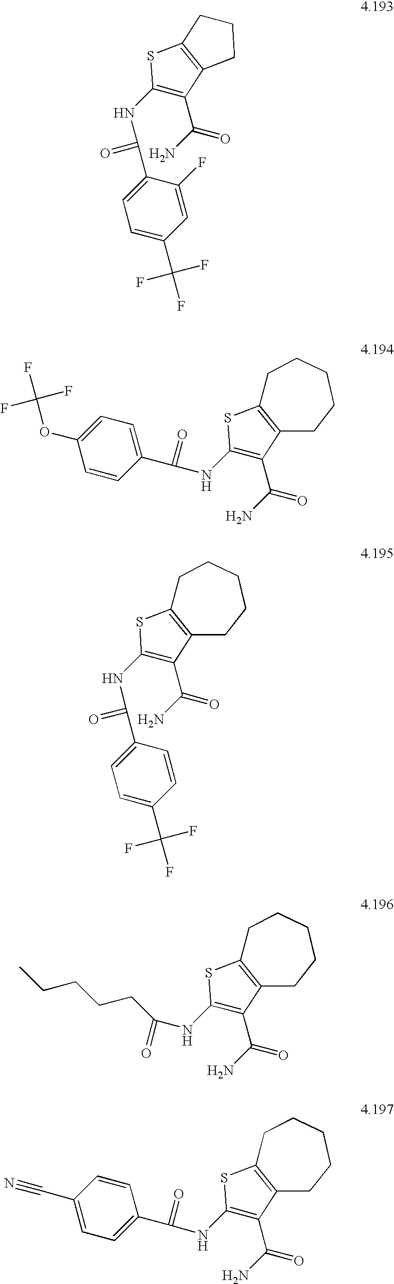 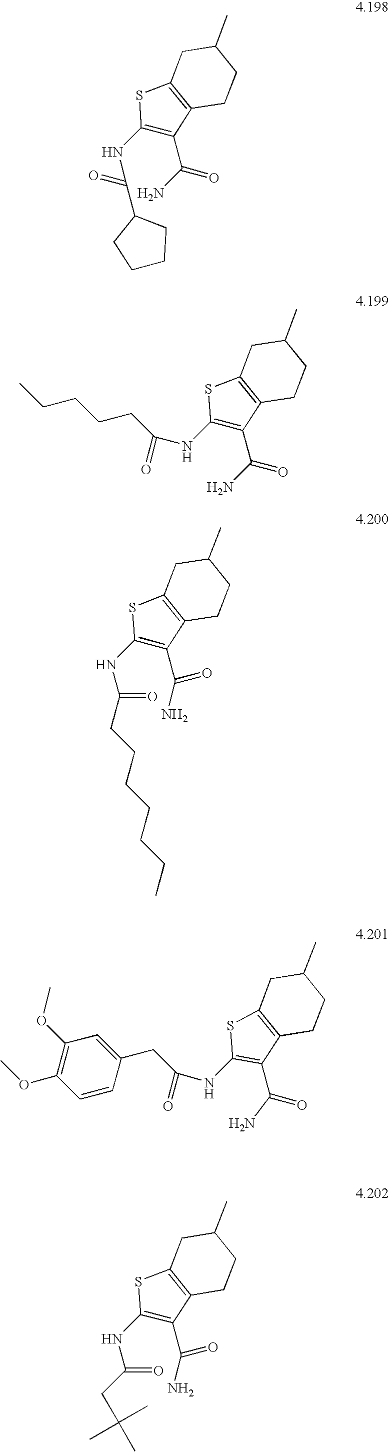 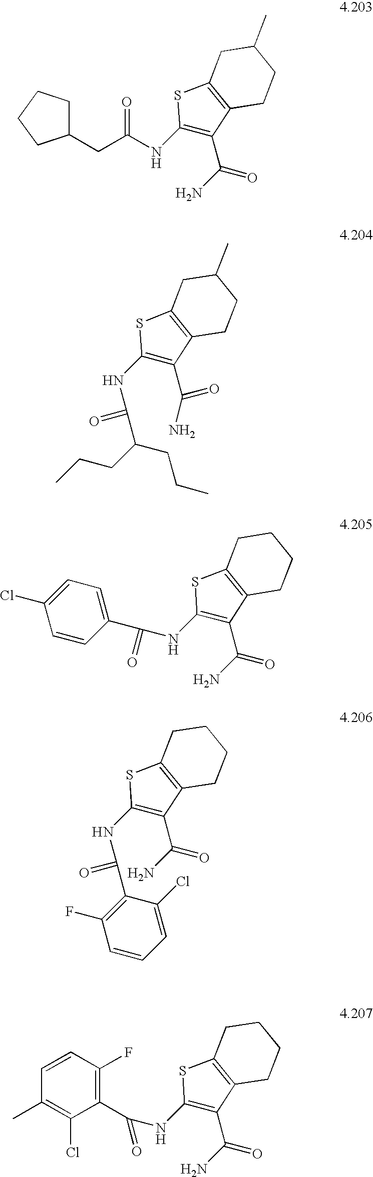 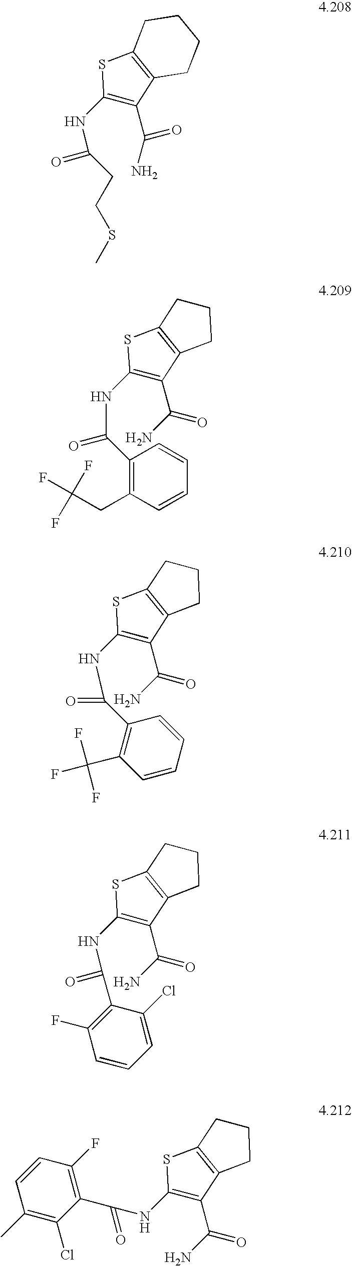 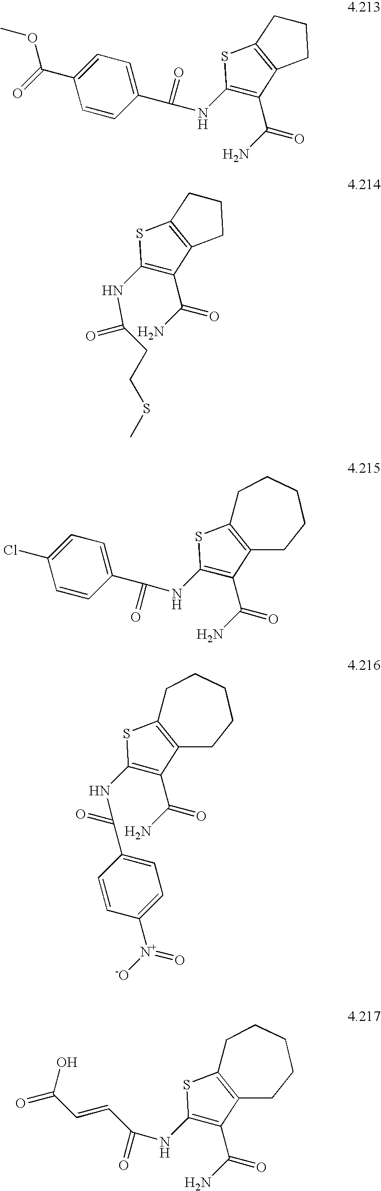 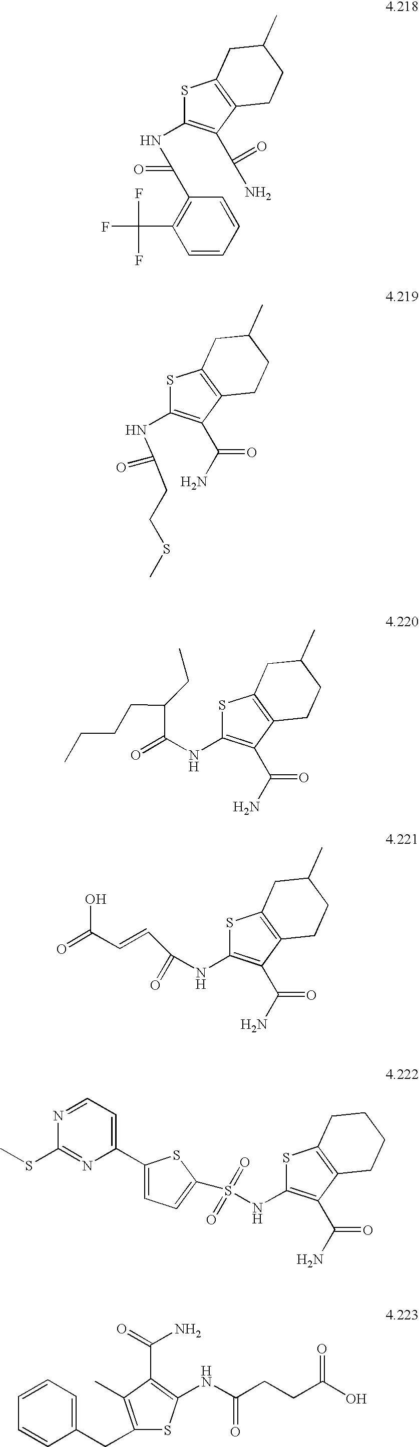 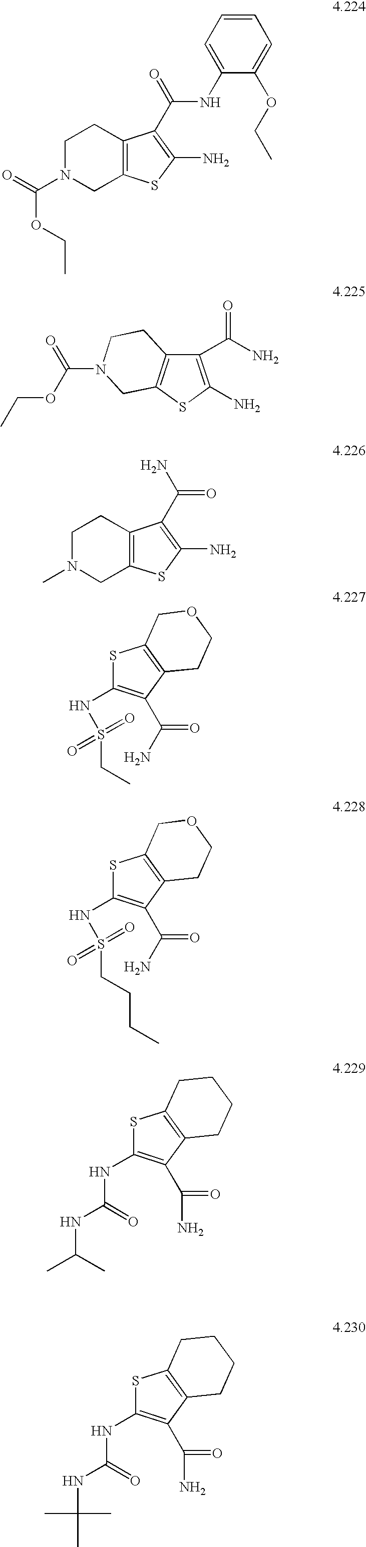 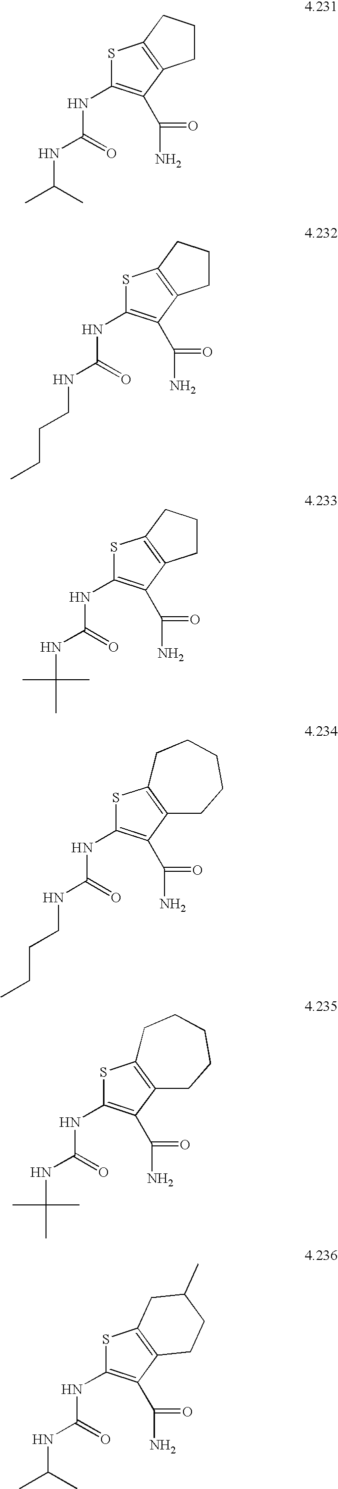 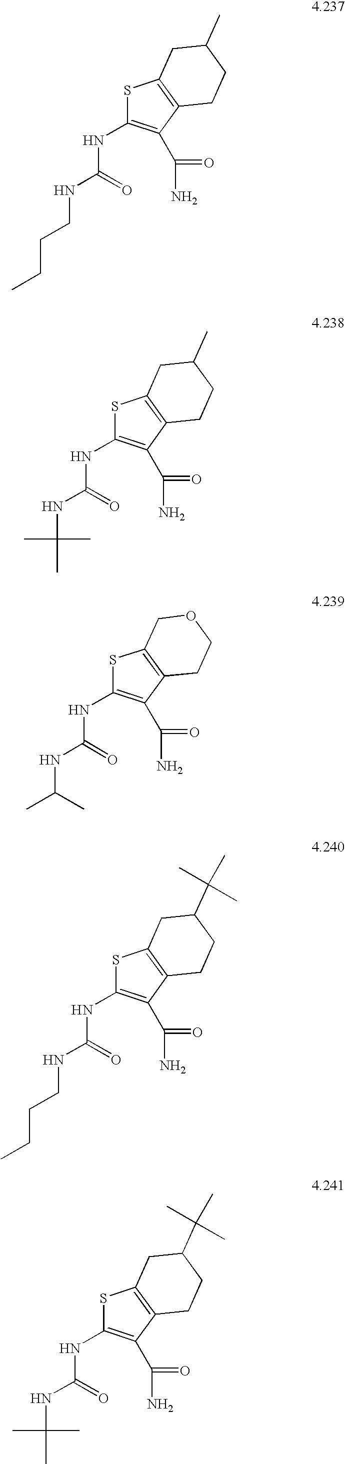 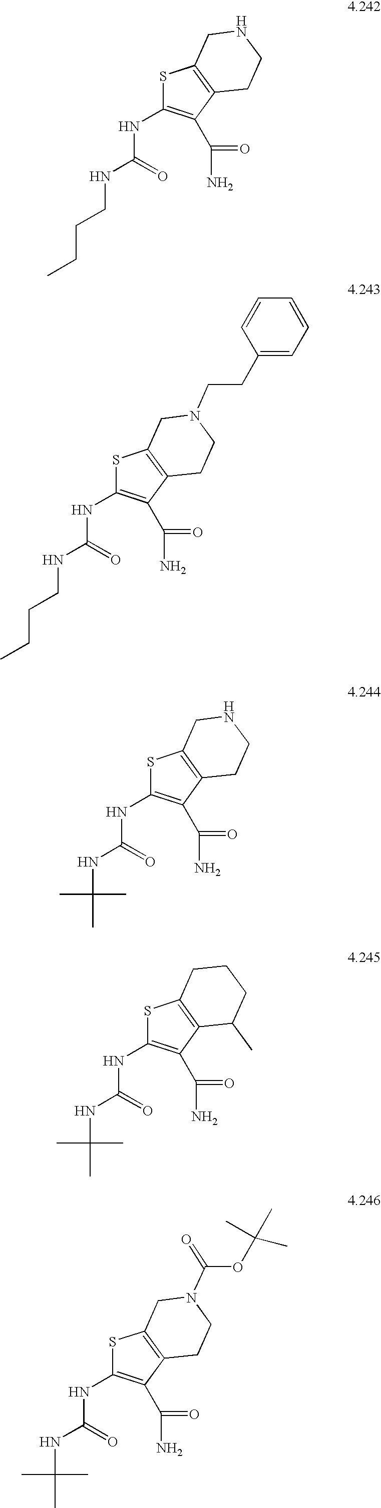 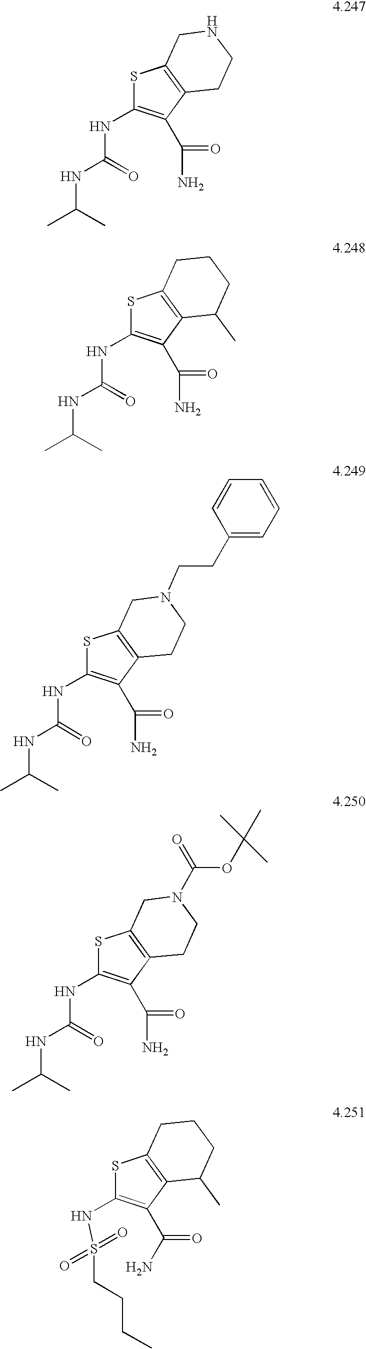 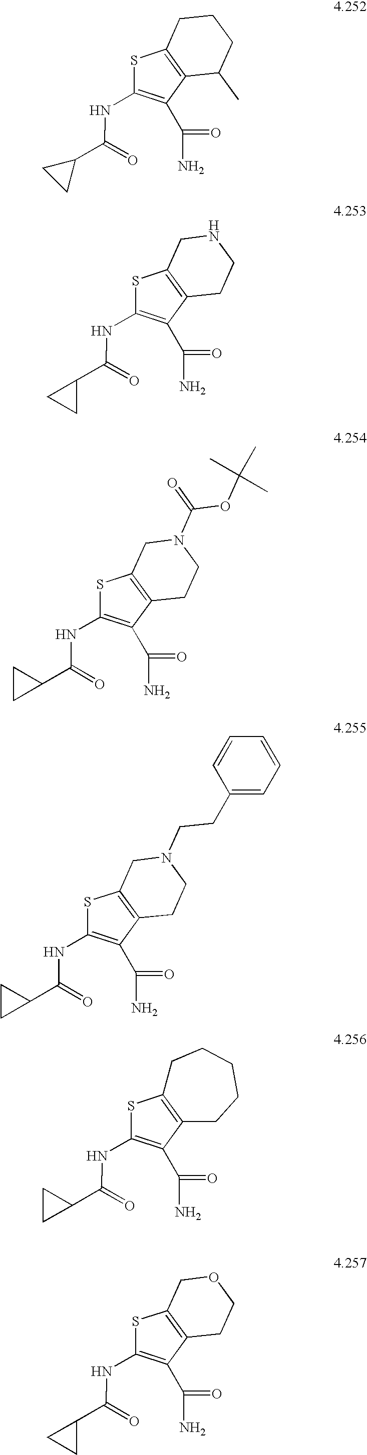 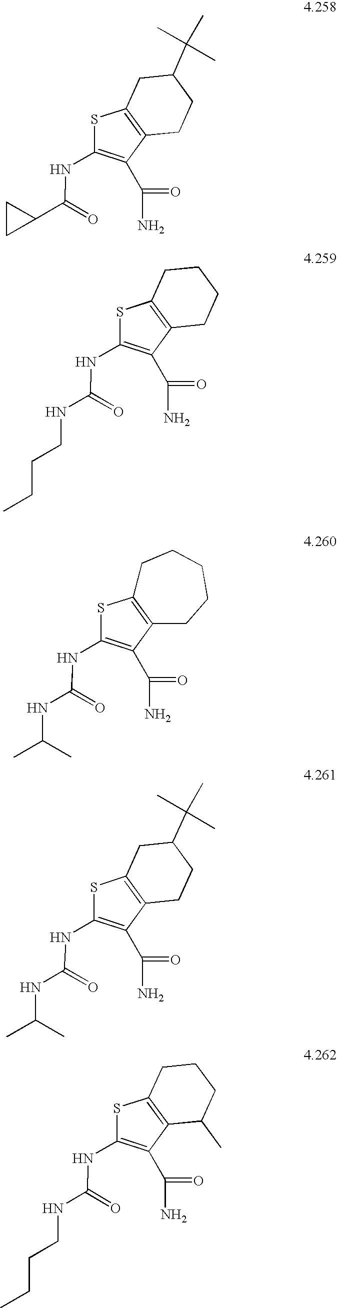 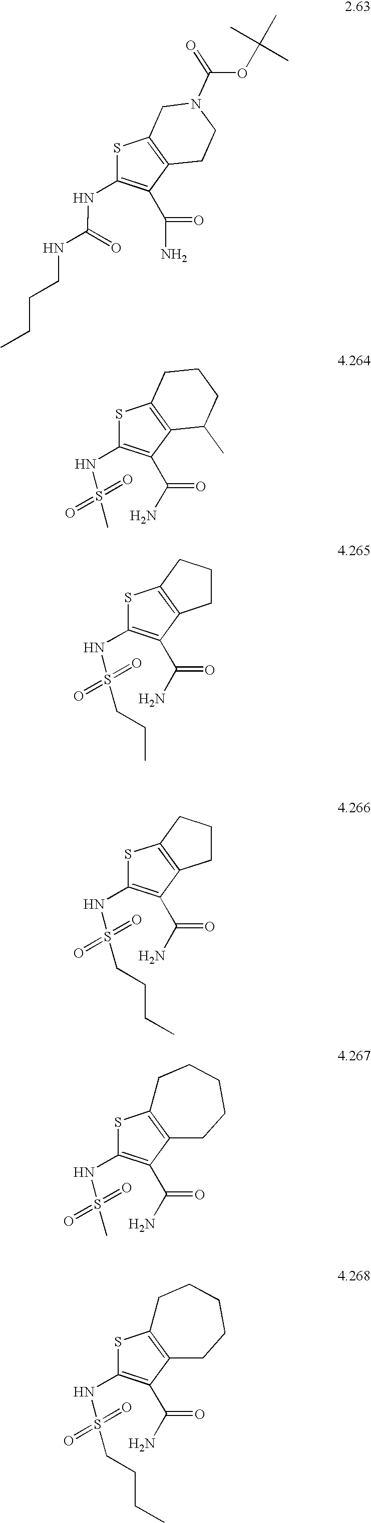 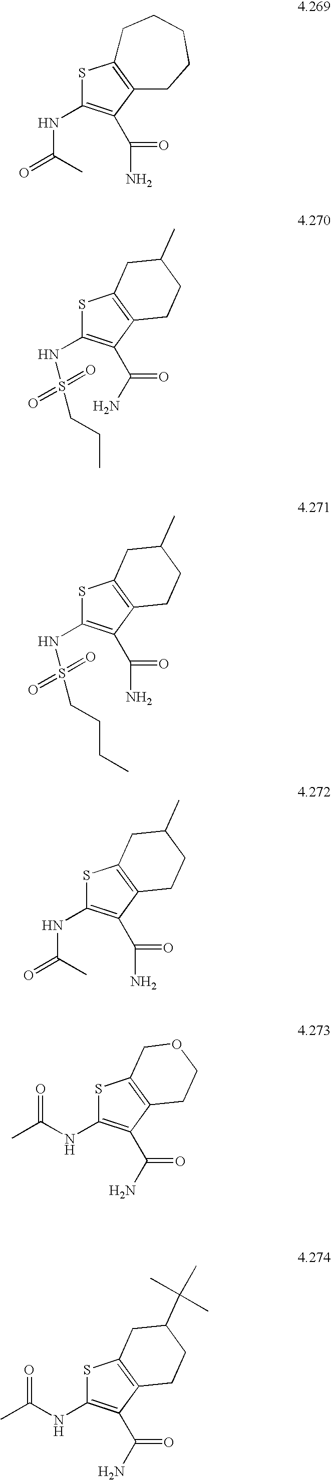 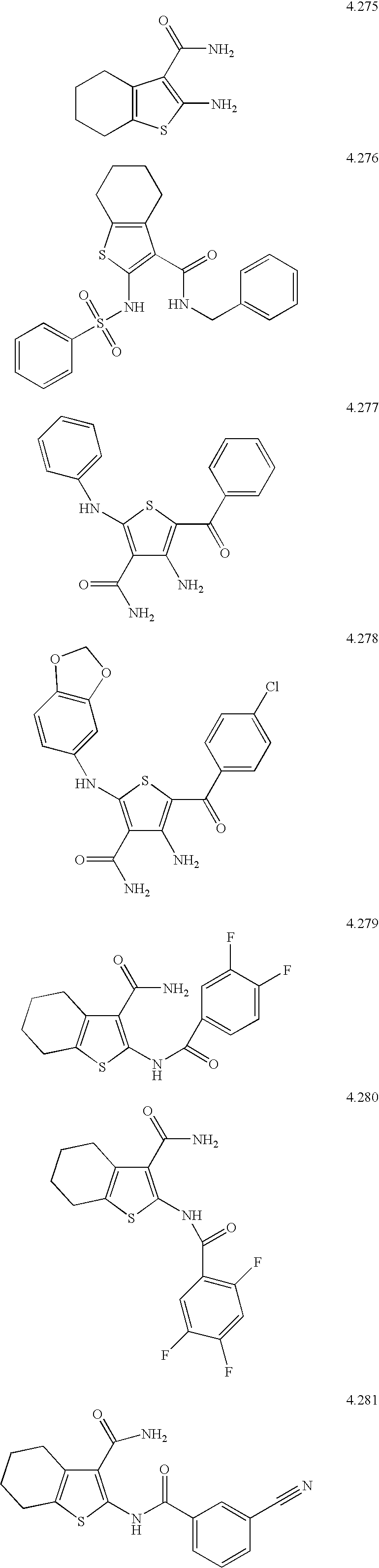 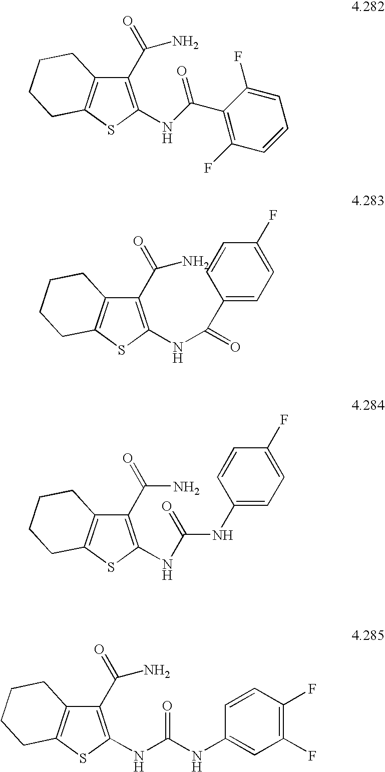
a stereoisomer thereof, a pharmaceutically acceptable salt thereof, a hydrate thereof, or a solvate of any of the foregoing.

121. The compound of claim 108, wherein the at least one ATP-utilizing enzyme is chosen from a human protein kinase.

122. The compound of claim 121, wherein the human protein kinase is chosen from ABL-1, ABL-T315I, AKT1, AKT2, AKT3, AURORA-A, BMX, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, HCK, INSR, KIT, LCK, LYNA, MAPKAPK-2, MET, MSK1, MSK2, NEK2, P38-α, P38-γ, P38-β, P38-δ, P70S6K1, PAK2, PDGFR-α, PDK1, PRAK, ROCK2, SGK1, SRC, SYK, TRKB, and ZAP70.

123. A pharmaceutical composition comprising at least one pharmaceutically acceptable excipient, and a therapeutically effective amount of at least one compound according to claim 108.

124. A pharmaceutical composition comprising at least one pharmaceutically acceptable excipient, and a therapeutically effective amount of at least one compound according to claim 120.

125. The pharmaceutical composition of claim 123, wherein the at least one compound is present in an amount effective for the treatment in a patient of at least one disease chosen from Alzheimer's disease, stroke, diabetes, obesity, inflammation, and cancer.

126. The pharmaceutical composition of claim 125, wherein cancer is chosen from at least one of glioblastoma, ovarian, breast, endometrial, hepatocellular carcinoma, melanoma, digestive tract, lung, renal-cell carcinoma, thyroid, lymphoid, prostate, and pancreatic cancer.

127. A method of treating a disease in a patient in need of such treatment comprising administering to the patient a therapeutically effective amount of at least one compound according to claim 108.

128. A method of treating a disease in a patient in need of such treatment comprising administering to the patient a therapeutically effective amount of at least one compound according to claim 120.

129. The method of claim 127, wherein the at least one disease is chosen from Alzheimer's disease, stroke, diabetes, obesity, inflammation, and cancer.

130. The method of claim 129, wherein cancer is chosen from at least one of glioblastoma, ovarian, breast, endometrial, hepatocellular carcinoma, melanoma, digestive tract, lung, renal-cell carcinoma, thyroid, lymphoid, prostate, and pancreatic cancer.

131. A method of inhibiting at least one ATP-utilizing enzyme in a subject comprising administering to the subject at least one compound according to claim 108.

132. A method of inhibiting at least one ATP-utilizing enzyme in a subject comprising administering to the subject at least one compound according to claim 120.

133. The method of claim 131, wherein the ATP-utilizing enzyme is chosen from a human protein kinase.

134. The method of claim 131, wherein the human protein kinase is chosen from ABL-1, ABL-T315I, AKT1, AKT2, AKT3, AURORA-A, BMX, CDK, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, HCK, INSR, KIT, LCK, LYNA, MAPKAPK-2, MET, MSK1, MSK2, NEK2, P38-α, P38-β, P38-β, P38-δ, P70S6K1, PAK2, PDGFR-α, PDK1, PRAK, ROCK2, SGK1, SRC, SYK, TRKB, and ZAP70.

135. A method of inhibiting at least one ATP-utilizing enzyme comprising contacting the ATP-utilizing enzyme with at least one compound according to claim 108.

136. A method of inhibiting at least one ATP-utilizing enzyme comprising contacting the ATP-utilizing enzyme with at least one compound according to claim 120.

137. The method of claim 135, wherein the ATP-utilizing enzyme is chosen from a human protein kinase.

138. The method of claim 137, wherein human protein kinase is chosen from ABL-1, ABL-T315I, AKT1, AKT2, AKT3, AURORA-A, BMX, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, HCK, INSR, KIT, LCK, LYNA, MAPKAPK-2, MET, MSK1, MSK2, NEK2, P38-α, P38-γ, P38-β, P38-δ, P70S6K1, PAK2, PDGFR-α, PDK1, PRAK, ROCK2, SGK1, SRC, SYK, TRKB, and ZAP70.

139. A method of treating a disease regulated by at least one ATP-utilizing enzyme in a subject in need of such treatment comprising administering to the subject a therapeutically effective amount of at least one compound according to claim 108.

140. A method of treating a disease regulated by at least one ATP-utilizing enzyme in a subject in need of such treatment comprising administering to the subject a therapeutically effective amount of at least one compound according to claim 120.

141. The method of claim 139, wherein the ATP-utilizing enzyme is chosen from a human protein kinase.

142. The method of claim 141, wherein the protein kinase is chosen from ABL-1, ABL-T315I, AKT1, AKT2, AKT3, AURORA-A, BMX, CDK, CDK1, CDK2/cyclinA, CDK2/cyclinE, CDK5, CHEK1, CHEK2, CK1, CK2, CSK, c-TAK1, DAPK1, DYRK2, FLT-3, FYN, GSK-3α, GSK-3β, HCK, INSR, KIT, LCK, LYNA, MAPKAPK-2, MET, MSK1, MSK2, NEK2, P38-α, P38-γ, P38-β, P38-δ, P70S6K1, PAK2, PDGFR-α, PDK1, PRAK, ROCK2, SGK1, SRC, SYK, TRKB, and ZAP70.
